(19) United States
(12) Patent Application Publication
(10) Pub. No.: US 2008/0102065 A1
(43) Pub. Date: May 1, 2008

(54) Erythropoietin receptor extended duration limited agonists

(76) Inventors: Luis G. Borges, Bainbridge Island, WA (US); Graham Molineux, Moorpark, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/787,181

(22) Filed: Apr. 13, 2007

Related U.S. Application Data
(60) Provisional application No. 60/792,131, filed on Apr. 14, 2006.

(51) Int. Cl. A61K 39/395; A61P 7/06; C07K 16/18

(52) U.S. Cl. 424/130.1; 530/389.1

(57) ABSTRACT

A genus of erythropoietin (Epo) receptor agonists having unique structural, biochemical, and physiological characteristics has been discovered and is referred to herein as Erythropoietin Receptor extended Duration Limited Agonist (EREDLA).

This application claims priority benefit of U.S. Provisional Application No. 60/792,131, filed Apr. 14, 2006. The entire contents of U.S. Provisional Application No. 60/792,131 is specifically incorporated herein by reference in its entirety.

### FIELD

The present teachings generally relate to a genus of erythropoietin receptor agonists having unique structural, biochemical, and physiological characteristics and methods of using said agonists.

### BACKGROUND

Erythropoietin (Epo) is a glycoprotein hormone involved in the growth and maturation of erythroid progenitor cells into erythrocytes. EPO is produced by the liver during fetal life and by the kidney of adults and stimulates the production of red blood cells from erythroid precursors. Relatively decreased production of EPO, which commonly occurs in adults as a result of renal failure, leads to anemia. EPO has been produced by genetic engineering techniques involving expression and secretion of the protein from a host cell transfected with the gene encoding erythropoietin. Administration of recombinant EPO has been effective in the treatment of anemia. For example, Eschbach et al. (N. Engl J Med 316, 73 (1987)) describe the use of EPO to correct anemia resulting from chronic renal failure.

The purification of human urinary EPO was described by Miyake et al. (J. Biol. Chem. 252, 5558 (1977)). The identification, cloning, and expression of genes encoding erythropoietin is described in U.S. Pat. No. 4,703,008 to Lin. A description of a method for purification of recombinant EPO from cell medium is included in U.S. Pat. No. 4,667,016 to Lai et al. The erythropoietin receptor (EPO-R) is thought to exist as a multimeric complex. Sedimentation studies suggested its molecular weight is 330 +/−48 kDa (Mayeux et al. Eur. J. Biochem. 194, 271 (1990)). Crosslinking studies indicated that the receptor complex includes multiple distinct polypeptides, a 66-72 kDa species, and 85 and 100 kDa species (Mayeux et al. J. Biol. Chem. 266, 23380 (1991)); McCaffery et al. J. Biol. Chem. 264, 10507 (1991)). A distinct 95 kDa protein was also detected by immunoprecipitation of EPO receptor (Miura & Ihle Blood 81, 1739 (1993)). Another crosslinking study revealed three EPO containing complexes of 110, 130 and 145 kDa. The 110 and 145 kDa complexes contained EPO receptor since they could be immunoprecipitated with antibodies raised against the receptor (Miura & Ihle, supra). Expression of a carboxy-terminal truncated EPO receptor resulted in detection of the 110 kDa complex but not the 145 kDa complex. This suggests that the higher molecular weight complex contains polypeptides present in the 110 kDa complex and an additional 35 kDa protein.

Further insight into the structure and function of the EPO receptor complex was obtained upon cloning and expression of the mouse and human EPO receptors (D'Andrea et al. Cell 57, 277 (1989); Jones et al. Blood 76, 31 (1990); Winkelmann et al. Blood 76, 24 (1990); PCT Application No. WO90/08822; U.S. Pat. No. 5,278,065 to D'Andrea et al.) The full-length human EPO receptor is a 483 amino acid transmembrane protein with an approximately 224 amino acid extracellular domain and a 25 amino acid signal peptide. The human receptor shows about an 82% amino acid sequence homology with the mouse receptor. The cloned full-length EPO receptor expressed in mammalian cells (66-72 KDa) has been shown to bind EPO with an affinity similar to that of the native receptor on erythroid progenitor cells. Thus, this form is thought to contain the main EPO binding determinant. The 85 and 100 KDa proteins observed as part of a cross-linked complex are distinct from the EPO receptor but are probably in close proximity to EPO because EPO can be crosslinked to them. The 85 and 100 KDa proteins are related to each other and the 85 KDa protein may be a proteolytic cleavage product of the 100 KDa species (Sawyer J. Biol. Chem. 264, 13343 (1989)).

A soluble (truncated) form of the EPO receptor containing only the extracellular domain has been produced and found to bind EPO with an affinity of about 1 nM, or about 3 to 10-fold lower than the full-length receptor (Harris et al. J. Biol. Chem. 267, 15205 (1992); Yang & Jones Blood 82, 1713 (1993)).

Activation of cell membrane-bound EPO receptor results in several biological effects. Three of the activities include stimulation of proliferation in immature erythroblasts, stimulation of differentiation in immature erythroblasts, and inhibition of apoptosis in erythroid progenitor cells (Liboi et al. Proc. Natl. Acad. Sci. USA 90, 11351 (1993); Koury Science 248, 378 (1990)). The signal transduction pathways resulting in stimulation of proliferation and stimulation of differentiation appear to be separable (Noguchi et al. Mol. Cell. Biol. 8, 2604 (1988); Patel et al. J. Biol. Chem. 267, 21300 (1992); Liboi et al. ibid).

Since the introduction of EPOGEN® in 1989, anemia associated with a variety of disease states has been treated safely and effectively with erythropiesis stimulating proteins. The approval of ARANESP® offered patients a more potent stimulator of erythropoiesis together with the convenience of less frequent dosing compared to epoeitins.

### SUMMARY

In certain embodiments, an Erythropoietin Receptor Extended Duration Limited Agonist is provided. In certain embodiments, the Erythropoietin Receptor Extended Duration Limited Agonist comprises an antibody that: (a) binds the erythropoietin receptor in a population of cells expressing the erythropoietin receptor and activates the erythropoietin receptor to a lesser degree than erythropoietin, or recombinant equivalents or analogs of erythropoietin, when used at the same or higher concentrations than erythropoietin, or recombinant equivalents or analogs of erythropoietin; (b) binds to the human erythropoietin receptor with a lower affinity than erythropoietin; (c) raises the hemoglobin concentration in a treated mammal and induces an initial peak concentration of erythropoietin that is comparable to the peak hemoglobin attainable with erythropoietin, or recombinant equivalents or analogs of erythropoietin, but maintains the hemoglobin concentration in said mammal over a period of time that is longer than that attainable with erythropoietin, or recombinant equivalents or analogs of erythropoietin; and (d) possesses an extended half-life in vivo beyond that of erythropoietin, or recombinant equivalents or analogs of erythropoietin.

In certain embodiments, (a) is the EC₅₀ ratio of: the EC₅₀ values derived from an in vitro assay measuring the relative readout of Epo, or recombinant equivalents or analogs of Epo, activating the erythropoietin receptor/the EC₅₀ values derived from said assay measuring the relative readout of an Erythropoietin Receptor Extended Duration Limited Agonist activating the erythropoietin receptor, wherein the ratio is less than 1. In certain embodiments, the EC₅₀ ratio is in the range of about 0.001 to about 0.623. In certain embodiments, in (a) about 200 to 2,000 fold more of the Erythropoietin Receptor Extended Duration Limited Agonist is required to achieve maximum colony number in a Burst Forming Unit-Erythroid assay in relation to the amount of erythropoietin, or recombinant equivalents or analogs of erythropoietin, required to achieve maximum colony number in said assay. In certain embodiments, in (a) the Erythropoietin Receptor Extended Duration Limited Agonist elicits about 15 to 50% of the maximum colony number in a Burst Forming Unit-Erythroid assay in relation to the maximum colony number achieved by erythropoietin, or recombinant equivalents or analogs of erythropoietin, in said assay. In certain embodiments, the colonies elicited in a Burst Forming Unit-Erythroid assay by the Erythropoietin Receptor Extended Duration Limited Agonist are at least 25% smaller in diameter than the colonies achieved by erythropoietin, or recombinant equivalents or analogs of erythropoietin, in said assay.

In certain embodiments, in (b) the Kd is greater than 0.25 nM. In certain embodiments, in (b) the Kd is from about 1.1 nM to 14,900 nM.

In certain embodiments, in (c) the Erythropoietin Receptor Extended Duration Limited Agonist maintains in vivo hemoglobin concentrations above baseline at least about 200 to 300% longer than erythropoietin, or recombinant equivalents or analogs of erythropoietin. In certain embodiments, in (c) the Erythropoietin Receptor Extended Duration Limited Agonist maintains in vivo hemoglobin concentrations above baseline about 120 days +/−20 days. In certain embodiments, in (c) the Erythropoietin Receptor Extended Duration Limited Agonist maintains in vivo hemoglobin concentrations above baseline for about two to four months.

In certain embodiments, in (d) the Erythropoietin Receptor Extended Duration Limited Agonist has an in vivo half-life that is about 13 to 80 times longer than erythropoietin, or recombinant equivalents or analogs of erythropoietin.

In certain embodiments, a composition comprising an Erythropoietin Receptor Extended Duration Limited Agonist and at least one pharmaceutically acceptable vehicle, buffer, excipient, or carrier is provided.

In certain embodiments, a method of activating endogenous activity of an erythropoietin receptor in a patient in need thereof is provided. In certain embodiments, the method comprises administering an effective amount of an Erythropoietin Receptor Extended Duration Limited Agonist.

In certain embodiments, a method of treating anemia in a patient in need thereof is provided. In certain embodiments, the method comprises administering an Erythropoietin Receptor Extended Duration Limited Agonist. In certain embodiments, the anemia is associated with a chronic disease or condition. In certain embodiments, the chronic disease or condition is chronic kidney disease, congestive heart failure, or myelodysplastic syndrome. In certain embodiments, the anemia is associated with cancer. In certain embodiments, the anemia associated with cancer is chemotherapy-induced anemia or cancer-induced anemia. In certain embodiments, the anemia is anemia of the elderly, anemia due to infection, anemia associated with inflammation, anemia associated with iron deficiency, anemia associated with blood loss, anemia associated with hemolysis, anemia associated with secondary hyperparathyroidism, anemia associated with inadequate dialysis, anemia associated with protein energy malnutrition, anemia associated with vitamin deficiencies, or anemia associated with metal toxicity.

In certain embodiments, a method of treating pure red blood cell aplasia in a patient in need thereof is provided. In certain embodiments, the method comprises administering an effective amount of an Erythropoietin Receptor Extended Duration Limited Agonist.

In certain embodiments, a method of promoting tissue protection in erythropoietin-responsive cells, tissues, and organs in a patient in need thereof is provided. In certain embodiments, the method comprises administering an Erythropoietin Receptor Extended Duration Limited Agonist.

In certain embodiments, a method of activating endogenous activity of an erythropoietin receptor in a patient comprises administering an effective amount of the Erythropoietin Receptor Extended Duration Limited Agonist, wherein the Erythropoietin Receptor Extended Duration Limited Agonist is administered to said patient less frequently than epoietin alfa, epoietin beta, darbepoietin alfa, or derivatives thereof. In certain embodiments, an Erythropoietin Receptor Extended Duration Limited Agonist is administered to a patient as needed according to the schedule of: once per month, once every two months, once every three months, once every four months, once every five months, or once every six months.

In certain embodiments, a method of treating anemia in a patient comprises administering an Erythropoietin Receptor Extended Duration Limited Agonist, wherein the Erythropoietin Receptor Extended Duration Limited Agonist is administered to a patient less frequently than epoietin alfa, epoietin beta, darbepoietin alfa, or derivatives thereof. In certain embodiments, an Erythropoietin Receptor Extended Duration Limited Agonist is administered to a patient as needed according to the schedule of: once per month, once every two months, once every three months, or once every four months, once every five months, or once every six months. In certain embodiments, the anemia is associated with a chronic disease or condition. In certain embodiments, the chronic disease or condition is chronic kidney disease, congestive heart failure, or myelodysplastic syndrome. In certain embodiments, the anemia is associated with cancer. In certain embodiments, the anemia associated with cancer is chemotherapy-induced anemia or cancer-induced anemia. In certain embodiments, the anemia is anemia of the elderly, anemia due to infection, anemia associated with inflammation, anemia associated with iron deficiency, anemia associated with blood loss, anemia associated with hemolysis, anemia associated with secondary hyperparathyroidism, anemia associated with inadequate dialysis, anemia associated with protein energy malnutrition, anemia associated with vitamin deficiencies, or anemia associated with metal toxicity.

In certain embodiments, a method of treating pure red blood cell aplasia in a patient comprises administering an effective amount of an Erythropoietin Receptor Extended Duration Limited Agonist, wherein, the Erythropoietin Receptor Extended Duration Limited Agonist is administered to a patient less frequently than epoietin alfa, epoietin beta, darbepoietin alfa, or derivatives thereof. In certain embodiments, an Erythropoietin Receptor Extended Duration Limited Agonist is administered to a patient as needed according to the schedule of: once per month, once every two months, once every three months, or once every four months, once every five months, or once every six months.

In certain embodiments, a method of promoting tissue protection in erythropoietin-responsive cells, tissues, and organs in a patient comprises administering an Erythropoietin Receptor Extended Duration Limited Agonist, wherein, an Erythropoietin Receptor Extended Duration Limited Agonist is administered to a patient less frequently than epoietin alfa, epoietin beta, darbepoietin alfa, or derivatives thereof. In certain embodiments, an Erythropoietin Receptor Extended Duration Limited Agonist is administered to a patient as needed according to the schedule of: once per month, once every two months, once every three months, or once every four months, once every five months, or once every six months.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flow chart of steps for screening EpoR agonistic antibodies from human scFv phage display libraries according to work discussed in Example 1.

FIG. 2 shows a schematic diagram describing the streamline conversion of phage scFv clones from phage display libraries to an scFv-Fc format in a mammalian expression construct, pDC409a-huG1Fc according to work discussed in Example 2. NcoI and PciI create a cohesive end for ligation. The process of batchwise conversion of scFv NcoI/NotI restriction fragments to PciI/NotI restricted pDC409a-huG1 Fc vector is highly efficient.

FIG. 3 shows FACS analysis of antibodies binding to cells according to work discussed in Example 3. Antibody and Epo concentration used for staining are 5 μg/ml. Panel A shows fluorescence intensity of UT-7 cells upon binding of clone 2, clone 5, clone 7, clone 10 or clone 30 in scFv-Fc in the presence (solid line) and absence (dashed line) of human Epo during staining. Antibody and Epo concentration used are both at 5 μg/ml. The shaded curves are from staining only with phycoerythrin-conjugated goat anti human F(ab′)2 without any primary antibody. Panel B shows fluorescence intensity of COS-1 cells upon binding of clone 2, clone 5, clone 7, clone 10 or clone 30 in scFv-Fc (solid lines). The shaded curves are from staining only with phycoerythrin-conjugated goat anti human F(ab′)2 without any primary antibody.

FIG. 4 shows competition binding of clone numbers 2, 5, 7, 10 and 30 to soluble huEpoR by ELISA according to work discussed in Example 5. Panel A shows competitive binding between clone 5 phage and clone 2, clone 5, clone 7, clone 10, or clone 30 in scFv-Fc format. Panel B shows competitive binding between clone 30 phage and clone 2, clone 5, clone 7, clone 10, and clone 30 in scFv-Fc format.

FIG. 5 shows clone 2, clone 5, clone 7, clone 10, or clone 30 antibodies binding to mouse EpoR (muEpoR) protein by ELISA according to work discussed in Example 6. Hatched bars show binding in scFv-Fc format. Open bars show binding in IgG2 format.

FIG. 6 shows BIAcore sensograms of huEpoR protein to clone 2, clone 5, clone 7, clone 10 and clone 30 scFv-Fc proteins captured on a CM4 chip according to work discussed in Example 7.

FIG. 7 shows dose-titration curves of huEpoR activation for maxibodies Mxb 2, Mxb 5, Mxb 7, Mxb 10, and Mxb 30 according to work discussed in Example 8. UT-7-Luc cells (UT-7 cells containing the luciferase reporter gene) were treated for six hours with serially diluted maxibodies in 96-well plates, in triplicate, for a final concentrations of 1000, 333, 111, 37.04, 12.35, 4.115, 1.372, 0.457, 0.152, 0.051, 0.017, and 0.006 nM for Mxb 5, Mxb 10, and Mxb 30, and 2500, 1250, 625, 312.5, 156.25, 78.125, 39.0625, 19.53125, 9.765625, 4.882813, 2.441-406, 1.220703, 0.610352, 0.3051758, 0.1525879, 0.76294, 0.038147, 0.019073, 0.009537, 0.004768, 0.002384, 0.001192, 0.000596, 0.000298 nM for Mxb 2 and Mxb 7. Recombinant human Epo was used as a reference standard and was serially diluted in the same plate used to test each maxibody. Each Epo dilution was run in triplicate at the following concentrations for Mxb 2, Mxb 5, Mxb 10, and Mxb 30: 100, 10.1, 0.1, 0.01, and 0.001 nM, and at the following concentrations for Mxb 7: 1488, 744, 372, 186, 93, 46.5, 23.2, 11.6, 5.8, 2.9, 1.5, 0.71, 0.36, 0.18, 0.09, 0.045, 0.023, 0.011, 0.006, 0.003, 0.0015, 0.0007, 0.0004, 0.0002 nM. Following the addition of the luciferase substrate, luciferase activity was read on a 96-well plate luminometer. Raw data was processed by subtracting the background luminescence (values from wells containing media only) and presented as the average of three values ± the standard deviation.

FIG. 8 shows a comparison of the maximal activity levels for the IgG₂ proteins (Ab) and scFv-Fc proteins (Mxb) in the induction of the huEpoR according to work discussed in Example 9. The maximal luciferase activity for each test reagent was the highest value taken from the dose titration curve of each scFv-Fc protein and IgG₂ protein divided by the maximal luciferase activity for the rHuEpo standard taken from the dose titration curve of rHuEpo on each individual plate. This ratio is represented above and is the average of three values the standard deviation.

FIG. 9 shows the activation of UT-7 cells by rHuEpo, Mxb 2, and IgG₂ 2 as indicated by phosphorylation of the signaling molecules Stat5 and Akt according to work discussed in Example 10.

FIG. 10 shows scFv-Fc proteins Mxb 2, Mxb 5, Mxb 7, and Mxb 30 activate CD34+ human peripheral blood progenitor cells (CD34+PBPC) and stimulate the production of BFU-E derived colonies according to work discussed in Example 11.

FIG. 11 shows a single injection of Mxb 5 produces an increase in reticulocyte numbers that is dose-dependent and sustained over a period of time significantly longer than in the animals treated with PEG-NESP according to work discussed in Example 12A.

FIG. 12 shows a single injection of Mxb 5 produces an increase in hemoglobin levels that is dose-dependent and sustained over a period of time significantly longer than in the animals treated with PEG-NESP according to work discussed in Example 12A.

FIG. 13 shows a single injection of Mxb 7 produces an increase in reticulocyte numbers that is dose-dependent and sustained over a period of time significantly longer than in the animals treated with PEG-NESP according to work discussed in Example 12B.

FIG. 14 shows a single injection of Mxb 7 produces an increase in hemoglobin levels that is dose-dependent and sustained over a period of time significantly longer than in the animals treated with PEG-NESP according to work discussed in Example 12B.

FIG. 15 shows a single injection of Mxb 10 produces an increase in reticulocyte numbers that is dose-dependent and sustained over a period of time significantly longer than in the animals treated with PEG-NESP according to work discussed in Example 12C.

FIG. 16 shows a single injection of Mxb 10 produces an increase in hemoglobin levels that is dose-dependent and sustained over a period of time significantly longer than in the animals treated with PEG-NESP according to work discussed in Example 12C.

FIG. 17 shows a single injection of Mxb 2 produces an increase in reticulocytes number that is sustained over a period of time similar to that measured in the animals treated with PEG-NESP according to work discussed in Example 12D.

FIG. 18 shows a single injection of Mxb 2 produces an increase in hemoglobin levels that is sustained over a period of time significantly longer than in the animals treated with PEG-NESP according to work discussed in Example 12D.

FIG. 19 shows the change in serum concentration of Mxb 5 (“#5 Scfv-Fc”) and IgG₁5 (“#5 IgG₁”) over time according to work discussed in Example 13.

FIG. 20 shows the pharmacokinetic parameters of IgG₁5 and Mxb 5 in mice according to the work discussed in Example 13.

FIG. 21 shows CDRs from Mxb 2, Mxb 5, Mxb 7, Mxb 10, and Mxb 30.

FIG. 22 shows a FACS analysis of certain scFv-Fc proteins binding to cells according to work discussed in Example 15. Antibody and Epo concentrations used for staining are 5 μg/ml. The shaded curves are from staining only with phycoerythrin-conjugated goat anti-human F(ab′)2 without any primary antibody. Panel A: Fluorescence intensity of UT-7 cells upon binding of Mxb 13, Mxb 15, Mxb 16, Mxb 29, or Mxb 34 in the presence (solid line) and absence (dashed line) of human Epo during staining. Panel B. Fluorescence intensity of COS-1 cells upon binding of Mxb 13, Mxb 15, Mxb 16, Mxb 29, or Mxb 34 (solid line).

FIG. 23 shows EpoR binding and competition binding of scFv-Fc proteins according to work discussed in Examples 15, 16, and 17. EpoR binding to human (hu), mouse (mu) and cynomolgus monkey (cyno) was tested by ELISA and FACS. The ability of Epo to compete with clone 2, clone 5, clone 7, clone 10, clone 13, clone 15, clone 16, clone 29, clone 30, or clone 34 for binding to the EpoR was tested by FACS in UT-7 cells. The ability of Epo to compete with clone 201, clone 276, clone 295, clone 307, clone 318, clone 319, clone 323, clone 330, clone 352, or clone 378 for binding to the EpoR was tested by competition ELISA. The ability of clone 5 to compete with clone 2, clone 5, clone 7, clone 10, clone 13, clone 15, clone 16, clone 29, clone 30, or clone 34 for binding to the EpoR was tested by plate-based ELISA. The ability of clone 30 to compete with clone 2, clone 5, clone 7, clone 10, clone 13, clone 15, clone 16, clone 29, clone 30, or clone 34 for binding to the EpoR was tested by plate-based ELISA.

FIG. 24 shows that a single injection of Mxb 276_G1 MB produced an increase in reticulocyte numbers that is sustained over a period of time according to work discussed in Example 20. The increase is sustained longer than in animals treated with PEG-NESP.

FIG. 25 shows that a single injection of Mxb 276_G1 MB produced an increase in hemoglobin that is sustained over a period of time according to work discussed in Example 20. The increase in hemoglobin is sustained significantly longer than in animals treated with PEG-NESP.

FIG. 26A shows absolute reticulocyte numbers in cynomolgus monkeys after administration of Mxb 5 human point mutant Fc (un-glycosylated Fc) (“huMxb#5” in the Figure), a Mxb 5 cynomolgus point mutant Fc (un-glycosylated Fc) (“cynoMxb#5” in the Figure), a Mxb 10 human point mutant Fc (un-glycosylated Fc) (“huMxb#10” in the Figure), and a Mxb 30 human point mutant Fc (un-glycosylated Fc) (“huMxb#30” in the Figure), or control injections (“Peg-NESP” and “Vehicle” in the Figure) according to work discussed in Example 22. Each monkey was dosed twice by IV injection, the first administration of injections occurred on day 1 and the second one on day 15. The scFv-Fc proteins were dosed at 0.5 mg/kg for the first administration on day 1 and at 5 mg/kg for the second administration on day 15. Peg-Nesp was dosed at 0.03 mg/kg for both injections. The vehicle control (“Vehicle” in the figure) (10 mM potassium phosphate, 161 mM L-Arginine, pH 7.5) was dosed at 1 ml/kg for both injections. FIG. 26B shows reticulocyte numbers graphed as a percentage of baseline reticulocyte levels for each group after administration of huMxb#5, cynoMxb#5, huMxb#10, and huMxb#30 or control injections according to work discussed in Example 22. The baseline reticulocyte levels were obtained from the analysis of blood collected on day 1 prior to the first administration. Each monkey was dosed twice by IV injection, the first administration of test articles occurred on day 1 and the second one on day 15. The scFv-Fc proteins were dosed at 0.5 mg/kg for the first administration on day 1 and at 5 mg/kg for the second administration on day 15. Peg-Nesp was dosed at 0.03 mg/kg for both injections. The vehicle control was dosed at 1 ml/kg for both injections.

FIG. 27 shows certain PCR reaction conditions used to make constructs according to work discussed in Example 21.

FIGS. 28A, B, C, and D show amino acid sequences that were used as templates for the N 297 S glycosylation site mutagenesis in human and cynomolgus Fc's according to work discussed in Example 21. The amino acid highlighted in red shows where the N 297 S mutation takes place. The yellow portion is the VH5 leader sequence, the green is the scFv and the blue is the Fc region. The portion in white in FIGS. 28A, 28B and 28C includes a G from the original scFv library and amino acids from the introduction of a restriction site to facilitate cloning.

FIGS. 29A, B, C, and D shows the final clones and sequences of the mutated, scFv-Fc proteins Mxb#5 human point mutant Fc, Mxb#10 human point mutant Fc, Mxb#30 human point mutant Fc, Mxb#5 cynomolgus point mutant Fc) according to work discussed in Example 21. The amino acid highlighted in red shows the N 297 S mutation. The yellow portion is the VH5 leader sequence, the green is the scFv and the blue is the Fc region. The portion in white includes a G, from the original scFv library and amino acids from the introduction of a restriction site to facilitate cloning.

FIG. 30 shows an ELISA binding assay for mutant EpoR protein binding to Mxb 10 according to work discussed in Example 23. E62A, F93A and M150A diminish binding relative to WT and are likely part of the Mxb 10 binding epitope.

FIG. 31 shows a LANCE assay for Mxb 10 binding to mutant EpoR proteins according to work discussed in Example 23. E62A, F93A and M150A diminish binding relative to WT and are likely part of the Mxb 10 binding epitope.

FIG. 32 shows a comparison of Mxb 10 binding to arginine and alanine EpoR mutants according to work discussed in Example 23. FIG. 32A shows that a mutation of W64 to arginine or alanine did not diminish the binding relative to WT. W64A appears not to be part of the Mxb 10 epitope. FIG. 32B shows a mutation of M150 to alanine diminished binding of Mxb 10. Mutation of M150 to arginine greatly diminished binding suggesting that M150 is part of the Mxb 10 binding epitope.

FIG. 33 shows sequence alignments of the A) variable heavy chain CDR regions and B) variable light chain CDR regions according to work discussed in Example 24. Sequence alignments were based on the MiniPileup program using electronically spliced CDR regions. Alignments are color coded to indicate polar (blue), apolar (red), acidic (green) and basic (yellow) amino acids. The symbol “*” represents a linker region separating the CDR1, CDR2 and CDR3.

FIG. 34 shows a phylogenetic analysis of A) variable heavy chain CDR regions and B) variable light chain CDR regions according to work discussed in Example 24. Trees are based on neighbor joining analysis of the amino acid sequences of the CDR regions. EREDLAs Mxb 2, Mxb 5, Mxb 7, Mxb 10, Mxb 13, Mxb 15, Mxb 16, Mxb 29, Mxb 30, Mxb 34, Mxb 201, Mxb 276, Mxb 295, Mxb 307, Mxb 318, Mxb 319, Mxb 323, Mxb 330, Mxb 352, and Mxb 378 are illustrated. By way of example, the nomenclature used in FIG. 34 is: 13VH_spliced, which describes the clone name (i.e., Mxb 13) followed by “VH” or “VL”, wherein VH means Variable Heavy and VL means Variable Light. The term “spliced” means that the CDRs were “spliced” together using the linker depicted in FIG. 33.

FIG. 35 shows consensus sequences in the CDRs of the variable heavy chains and the variable light chains in the sequence alignment of FIG. 33, according to work discussed in Example 24. The symbol “X” represents an amino acid that may vary in the consensus sequence. The subscript next to the “X” represents the position of amino acid in the sequence, e.g., “X₁” represents the first amino acid in a consensus sequence.

FIG. 36A shows the full length amino acid sequence of the Epo Receptor. FIG. 36B shows the amino acid sequence of the extracellular domain of the Epo Receptor. The amino acid sequence of the extracellular domain was used to identify amino acids in the epitope mapping experiments described in Example 23 and FIGS. 30 to 32. The extracellular domain lacks the first 24 amino acids present in the amino acid sequence of the full length Epo Receptor. The extracellular domain also lacks amino acids 251 to 508 of the full length Epo Receptor.

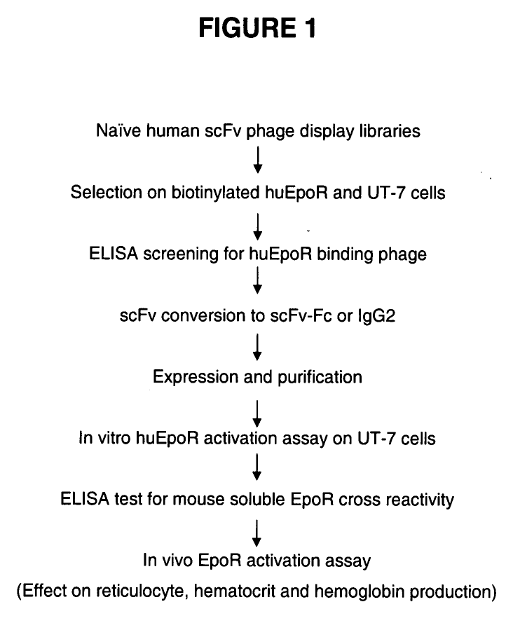

### DETAILED DESCRIPTION OF CERTAIN EMBODIMENTS

The section headings used herein are for organizational purposes only and are not to be construed as limiting the subject matter described. All documents or portions of documents cited in this application, including but not limited to patents, patent applications, articles, books, and treatises, are expressly incorporated by reference herein in their entirety for any purpose. In the event that one or more of the documents incorporated by reference defines a term that contradicts that term's definition in this application, this application controls.

A genus of erythropoietin (Epo) receptor agonists having unique structural, biochemical, and physiological characteristics has been discovered and is referred to herein as Epo Receptor Extended Duration Limited Agonists (also referred to as EREDLA).

Thus, aspects of the invention relate to a genus of EREDLAs, which are defined as compounds that (a) bind the Epo receptor in a population of cells expressing the Epo receptor and activate the Epo receptor to a lesser degree than Epo, or recombinant equivalents or analogs of Epo, when used at the same or higher concentrations than Epo, or recombinant equivalents or analogs of Epo; (b) bind to the human Epo receptor with a lower affinity than Epo; (c) raise hemoglobin concentration in a treated mammal and induce an initial peak concentration of Epo that is comparable to the peak hemoglobin attainable with Epo, or recombinant equivalents or analogs of Epo, but maintain the hemoglobin concentration in said mammal over a longer period of time than that attainable with recombinant Epo, or recombinant equivalents or analogs of Epo; and/or (d) possess an extended half-life in vivo beyond that of Epo, or recombinant equivalents or analogs of Epo. It is understood that the unique functional attributes of an EREDLA are relative to comparable dosing of Epo, or recombinant equivalents or analogs of Epo, and an EREDLA (e.g., amount, frequency, route of administration, etc.).

The compounds mentioned immediately above constitute a genus comprising Epo receptor-specific antibodies, such as but not limited to the antibodies as variously defined and exemplified herein. The definition of antibodies includes Epo receptor-specific maxibodies, such as but not limited to, the maxibodies and other antibody-like structures variously defined and exemplified herein.

Exemplified species of the EREDLA genus include but are not limited to:

An EREDLA comprising the sequences:

(SEQ ID. NO.:1)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS.,
and

(SEQ ID. NO.:2)

QSVLTQPPSASGSPGQSVTISCTGTSSDVGGYNYVSWYQQHPGKAPKLMI
YEVSKRPSGVPDRFSGSKSGNTASLTVSGLQPEDEADYYCSSYAGRNWVF
GGGTQLTVL.

An EREDLA comprising the sequences:

(SEQ ID. NO.:3)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS,
and

(SEQ ID. NO.:4)

QSALTQPASVSGSPGQSITISCTGTSSDVGGYIYVSWYQQHPGKAPKLMI
YDVSRRPSGISDRFSGSKSGNTASLTISGLQAEDEADYYCNSYTTLSTWL
FGGGTKVTVL.

An EREDLA comprising the sequences:

(SEQ ID. NO.:5)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSS,
and

(SEQ ID. NO.:6)

QSALTQPASVSGSPGQSIIISCTGTRSDIGGYNYVSWYQHHPGRAPKLII
FDVNNRPSGVSHRFSGSKSGNTASLTISGLQAEDEADYYCNSFTDSRTWL
FGGGTKLTVL.

An EREDLA comprising the sequences:

(SEQ ID. NO.:7)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSS,
and

(SEQ ID. NO.:8)

QSVLTQPPSVSEAPGQRVTIACSGSSSNIGNNAVSWYQQLPGKAPTLLIY
YDNLLPSGVSDRFSGSKSGTSASLAISGLQSEDEADYYCAAWDDSLNDWV
FGGGTKVTVL.

An EREDLA comprising the sequences:

(SEQ ID. NO.:9)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSA,
and

(SEQ ID. NO.:10)

QAVLTQPSSVSGAPGQRVTISCTGSSSNLGTGYDVHWYQQLPGTAPKLLI
YGNSNRPSGVPDRFSGSKSDTSGLLAITGLQAEDEATYYCQSYDFSLSAM
VFGGGTKVTVL.

An EREDLA comprising the sequences:

(SEQ ID NO.:56)

QVQLQQSGGGVVQPGRSLRLSCAASGFTFSDYAMHWVRQAPGKGLEWVAV
ISNHGKSTYYADSVKGRFTISRDNSKHMLYLQMNSLRADDTALYYCARDI
ALAGDYWGQGTLVTVSA,
and

(SEQ ID NO.:58)

DIQMTQSPSSLSASVGDRVTITCRASQSISSYLNWYQQLPGKVPKLLIYG
ASKLQSGVPSRFSGSGSGTDFTLTISSLQPEDFATYYCLQDYNYPLTFGP
GTRLEIK.

An EREDLA comprising the sequences:

(SEQ ID NO.:60)

QVQLQESGPGLVRPSGTLSLTCAVSGGSIGSSNWWSWVRQAPGKGLEWIG
EISQSGSTNYNPSLKGRVTISLDRSRNQLSLKLSSVTAADTAVYYCARQL
RSIDAFDIWGPGTTVTVSA,
and

(SEQ ID NO.:62)

SYVLTQPPSVSVSPGLTATITCSGDKLGDKYASWYQQKPGQSPVLVIYQD
RKRPSGIPERFSGSNSGNTATLTISGTQAVDEADYYCQAWDSDTSYVFGT
GTQLTVL.

An EREDLA comprising the sequences:

(SEQ ID NO.:64)

QVQLQESGPGLVKPSETLSLTCTVSGGYINNYYWSWIRQPPGKGLEWIGY
IHYSGSTYYNPSLKSRVTISEDTSKNQFSLKLSSATAADTAVYYCARVGY
YYDSSGYNLAWYFDLWGRGTLVTVSA,
and

(SEQ ID NO.:66)

SSELTQDPAVSVALGQTVRITCQGDNLRSYSATWYQQKPGQAPVLVLFGE
NNRPSGIPDRFSGSKSGDTAVLTITGTQTQDEADYYCTSRVNSGNHLGVF
GPGTQLTVL.

An EREDLA comprising the sequences:

(SEQ ID NO.:68)

EVQLVESGAEVKKPGASVKVSCKASGYTFTGYYMHWVRQAPGQGLEWMGW
INPNSGGTNYAQKFQGRVTMTRDTSISTAYMELSRLRSDDTAVYYCARGG
HMTTVTRDAFDIWGQGTMVTVSA,
and

(SEQ ID NO.:70)

SSELTQDPAVSVALGQTIRITCQGDSLRYYYATWYQQKPGQAPILVIYGQ
NNRPSGVPDRFSGSSSGNTASLTITGAQAEDEADYYCGTWDSSVSASWVF
GGGTKVTVL.

ID NO.: 70).

An EREDLA comprising the sequences:

(SEQ ID NO.:72)

QVQLQQSGAEVKKPGASVKVSCKASGYTFSGYYMHWVRQAPGQGLEWMGW
INPNSGSTNYAQKFLGRVTMTRDTSISTAYMELSSLRSDDTAVYYCARGH
SGDYFDYWGQGTLVTVSA,
and

(SEQ ID NO.:74)

EIVLTQSPSSLSASVGDRVTITCRASQSVSSWLAWYQQRPGQAPKLLIYA
ARLRGGGPSRFSGSGSGTEFTLTISSLQPEDFATYFCQQSYSTPISFGGG
TKLEIK.

An EREDLA comprising the sequences:

(SEQ ID NO.:76)

QVQLQESGSGLARPSQTLSLTCAVSGGSISSSAFSWNWIRQPPGKGLEWI
GYIYHTGITDYNPSLKSRVTISVDRSKNQFSLNVNSVTAADTAVYYCARG
HGSDPAWFDPWGKGTLVTVSS,
and

(SEQ ID NO.:78)

QSVLTQPPSVSVSPGQTASITCSGDKLGDKYASWYQQRPGQSPVLVIYRD
TKRPSGIPERFSGSNSGNTATLTISGTQAVDEADYYCQAWDSTTSLVFGG
GTKLTVL.

An EREDLA comprising the sequences:

(SEQ ID NO.:80)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGRGTMVTVSS,
and

(SEQ ID NO.:82)

QSVLTQPPSASGSPGQSVTISCTGTSSDVGGFNYVSWYQKYPGKAPKLVI
YEVSKRPSGVPDRFSGSKSGNTASLTVSGLQAEDEADYYCSSWAPGKNLF
GGGTKLTVL.

An EREDLA comprising the sequences:

(SEQ ID NO.:84)

EVQLVESGGGLVQPGGSLRLSCAASGFTFSSYAMSWVRQAPGKGLEWVSG
ISGSGSSEGGTYYADSVKGRFTLSRDNSKNTLYLQMNSLRAEDTALYYCV
KDRPSRYSFGYYFDYWGRGTLVTVSS,
and

(SEQ ID NO.:86)

LPVLTQPPSVSVSPGQTASIACSGNKLGDKYVSWYQQKPGQSPLLVIYQD
TKRPSGIPERFSGSNSGNTATLTISGTQAMDEADYYCQAWDSSTDVVFGG
GTKLTVL.

An EREDLA comprising the sequences:

(SEQ ID NO.:88)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTMVTVSS,
and

(SEQ ID NO.:90)

QSVLTQPPSASGSPGQSVTISCTGTSSDVGGYNYVSWYQQHPDKAPRLMI
YDVNKRPSGVPDRFSGSKSGNTASLTVSGLQAEDEAHYYCNSYAGSNNWV
FGGGTQLTVL.

An EREDLA comprising the sequences:

(SEQ ID NO.:92)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:94)

QSVLTQPPSASGSPGQSVTISCTGTSSDVGGYNYVSWYQQHPGRAPKLII
YEVSKRPSGVPDRFSGSKSGNTASLTVSGLQADDEADYYCNSYAGSIYVF
GSGTKVTVL.

An EREDLA comprising the sequences:

(SEQ ID NO.:96)

QVQLVQSGAEIKKPGASVKVSCKTFGSPFSTNDIHWVRQAPGQGLEWMGI
IDTSGAMTRYAQKFQGRVTVTRETSTSTVYMELSSLKSEDTAVYYCAREG
CTNGVCYDNGFDIWGQGTLVTVSS,
and

(SEQ ID NO.:98)

DIQMTQSPSTLSASIGDRVTITCRASEGIYHWLAWYQQKPGKAPKLLIYK
ASSLASGAPSRFSGSGSGTDFTLTISSLQPDDFATYYCQQYSNYPLTFGG
GTKLEIK.

An EREDLA comprising the sequences:

(SEQ ID NO.:100)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGRGTMVTVSS,
and

(SEQ ID NO.:102)

QSALTQPASVSGSPGQSITISCTGTSSDVGSYNLVSWYQQHPGKVPKLII
YEVSNRPSGVSHRFSGSKSGNTASLTISGLQAEDEADYYCSSLTSSGTWV
FGGGTKVTVL.

An EREDLA comprising the sequences:

(SEQ ID NO.:104)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:106)

QSALTQPPSASGSPGQSVTISCTGTSSDVGAYNYVSWYQQHPGKAPKLMI
YEVARRPSGVPDRFSGSKSGNTASLTVSGLQAEDEADYYCSSYAGSNNFA
VFGRGTKLTVL.

An EREDLA comprising the sequences:

(SEQ ID NO.:108)

EVQLVQSGGGLVQPGGSLRLSCAASGFRFSSYWMTWVRQAPGKGLEWVAN
IKPDGSEKYYVDSVKGRFTMSRDNAKNSVYLQMNSLRAEDTAVYYCARVS
RGGSFSDWGQGTLVTVSS,
and

(SEQ ID NO.:110)

QSALTQPASVSGSPGQSITIPCTGTSSDIGTYDYVSWYQQHPGKVPKVII
YEVTNRPSGVSNRFSGSKSGNTASLTISGLQADDEADYYCNSFTKNNTWV
FGGGTKLTVL.

An EREDLA comprising the sequences:

(SEQ ID NO.:112)

QVQLVESGGGLVQPGRSLILSCAVSGFTFSKYWMTWVRQAPGKGLEWVAN
IKPDGSEKYYVESVKGRFTISRDNAKNSVYLQMNSVRAEDTAVYYCARVS
RGGSFSDWSQGTLVTVSS,
and

(SEQ ID NO.:114)

QSALTQPPSASGSPGQSVTISCTGTSGDVGAYNYVSWYQQYPGKAPKLMI
YEVSKRPSGVPDRFSGSKSGNTASLTVSGLQAEDEADYYCNSYRGSNGPW
VFGGGTKVTVL.

An EREDLA comprising the sequences:

| | |
|---|---|
| SYWMS; | (SEQ ID NO.:11) |
| NIKPDGSEKYYVDSVKG; | (SEQ ID NO.:12) |
| and | |
| VSRGGSYSD. | (SEQ ID NO.:13) |

An EREDLA comprising the sequences:

| | |
|---|---|
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

An EREDLA comprising the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

An EREDLA comprising the sequences:

| | |
|---|---|
| TGTSSDVGGYIYVS; | (SEQ ID NO.:17) |
| DVSRRPS; | (SEQ ID NO.:18) |
| and | |
| NSYTTLSTWL. | (SEQ ID NO.:19) |

An EREDLA comprising the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYIYVS; | (SEQ ID NO.:17) |
| DVSRRPS; | (SEQ ID NO.:18) |
| and | |
| NSYTTLSTWL. | (SEQ ID NO.:19) |

An EREDLA comprising the sequences:

| | |
|---|---|
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

An EREDLA comprising the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

An EREDLA comprising the sequences:

| | |
|---|---|
| SYAMS; | (SEQ ID NO.:23) |
| AISGSGGSTYYADSVKG; | (SEQ ID NO.:24) |
| and | |
| DRVAVAGKGSYYFDS. | (SEQ ID NO.:25) |

An EREDLA comprising the sequences:

| | |
|---|---|
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

An EREDLA comprising the sequences:

| | |
|---|---|
| SYAMS; | (SEQ ID NO.:23) |
| AISGSGGSTYYADSVKG; | (SEQ ID NO.:24) |
| DRVAVAGKGSYYFDS; | (SEQ ID NO.:25) |
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

An EREDLA comprising the sequences:

| | |
|---|---|
| SNSAAWN; | (SEQ ID NO.:29) |
| RTYYRSKWYNDYAVSKS; | (SEQ ID NO.:30) |
| and | |
| DEGPLDY. | (SEQ ID NO.:31) |

An EREDLA comprising the sequences:

| | |
|---|---|
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

An EREDLA comprising the sequences:

| | |
|---|---|
| SNSAAWN; | (SEQ ID NO.:29) |
| RTYYRSKWYNDYAVSKS; | (SEQ ID NO.:30) |
| DEGPLDY | (SEQ ID NO.:31) |
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

An EREDLA comprising the sequence:

| | |
|---|---|
| DYAMH; | (SEQ ID NO.:123) |
| VISNHGKSTYYADSVKG; | (SEQ ID NO.:124) |
| and | |
| DIALAGDY. | (SEQ ID NO.:125) |

An EREDLA comprising the sequence:

| | |
|---|---|
| RASQSISSYLN; | (SEQ ID NO.:126) |
| GASKLQS; | (SEQ ID NO.:127) |
| and | |
| LQDYNYPLT. | (SEQ ID NO.:128) |

An EREDLA comprising the sequence:

| | |
|---|---|
| DYAMH; | (SEQ ID NO.:123) |
| VISNHGKSTYYADSVKG; | (SEQ ID NO.:124) |
| DIALAGDY; | (SEQ ID NO.:125) |
| RASQSISSYLN; | (SEQ ID NO.:126) |
| GASKLQS; | (SEQ ID NO.:127) |
| and | |
| LQDYNYPLT. | (SEQ ID NO.:128) |

An EREDLA comprising the sequence:

| | |
|---|---|
| SSNWWS; | (SEQ ID NO.:129) |
| EISQSGSTNYNPSLKG; | (SEQ ID NO.:130) |
| and | |
| QLRSIDAFDI. | (SEQ ID NO.:131) |

An EREDLA comprising the sequence:

| | |
|---|---|
| DKYAS; | (SEQ ID NO.:132) |
| YQDRKRPSGI; | (SEQ ID NO.:133) |
| and | |
| WDSDTSYV;. | (SEQ ID NO.:134) |

An EREDLA comprising the sequence:

| | |
|---|---|
| SSNWWS; | (SEQ ID NO.:129) |
| EISQSGSTNYNPSLKG; | (SEQ ID NO.:130) |
| QLRSIDAFDI; | (SEQ ID NO.:131) |
| DKYAS; | (SEQ ID NO.:132) |
| YQDRKRPSGI; | (SEQ ID NO.:133) |
| and | |
| WDSDTSYV. | (SEQ ID NO.:134) |

An EREDLA comprising the sequence:

| | |
|---|---|
| NYYWS; | (SEQ ID NO.:135) |
| YIHYSGSTYYNPSLKSR; | (SEQ ID NO.:136) |
| and | |
| VGYYYDSSGYNLAWYFDL. | (SEQ ID NO.:212) |

An EREDLA comprising the sequence:

| | |
|---|---|
| QGDNLRSYSAT; | (SEQ ID NO.:137) |
| GENNRPS; | (SEQ ID NO.:138) |
| and | |
| TSRVNSGNHLGV. | (SEQ ID NO.:139) |

An EREDLA comprising the sequence:

| | |
|---|---|
| NYYWS; | (SEQ ID NO.:135) |
| YIHYSGSTYYNPSLKSR; | (SEQ ID NO.:136) |
| VGYYYDSSGYNLAWYFDL; | (SEQ ID NO.:212) |
| QGDNLRSYSAT; | (SEQ ID NO.:137) |
| GENNRPS; | (SEQ ID NO.:138) |
| and | |
| TSRVNSGNHLGV. | (SEQ ID NO.:139) |

An EREDLA comprising the sequence:

| | |
|---|---|
| GYYMH; | (SEQ ID NO.:140) |
| WINPNSGGTNYAQKFQGR; | (SEQ ID NO.:141) |
| and | |
| GGHMTTVTRDAFDI. | (SEQ ID NO.:142) |

An EREDLA comprising the sequence:

| | |
|---|---|
| QGDSLRYYYAT; | (SEQ ID NO.:143) |
| GQNNRPS; | (SEQ ID NO.:144) |
| and | |
| GTWDSSVSASWV. | (SEQ ID NO.:145) |

An EREDLA comprising the sequence:

| | |
|---|---|
| GYYMH; | (SEQ ID NO.:140) |
| WINPNSGGTNYAQKFQGR; | (SEQ ID NO.:141) |
| GGHMTTVTRDAFDI; | (SEQ ID NO.:142) |
| QGDSLRYYYAT; | (SEQ ID NO.:143) |
| GQNNRPS; | (SEQ ID NO.:144) |
| and | |
| GTWDSSVSASWV. | (SEQ ID NO.:145) |

An EREDLA comprising the sequence:

| | |
|---|---|
| GYYMH; | (SEQ ID NO.:146) |
| WINPNSGSTNYAQKFLG; | (SEQ ID NO.:147) |
| and | |
| GHSGDYFDY. | (SEQ ID NO.:148) |

An EREDLA comprising the sequence:

| | |
|---|---|
| RASQSVSSWLA; | (SEQ ID NO.:149) |
| AARLRG; | (SEQ ID NO.:150) |
| and | |
| QQSYSTPIS. | (SEQ ID NO.:151) |

An EREDLA comprising the sequence:

| | |
|---|---|
| GYYMH; | (SEQ ID NO.:146) |
| WINPNSGSTNYAQKFLG; | (SEQ ID NO.:147) |
| GHSGDYFDY; | (SEQ ID NO.:148) |
| RASQSVSSWLA; | (SEQ ID NO.:149) |
| AARLRG; | (SEQ ID NO.:150) |
| and | |
| QQSYSTPIS. | (SEQ ID NO.:151) |

An EREDLA comprising the sequence:

| | |
|---|---|
| SSAFSWN; | (SEQ ID NO.:152) |
| YIYHTGITDYNPSLKS; | (SEQ ID NO.:153) |
| and | |
| GHGSDPAWFDP. | (SEQ ID NO.:154) |

An EREDLA comprising the sequence:

| | |
|---|---|
| SGDKLGDKYAS; | (SEQ ID NO.:155) |
| RDTKRPS; | (SEQ ID NO.:156) |
| and | |
| QAWDSTTSLV. | (SEQ ID NO.:157) |

An EREDLA comprising the sequence:

| | |
|---|---|
| SSAFSWN; | (SEQ ID NO.:152) |
| YIYHTGITDYNPSLKS; | (SEQ ID NO.:153) |
| GHGSDPAWFDP; | (SEQ ID NO.:154) |
| SGDKLGDKYAS; | (SEQ ID NO.:155) |
| RDTKRPS; | (SEQ ID NO.:156) |
| and | |
| QAWDSTTSLV. | (SEQ ID NO.:157) |

An EREDLA comprising the sequence:

| | |
|---|---|
| SYWMS; | (SEQ ID NO.:158) |
| NIKPDGSEKYYVDSVKG; | (SEQ ID NO.:159) |
| and | |
| VSRGGSYSD. | (SEQ ID NO.:160) |

An EREDLA comprising the sequence:

| | |
|---|---|
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

An EREDLA comprising the sequence:

| | |
|---|---|
| SYWMS; | (SEQ ID NO.:158) |
| NIKPDGSEKYYVDSVKG; | (SEQ ID NO.:159) |
| VSRGGSYSD; | (SEQ ID NO.:160) |
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

An EREDLA comprising the sequence:

| | |
|---|---|
| SYAMS; | (SEQ ID NO.:164) |
| GISGSGSSEGGTYYADSVKG; | (SEQ ID NO.:165) |
| and | |
| DRPSRYSFGYYFDY. | (SEQ ID NO.:166) |

An EREDLA comprising the sequence:

| | |
|---|---|
| SGNKLGDKYVS; | (SEQ ID NO.:167) |
| QDTKRPS; | (SEQ ID NO.:168) |
| and | |
| QAWDSSTDVV. | (SEQ ID NO.:169) |

An EREDLA comprising the sequence:

| | |
|---|---|
| SYAMS; | (SEQ ID NO.:164) |
| GISGSGSSEGGTYYADSVKG; | (SEQ ID NO.:165) |
| DRPSRYSFGYYFDY; | (SEQ ID NO.:166) |
| SGNKLGDKYVS; | (SEQ ID NO.:167) |
| QDTKRPS; | (SEQ ID NO.:168) |
| and | |
| QAWDSSTDVV. | (SEQ ID NO.:169) |

An EREDLA comprising the sequence:

| | |
|---|---|
| KYWMT; | (SEQ ID NO.:170) |
| NIKPDGSEKYYVESVKG; | (SEQ ID NO.:171) |
| and | |
| VSRGGSFSD. | (SEQ ID NO.:172) |

An EREDLA comprising the sequence:

| | |
|---|---|
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

An EREDLA comprising the sequence:

| | |
|---|---|
| KYWMT; | (SEQ ID NO.:170) |
| NIKPDGSEKYYVESVKG; | (SEQ ID NO.:171) |
| VSRGGSFSD; | (SEQ ID NO.:172) |
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

An EREDLA comprising the sequence:

| | |
|---|---|
| KYWMT; | (SEQ ID NO.:176) |
| NIKPDGSEKYYVESVKG; | (SEQ ID NO.:177) |
| and | |
| VSRGGSFSD. | (SEQ ID NO.:178) |

An EREDLA comprising the sequence:

| | |
|---|---|
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

An EREDLA comprising the sequence:

| | |
|---|---|
| KYWMT; | (SEQ ID NO.:176) |
| NIKPDGSEKYYVESVKG; | (SEQ ID NO.:177) |
| VSRGGSFSD; | (SEQ ID NO.:178) |
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

An EREDLA comprising the sequence:

| | |
|---|---|
| TNDIH; | (SEQ ID NO.:182) |
| IIDTSGAMTRYAQKFQG; | (SEQ ID NO.:183) |
| and | |
| EGCTNGVCYDNGFDI. | (SEQ ID NO.:184) |

An EREDLA comprising the sequence:

| | |
|---|---|
| RASEGIYHWLA; | (SEQ ID NO.:185) |
| KASSLAS; | (SEQ ID NO.:186) |
| and | |
| QQYSNYPLT. | (SEQ ID NO.:187) |

An EREDLA comprising the sequence:

| | |
|---|---|
| TNDIH; | (SEQ ID NO.:182) |
| IIDTSGAMTRYAQKFQG; | (SEQ ID NO.:183) |
| EGCTNGVCYDNGFDI; | (SEQ ID NO.:184) |
| RASEGIYHWLA; | (SEQ ID NO.:185) |
| KASSLAS; | (SEQ ID NO.:186) |
| and | |
| QQYSNYPLT. | (SEQ ID NO.:187) |

An EREDLA comprising the sequence:

| | |
|---|---|
| KYWMT; | (SEQ ID NO.:188) |
| NIKPDGSEKYYVESVKG; | (SEQ ID NO.:189) |
| and | |
| VSRGGSFSD. | (SEQ ID NO.:190) |

An EREDLA comprising the sequence:

| | |
|---|---|
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

An EREDLA comprising the sequence:

| | |
|---|---|
| KYWMT; | (SEQ ID NO.:188) |
| NIKPDGSEKYYVESVKG; | (SEQ ID NO.:189) |
| VSRGGSFSD; | (SEQ ID NO.:190) |
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

An EREDLA comprising the sequence:

| | |
|---|---|
| KYWMT; | (SEQ ID NO.:194) |
| NIKPDGSEKYYVESVKG; | (SEQ ID NO.:195) |
| and | |
| VSRGGSFSD. | (SEQ ID NO.:196) |

An EREDLA comprising the sequence:

| | |
|---|---|
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

An EREDLA comprising the sequence:

| | |
|---|---|
| KYWMT; | (SEQ ID NO.:194) |
| NIKPDGSEKYYVESVKG; | (SEQ ID NO.:195) |
| VSRGGSFSD; | (SEQ ID NO.:196) |
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

An EREDLA comprising the sequence:

| | |
|---|---|
| SYWMT; | (SEQ ID NO.:200) |
| NIKPDGSEKYYVDSVKG; | (SEQ ID NO.:201) |
| and | |
| VSRGGSFSD. | (SEQ ID NO.:202) |

An EREDLA comprising the sequence:

| | |
|---|---|
| TGTSSDIGTYDYVS; | (SEQ ID NO.:203) |
| EVTNRPS; | (SEQ ID NO.:204) |
| and | |
| NSFTKNNTWV. | (SEQ ID NO.:205) |

An EREDLA comprising the sequence:

| | |
|---|---|
| SYWMT; | (SEQ ID NO.:200) |
| NIKPDGSEKYYVDSVKG; | (SEQ ID NO.:201) |
| VSRGGSFSD; | (SEQ ID NO.:202) |
| TGTSSDIGTYDYVS; | (SEQ ID NO.:203) |
| EVTNRPS; | (SEQ ID NO.:204) |
| and | |
| NSFTKNNTWV. | (SEQ ID NO.:205) |

In certain embodiments, an antibody is provided which comprises the sequences:

| | |
|---|---|
| KYWMT; | (SEQ ID NO.:206) |
| NIKPDGSEKYYVESVKG; | (SEQ ID NO.:207) |
| and | |
| VSRGGSFSD. | (SEQ ID NO.:208) |

An EREDLA comprising the sequence:

| | |
|---|---|
| TGTSGDVGAYNYVS; | (SEQ ID NO.:209) |
| EVSKRPS; | (SEQ ID NO.:210) |
| and | |
| NSYRGSNGPWV. | (SEQ ID NO.:211) |

An EREDLA comprising the sequence:

| | |
|---|---|
| KYWMT; | (SEQ ID NO.:206) |
| NIKPDGSEKYYVESVKG; | (SEQ ID NO.:207) |
| VSRGGSFSD; | (SEQ ID NO.:208) |
| TGTSGDVGAYNYVS; | (SEQ ID NO.:209) |
| EVSKRPS; | (SEQ ID NO.:210) |
| and | |
| NSYRGSNGPWV. | (SEQ ID NO.:211) |

An EREDLA comprising the sequence:

(SEQ ID NO.:45)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSVLTQPPSASGSPGQ
SVTISCTGTSSDVGGYNYVSWYQQHPGKAPKLMIYEVSKRPSGVPDRFSG
SKSGNTASLTVSGLQPEDEADYYCSSYAGRNWVFGGGTQLTVLGAAAEPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK.

An EREDLA comprising the sequence:

(SEQ ID NO.:46)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSALTQPASVSGSPGQ
SITISCTGTSSDVGGYIYVSWYQQHPGKAPKLMIYDVSRRPSGISDRFSG
SKSGNTASLTISGLQAEDEADYYCNSYTTLSTWLFGGGTKVTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

An EREDLA comprising the sequence:

(SEQ ID NO.:47)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSSGGGGSGGGGSGGGGSAQSALTQPASVSGSPGQ
SIIISCTGTRSDIGGYNYVSWYQHHPGRAPKLIIFDVNNRPSGVSHRFSG
SKSGNTASLTISGLQAEDEADYYCNSFTDSRTWLFGGGTKLTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

An EREDLA comprising the sequence:

(SEQ ID NO.:48)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSSGGGGSGGGGSGGGGSAQSVLTQPPSV
SEAPGQRVTIACSGSSSNIGNNAVSWYQQLPGKAPTLLIYYDNLLPSGVS
DRFSGSKSGTSASLAISGLQSEDEADYYCAAWDDSLNDWVFGGGTKVTVL
GAAAEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLT
VDKSRWQQGNVFSCSVMHEALHNHYTQKSLSLSPGK.

An EREDLA comprising the sequence:

(SEQ ID NO.:49)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSAGGGGSGGGGSGGGGSGAPQAVLTQPSSVSGA
PGQRVTISCTGSSSNLGTGYDVHWYQQLPGTAPKLLIYGNSNRPSGVPDR
FSGSKSDTSGLLAITGLQAEDEATYYCQSYDFSLSAMVFGGGTKVTVLAA
AEPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDK
SRWQQGNVFSCSVMHEALHNHYTQKSLSLSPGK.

EREDLAs bind the Epo receptor, as shown in Example 3. EREDLAs may be screened for Epo receptor binding activity using the assay described in Example 3 or any other conventional Epo receptor-binding assay known in the art. Additionally, EREDLAs activate the Epo receptor (see Example 8), but with the unique characteristics described below. Preliminary screening of EREDLAs for Epo receptor activation may be performed using the assay described in Example 8 or any other conventional Epo receptor activation assay known in the art.

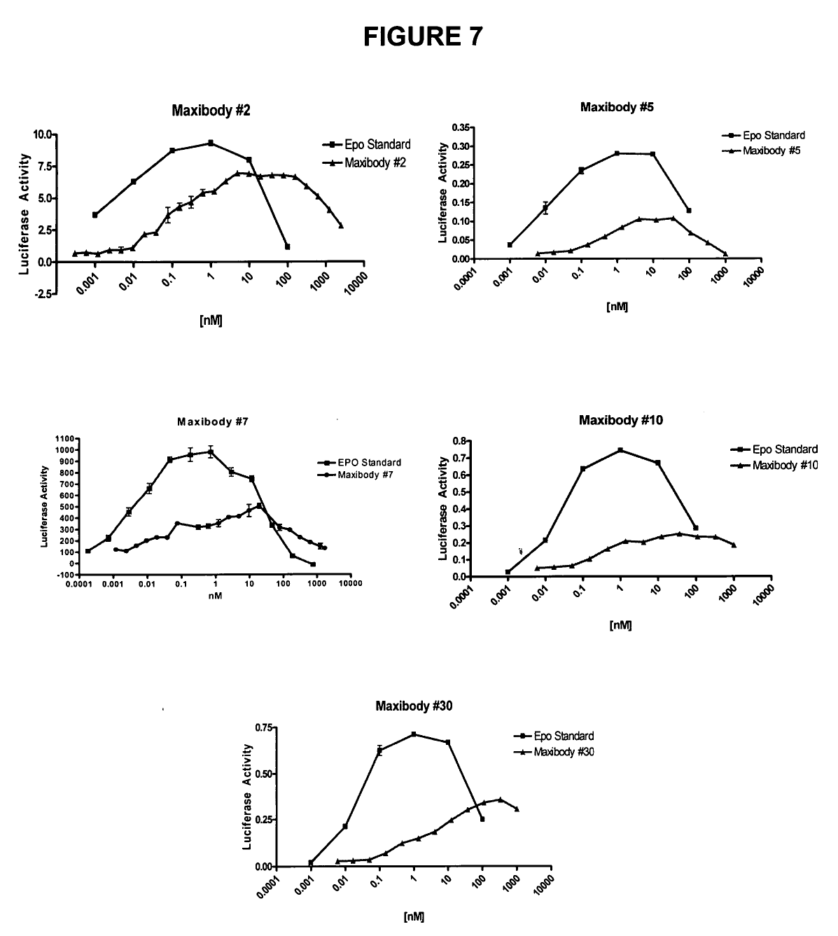

EREDLAs bind the Epo receptor in a population of cells expressing the Epo receptor and activate the Epo receptor to a lesser degree than Epo, or recombinant equivalents or analogs of Epo, when used at the same or higher concentrations than Epo, or recombinant equivalents or analogs of Epo (such EREDLAs are sometimes characterized herein as low potency agonists). Members of the genus may be screened and identified using the in vitro and in vivo methods described herein, as well as any other suitable assays and models known in the art. Exemplary species of the EREDLA genus were tested and shown to activate the Epo receptor in a population of cells to a lesser extent than Epo, or recombinant equivalents or analogs of Epo. Examples 8 and 19 describe versions of an assay that may be used to identify and characterize EREDLAs. As shown in FIG. 7, species of the genus did not activate the Epo receptor to the same extent as the Epo standard in a UT-7-Luciferase-based assay even though equivalent or excessive concentrations of the EREDLA (in relation to the Epo standard) were titrated in the assay. Therefore, compounds having profiles similar to the EREDLAs shown in FIG. 7 may constitute an EREDLA, while Epo-activating molecules having a profile similar to the Epo standard, are not considered an EREDLA.

In addition, objective criteria for distinguishing a member of the EREDLA genus from a nonmember may include a ratio of the EC₅₀ values derived from an in vitro assay measuring the relative readout of Epo, or recombinant equivalents or analogs of Epo, activating the erythropoietin receptor/the EC₅₀ values derived from said assay measuring the relative readout of an Erythropoietin Receptor Extended Duration Limited Agonist activating the erythropoietin receptor, wherein the ratio is always less than 1. Examples 8 and 19 describe versions of such an assay, but it is understood that any comparable assay known in the art may be used and from such assays said ratio could be derived and members of the EREDLA genus identified. As shown in Table 5 in Example 19, the EC₅₀ ratios for the various species of the EREDLA genus all have ratios less than 1, with one exception: clone #330 which would not be considered a species of the EREDLA genus using the EC₅₀ ratio criteria, but may be considered a species of the EREDLA genus if clone #330 satisfies one or more of the other EREDLA criteria described herein.

It is understood that the relative activity of an EREDLA versus Epo, or recombinant equivalents or analogs of Epo, may be evaluated and identified in numerous ways and in various assays; the nature of the invention is not limited by the assay used to characterize a member of the EREDLA genus. Of course, it is also understood that absolute ratio values are relative to the assay being used and its particular readout. Regardless of the assay used, the ratio of the EC₅₀ value derived from an in vitro assay measuring the relative readout of Epo, or recombinant equivalents or analogs of Epo, activating the erythropoietin receptor/the EC₅₀ value derived from said assay measuring the relative readout of an Erythropoietin Receptor Extended Duration Limited Agonist activating the erythropoietin receptor is always less than 1.

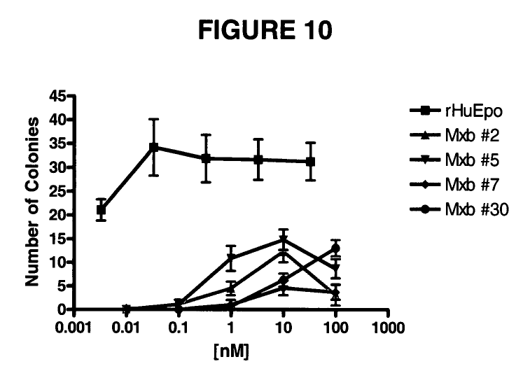

EREDLAs have the unique capacity to stimulate a population of human CD34+ peripheral blood progenitor cells to stimulate the production of erythroid colonies to a lesser extent than Epo, or recombinant equivalents or analogs of Epo. Example 11 describes testing several EREDLAs in a standard Burst Forming Unit-Erythroid (BFU-E) assay. All species tested induced the formation of hemoglobin-containing erythroid colonies. But, the EREDLAs were significantly less potent than the Epo standard at inducing BFU-E-derived colonies, and the maximal number of colonies was induced at significantly higher concentrations using an EREDLA than for the Epo standard, as shown in FIG. 10. In addition, the maximal number of colonies induced by any of the ERELDAs was always significantly lower than the maximal number of the colonies induced by the Epo standard. These data demonstrate that certain EREDLAs are low potency agonists of the Epo receptor compared to the natural Epo ligand.

EREDLAs may be distinguished by their activity relative to Epo, or recombinant equivalents or analogs of Epo, in a BFU-E assay. In a standard BFU-E assay, such as that described herein and known in the art, an EREDLA may require from about 10× to 2,000×, 20× to 1,000×, 30× to 500×, 40× to 400×, 50× to 300×, 60× to 200×, 70× to 100×, or from about 200× to 2000× more EREDLA to achieve maximum colony formation relative to the amount of an Epo standard required to achieve maximum colony formation. In addition, an EREDLA will elicit only from about 15%, 20%, 25%, 30%, 35%, 40%, 45%, 50%, 55%, or 60% as many colonies as an Epo standard in the BFU-E assay relative to an Epo standard.

In addition, the size of the BFU-E colonies induced by an EREDLA are significantly smaller than the size of colonies induce by Epo, or recombinant equivalents or analogs of Epo. An Epo standard may be Epo, or recombinant equivalents or analogs of Epo. Thus, this is another distinguishing characteristic of an EREDLA versus a non-EREDLA. An EREDLA may have an average BFU-E colony that is about 15%, 20%, 25%, 30%, 35%, 40%, 45%, 50%, 55%, 60%, 65%, 70%, or 75% smaller in diameter relative to Epo, or recombinant equivalents or analogs of Epo.

Embodiments of EREDLAs may comprise low affinity partial agonists and high affinity partial agonists to the Epo receptor. When referring to low affinity and high affinity partial agonists it is understood that affinity is relative to the approximate Kd of human Epo, or recombinant equivalents or analogs of Epo. In the generic sense, a partial agonist is typically defined as a compound that possesses affinity for a receptor, but unlike a full agonist, will elicit only a small degree of the pharmacological response peculiar to the nature of the receptor involved, even if a high proportion of receptors are occupied by the compound. Certain embodiments of the EREDLAs, e.g., several of the species exemplified in the antibodies and maxibodies described herein, may be considered low affinity partial agonists. Without being bound by theory, certain embodiments of the genus bind the Epo receptor in an agonistic manner and their binding to the Epo receptor can block the binding of Epo (or recombinant equivalents or analogs of Epo) to the Epo receptor, partially block binding of Epo (or recombinant equivalents or analogs of Epo) to the Epo receptor, or do not block binding of Epo (or recombinant equivalents or analogs of Epo) to the Epo receptor. Binding of an ERELDA to Epo receptor can have an agonistic or antagonistic effect depending on the concentration of the ERELDA. For example, a population of cells expressing the Epo receptor exposed to an EREDLA at low concentrations may result in a percentage of Epo receptors being dimerized and activated, but as the concentration of the EREDLA increases significantly beyond receptor saturation levels, a single EREDLA molecule may engage a single receptor subunit, thus preventing two receptor subunits from dimerizing and being activated.

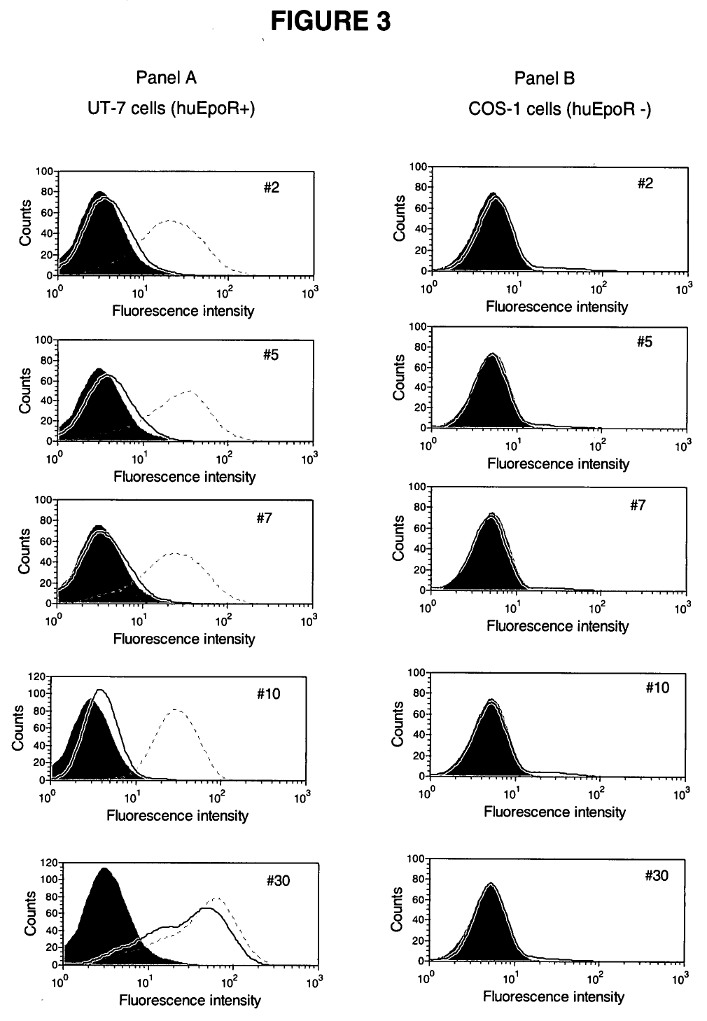

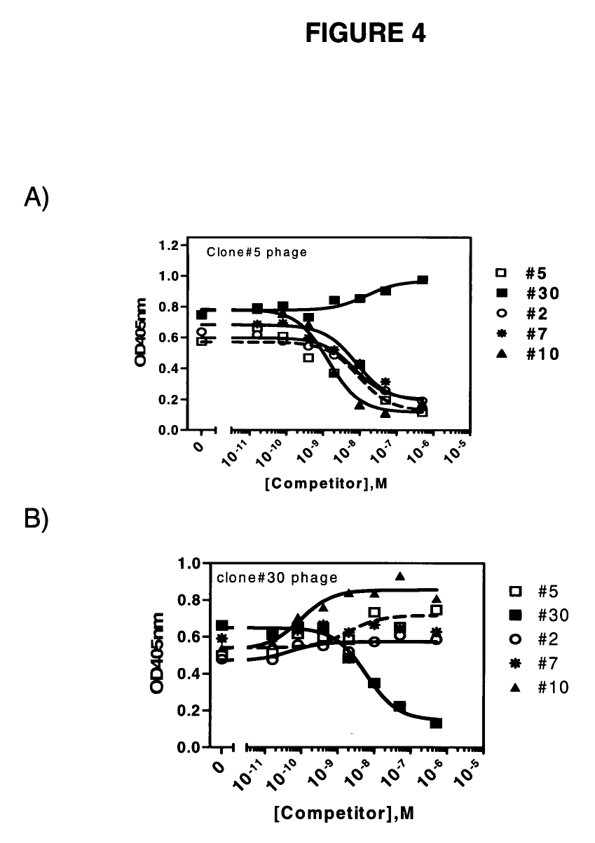

As described above, embodiments include EREDLAs that may or may not bind to the Epo-engaging domain of the Epo receptor and may or may not displace Epo binding. Species of EREDLAs that bind the Epo-engaging domain of the Epo receptor include, but are not limited to, clones 2, 5, 7, and 10 (see Example 3 and FIG. 3A). A species that does not bind the Epo-binding domain of the Epo receptor is exemplified by clone 30, which as described in Example 3, binds to the Epo receptor but does not competitively block binding of Epo ligand to the Epo receptor (FIG. 3A). As further evidence of an EREDLA that does not bind to the Epo-engaging domain of the Epo receptor, Example 5 demonstrates that clone 30 binds to an epitope on the Epo receptor that is distinct from clones 2, 5, 7, and 10 (see FIGS. 4A and 4B).

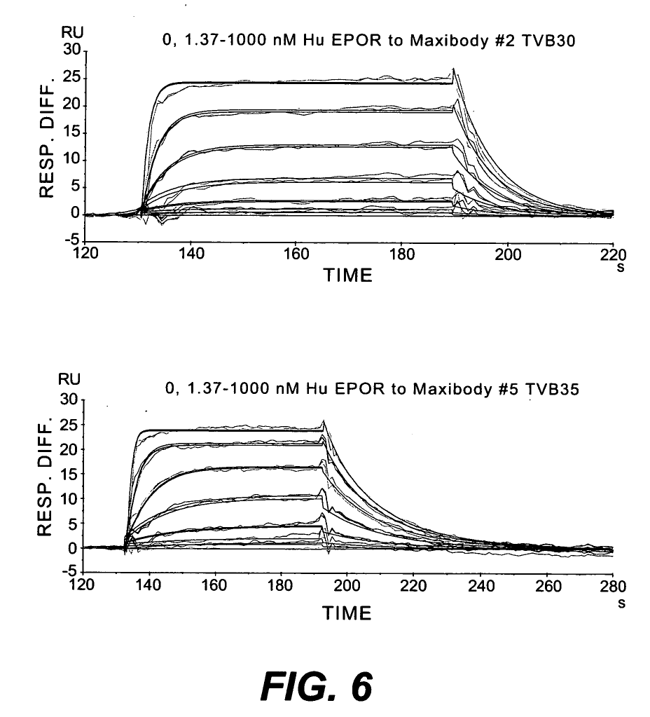

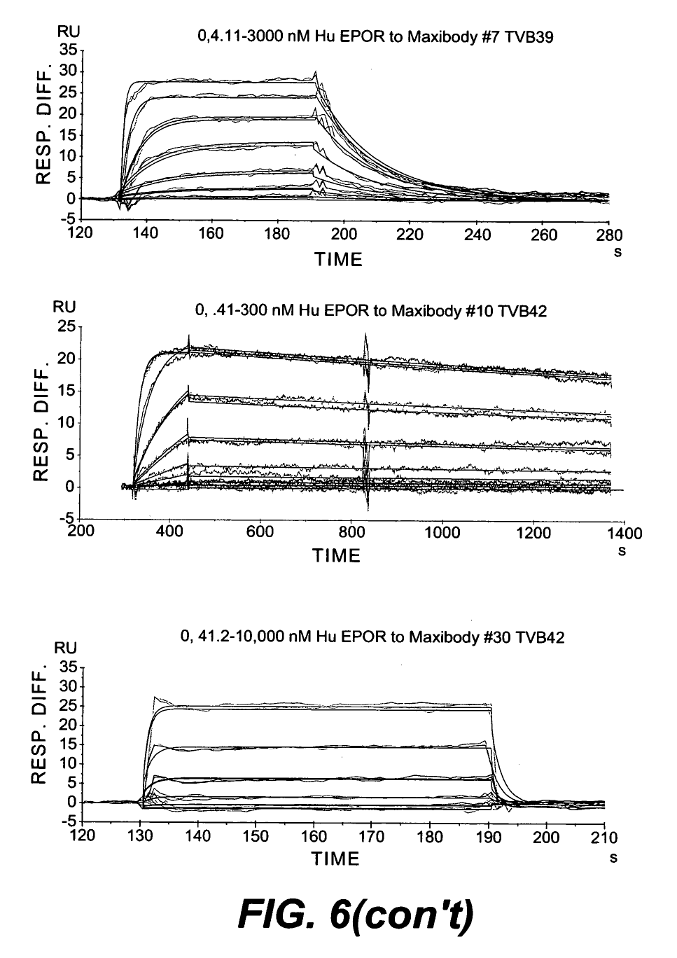

Embodiments of the EREDLA genus have an affinity (Kd) for the Epo receptor that is lower than the affinity of Epo, or recombinant equivalents or analogs of Epo. For example, the Kd for human Epo has been reported to be approximately 0.25 nM (see, Ahaded A, et al., Prep Biochem Biotechnol. 1999 May; 29(2):163-76). Therefore, an EREDLA may have a Kd greater than approximately 0.25 nM; in other embodiments an EREDLA may have a Kd in the range of about 0.26 nM to 20,000 nM, other embodiments may have a Kd in the range of about 0.5 nM to 18,000 nM, other embodiments may have a Kd in the range of about 0.75 nM to 16,000 nM, and in yet still other embodiments has a Kd of about 1.1 nM to 14,900 nM. Exemplified embodiments include but are not limited to the EREDLAs having the Kds described in Example 7, Example 18, Table 2, and Table 3. The Kd of EREDLAs may be measured relative to Epo in any standard assay known in the art, such as a variety of ELISA formats and Scatchard analysis or by BIACOR® technology, as demonstrated in Example 7 (FIG. 6).

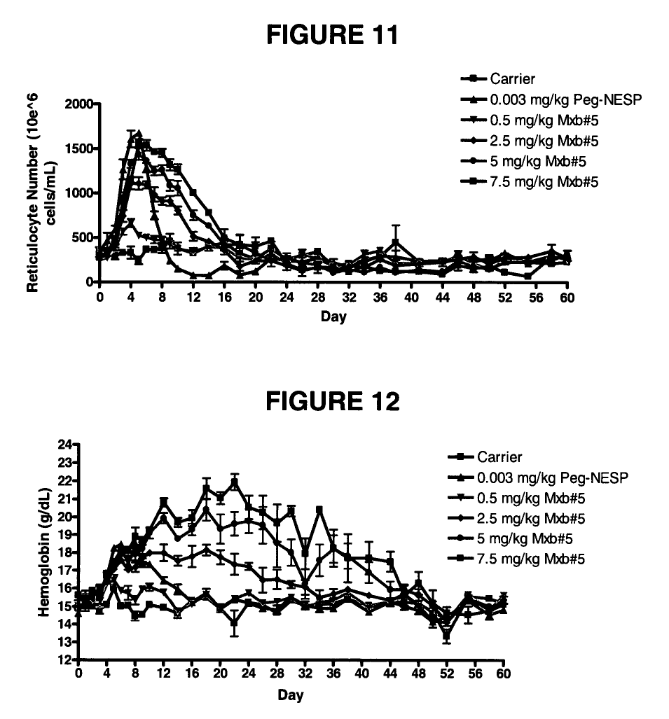

EREDLAs possess extended pharmacodynamic properties beyond that of Epo, or recombinant equivalents or analogs of Epo. As described in Example 12 and FIGS. 11-18, EREDLAS elicit initial reticulocyte increases in mammals that is significantly longer in duration than Epo, or recombinant equivalents or analogs of Epo, and an EREDLA elicits hemoglobin responses in a mammal that is of extended duration and magnitude compared to Epo, or recombinant equivalents or analogs of Epo. For example, the activity profile of maxibody 5 (Mxb 5, a species of the EREDLA genus) is dramatically different from that of the Epo standard (PEG-NESP). The peak reticulocyte number was achieved on day 4 after an injection of either PEG-NESP or Mxb 5, but the duration of the reticulocyte response was significantly increased in the mice that received doses of Mxb 5 between 2.5 and 7.5 mg/kg. The reticulocyte numbers returned to baseline on day 8 in the PEG-NESP-treated mice, but it took 14 to 18 days for the reticulocytes to return to baseline in the Mxb 5-treated mice. In mice injected with Mxb 5 at doses between 5 and 7.5 mg/kg, the hemoglobin levels stayed above baseline for 46 to 52 days. In contrast, the hemoglobin level in the PEG-NESP-treated mice returned to baseline at day 16, thus showing a very significant difference in the duration and magnitude of the hemoglobin response in the mice treated with Mxb 5 or PEG-NESP.

In a further example of a species of the EREDLA genus, a single subcutaneous (SC) injection of Mxb 7 at 7.5 mg/kg, the reticulocyte numbers stayed above baseline for 12 days while in the mice injected with PEG-NESP, the reticulocyte numbers stayed above baseline for 8 days. Hemoglobin levels were measured for 24 days, and during this time, the increase in hemoglobin was sustained at higher levels and for a longer period of time in the mice that received Mxb 7 at 7.5 mg/kg compared to the PEG-NESP-treated mice. After a single PEG-NESP injection, the hemoglobin peak was reached on day 5, and hemoglobin was back to baseline on day 14. In contrast, after a single injection of Mxb 7 (7.5 mg/kg), the hemoglobin peak was reached on day 12, and hemoglobin returned to baseline on day 24. This experiment indicates that Mxb 7 has very different properties from the erythropoietic agent PEG-NESP. After a single administration, the mice treated with Mxb 7 had a longer-duration erythropoietic response than PEG-NESP-treated mice as demonstrated by the increase in reticulocyte numbers and hemoglobin levels.

As demonstrated herein, embodiments of EREDLAs increase hemoglobin levels above baseline for a period of time that is longer than the total life span of erythrocytes in test subjects (e.g., 40 days in mice). Importantly, this is far longer than the Epo standard used in the animal models. The life span of erythrocytes in humans is about 120 days, and consequently an EREDLA may extend hemoglobin levels above baseline in humans longer than 120 days. Thus, a single administration of an EREDLA may be enough to correct anemia in a human (i.e., increase circulating hemoglobin levels above a patient's baseline value) over a period of about 1 to 6 months, about 2 to 6 months, about 3 to 6 months, about 4 to 6 months, or about 5 to 6 months.

An EREDLA may be distinguished from a non-EREDLA by its pharmacodynamics. The assays and animal models described herein, or other suitable assays and animal models known in the art, may be used to identify an EREDLA. As described above, an EREDLA maintains hemoglobin concentrations above baseline in vivo at least about 10, 15, 20, 25, 30, 35, 40, 45, 50, 55, 60, 65, 70, 75, 80, 90, 100, 110, 115, 120, 125, 130, 135, 140, 145, 150, 155, 160, 165, 170, 175, 180, 185, 190, 195, 200, 210, 215, 220, 225, 230, 235, 240, 245, 250, 255, 260, 265, 270, 275, 280, 285, 290, 295, 300, 310, 315, 320, 325, 330, 335, 340, 345, 350, 355, 360, 365, 370, 375, 380, 385, 390, 395, 400, 410, 415, 420, 425, 430, 435, 440, 445, 450, 455, 460, 465, 470, 475, 480, 485, 490, 495, 500, 510, 515, 520, 525, 530, 535, 540, 545, 550, 555, 560, 565, 570, 575, 580, 585, 590, 595, 600, 610, 615, 620, 625, 630, 635, 640, 645, 650, 655, 660, 665, 670, 675, 680, 685, 690, 695, 700% longer than Epo, or recombinant equivalents or analogs of Epo.

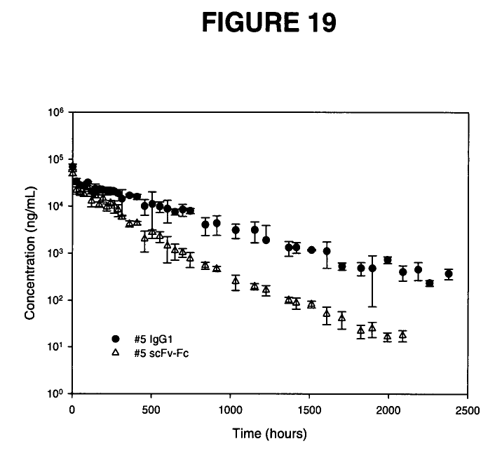

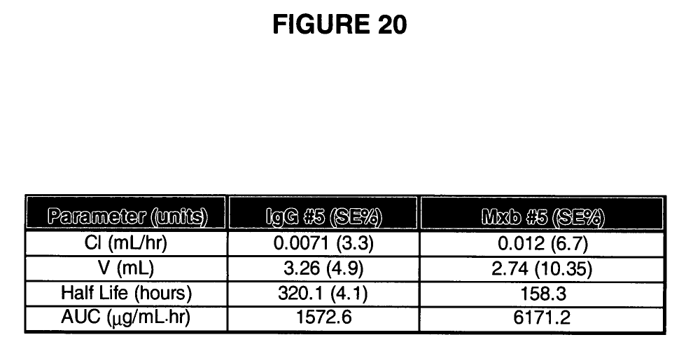

EREDLAs have pharmacokinetic (pK) properties greater than Epo, or recombinant equivalents or analogs of Epo. EREDLAs have extended in vivo half-lives greater than that of Epo, or recombinant equivalents or analogs of Epo. Example 13 (FIGS. 19-21) describes a pharmacokinetic (pK) study of two members of the EREDLA genus and provides a comparison of a representative species relative to various forms of Epo, or recombinant equivalents or analogs of Epo. Pharmacokinetic analysis demonstrated that an EREDLA has a half-life that is about 13 to 80 times longer than various forms of Epo, or recombinant equivalents or analogs of Epo. The pK, as well as other characteristics of EREDLAs, may be enhanced by converting an EREDLA from a maxibody framework to an antibody framework, or other traditional methods of enhancing pK, such as those described herein. In one particular example, maxibody 5 had a half-life of about 158 hours, whereas the IgG #5 version had a half-life of about 320 hours (FIG. 20).

Therefore, an EREDLA has a half-life that is significantly longer than Epo, or recombinant equivalents or analogs of Epo, and have in vivo half-lives that are at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 times longer than Epo, or recombinant equivalents or analogs of Epo.

### DEFINITIONS

Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, delivery, and treatment of patients.

In this application, the use of the singular includes the plural unless specifically stated otherwise. In this application, the use of “or” means “and/or” unless stated otherwise. In the context of a multiple dependent claim, the use of “or” refers back to more than one preceding independent or dependent claim in the alternative only. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. When the term “having” is used herein, for example in the claims, it is understood that the term “having” is equivalent to the term “comprising” and is not meant to be limiting, such as to denote “consisting of.”

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The terms “polynucleotide” and “oligonucleotide” are used interchangeably, and as referred to herein mean a polymeric form of nucleotides of at least 2 bases in length. In certain embodiments, the bases may comprise at least one of ribonucleotides, deoxyribonucleotides, and a modified form of either type of nucleotide. The term includes single and double stranded forms of DNA. In certain embodiments, polynucleotides complementary to specific polynucleotides that encode certain polypeptides described herein are provided.

The term “naturally occurring nucleotides” includes deoxyribonucleotides and ribonucleotides. Deoxyribonucleotides include, but are not limited to, adenosine, guanine, cytosine, and thymidine. Ribonucleotides include, but are not limited to, adenosine, cytosine, thymidine, and uracil. The term “modified nucleotides” includes, but is not limited to, nucleotides with modified or substituted sugar groups and the like. The term “polynucleotide linkages” includes, but is not limited to, polynucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). In certain embodiments, a polynucleotide can include a label for detection.

The term “isolated polypeptide” refers to any polypeptide that (1) is free of at least some proteins with which it would normally be found, (2) is essentially free of other proteins from the same source, e.g., from the same species, (3) is expressed by a cell from a different species, or (4) does not occur in nature.

The terms “polypeptide,” “peptide,” and “protein” are used interchangeably herein and refer to a polymer of two or more amino acids joined to each other by peptide bonds or modified peptide bonds, i.e., peptide isosteres. The terms apply to amino acid polymers containing naturally occurring amino acids as well as amino acid polymers in which one or more amino acid residues is a non-naturally occurring amino acid or a chemical analogue of a naturally occurring amino acid. An amino acid polymer may contain one or more amino acid residues that has been modified by one or more natural processes, such as post-translational processing, and/or one or more amino acid residues that has been modified by one or more chemical modification techniques known in the art.

A “fragment” of a reference polypeptide refers to a contiguous stretch of amino acids from any portion of the reference polypeptide. A fragment may be of any length that is less than the length of the reference polypeptide.

A “variant” of a reference polypeptide refers to a polypeptide having one or more amino acid substitutions, deletions, or insertions relative to the reference polypeptide. In certain embodiments, a variant of a reference polypeptide has an altered post-translational modification site (i.e., a glycosylation site). In certain embodiments, both a reference polypeptide and a variant of a reference polypeptide are specific binding agents. In certain embodiments, both a reference polypeptide and a variant of a reference polypeptide are antibodies.

Variants of a reference polypeptide include, but are not limited to, glycosylation variants. Glycosylation variants include variants in which the number and/or type of glycosylation sites have been altered as compared to the reference polypeptide. In certain embodiments, glycosylation variants of a reference polypeptide comprise a greater or a lesser number of N-linked glycosylation sites than the reference polypeptide. In certain embodiments, an N-linked glycosylation site is characterized by the sequence Asn-X-Ser or Asn-X-Thr, wherein the amino acid residue designated as X may be any amino acid residue except proline. In certain embodiments, glycosylation variants of a reference polypeptide comprise a rearrangement of N-linked carbohydrate chains wherein one or more N-linked glycosylation sites (typically those that are naturally occurring) are eliminated and one or more new N-linked sites are created.

Variants of a reference polypeptide include, but are not limited to, cysteine variants. In certain embodiments, cysteine variants include variants in which one or more cysteine residues of the reference polypeptide are replaced by one or more non-cysteine residues; and/or one or more non-cysteine residues of the reference polypeptide are replaced by one or more cysteine residues. Cysteine variants may be useful, in certain embodiments, when a particular polypeptide must be refolded into a biologically active conformation, e.g., after the isolation of insoluble inclusion bodies. In certain embodiments, cysteine variants of a reference polypeptide have fewer cysteine residues than the reference polypeptide. In certain embodiments, cysteine variants of a reference polypeptide have an even number of cysteines to minimize interactions resulting from unpaired cysteines. In certain embodiments, cysteine variants have more cysteine residues than the native protein.

A “derivative” of a reference polypeptide refers to: a polypeptide: (1) having one or more modifications of one or more amino acid residues of the reference polypeptide; and/or (2) in which one or more peptidyl linkages has been replaced with one or more non-peptidyl linkages; and/or (3) in which the N-terminus and/or the C-terminus has been modified. Certain exemplary modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In certain embodiments, both a reference polypeptide and a derivative of a reference polypeptide are specific binding agents. In certain embodiments, both a reference polypeptide and a derivative of a reference polypeptide are antibodies.

Polypeptides include, but are not limited to, amino acid sequences modified either by natural processes, such as post-translational processing, or by chemical modification techniques that are well known in the art. In certain embodiments, modifications may occur anywhere in a polypeptide, including the peptide backbone, the amino acid side-chains and the amino or carboxyl termini. In certain such embodiments, the modifications may be present to the same or varying degrees at several sites in a given polypeptide. In certain embodiments, a given polypeptide contains many types of modifications such as deletions, additions, and/or substitutions of one or more amino acids of a native sequence. In certain embodiments, polypeptides may be branched and/or cyclic. Cyclic, branched and branched cyclic polypeptides may result from post-translational natural processes (including, but not limited to, ubiquitination) or may be made by synthetic methods. In certain embodiments, certain polypeptide sequences comprise at least one complementarity determining region (CDR).

The term “naturally-occurring” as applied to an object means that an object can be found in nature. For example, a polypeptide or polynucleotide that is present in an organism (including viruses) that can be isolated from a source in nature and which has not been intentionally modified by man in the laboratory or otherwise is naturally-occurring.

The term “operably linked” as used herein refers to components that are in a relationship permitting them to function in their intended manner. For example, in the context of a polynucleotide sequence, a control sequence may be “operably linked” to a coding sequence when the control sequence and coding sequence are in association with each other in such a way that expression of the coding sequence is achieved under conditions compatible with the functioning of the control sequence.

The term “control sequence” refers to polynucleotide sequences which may effect the expression and processing of coding sequences with which they are in association. The nature of such control sequences may differ depending upon the host organism. Certain exemplary control sequences for prokaryotes include, but are not limited to, promoters, ribosomal binding sites, and transcription termination sequences. Certain exemplary control sequences for eukaryotes include, but are not limited to, promoters, enhancers, and transcription termination sequences. In certain embodiments, “control sequences” can include leader sequences and/or fusion partner sequences.

In certain embodiments, a first polynucleotide coding sequence is operably linked to a second polynucleotide coding sequence when the first and second polynucleotide coding sequences are transcribed into a single contiguous mRNA that can be translated into a single contiguous polypeptide.

In the context of polypeptides, two or more polypeptides are “operably linked” if each linked polypeptide is able to function in its intended manner. A polypeptide that is able to function in its intended manner when operably linked to another polypeptide may or may not be able to function in its intended manner when not operably linked to another polypeptide. For example, in certain embodiments, a first polypeptide may be unable to function in its intended manner when unlinked, but may be stabilized by being linked to a second polypeptide such that it becomes able to function in its intended manner. Alternatively, in certain embodiments, a first polypeptide may be able to function in its intended manner when unlinked, and may retain that ability when operably linked to a second polypeptide.

As used herein, two or more polypeptides are “fused” when the two or more polypeptides are linked to form a single contiguous molecule. In certain embodiments, two or more polypeptides are fused by translating them as a single contiguous polypeptide sequence or by synthesizing them as a single contiguous polypeptide sequence. In certain embodiments, two or more fused polypeptides may have been translated in vivo from two or more operably linked polynucleotide coding sequences. In certain embodiments, two or more fused polypeptides may have been translated in vitro from two or more operably linked polynucleotide coding sequences. In certain embodiments, two or more polypeptides are fused if the two polypeptides are linked by a polypeptide or non-polypeptide linker.

As used herein, two or more polypeptides are “operably fused” if each linked polypeptide is able to function in its intended manner.

In certain embodiments, a first polypeptide that contains two or more distinct polypeptide units is considered to be linked to a second polypeptide so long as at least one of the distinct polypeptide units of the first polypeptide is linked to the second polypeptide. As a non-limiting example, in certain embodiments, an antibody is considered linked to a second polypeptide in all of the following instances: (a) the second polypeptide is linked to one of the heavy chain polypeptides of the antibody; (b) the second polypeptide is linked to one of the light chain polypeptides of the antibody; (c) a first molecule of the second polypeptide is linked to one of the heavy chain polypeptides of the antibody and a second molecule of the second polypeptide is linked to one of the light chain polypeptides of the antibody; and (d) first and second molecules of the second polypeptide are linked to the first and second heavy chain polypeptides of the antibody and third and fourth molecules of the second polypeptide are linked to first and second light chain polypeptides of the antibody.

In certain embodiments, the language “a first polypeptide linked to a second polypeptide” encompasses situations where: (a) only one molecule of a first polypeptide is linked to only one molecule of a second polypeptide; (b) only one molecule of a first polypeptide is linked to more than one molecule of a second polypeptide; (c) more than one molecule of a first polypeptide is linked to only one molecule of a second polypeptide; and (d) more than one molecule of a first polypeptide is linked to more than one molecule of a second polypeptide. In certain embodiments, when a linked molecule comprises more than one molecule of a first polypeptide and only one molecule of a second polypeptide, all or fewer than all of the molecules of the first polypeptide may be covalently or noncovalently linked to the second polypeptide. In certain embodiments, when a linked molecule comprises more than one molecule of a first polypeptide, one or more molecules of the first polypeptide may be covalently or noncovalently linked to other molecules of the first polypeptide.

As used herein, a “flexible linker” refers to any linker that is not predicted, according to its chemical structure, to be fixed in three-dimensional space. One skilled in the art can predict whether a particular linker is flexible in its intended context. In certain embodiments, a peptide linker comprising 3 or more amino acids is a flexible linker.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)). In certain embodiments, one or more unconventional amino acids may be incorporated into a polypeptide. The term “unconventional amino acid” refers to any amino acid that is not one of the twenty conventional amino acids. The term “non-naturally occurring amino acids” refers to amino acids that are not found in nature. Non-naturally occurring amino acids are a subset of unconventional amino acids. Unconventional amino acids include, but are not limited to, stereoisomers (e.g., D-amino acids) of the twenty conventional amino acids, unnatural amino acids such as α-, α-disubstituted amino acids, N-alkyl amino acids, lactic acid, homoserine, homocysteine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline) known in the art. In the polypeptide notation used herein, the left-hand direction is the amino terminal direction and the right-hand direction is the carboxy-terminal direction, in accordance with standard usage and convention.

In certain embodiments, conservative amino acid substitutions include substitution with one or more unconventional amino acid residues. In certain embodiments, unconventional amino acid residues are incorporated by chemical peptide synthesis rather than by synthesis in biological systems.

The term “acidic residue” refers to an amino acid residue in D- or L-form that comprises at least one acidic group when incorporated into a polypeptide between two other amino acid residues that are the same or different. In certain embodiments, an acidic residue comprises a sidechain that comprises at least one acidic group. Exemplary acidic residues include, but are not limited to, aspartic acid (D) and glutamic acid (E). In certain embodiments, an acidic residue may be an unconventional amino acid.

The term “aromatic residue” refers to an amino acid residue in D- or L-form that comprises at least one aromatic group. In certain embodiments, an aromatic residue comprises a sidechain that comprises at least one aromatic group. Exemplary aromatic residues include, but are not limited to, phenylalanine (F), tyrosine (Y), and tryptophan (W). In certain embodiments, an aromatic residue may be an unconventional amino acid.

The term “basic residue” refers to an amino acid residue in D- or L-form that may comprise at least one basic group when incorporated into a polypeptide next to one or more amino acid residues that are the same or different. In certain embodiments, a basic residue comprises a sidechain that comprises at least one basic group. Exemplary basic residues include, but are not limited to, histidine (H), lysine (K), and arginine (R). In certain embodiments, a basic residue may be an unconventional amino acid.

The term “neutral hydrophilic residue” refers to an amino acid residue in D- or L-form that comprises at least one hydrophilic and/or polar group, but does not comprise an acidic or basic group when incorporated into a polypeptide next to one or more amino acid residues that are the same or different. Exemplary neutral hydrophilic residues include, but are not limited to, alanine (A), cysteine (C), serine (S), threonine (T), asparagine (N), and glutamine (Q). In certain embodiments, a neutral hydrophilic residue may be an unconventional amino acid.

The terms “lipophilic residue” and “Laa” refer to an amino acid residue in D- or L-form having at least one uncharged, aliphatic and/or aromatic group. In certain embodiments, a lipophilic residue comprises a side chain that comprises at least one uncharged, aliphatic, and/or aromatic group. Exemplary lipophilic sidechains include, but are not limited to, alanine (A), phenylalanine (F), isoleucine (I), leucine (L), norleucine (Nle), methionine (M), valine (V), tryptophan (W), and tyrosine (Y). In certain embodiments, a lipophilic residue may be an unconventional amino acid.

The term “amphiphilic residue” refers to an amino acid residue in D- or L-form that is capable of being either a hydrophilic or lipophilic residue. An exemplary amphiphilic residue includes, but is not limited to, alanine (A). In certain embodiments, an amphiphilic residue may be an unconventional amino acid.

The term “nonfunctional residue” refers to an amino acid residue in D- or L-form that lacks acidic, basic, and aromatic groups when incorporated into a polypeptide next to one or more amino acid residues that are the same or different. Exemplary nonfunctional amino acid residues include, but are not limited to, methionine (M), glycine (G), alanine (A), valine (V), isoleucine (I), leucine (L), and norleucine (Nle). In certain embodiments, a nonfunctional residue may be an unconventional amino acid.

In certain embodiments, glycine (G) and proline (P) are considered amino acid residues that can influence polypeptide chain orientation.

In certain embodiments, a conservative substitution may involve replacing a member of one residue type with a member of the same residue type. As a non-limiting example, in certain embodiments, a conservative substitution may involve replacing an acidic residue, such as D, with a different acidic residue, such as E. In certain embodiments, a non-conservative substitution may involve replacing a member of one residue type with a member of a different residue type. As a non-limiting example, in certain embodiments, a non-conservative substitution may involve replacing an acidic residue, such as D, with a basic residue, such as K. In certain embodiments, a cysteine residue is substituted with another amino acid residue to prevent disulfide bond formation with that position in the polypeptide.

In making conservative or non-conservative substitutions, according to certain embodiments, the hydropathic index of amino acids may be considered. Each amino acid has been assigned a hydropathic index on the basis of its hydrophobicity and charge characteristics. The hydropathic indices of the 20 naturally-occurring amino acids are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known in certain instances that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the polypeptide.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. In certain instances, one may also identify epitopes from primary amino acid sequences on the basis of hydrophilicity. These regions are also referred to as “epitopic core regions.”

Exemplary amino acid substitutions are set forth in Table 1.

TABLE 1
Amino Acid Substitutions

| | | More specific |
|---|---|---|
| Original | Exemplary | exemplary |
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

Similarly, as used herein, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to herein as the transcription direction; sequence regions on the DNA strand having the same sequence as the RNA and which are 5′ to the 5′ end of the RNA transcript are referred to herein as “upstream sequences”; sequence regions on the DNA strand having the same sequence as the RNA and which are 3′ to the 3′ end of the RNA transcript are referred to herein as “downstream sequences.”

In certain embodiments, conservative amino acid substitutions encompass non-naturally occurring amino acid residues, which are typically incorporated by chemical peptide synthesis or by synthesis in biological systems. Those non-naturally occurring amino acid residues include, but are not limited to, peptidomimetics and other reversed or inverted forms of amino acid moieties.

A skilled artisan will be able to determine suitable substitution variants of a reference polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity, including, but not limited to, the CDRs of an antibody, or that may be important for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity and/or structure. In view of such a comparison, in certain embodiments, one can predict the importance of amino acid residues in a polypeptide that correspond to amino acid residues which are important for activity or structure in similar polypeptides. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules. Moreover, in certain embodiments, one skilled in the art may generate test variants containing a single amino acid substitution at each desired amino acid residue. In certain embodiments, the variants can then be screened using activity assays known to those skilled in the art. For example, in certain embodiments, the variants can be screened for their ability to bind an antibody. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided, either alone or in combination with other mutations.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested that there are a limited number of folds in a given polypeptide or protein and that once a critical number of structures have been resolved, structural prediction will become dramatically more accurate. See, e.g., Brenner et al., Curr. Op. Struct. Biol., 7(3):369-376 (1997).

Additional exemplary methods of predicting secondary structure include, but are not limited to, “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, the identity and similarity of related polypeptides can be readily calculated by known methods. Such methods include, but are not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part 1, Griffin, A. M., and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., M. Stockton Press, New York (1991); and Carillo et al., SIAM J. Applied Math., 48:1073 (1988). In certain embodiments, a substantially identical polypeptide has an amino acid sequence that is about 90 percent, or about 95 percent, or about 96 percent, or about 97 percent, or about 98 percent, or about 99 percent identical to a reference amino acid sequence.

In certain embodiments, methods to determine identity are designed to give the largest match between the sequences tested. In certain embodiments, certain methods to determine identity are described in publicly available computer programs. Certain computer program methods to determine identity between two sequences include, but are not limited to, the GCG program package, including GAP (Devereux et al., Nucl. Acid. Res., 12:387 (1984); Genetics Computer Group, University of Wisconsin, Madison, Wis., BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra (1990)). In certain embodiments, the Smith Waterman algorithm, which is known in the art, may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in certain embodiments, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix is also used by the algorithm. See, e.g., Dayhoff et al., Atlas of Protein Sequence and Structure, 5(3)(1978) for the PAM 250 comparison matrix; Henikoff et al., Proc. Natl. Acad. Sci. USA, 89:10915-10919 (1992) for the BLOSUM 62 comparison matrix.

In certain embodiments, the parameters for a polypeptide sequence comparison include the following:

Algorithm: Needleman et al., J. Mol. Biol., 48:443-453 (1970);

Comparison matrix: BLOSUM 62 from Henikoff et al., supra (1992);

Gap Penalty: 12

Gap Length Penalty: 4

Threshold of Similarity: 0

In certain embodiments, the GAP program may be useful with the above parameters. In certain embodiments, the aforementioned parameters are the default parameters for polypeptide comparisons (along with no penalty for end gaps) using the GAP algorithm.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physicochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts).

In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described, e.g., in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

The term “polypeptide fragment” as used herein refers to a polypeptide that has an amino-terminal and/or carboxy-terminal deletion. In certain embodiments, fragments are at least 2 to 1,000 amino acids long. It will be appreciated that in certain embodiments, fragments are at least 5, 6, 8, 10, 14, 20, 50, 70, 100, 150, 200, 250, 300, 350, 400, 450, 500, or 1,000 amino acids long.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res. 15:29 (1986); Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as a human antibody, but have one or more peptide linkages optionally replaced by a linkage selected from: —CH₂ NH—, —CH₂ S—, —CH₂—CH₂—, —CH═CH-(cis and trans), —COCH₂—, —CH(OH)CH₂—, and —CH₂ SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, and not limitation, by adding internal cysteine residues capable of forming intramolecular disulfide bridges which cyclize the peptide.

The term “specifically binds” refers to the ability of an antibody to bind to a target with greater affinity than it binds to a non-target. In certain embodiments, specific binding refers to binding to a target with an affinity that is at least 10, 50, 100, 250, 500, or 1000 times greater than the affinity for a non-target. In certain embodiments, affinity is determined by an affinity ELISA assay. In certain embodiments, affinity is determined by a BIAcore assay. In certain embodiments, affinity is determined by a kinetic method. In certain embodiments, affinity is determined by an equilibrium/solution method.

“Antibody” or “antibody peptide(s)” both refer to an intact antibody, or an antigen-binding fragment thereof. In certain embodiments, the antigen-binding fragment includes contiguous portions of an intact antibody. In certain embodiments, the antigen-binding fragment includes non-contiguous portions of an intact antibody. In certain embodiments, an antibody comprises a scFv. In certain embodiments, an antibody comprises a polypeptide comprising at least one CDR. In certain embodiments, an antibody comprises a polypeptide comprising at least one CDR3. In certain embodiments, an antibody comprises a polypeptide comprising at least a CDR1 domain, a CDR2 domain, and a CDR3 domain. In certain embodiments, an antibody comprises a polypeptide comprising a V_{H} domain. In certain embodiments, an antibody comprises a polypeptide comprising a V_{L} domain. In certain embodiments, an antibody comprises a polypeptide comprising a V_{H} domain and a V_{L} domain. In certain embodiments, the antibody fragment may be an antigen-binding fragment that competes with the intact antibody for specific binding. The term “antibody” also encompasses polyclonal antibodies and monoclonal antibodies. In certain embodiments, antigen-binding fragments are produced by recombinant DNA techniques. In certain embodiments, antigen-binding fragments are produced by enzymatic or chemical cleavage of intact antibodies. In certain embodiments, antigen-binding fragments are produced by recombinant DNA techniques. Antigen-binding fragments include, but are not limited to, Fab, Fab′, F(ab′)2, Fv, scFv, scFv-Fc (maxibodies), and single-chain antibodies. Non-antigen binding fragments include, but are not limited to, Fc fragments. The term “antibody” also encompasses anti-idiotypic antibodies that specifically bind to the variable region of another antibody. In certain embodiments, anti-idiotypic antibodies may be used to detect the presence of a particular antibody in a sample or to block the activity of an antibody. In addition, an “antibody” comprises all types of antibodies, fragments, and derivatives thereof described below and throughout this specification.

Certain assays for determining the specificity of an antibody are well known to the skilled artisan and include, but are not limited to, ELISA, ELISPOT, western blots, BIAcore assays, and solution affinity binding assays.

The term “isolated antibody” as used herein means an antibody which (1) is free of at least some proteins with which it would normally be found, (2) is essentially free of other proteins from the same source, e.g., from the same species, (3) is expressed by a cell from a different species, or (4) does not occur in nature.

The term “polyclonal antibody” refers to a heterogeneous mixture of antibodies that bind to different epitopes of the same antigen.

The term “monoclonal antibodies” refers to a collection of antibodies encoded by the same nucleic acid molecule. In certain embodiments, monoclonal antibodies are produced by a single hybridoma or other cell line, or by a transgenic mammal. Monoclonal antibodies typically recognize the same epitope. The term “monoclonal” is not limited to any particular method for making an antibody.

The term “CDR grafted antibody” refers to an antibody in which the CDR from one antibody is inserted into the framework of another antibody. In certain embodiments, the antibody from which the CDR is derived and the antibody from which the framework is derived are of different species. In certain embodiments, the antibody from which the CDR is derived and the antibody from which the framework is derived are of different isotypes.

The term “multi-specific antibody” refers to an antibody wherein two or more variable regions bind to different epitopes. The epitopes may be on the same or different targets. In certain embodiments, a multi-specific antibody is a “bi-specific antibody,” which recognizes two different epitopes on the same or different antigens.

The term “catalytic antibody” refers to an antibody in which one or more catalytic moieties is attached. In certain embodiments, a catalytic antibody is a cytotoxic antibody, which comprises a cytotoxic moiety.

The term “humanized antibody” refers to an antibody in which all or part of an antibody framework region is derived from a human, but all or part of one or more CDR regions is derived from another species, for example a mouse. In certain embodiments, humanization can be performed following methods known in the art (See, e.g., Jones et al., Nature 321, 522-525 (1986); Riechmann et al., Nature, 332, 323-327 (1988); Verhoeyen et al., Science 239, 1534-1536 (1988)), by substituting rodent complementarily-determining regions (CDRs) for the corresponding regions of a human antibody.

The terms “human antibody” and “fully human antibody” are used interchangeably and refer to an antibody in which both the CDR and the framework comprise substantially human sequences. In certain embodiments, fully human antibodies are produced in non-human mammals, including, but not limited to, mice, rats, and lagomorphs. In certain embodiments, fully human antibodies are produced in hybridoma cells. In certain embodiments, fully human antibodies are produced recombinantly.

“Chimeric antibody” refers to an antibody that has an antibody variable region of a first species fused to another molecule, for example, an antibody constant region of another second species. See, e.g., U.S. Pat. No. 4,816,567 and Morrison et al., Proc Natl Acad Sci (USA), 81:6851-6855 (1985). In certain embodiments, the first species may be different from the second species. In certain embodiments, the first species may be the same as the second species. In certain embodiments, chimeric antibodies may be made through mutagenesis or CDR grafting. CDR grafting typically involves grafting the CDRs from an antibody with desired specificity onto the framework regions (FRs) of another antibody.

A bivalent antibody other than a “multispecific” or “multifunctional” antibody, in certain embodiments, typically is understood to have each of its binding sites be identical.

An antibody substantially inhibits adhesion of a ligand to a receptor when an excess of antibody reduces the quantity of receptor bound to the ligand by at least about 20%, 25%, 30%, 35%, 40%, 45%, 50%, 55%, 60%, 65%, 70%, 75%, 80%, 85%, 90%, 95%, or more (as measured in an in vitro competitive binding assay).

The term “epitope” refers to a portion of a molecule capable of being bound by a specific binding agent. Exemplary epitopes may comprise any polypeptide determinant capable of specific binding to a target. Exemplary epitope determinants include, but are not limited to, chemically active surface groupings of molecules, for example, but not limited to, amino acids, sugar side chains, phosphoryl groups, and sulfonyl groups. In certain embodiments, epitope determinants may have specific three dimensional structural characteristics, and/or specific charge characteristics. In certain embodiments, an epitope is a region of an antigen that is bound by an antibody. Epitopes may be contiguous or non-contiguous. In certain embodiments, epitopes may be mimetic in that they comprise a three dimensional structure that is similar to an epitope used to generate the antibody, yet comprise none or only some of the amino acid residues found in that epitope used to generate the antibody.

The term “inhibiting and/or neutralizing epitope” refers to an epitope, which when bound by a specific binding agent results in a decrease in a biological activity in vivo, in vitro, and/or in situ. In certain embodiments, a neutralizing epitope is located on or is associated with a biologically active region of a target.

The term “activating epitope” refers to an epitope, which when bound by a specific binding agent results in activation or maintenance of a biological activity in vivo, in vitro, and/or in situ. In certain embodiments, an activating epitope is located on or is associated with a biologically active region of a target.

The term “agent” is used herein to denote a chemical, compound, a mixture of chemical compounds, a biological macromolecule, or an extract made from biological materials.

The term “pharmaceutical agent or drug” as used herein refers to a chemical compound or composition capable of inducing a desired therapeutic effect when properly administered to a patient.

The term “modulator,” as used herein, is a compound that changes or alters the activity or function of a molecule. For example, a modulator may cause an increase or decrease in the magnitude of a certain activity or function of a molecule compared to the magnitude of the activity or function observed in the absence of the modulator. In certain embodiments, a modulator is an inhibitor or antagonist, which decreases the magnitude of at least one activity or function of a molecule. In certain embodiments, a modulator is an agonist, which increases the magnitude of at least one activity or function of a molecule. Certain exemplary activities and functions of a molecule include, but are not limited to, binding affinity, enzymatic activity, and signal transduction. Certain exemplary inhibitors include, but are not limited to, proteins, peptides, antibodies, peptibodies, carbohydrates, and small organic molecules. Exemplary peptibodies are described, e.g., in WO 01/83525.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (contaminant species cannot be detected in the composition by conventional detection methods) wherein the composition consists essentially of a single macromolecular species.

The term “patient” includes human and animal subjects.

“Aggregation” refers to the formation of multimers of individual protein molecules through non-covalent or covalent interactions. Aggregation can be reversible or irreversible. In certain instances, when the loss of tertiary structure or partial unfolding occurs, hydrophobic amino acid residues which are typically hidden within the folded protein structure are exposed to the solution. In certain instances, this promotes hydrophobic-hydrophobic interactions between individual protein molecules, resulting in aggegation. Srisialam et al J Am Chem Soc 124 (9):1884-8 (2002), for example, has determined that certain conformational changes of a protein accompany aggregation, and that certain regions of specific proteins can be identified as particularly responsible for the formation of aggregates. In certain instances, protein aggregation can be induced by heat (Sun et al. J Agric Food Chem 50(6): 1636-42 (2002)), organic solvents (Srisailam et al., supra), and reagents such as SDS and lysophospholipids (Hagihara et al., Biochem 41(3): 1020-6 (2002)). Aggregation can be a significant problem in in vitro protein purification and formulation. In certain instances, after formation of aggregates, solubilization with strong denaturating solutions followed by renaturation and proper refolding may be needed before biological activity is restored.

Antibody structural units typically comprise a tetramer. Each such tetramer typically is composed of two identical pairs of polypeptide chains, each pair having one full-length “light” chain (in certain embodiments, about 25 kDa) and one full-length “heavy” chain (in certain embodiments, about 50-70 kDa). The term “heavy chain” includes any polypeptide having sufficient variable region sequence to confer specificity for a particular antigen. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carboxy-terminus. The term “heavy chain”, as used herein, encompasses a full-length antibody heavy chain and fragments thereof.

The term “light chain” includes any polypeptide having sufficient variable region sequence to confer specificity for a particular antigen. A full-length light chain includes a variable region domain, V_{L}, and a constant region domain, C_{L}. Like the heavy chain, the variable region domain of the light chain is at the amino-terminus of the polypeptide. The term “light chain”, as used herein, encompasses a full-length light chain and fragments thereof.

The amino-terminal portion of each chain typically includes a variable region (V_{H} in the heavy chain and V_{L} in the light chain) of about 100 to 110 or more amino acids that typically is responsible for antigen recognition. The carboxy-terminal portion of each chain typically defines a constant region (CH domains in the heavy chain and C_{L} in the light chain) that may be responsible for effector function. Antibody effector functions include activation of complement and stimulation of opsonophagocytosis. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology Ch. 7 (Paul, W., ed., 2nd ed. Raven Press, N.Y. (1989)). The variable regions of each light/heavy chain pair typically form the antigen binding site.

The variable regions typically exhibit the same general structure of relatively conserved framework regions (FR) joined by three hypervariable regions, also called complementarity determining regions or CDRs. The CDRs from the heavy and light chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

As discussed above, there are several types of antibody fragments. A Fab fragment is comprised of one light chain and the C_{H}1 and variable regions of one heavy chain. The heavy chain of a Fab molecule cannot form a disulfide bond with another heavy chain molecule. A Fab′ fragment contains one light chain and one heavy chain that contains more of the constant region, between the C_{H}1 and C_{H}2 domains, such that an interchain disulfide bond can be formed between two heavy chains to form a F(ab′)2 molecule. A Fab fragment is similar to a F(ab′)2 molecule, except the constant region in the heavy chains of the molecule extends to the end of the C_{H}2 domain. The Fv region comprises the variable regions from both the heavy and light chains, but lacks the constant regions. A single chain variable fragment (scFv) comprises variable regions from both a heavy and a light chain wherein the heavy and light chain variable regions are fused to form a single molecule which forms an antigen-binding region. In certain embodiments, a scFv comprises a single polypeptide chain. A single-chain antibody comprises a scFv. In certain embodiments, a single-chain antibody comprises additional polypeptides fused to the scFv, such as, for example and not limitation, one or more constant regions. Exemplary single chain antibodies are discussed, e.g., in WO 88/01649 and U.S. Pat. Nos. 4,946,778, 5,260,203, and 5,869,620. A Fc fragment contains the C_{H}2 and C_{H}3 domains of a heavy chain and contains all or part of the constant region between the C_{H}1 and C_{H}2 domains. In certain embodiments, the all or part of the constant region between the C_{H}1 and C_{H}2 domains comprises one or more cysteines which allows for formation of one or more interchain disulfide bonds between Fc fragments.

In certain embodiments, a single chain antibody is a maxibody. The term “maxibody” includes a scFv fused (may be by a linker or direct attachment) to an Fc or an Fc fragment. In certain embodiments, a single chain antibody is a maxibody that binds huEpoR (“a huEpoR maxibody”). In certain embodiments, a single chain antibody is a maxibody that binds to and activates huEpoR. Exemplary Ig-like domain-Fc fusions are disclosed in U.S. Pat. No. 6,117,655.

In certain embodiments, antibodies can be generated using alternative scaffolds. The term “alternative scaffold” refers to a framework other than the traditional antibody framework of two light chains and two heavy chains, wherein the framework can carry one or more altered amino acids and/or one or more sequence insertions (such as CDR sequences) that confer on the resulting protein the ability to specifically bind at least one target. In certain embodiments, an alternative scaffold carries one or more CDRs to generate an antibody. In certain embodiments, an alternative scaffold is based on a human protein. In certain embodiments, an alternative scaffold is based on a mammalian protein. In certain embodiments, an alternative scaffold is based on a protein from a eukaryote. In certain embodiments, an alternative scaffold is based on a prokaryotic protein.

Certain examples of antibodies with alternative scaffolds include, but are not limited to, nanobodies, affibodies, microbodies, evibodies, and domain antibodies. Certain examples of alternative scaffolds useful for creating antibodies include, but are not limited to, single domain antibodies from camelids; protease inhibitors; human serum transferrin; CTLA-4; fibronectin, including, but not limited to, the fibronectin type III domain; C-type lectin-like domains; lipocalin family proteins; ankyrin repeat proteins; the Z-domain of Protein A; γ-crystallin; Tendamistat; Neocarzinostatin; CBM4-2; the T-cell receptor; Im9; designed AR proteins; designed TPR proteins; zinc finger domains; pVIII; Avian Pancreatic Polypeptide; GCN4; WW domains; Src Homology 3 (SH3) domains; Src Homology 2 (SH2) domains; PDZ domains; TEM-1 β-lactamase; GFP; Thioredoxin; Staphylcoccal nuclease; PHD-finger domains; Cl-2; BPTI; APPI; HPSTI; Ecotin; LACI-D1; LDTI; MTI-II; scorpion toxins; Insect Defensin A Peptide; EETI-II; Min-23; CBD; PBP; Cytochrome b₅₆₂; Transferrin; LDL Receptor Domain A; and ubiquitin. Certain examples of alternative scaffolds are discussed in Hey et al., “Artifical, non-antibody binding proteins for pharmaceutical and industrial applications” Trends in Biotechnology, 23:514-22 (2005) and Binz et al., “Engineering novel binding proteins from nonimmunoglobulin domains” Nature Biotechnology, 23:1257-68 (2005).

In certain embodiments, functional domains, C_{H}1, C_{H}2, C_{H}3, and intervening sequences can be shuffled to create a different antibody constant region. For example, in certain embodiments, such hybrid constant regions can be optimized for half-life in serum, for assembly and folding of the antibody tetramer, and/or for improved effector function. In certain embodiments, modified antibody constant regions may be produced by introducing single point mutations into the amino acid sequence of the constant region and testing the resulting antibody for improved qualities, e.g., one or more of those listed above.

In certain embodiments, an antibody of one isotype is converted to a different isotype by isotype switching without losing its specificity for a particular target molecule. Methods of isotype switching include, but are not limited to, direct recombinant techniques (see e.g., U.S. Pat. No. 4,816,397) and cell-cell fusion techniques (see e.g., U.S. Pat. No. 5,916,771), among others. In certain embodiments, an antibody can be converted from one subclass to another subclass using techniques described above or otherwise known in the art without losing its specificity for a particular target molecule, including, but not limited to, conversion from an IgG2 subclass to an IgG1, IgG3, or IgG4 subclass.

In certain embodiments, chimeric antibodies that comprise at least a portion of a human sequence and another species' sequence are provided. In certain embodiments, such a chimeric antibody may result in a reduced immune response in a host than an antibody without that host's antibody sequences. For example, in certain instances, an animal of interest may be used as a model for a particular human disease. To study the effect of an antibody on that disease in the animal host, one could use an antibody from a different species. But, in certain instances, such antibodies from another species, may elicit an immune response to the antibodies themselves in the host animal, thus impeding evaluation of these antibodies. In certain embodiments, replacing part of the amino acid sequence of an antibody with antibody amino acid sequence from the host animal may decrease the magnitude of the host animal's anti-antibody response.

In certain embodiments, a chimeric antibody comprises a heavy chain and a light chain, wherein the variable regions of the light chain and the heavy chain are from a first species and the constant regions of the light chain and the heavy chain are from a second species. In certain embodiments, the antibody heavy chain constant region is an antibody heavy chain constant region of a species other than human. In certain embodiments, the antibody light chain constant region is an antibody light chain constant region of a species other than human. In certain embodiments, the antibody heavy chain constant region is a human antibody heavy chain constant region, and the antibody heavy chain variable region is an antibody heavy chain variable region of a species other than human. In certain embodiments, the antibody light chain constant region is a human antibody light chain constant region, and the antibody light chain variable region is an antibody light chain variable region of a species other than human. Exemplary antibody constant regions include, but are not limited to, a human antibody constant region, a cynomolgus monkey antibody constant region, a mouse antibody constant region, and a rabbit antibody constant region. Exemplary antibody variable regions include, but are not limited to, a human antibody variable region, a mouse antibody variable region, a pig antibody variable region, a guinea pig antibody variable region, a cynomolgus monkey antibody variable region, and a rabbit antibody variable region. In certain embodiments, the framework regions of the variable region in the heavy chain and light chain may be replaced with framework regions derived from other antibody sequences.

Certain exemplary chimeric antibodies may be produced by methods well known to those of ordinary skill in the art. In certain embodiments, the polynucleotide of the first species encoding the heavy chain variable region and the polynucleotide of the second species encoding the heavy chain constant region can be fused. In certain embodiments, the polynucleotide of the first species encoding the light chain variable region and the nucleotide sequence of the second species encoding the light chain constant region can be fused. In certain embodiments, these fused nucleotide sequences can be introduced into a cell either in a single expression vector (e.g., a plasmid) or in multiple expression vectors. In certain embodiments, a cell comprising at least one expression vector may be used to make polypeptide. In certain embodiments, these fused nucleotide sequences can be introduced into a cell either in separate expression vectors or in a single expression vector. In certain embodiments, the host cell expresses both the heavy chain and the light chain, which combine to produce an antibody. In certain embodiments, a cell comprising at least one expression vector may be used to make an antibody. Exemplary methods for producing and expressing antibodies are discussed below.

In certain embodiments, conservative modifications to the heavy and light chains of an antibody (and corresponding modifications to the encoding nucleotides) will produce antibodies having functional and chemical characteristics similar to those of the original antibody. In contrast, in certain embodiments, substantial modifications in the functional and/or chemical characteristics of an antibody to may be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

Certain desired amino acid substitutions (whether conservative or non-conservative) can be determined by those skilled in the art at the time such substitutions are desired. In certain embodiments, amino acid substitutions can be used to identify important residues of antibodies, such as those which may increase or decrease the affinity of the antibodies or the effector function of the antibodies.

Various antibodies specific to an antigen may be produced in a number of ways. In certain embodiments, an antigen containing an epitope of interest may be introduced into an animal host (e.g., a mouse), thus producing antibodies specific to that epitope. In certain instances, antibodies specific to an epitope of interest may be obtained from biological samples taken from hosts that were naturally exposed to the epitope. In certain instances, introduction of human immunoglobulin (Ig) loci into mice in which the endogenous Ig genes have been inactivated offers the opportunity to obtain human monoclonal antibodies (MAbs). In certain embodiments, antibodies specific to an epitope of interest may be obtained by in vitro screening with light and heavy chain libraries, e.g., phage display.

A bispecific or bifunctional antibody comprises two different heavy/light chain pairs and two different binding sites. Bispecific antibodies may be produced by a variety of methods including, but not limited to, fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992).

In certain embodiments, antibodies can be expressed in cell lines other than hybridoma cell lines. In certain embodiments, sequences encoding particular antibodies, including chimeric antibodies, can be used for transformation of a suitable mammalian host cell. According to certain embodiments, transformation can be by any known method for introducing polynucleotides into a host cell, including, for example packaging the polynucleotide in a virus (or into a viral vector) and transducing a host cell with the virus or by transfecting a vector using procedures known in the art, as exemplified by U.S. Pat. Nos. 4,399,216; 4,912,040; 4,740,461; and 4,959,455.

In certain embodiments, an expression vector comprises a polynucleotide sequence encoding an antibody. In certain embodiments, a method of making a polypeptide comprising producing the polypeptide in a cell comprising an expression vector in conditions suitable to express the polynucleotide contained therein to produce the polypeptide is provided.

In certain embodiments, a method of making an antibody comprising producing the antibody in a cell comprising at least one of expression vectors in conditions suitable to express the polynucleotides contained therein to produce the antibody is provided.

In certain embodiments, a scFv-Fc protein is expressed from a host cell. In certain embodiments, an scFv protein expressed from a host cell is an EREDLA. In certain embodiments, at least some of the scFv-Fc proteins expressed in a host cell form multimers, including, but not limited to, dimers. In certain embodiments, scFV-Fc proteins expressed in a host cell include monomers and multimers.

In certain embodiments, a vector is transfected into a cell. In certain embodiments, the transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, HeLa cells, baby hamster kidney (BHK) cells, monkey kidney cells (COS), human hepatocellular carcinoma cells (e.g., Hep G2), NS0 cells, SP20 cells, Per C6 cells, 293 cells, and a number of other cell lines. In certain embodiments, cell lines may be selected through determining which cell lines have high expression levels and produce antibodies with constitutive antigen binding properties.

In certain embodiments, the vectors that may be transfected into a host cell comprising control sequences that are operably linked to a polynucleotide encoding an antibody. In certain embodiments, control sequences facilitate expression of the linked polynucleotide, thus resulting in the production of the polypeptide encoded by the linked polynucleotide. In certain embodiments, the vector also comprises polynucleotide sequences that allow chromosome-independent replication in the host cell. Exemplary vectors include, but are not limited to, plasmids (e.g., BlueScript, puc, etc.), cosmids, and YACS.

In certain embodiments, an antibody is provided which comprises the sequences:

(SEQ ID NO.: 1)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWQGTLVTVSS,
and

(SEQ ID NO.: 2)

QSVLTQPPSASGSPGQSVTISCTGTSSDVGGYNYVSWYQQHPGKAPKLMI
YEVSKRPSGVPDRFSGSKSGNTASLTVSGLQPEDEADYYCSSYAGRNWVF
GGGTQLTVL.

In certain embodiments, an antibody is provided which comprises the sequences:

(SEQ ID. NO.:3)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS,
and

(SEQ ID. NO.:4)

QSALTQPASVSGSPGQSITISCTGTSSDVGGYIYVSWYQQHPGKAPKLMI
YDVSRRPSGISDRFSGSKSGNTASLTISGLQAEDEADYYCNSYTTLSTWL
FGGGTKVTVL.

In certain embodiments, an antibody is provided which comprises the sequences:

(SEQ ID. NO.:5)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSS,
and

(SEQ ID. NO.:6)

QSALTQPASVSGSPGQSIIISCTGTRSDIGGYNYVSWYQHHPGRAPKLII
FDVNNRPSGVSHRFSGSKSGNTASLTISGLQAEDEADYYCNSFTDSRTWL
FGGGTKLTVL.

In certain embodiments, an antibody is provided which comprises the sequences:

(SEQ ID. NO.:7)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSS,
and

(SEQ ID. NO.:8)

QSVLTQPPSVSEAPGQRVTIACSGSSSNIGNNAVSWYQQLPGKAPTLLIY
YDNLLPSGVSDRFSGSKSGTSASLAISGLQSEDEADYYCAAWDDSLNDWV
FGGGTKVTVL.

In certain embodiments, an antibody is provided which comprises the sequences:

(SEQ ID. NO.:9)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSA,
and

(SEQ ID. NO.:10)

QAVLTQPSSVSGAPGQRVTISCTGSSSNLGTGYDVHWYQQLPGTAPKLLI
YGNSNRPSGVPDRFSGSKSDTSGLLAITGLQAEDEATYYCQSYDFSLSAM
VFGGGTKVTVL.

In certain embodiments, an antibody is provided which comprises the sequences:

(SEQ ID NO.:56)

QVQLQQSGGGVVQPGRSLRLSCAASGFTFSDYAMHWVRQAPGKGLEWVAV
ISNHGKSTYYADSVKGRFTISRDNSKHMLYLQMNSLRADDTALYYCARDI
ALAGDYWGQGTLVTVSA,
and

(SEQ ID NO.:58)

DIQMTQSPSSLSASVGDRVTITCRASQSISSYLNWYQQLPGKVPKLLIYG
ASKLQSGVPSRFSGSGSGTDFTLTISSLQPEDFATYYCLQDYNYPLTFGP
GTRLEIK.

In certain embodiments, an antibody is provided which comprises the sequences:

(SEQ ID NO.:60)

QVQLQESGPGLVRPSGTLSLTCAVSGGSIGSSNWWSWVRQAPGKGLEWIG
EISQSGSTNYNPSLKGRVTISLDRSRNQLSLKLSSVTAADTAVYYCARQL
RSIDAFDIWGPGTTVTVSA,
and

(SEQ ID NO.:62)

SYVLTQPPSVSVSPGLTATITCSGDKLGDKYASWYQQKPGQSPVLVIYQD
RKRPSGIPERFSGSNSGNTATLTISGTQAVDEADYYCQAWDSDTSYVFGT
GTQLTVL.

In certain embodiments, an antibody is provided which comprises the sequences:

(SEQ ID NO.:64)

QVQLQESGPGLVKPSETLSLTCTVSGGYINNYYWSWIRQPPGKGLEWIGY
IHYSGSTYYNPSLKSRVTISEDTSKNQFSLKLSSATAADTAVYYCARVGY
YYDSSGYNLAWYFDLWGRGTLVTVSA,
and

(SEQ ID NO.:66)

SSELTQDPAVSVALGQTVRITCQGDNLRSYSATWYQQKPGQAPVLVLFGE
NNRPSGIPDRFSGSKSGDTAVLTITGTQTQDEADYYCTSRVNSGNHLGVF
GPGTQLTVL.

In certain embodiments, an antibody is provided which comprises the sequences:

(SEQ ID NO.:68)

EVQLVESGAEVKKPGASVKVSCKASGYTFTGYYMHWVRQAPGQGLEWMGW
INPNSGGTNYAQKFQGRVTMTRDTSISTAYMELSRLRSDDTAVYYCARGG
HMTTVTRDAFDIWGQGTMVTVSA,
and

(SEQ ID NO.:70)

SSELTQDPAVSVALGQTIRITCQGDSLRYYYATWYQQKPGQAPILVIYGQ
NNRPSGVPDRFSGSSSGNTASLTITGAQAEDEADYYCGTWDSSVSASWVF
GGGTKVTVL.

In certain embodiments, an antibody is provided which comprises the sequences:

(SEQ ID NO.:72)

QVQLQQSGAEVKKPGASVKVSCKASGYTFSGYYMHWVRQAPGQGLEWMGW
INPNSGSTNYAQKFLGRVTMTRDTSISTAYMELSSLRSDDTAVYYCARGH
SGDYFDYWGQGTLVTVSA,
and

(SEQ ID NO.:74)

EIVLTQSPSSLSASVGDRVTITCRASQSVSSWLAWYQQRPGQAPKLLIYA
ARLRGGGPSRFSGSGSGTEFTLTISSLQPEDFATYFCQQSYSTPISFGGG
TKLEIK.

In certain embodiments, an antibody is provided which comprises the sequences:

(SEQ ID NO.:76)

QVQLQESGSGLARPSQTLSLTCAVSGGSISSSAFSWNWIRQPPGKGLEWI
GYIYHTGITDYNPSLKSRVTISVDRSKNQFSLNVNSVTAADTAVYYCARG
HGSDPAWFDPWGKGTLVTVSS,
and

(SEQ ID NO.:78)

QSVLTQPPSVSVSPGQTASITCSGDKLGDKYASWYQQRPGQSPVLVIYRD
TKRPSGIPERFSGSNSGNTATLTISGTQAVDEADYYCQAWDSTTSLVFGG
GTKLTVL.

In certain embodiments, an antibody is provided which comprises the sequences:

(SEQ ID NO.:80)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGRGTMVTVSS,
and

(SEQ ID NO.:82)

QSVLTQPPSASGSPGQSVTISCTGTSSDVGGFNYVSWYQKYPGKAPKLVI
YEVSKRPSGVPDRFSGSKSGNTASLTVSGLQAEDEADYYCSSWAPGKNLF
GGGTKLTVL.

In certain embodiments, an antibody is provided which comprises the sequences:

(SEQ ID NO.:84)

EVQLVESGGGLVQPGGSLRLSCAASGFTFSSYAMSWVRQAPGKGLEWVSG
ISGSGSSEGGTYYADSVKGRFTLSRDNSKNTLYLQMNSLRAEDTALYYCV
KDRPSRYSFGYYFDYWGRGTLVTVSS,
and

(SEQ ID NO.:86)

LPVLTQPPSVSVSPGQTASIACSGNKLGDKYVSWYQQKPGQSPLLVIYQD
TKRPSGIPERFSGSNSGNTATLTISGTQAMDEADYYCQAWDSSTDVVFGG
GTKLTVL.

In certain embodiments, an antibody is provided which comprises the sequences:

(SEQ ID NO.:88)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTMVTVSS,
and

(SEQ ID NO.:90)

QSVLTQPPSASGSPGQSVTISCTGTSSDVGGYNYVSWYQQHPDKAPRLMI
YDVNKRPSGVPDRFSGSKSGNTASLTVSGLQAEDEAHYYCNSYAGSNNWV
FGGGTQLTVL.

In certain embodiments; an antibody is provided which comprises the sequences:

(SEQ ID NO.:92)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:94)

QSVLTQPPSASGSPGQSVTISCTGTSSDVGGYNYVSWYQQHPGRAPKLII
YEVSKRPSGVPDRFSGSKSGNTASLTVSGLQADDEADYYCNSYAGSIYVF
GSGTKVTVL.

In certain embodiments, an antibody is provided which comprises the sequences:

(SEQ ID NO.:96)

QVQLVQSGAEIKKPGASVKVSCKTFGSPFSTNDIHWVRQAPGQGLEWMGI
IDTSGAMTRYAQKFQGRVTVTRETSTSTVYMELSSLKSEDTAVYYCAREG
CTNGVCYDNGFDIWGQGTLVTVSS,
and

(SEQ ID NO.:98)

DIQMTQSPSTLSASIGDRVTITCRASEGIYHWLAWYQQKPGKAPKLLIYK
ASSLASGAPSRFSGSGSGTDFTLTISSLQPDDFATYYCQQYSNYPLTFGG
GTKLEIK.

In certain embodiments, an antibody is provided which comprises the sequences:

(SEQ ID NO.:100)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGRGTMVTVSS,
and

(SEQ ID NO.:102)

QSALTQPASVSGSPGQSITISCTGTSSDVGSYNLVSWYQQHPGKVPKLII
YEVSNRPSGVSHRFSGSKSGNTASLTISGLQAEDEADYYCSSLTSSGTWV
FGGGTKVTVL.

In certain embodiments, an antibody is provided which comprises the sequences:

(SEQ ID NO.:104)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:106)

QSALTQPPSASGSPGQSVTISCTGTSSDVGAYNYVSWYQQHPGKAPKLMI
YEVARRPSGVPDRFSGSKSGNTASLTVSGLQAEDEADYYCSSYAGSNNFA
VFGRGTKLTVL.

In certain embodiments, an antibody is provided which comprises the sequences:

(SEQ ID NO.:108)

EVQLVQSGGGLVQPGGSLRLSCAASGFRFSSYWMTWVRQAPGKGLEWVAN
IKPDGSEKYYVDSVKGRFTMSRDNAKNSVYLQMNSLRAEDTAVYYCARVS
RGGSFSDWGQGTLVTVSS,
and

(SEQ ID NO.:110)

QSALTQPASVSGSPGQSITIPCTGTSSDIGTYDYVSWYQQHPGKVPKVII
YEVTNRPSGVSNRFSGSKSGNTASLTISGLQADDEADYYCNSFTKNNTWV
FGGGTKLTVL.

In certain embodiments, an antibody is provided which comprises the sequences:

(SEQ ID NO.:112)

QVQLVESGGGLVQPGRSLILSCAVSGFTFSKYWMTWVRQAPGKGLEWVAN
IKPDGSEKYYVESVKGRFTISRDNAKNSVYLQMNSVRAEDTAVYYCARVS
RGGSFSDWSQGTLVTVSS,
and

(SEQ ID NO.:114)

QSALTQPPSASGSPGQSVTISCTGTSGDVGAYNYVSWYQQYPGKAPKLMI
YEVSKRPSGVPDRFSGSKSGNTASLTVSGLQAEDEADYYCNSYRGSNGPW
VFGGGTKVTVL.

In certain embodiments, a single chain variable fragment fused to an Fc is provided which comprises the sequences:

(SEQ ID. NO.:1)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS.,
and

(SEQ ID. NO.:2)

QSVLTQPPSASGSPGQSVTISCTGTSSDVGGYNYVSWYQQHPGKAPKLMI
YEVSKRPSGVPDRFSGSKSGNTASLTVSGLQPEDEADYYCSSYAGRNWVF
GGGTQLTVL.

In certain embodiments, a single chain variable fragment fused to an Fc is provided which comprises the sequences:

(SEQ ID. NO.:3)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS,
and

(SEQ ID. NO.:4)

QSALTQPASVSGSPGQSITISCTGTSSDVGGYIYVSWYQQHPGKAPKLMI
YDVSRRPSGISDRFSGSKSGNTASLTISGLQAEDEADYYCNSYTTLSTWL
FGGGTKVTVL.

In certain embodiments, a single chain variable fragment fused to an Fc is provided which comprises the sequences:

(SEQ ID. NO.:5)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSS,
and

(SEQ ID. NO.:6)

QSALTQPASVSGSPGQSIIISCTGTRSDIGGYNYVSWYQHHPGRAPKLII
FDVNNRPSGVSHRFSGSKSGNTASLTISGLQAEDEADYYCNSFTDSRTWL
FGGGTKLTVL.

In certain embodiments, a single chain variable fragment fused to an Fc is provided which comprises the sequences:

(SEQ ID. NO.:7)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSS,
and

(SEQ ID. NO.:8)

QSVLTQPPSVSEAPGQRVTIACSGSSSNIGNNAVSWYQQLPGKAPTLLIY
YDNLLPSGVSDRFSGSKSGTSASLAISGLQSEDEADYYCAAWDDSLNDWV
FGGGTKVTVL.

In certain embodiments, a single chain variable fragment fused to an Fc is provided which comprises the sequences:

(SEQ ID. NO.:9)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSA,
and

(SEQ ID. NO.:10)

QAVLTQPSSVSGAPGQRVTISCTGSSSNLGTGYDVHWYQQLPGTAPKLLI
YGNSNRPSGVPDRFSGSKSDTSGLLAITGLQAEDEATYYCQSYDFSLSAM
VFGGGTKVTVL.

In certain embodiments, a single chain variable fragment fused to an Fc is provided which comprises the sequences:

(SEQ ID NO.:56)

QVQLQQSGGGVVQPGRSLRLSCAASGFTFSDYAMHWVRQAPGKGLEWVAV
ISNHGKSTYYADSVKGRFTISRDNSKHMLYLQMNSLRADDTALYYCARDI
ALAGDYWGQGTLVTVSA,
and

(SEQ ID NO.:58)

DIQMTQSPSSLSASVGDRVTITCRASQSISSYLNWYQQLPGKVPKLLIYG
ASKLQSGVPSRFSGSGSGTDFTLTISSLQPEDFATYYCLQDYNYPLTFGP
GTRLEIK.

In certain embodiments, a single chain variable fragment fused to an Fc is provided which comprises the sequences:

(SEQ ID NO.:60)

QVQLQESGPGLVRPSGTLSLTCAVSGGSIGSSNWWSWVRQAPGKGLEWIG
EISQSGSTNYNPSLKGRVTISLDRSRNQLSLKLSSVTAADTAVYYCARQL
RSIDAFDIWGPGTTVTVSA,
and

(SEQ ID NO.:62)

SYVLTQPPSVSVSPGLTATITCSGDKLGDKYASWYQQKPGQSPVLVIYQD
RKRPSGIPERFSGSNSGNTATLTISGTQAVDEADYYCQAWDSDTSYVFGT
GTQLTVL.

In certain embodiments, a single chain variable fragment fused to an Fc is provided which comprises the sequences:

(SEQ ID NO.:64)

QVQLQESGPGLVKPSETLSLTCTVSGGYINNYYWSWIRQPPGKGLEWIGY
IHYSGSTYYNPSLKSRVTISEDTSKNQFSLKLSSATAADTAVYYCARVGY
YYDSSGYNLAWYFDLWGRGTLVTVSA,
and

(SEQ ID NO.:66)

SSELTQDPAVSVALGQTVRITCQGDNLRSYSATWYQQKPGQAPVLVLFGE
NNRPSGIPDRFSGSKSGDTAVLTITGTQTQDEADYYCTSRVNSGNHLGVE
GPGTQLTVL.

In certain embodiments, a single chain variable fragment fused to an Fc is provided which comprises the sequences:

(SEQ ID NO.:68)

EVQLVESGAEVKKPGASVKVSCKASGYTFTGYYMHWVRQAPGQGLEWMGW
INPNSGGTNYAQKFQGRVTMTRDTSISTAYMELSRLRSDDTAVYYCARGG
HMTTVTRDAFDIWGQGTMVTVSA,
and

(SEQ ID NO.:70)

SSELTQDPAVSVALGQTIRITCQGDSLRYYYATWYQQKPGQAPILVIYGQ
NNRPSGVPDRFSGSSSGNTASLTITGAQAEDEADYYCGTWDSSVSASWVF
GGGTKVTVL.

In certain embodiments, a single chain variable fragment fused to an Fc is provided which comprises the sequences:

(SEQ ID NO.:72)

QVQLQQSGAEVKKPGASVKVSCKASGYTFSGYYMHWVRQAPGQGLEWMGW
INPNSGSTNYAQKFLGRVTMTRDTSISTAYMELSSLRSDDTAVYYCARGH
SGDYFDYWGQGTLVTVSA,
and

(SEQ ID NO.:74)

EIVLTQSPSSLSASVGDRVTITCRASQSVSSWLAWYQQRPGQAPKLLIYA
ARLRGGGPSRFSGSGSGTEFTLTISSLQPEDFATYFCQQSYSTPISFGGG
TKLEIK.

In certain embodiments, a single chain variable fragment fused to an Fc is provided which comprises the sequences:

(SEQ ID NO.:76)

QVQLQESGSGLARPSQTLSLTCAVSGGSISSSAFSWNWIRQPPGKGLEWI
GYIYHTGITDYNPSLKSRVTISVDRSKNQFSLNVNSVTAADTAVYYCARG
HGSDPAWFDPWGKGTLVTVSS,
and

(SEQ ID NO.:78)

QSVLTQPPSVSVSPGQTASITCSGDKLGDKYASWYQQRPGQSPVLVIYRD
TKRPSGIPERFSGSNSGNTATLTISGTQAVDEADYYCQAWDSTTSLVFGG
GTKLTVL.

In certain embodiments, a single chain variable fragment fused to an Fc is provided which comprises the sequences:

(SEQ ID NO.:80)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGRGTMVTVSS,
and

(SEQ ID NO.:82)

QSVLTQPPSASGSPGQSVTISCTGTSSDVGGFNYVSWYQKYPGKAPKLVI
YEVSKRPSGVPDRFSGSKSGNTASLTVSGLQAEDEADYYCSSWAPGKNLF
GGGTKLTVL.

In certain embodiments, a single chain variable fragment fused to an Fc is provided which comprises the sequences:

(SEQ ID NO.:84)

EVQLVESGGGLVQPGGSLRLSCAASGFTFSSYAMSWVRQAPGKGLEWVSG
ISGSGSSEGGTYYADSVKGRFTLSRDNSKNTLYLQMNSLRAEDTALYYCV
KDRPSRYSFGYYFDYWGRGTLVTVSS,
and

(SEQ ID NO.:86)

LPVLTQPPSVSVSPGQTASIACSGNKLGDKYVSWYQQKPGQSPLLVIYQD
TKRPSGIPERFSGSNSGNTATLTISGTQAMDEADYYCQAWDSSTDVVFGG
GTKLTVL.

In certain embodiments, a single chain variable fragment fused to an Fc is provided which comprises the sequences:

(SEQ ID NO.:88)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTMVTVSS,
and

(SEQ ID NO.:90)

QSVLTQPPSASGSPGQSVTISCTGTSSDVGGYNYVSWYQQHPDKAPRLMI
YDVNKRPSGVPDRFSGSKSGNTASLTVSGLQAEDEAHYYCNSYAGSNNWV
FGGGTQLTVL.

In certain embodiments, a single chain variable fragment fused to an Fc is provided which comprises the sequences:

(SEQ ID NO.:92)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:94)

QSVLTQPPSASGSPGQSVTISCTGTSSDVGGYNYVSWYQQHPGRAPKLII
YEVSKRPSGVPDRFSGSKSGNTASLTVSGLQADDEADYYCNSYAGSIYVF
GSGTKVTVL.

In certain embodiments, a single chain variable fragment fused to an Fc is provided which comprises the sequences:

(SEQ ID NO.:96)

QVQLVQSGAEIKKPGASVKVSCKTFGSPFSTNDIHWVRQAPGQGLEWMGI
IDTSGAMTRYAQKFQGRVTVTRETSTSTVYMELSSLKSEDTAVYYCAREG
CTNGVCYDNGFDIWGQGTLVTVSS,
and

(SEQ ID NO.:98)

DIQMTQSPSTLSASIGDRVTITCRASEGIYHWLAWYQQKPGKAPKLLIYK
ASSLASGAPSRFSGSGSGTDFTLTISSLQPDDFATYYCQQYSNYPLTFGG
GTKLEIK.

In certain embodiments, a single chain variable fragment fused to an Fc is provided which comprises the sequences:

(SEQ ID NO.:100)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGRGTMVTVSS,
and

(SEQ ID NO.:102)

QSALTQPASVSGSPGQSITISCTGTSSDVGSYNLVSWYQQHPGKVPKLII
YEVSNRPSGVSHRFSGSKSGNTASLTISGLQAEDEADYYCSSLTSSGTWV
FGGGTKVTVL.

In certain embodiments, a single chain variable fragment fused to an Fc is provided which comprises the sequences:

(SEQ ID NO.:104)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS,
and

(SEQ ID NO.:106)

QSALTQPPSASGSPGQSVTISCTGTSSDVGAYNYVSWYQQHPGKAPKLMI
YEVARRPSGVPDRFSGSKSGNTASLTVSGLQAEDEADYYCSSYAGSNNFA
VFGRGTKLTVL.

In certain embodiments, a single chain variable fragment fused to an Fc is provided which comprises the sequences:

(SEQ ID NO.:108)

EVQLVQSGGGLVQPGGSLRLSCAASGFRFSSYWMTWVRQAPGKGLEWVAN
IKPDGSEKYYVDSVKGRFTMSRDNAKNSVYLQMNSLRAEDTAVYYCARVS
RGGSFSDWGQGTLVTVSS,
and

(SEQ ID NO.:110)

QSALTQPASVSGSPGQSITIPCTGTSSDIGTYDYVSWYQQHPGKVPKVII
YEVTNRPSGVSNRFSGSKSGNTASLTISGLQADDEADYYCNSFTKNNTWV
FGGGTKLTVL.

In certain embodiments, a single chain variable fragment fused to an Fc is provided which comprises the sequences:

(SEQ ID NO.:112)

QVQLVESGGGLVQPGRSLILSCAVSGFTFSKYWMTWVRQAPGKGLEWVAN
IKPDGSEKYYVESVKGRFTISRDNAKNSVYLQMNSVRAEDTAVYYCARVS
RGGSFSDWSQGTLVTVSS,
and

(SEQ ID NO.:114)

QSALTQPPSASGSPGQSVTISCTGTSGDVGAYNYVSWYQQYPGKAPKLMI
YEVSKRPSGVPDRFSGSKSGNTASLTVSGLQAEDEADYYCNSYRGSNGPW
VFGGGTKVTVL.

In certain embodiments, an antibody is provided which comprises the sequences:

| | |
|---|---|
| SYWMS; | (SEQ ID NO.:11) |
| NIKPDGSEKYYVDSVKG; | (SEQ ID NO.:12) |
| and | |
| VSRGGSYSD. | (SEQ ID NO.:13) |

In certain embodiments, an antibody is provided which comprises the sequences:

| | |
|---|---|
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

In certain embodiments, an antibody is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

In certain embodiments, an antibody is provided which comprises the sequences:

| | |
|---|---|
| TGTSSDVGGYIYVS; | (SEQ ID NO.:17) |
| DVSRRPS; | (SEQ ID NO.:18) |
| and | |
| NSYTTLSTWL; | (SEQ ID NO.:19) |

In certain embodiments, an antibody is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYIYVS; | (SEQ ID NO.:17) |
| DVSRRPS; | (SEQ ID NO.:18) |
| and | |
| NSYTTLSTWL. | (SEQ ID NO.:19) |

In certain embodiments, an antibody is provided which comprises the sequences:

| | |
|---|---|
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

In certain embodiments, an antibody is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

In certain embodiments, an antibody is provided which comprises the sequences:

| | |
|---|---|
| SYAMS; | (SEQ ID NO.:23) |
| AISGSGGSTYYADSVKG; | (SEQ ID NO.:24) |
| and | |
| DRVAVAGKGSYYFDS. | (SEQ ID NO.:25) |

In certain embodiments, an antibody is provided which comprises the sequences:

| | |
|---|---|
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

In certain embodiments, an antibody is provided which comprises the sequences:

| | |
|---|---|
| SYAMS; | (SEQ ID NO.:23) |
| AISGSGGSTYYADSVKG; | (SEQ ID NO.:24) |
| DRVAVAGKGSYYFDS; | (SEQ ID NO.:25) |
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

In certain embodiments, an antibody is provided which comprises the sequences:

| | |
|---|---|
| SNSAAWN; | (SEQ ID NO.:29) |
| RTYYRSKWYNDYAVSKS; | (SEQ ID NO.:30) |
| and | |
| DEGPLDY. | (SEQ ID NO.:31) |

In certain embodiments, an antibody is provided which comprises the sequences:

| | |
|---|---|
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

In certain embodiments, an antibody is provided which comprises the sequences:

| | |
|---|---|
| SNSAAWN; | (SEQ ID NO.:29) |
| RTYYRSKWYNDYAVSKS; | (SEQ ID NO.:30) |
| DEGPLDY; | (SEQ ID NO.:31) |
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

In certain embodiments, an antibody is provided which comprises the sequences:

| | |
|---|---|
| DYAMH; | (SEQ ID NO.:123) |
| VISNHGKSTYYADSVKG; | (SEQ ID NO.:124) |
| and | |
| DIALAGDY. | (SEQ ID NO.:125) |

In certain embodiments, an antibody is provided which comprises the sequences:

| | |
|---|---|
| RASQSISSYLN; | (SEQ ID NO.:126) |
| GASKLQS; | (SEQ ID NO.:127) |
| and | |
| LQDYNYPLT. | (SEQ ID NO.:128) |

In certain embodiments, an antibody is provided which comprises the sequences:

| | |
|---|---|
| DYAMH; | (SEQ ID NO.:123) |
| VISNHGKSTYYADSVKG; | (SEQ ID NO.:124) |
| DIALAGDY; | (SEQ ID NO.:125) |
| RASQSISSYLN; | (SEQ ID NO.:126) |
| GASKLQS; | (SEQ ID NO.:127) |
| and | |
| LQDYNYPLT. | (SEQ ID NO.:128) |

In certain embodiments, an antibody is provided which comprises the sequences:

| | |
|---|---|
| SSNWWS; | (SEQ ID NO.:129) |
| EISQSGSTNYNPSLKG; | (SEQ ID NO.:130) |
| and | |
| QLRSIDAFDI. | (SEQ ID NO.:131) |

In certain embodiments, an antibody is provided which comprises the sequences:

| | |
|---|---|
| DKYAS; | (SEQ ID NO.:132) |
| YQDRKRPSGI; | (SEQ ID NO.:133) |
| and | |
| WDSDTSYV;. | (SEQ ID NO.:134) |

In certain embodiments, an antibody is provided which comprises the sequences:

| | |
|---|---|
| SSNWWS; | (SEQ ID NO.:129) |
| EISQSGSTNYNPSLKG; | (SEQ ID NO.:130) |
| QLRSIDAFDI; | (SEQ ID NO.:131) |
| DKYAS; | (SEQ ID NO.:132) |
| YQDRKRPSGI; | (SEQ ID NO.:133) |
| and | |
| WDSDTSYV. | (SEQ ID NO.:134) |

In certain embodiments, an antibody is provided which comprises the sequences:

| | |
|---|---|
| NYYWS; | (SEQ ID NO.:135) |
| YIHYSGSTYYNPSLKSR; | (SEQ ID NO.:136) |
| and | |
| VGYYYDSSGYNLAWYFDL. | (SEQ ID NO.:212) |

In certain embodiments, an antibody is provided which comprises the sequences:

| | |
|---|---|
| QGDNLRSYSAT; | (SEQ ID NO.:137) |
| GENNRPS; | (SEQ ID NO.:138) |
| and | |
| TSRVNSGNHLGV. | (SEQ ID NO.:139) |

In certain embodiments, an antibody is provided which comprises the sequences:

| | |
|---|---|
| NYYWS; | (SEQ ID NO.:135) |
| YIHYSGSTYYNPSLKSR; | (SEQ ID NO.:136) |
| VGYYYDSSGYNLAWYFDL; | (SEQ ID NO.:212) |
| QGDNLRSYSAT; | (SEQ ID NO.:137) |
| GENNRPS; | (SEQ ID NO.:138) |
| and | |
| TSRVNSGNHLGV. | (SEQ ID NO.:139) |

In certain embodiments, an antibody is provided which comprises the sequences:

| | |
|---|---|
| GYYMH; | (SEQ ID NO.:140) |
| WINPNSGGTNYAQKFQGR; | (SEQ ID NO.:141) |
| and | |
| GGHMTTVTRDAFDI. | (SEQ ID NO.:142) |

In certain embodiments, an antibody is provided which comprises the sequences:

| | |
|---|---|
| QGDSLRYYYAT; | (SEQ ID NO.:143) |
| GQNNRPS; | (SEQ ID NO.:144) |
| and | |
| GTWDSSVSASWV. | (SEQ ID NO.:145) |

In certain embodiments, an antibody is provided which comprises the sequences:

| | |
|---|---|
| GYYMH; | (SEQ ID NO.:140) |
| WINPNSGGTNYAQKFQGR; | (SEQ ID NO.:141) |
| GGHMTTVTRDAFDI; | (SEQ ID NO.:142) |
| QGDSLRYYYAT; | (SEQ ID NO.:143) |
| GQNNRPS; | (SEQ ID NO.:144) |
| and | |
| GTWDSSVSASWV. | (SEQ ID NO.:145) |

In certain embodiments, an antibody is provided which comprises the sequences:

| | |
|---|---|
| GYYMH; | (SEQ ID NO.:146) |
| WINPNSGSTNYAQKFLG; | (SEQ ID NO.:147) |
| and | |
| GHSGDYFDY. | (SEQ ID NO.:148) |

In certain embodiments, an antibody is provided which comprises the sequences:

| | |
|---|---|
| RASQSVSSWLA; | (SEQ ID NO.:149) |
| AARLRG; | (SEQ ID NO.:150) |
| and | |
| QQSYSTPIS. | (SEQ ID NO.:151) |

In certain embodiments, an antibody is provided which comprises the sequences:

| | |
|---|---|
| GYYMH; | (SEQ ID NO.:146) |
| WINPNSGSTNYAQKFLG; | (SEQ ID NO.:147) |
| GHSGDYFDY; | (SEQ ID NO.:148) |
| RASQSVSSWLA; | (SEQ ID NO.:149) |
| AARLRG; | (SEQ ID NO.:150) |
| and | |
| QQSYSTPIS. | (SEQ ID NO.:151) |

In certain embodiments, an antibody is provided which comprises the sequences:

| | |
|---|---|
| SSAFSWN; | (SEQ ID NO.:152) |
| YIYHTGITDYNPSLKS; | (SEQ ID NO.:153) |
| and | |
| GHGSDPAWFDP. | (SEQ ID NO.:154) |

In certain embodiments, an antibody is provided which comprises the sequences:

| | |
|---|---|
| SGDKLGDKYAS; | (SEQ ID NO.:155) |
| RDTKRPS; | (SEQ ID NO.:156) |
| and | |
| QAWDSTTSLV. | (SEQ ID NO.:157) |

In certain embodiments, an antibody is provided which comprises the sequences:

| | |
|---|---|
| SSAFSWN; | (SEQ ID NO.:152) |
| YIYHTGITDYNPSLKS; | (SEQ ID NO.:153) |
| GHGSDPAWFDP; | (SEQ ID NO.:154) |
| SGDKLGDKYAS; | (SEQ ID NO.:155) |
| RDTKRPS; | (SEQ ID NO.:156) |
| and | |
| QAWDSTTSLV. | (SEQ ID NO.:157) |

In certain embodiments, an antibody is provided which comprises the sequences:

| | |
|---|---|
| SYWMS; | (SEQ ID NO.:158) |
| NIKPDGSEKYYVDSVKG; | (SEQ ID NO.:159) |
| and | |
| VSRGGSYSD. | (SEQ ID NO.:160) |

In certain embodiments, an antibody is provided which comprises the sequences:

| | |
|---|---|
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

In certain embodiments, an antibody is provided which comprises the sequences:

| | |
|---|---|
| SYWMS; | (SEQ ID NO.:158) |
| NIKPDGSEKYYVDSVKG; | (SEQ ID NO.:159) |
| VSRGGSYSD; | (SEQ ID NO.:160) |
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

In certain embodiments, an antibody is provided which comprises the sequences:

| | |
|---|---|
| SYAMS; | (SEQ ID NO.:164) |
| GISGSGSSEGGTYYADSVKG; | (SEQ ID NO.:165) |
| and | |
| DRPSRYSFGYYFDY. | (SEQ ID NO.:166) |

In certain embodiments, an antibody is provided which comprises the sequences:

| | |
|---|---|
| SGNKLGDKYVS; | (SEQ ID NO.:167) |
| QDTKRPS; | (SEQ ID NO.:168) |
| and | |
| QAWDSSTDVV. | (SEQ ID NO.:169) |

In certain embodiments, an antibody is provided which comprises the sequences:

| | |
|---|---|
| SYAMS; | (SEQ ID NO.:164) |
| GISGSGSSEGGTYYADSVKG; | (SEQ ID NO.:165) |
| DRPSRYSFGYYFDY; | (SEQ ID NO.:166) |
| SGNKLGDKYVS; | (SEQ ID NO.:167) |
| QDTKRPS; | (SEQ ID NO.:168) |
| and | |
| QAWDSSTDVV. | (SEQ ID NO.:169) |

In certain embodiments, an antibody is provided which comprises the sequences:

| | |
|---|---|
| KYWMT; | (SEQ ID NO.:170) |
| NIKPDGSEKYYVESVKG; | (SEQ ID NO.:171) |
| and | |
| VSRGGSFSD. | (SEQ ID NO.:172) |

In certain embodiments, an antibody is provided which comprises the sequences:

| | |
|---|---|
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

In certain embodiments, an antibody is provided which comprises the sequences:

| | |
|---|---|
| KYWMT; | (SEQ ID NO.:170) |
| NIKPDGSEKYYVESVKG; | (SEQ ID NO.:171) |
| VSRGGSFSD; | (SEQ ID NO.:172) |
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

In certain embodiments, an antibody is provided which comprises the sequences:

| | |
|---|---|
| KYWMT; | (SEQ ID NO.:176) |
| NIKPDGSEKYYVESVKG; | (SEQ ID NO.:177) |
| and | |
| VSRGGSFSD. | (SEQ ID NO.:178) |

In certain embodiments, an antibody is provided which comprises the sequences:

| | |
|---|---|
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

In certain embodiments, an antibody is provided which comprises the sequences:

| | |
|---|---|
| KYWMT; | (SEQ ID NO.:176) |
| NIKPDGSEKYYVESVKG; | (SEQ ID NO.:177) |
| VSRGGSFSD; | (SEQ ID NO.:178) |
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

In certain embodiments, an antibody is provided which comprises the sequences:

| | |
|---|---|
| TNDIH; | (SEQ ID NO.:182) |
| IIDTSGAMTRYAQKFQG; | (SEQ ID NO.:183) |
| and | |
| EGCTNGVCYDNGFDI. | (SEQ ID NO.:184) |

In certain embodiments, an antibody is provided which comprises the sequences:

| | |
|---|---|
| RASEGIYHWLA; | (SEQ ID NO.:185) |
| KASSLAS; | (SEQ ID NO.:186) |
| and | |
| QQYSNYPLT. | (SEQ ID NO.:187) |

In certain embodiments, an antibody is provided which comprises the sequences:

| | |
|---|---|
| TNDIH; | (SEQ ID NO.:182) |
| IIDTSGAMTRYAQKFQG; | (SEQ ID NO.:183) |
| EGCTNGVCYDNGFDI; | (SEQ ID NO.:184) |
| RASEGIYHWLA; | (SEQ ID NO.:185) |
| KASSLAS; | (SEQ ID NO.:186) |
| and | |
| QQYSNYPLT. | (SEQ ID NO.:187) |

In certain embodiments, an antibody is provided which comprises the sequences:

| | |
|---|---|
| KYWMT; | (SEQ ID NO.:188) |
| NIKPDGSEKYYVESVKG; | (SEQ ID NO.:189) |
| and | |
| VSRGGSFSD. | (SEQ ID NO.:190) |

In certain embodiments, an antibody is provided which comprises the sequences:

| | |
|---|---|
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

In certain embodiments, an antibody is provided which comprises the sequences:

| | |
|---|---|
| KYWMT; | (SEQ ID NO.:188) |
| NIKPDGSEKYYVESVKG; | (SEQ ID NO.:189) |
| VSRGGSFSD; | (SEQ ID NO.:190) |
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

In certain embodiments, an antibody is provided which comprises the sequences:

| | |
|---|---|
| KYWMT; | (SEQ ID NO.:194) |
| NIKPDGSEKYYVESVKG; | (SEQ ID NO.:195) |
| and | |
| VSRGGSFSD. | (SEQ ID NO.:196) |

In certain embodiments, an antibody is provided which comprises the sequences:

| | |
|---|---|
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

In certain embodiments, an antibody is provided which comprises the sequences:

| | |
|---|---|
| KYWMT; | (SEQ ID NO.:194) |
| NIKPDGSEKYYVESVKG; | (SEQ ID NO.:195) |
| VSRGGSFSD; | (SEQ ID NO.:196) |
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

In certain embodiments, an antibody is provided which comprises the sequences:

| | |
|---|---|
| SYWMT; | (SEQ ID NO.:200) |
| NIKPDGSEKYYVDSVKG; | (SEQ ID NO.:201) |
| and | |
| VSRGGSFSD. | (SEQ ID NO.:202) |

In certain embodiments, an antibody is provided which comprises the sequences:

| | |
|---|---|
| TGTSSDIGTYDYVS; | (SEQ ID NO.:203) |
| EVTNRPS; | (SEQ ID NO.:204) |
| and | |
| NSFTKNNTWV. | (SEQ ID NO.:205) |

In certain embodiments, an antibody is provided which comprises the sequences:

| | |
|---|---|
| SYWMT; | (SEQ ID NO.:200) |
| NIKPDGSEKYYVDSVKG; | (SEQ ID NO.:201) |
| VSRGGSFSD; | (SEQ ID NO.:202) |
| TGTSSDIGTYDYVS; | (SEQ ID NO.:203) |
| EVTNRPS; | (SEQ ID NO.:204) |
| and | |
| NSFTKNNTWV. | (SEQ ID NO.:205) |

In certain embodiments, an antibody is provided which comprises the sequences:

| | |
|---|---|
| KYWMT; | (SEQ ID NO.:206) |
| NIKPDGSEKYYVESVKG; | (SEQ ID NO.:207) |
| and | |
| VSRGGSFSD. | (SEQ ID NO.:208) |

In certain embodiments, an antibody is provided which comprises the sequences:

| | |
|---|---|
| TGTSGDVGAYNYVS; | (SEQ ID NO.:209) |
| EVSKRPS; | (SEQ ID NO.:210) |
| and | |
| NSYRGSNGPWV. | (SEQ ID NO.:211) |

In certain embodiments, an antibody is provided which comprises the sequences:

| | |
|---|---|
| KYWMT; | (SEQ ID NO.:206) |
| NIKPDGSEKYYVESVKG; | (SEQ ID NO.:207) |
| VSRGGSFSD; | (SEQ ID NO.:208) |
| TGTSGDVGAYNYVS; | (SEQ ID NO.:209) |
| EVSKRPS; | (SEQ ID NO.:210) |
| and | |
| NSYRGSNGPWV. | (SEQ ID NO.:211) |

In certain embodiments, a single chain variable fragment fused to an Fc is provided which comprises the sequences:

| | |
|---|---|
| SYWMS; | (SEQ ID NO.:11) |
| NIKPDGSEKYYVDSVKG; | (SEQ ID NO.:12) |
| and | |
| VSRGGSYSD. | (SEQ ID NO.:13) |

In certain embodiments, a single chain variable fragment fused to an Fc is provided which comprises the sequences:

| | |
|---|---|
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

In certain embodiments, a single chain variable fragment fused to an Fc is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYNYVS; | (SEQ ID NO.:14) |
| EVSKRPS; | (SEQ ID NO.:15) |
| and | |
| SSYAGRNWV. | (SEQ ID NO.:16) |

In certain embodiments, a single chain variable fragment fused to an Fc is provided which comprises the sequences:

| | |
|---|---|
| TGTSSDVGGYIYVS; | (SEQ ID NO.:17) |
| DVSRRPS; | (SEQ ID NO.:18) |
| and | |
| NSYTTLSTWL. | (SEQ ID NO.:19) |

In certain embodiments, a single chain variable fragment fused to an Fc is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTSSDVGGYIYVS; | (SEQ ID NO.:17) |
| DVSRRPS; | (SEQ ID NO.:18) |
| and | |
| NSYTTLSTWL. | (SEQ ID NO.:19) |

In certain embodiments, a single chain variable fragment fused to an Fc is provided which comprises the sequences:

| | |
|---|---|
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

In certain embodiments, a single chain variable fragment fused to an Fc is provided which comprises the sequences:

| | |
|---|---|
| SYWMS; | (SEQ ID NO.:11) |
| NIKPDGSEKYYVDSVKG; | (SEQ ID NO.:12) |
| VSRGGSYSD; | (SEQ ID NO.:13) |
| TGTRSDIGGYNYVS; | (SEQ ID NO.:20) |
| FDVNNRPS; | (SEQ ID NO.:21) |
| and | |
| NSFTDSRTWL. | (SEQ ID NO.:22) |

In certain embodiments, a single chain variable fragment fused to an Fc is provided which comprises the sequences:

| | |
|---|---|
| SYAMS; | (SEQ ID NO.:23) |
| AISGSGGSTYYADSVKG; | (SEQ ID NO.:24) |
| and | |
| DRVAVAGKGSYYFDS. | (SEQ ID NO.:25) |

In certain embodiments, a single chain variable fragment fused to an Fc is provided which comprises the sequences:

| | |
|---|---|
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

In certain embodiments, a single chain variable fragment fused to an Fc is provided which comprises the sequences:

| | |
|---|---|
| SYAMS; | (SEQ ID NO.:23) |
| AISGSGGSTYYADSVKG; | (SEQ ID NO.:24) |
| DRVAVAGKGSYYFDS; | (SEQ ID NO.:25) |
| SGSSSNIGNNAVS; | (SEQ ID NO.:26) |
| YDNLLPSG; | (SEQ ID NO.:27) |
| and | |
| AAWDDSLNDWV. | (SEQ ID NO.:28) |

In certain embodiments, a single chain variable fragment fused to an Fc is provided which comprises the sequences:

| | |
|---|---|
| SNSAAWN; | (SEQ ID NO.:29) |
| RTYYRSKWYNDYAVSKS; | (SEQ ID NO.:30) |
| and | |
| DEGPLDY. | (SEQ ID NO.:31) |

In certain embodiments, a single chain variable fragment fused to an Fc is provided which comprises the sequences:

| | |
|---|---|
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

In certain embodiments, a single chain variable fragment fused to an Fc is provided which comprises the sequences:

| | |
|---|---|
| SNSAAWN; | (SEQ ID NO.:29) |
| RTYYRSKWYNDYAVSKS; | (SEQ ID NO.:30) |
| DEGPLDY; | (SEQ ID NO.:31) |
| TGSSSNLGTGYDVH; | (SEQ ID NO.:32) |
| GNSNRPS; | (SEQ ID NO.:33) |
| and | |
| QSYDFSLSAMV. | (SEQ ID NO.:34) |

In certain embodiments, a single chain variable fragment fused to an Fc is provided which comprises the sequences:

| | |
|---|---|
| DYAMH; | (SEQ ID NO.:123) |
| VISNHGKSTYYADSVKG; | (SEQ ID NO.:124) |
| and | |
| DIALAGDY. | (SEQ ID NO.:125) |

In certain embodiments, a single chain variable fragment fused to an Fc is provided which comprises the sequences:

| | |
|---|---|
| RASQSISSYLN; | (SEQ ID NO.:126) |
| GASKLQS; | (SEQ ID NO.:127) |
| and | |
| LQDYNYPLT. | (SEQ ID NO.:128) |

In certain embodiments, a single chain variable fragment fused to an Fc is provided which comprises the sequences:

| | |
|---|---|
| DYAMH; | (SEQ ID NO.:123) |
| VISNHGKSTYYADSVKG; | (SEQ ID NO.:124) |
| DIALAGDY; | (SEQ ID NO.:125) |
| RASQSISSYLN; | (SEQ ID NO.:126) |
| GASKLQS; | (SEQ ID NO.:127) |
| and | |
| LQDYNYPLT. | (SEQ ID NO.:128) |

In certain embodiments, a single chain variable fragment fused to an Fc is provided which comprises the sequences:

| | |
|---|---|
| SSNWWS; | (SEQ ID NO.:129) |
| EISQSGSTNYNPSLKG; | (SEQ ID NO.:130) |
| and | |
| QLRSIDAFDI. | (SEQ ID NO.:131) |

In certain embodiments, a single chain variable fragment fused to an Fc is provided which comprises the sequences:

| | |
|---|---|
| DKYAS; | (SEQ ID NO.:132) |
| YQDRKRPSGI; | (SEQ ID NO.:133) |
| and | |
| WDSDTSYV;. | (SEQ ID NO.:134) |

In certain embodiments, a single chain variable fragment fused to an Fc is provided which comprises the sequences:

| | |
|---|---|
| SSNWWS; | (SEQ ID NO.:129) |
| EISQSGSTNYNPSLKG; | (SEQ ID NO.:130) |
| QLRSIDAFDI; | (SEQ ID NO.:131) |
| DKYAS; | (SEQ ID NO.:132) |
| YQDRKRPSGI; | (SEQ ID NO.:133) |
| and | |
| WDSDTSYV. | (SEQ ID NO.:134) |

In certain embodiments, a single chain variable fragment fused to an Fc is provided which comprises the sequences:

| | |
|---|---|
| NYYWS; | (SEQ ID NO.:135) |
| YIHYSGSTYYNPSLKSR; | (SEQ ID NO.:136) |
| and | |
| VGYYYDSSGYNLAWYFDL. | (SEQ ID NO.:212) |

In certain embodiments, a single chain variable fragment fused to an Fc is provided which comprises the sequences:

| | |
|---|---|
| QGDNLRSYSAT; | (SEQ ID NO.:137) |
| GENNRPS; | (SEQ ID NO.:138) |
| and | |
| TSRVNSGNHLGV. | (SEQ ID NO.:139) |

In certain embodiments, a single chain variable fragment fused to an Fc is provided which comprises the sequences:

| | |
|---|---|
| NYYWS; | (SEQ ID NO.:135) |
| YIHYSGSTYYNPSLKSR; | (SEQ ID NO.:136) |
| VGYYYDSSGYNLAWYFDL; | (SEQ ID NO.:212) |
| QGDNLRSYSAT; | (SEQ ID NO.:137) |
| GENNRPS; | (SEQ ID NO.:138) |
| and | |
| TSRVNSGNHLGV. | (SEQ ID NO.:139) |

In certain embodiments, a single chain variable fragment fused to an Fc is provided which comprises the sequences:

| | |
|---|---|
| GYYMH; | (SEQ ID NO.:140) |
| WINPNSGGTNYAQKFQGR; | (SEQ ID NO.:141) |
| and | |
| GGHMTTVTRDAFDI. | (SEQ ID NO.:142) |

In certain embodiments, a single chain variable fragment fused to an Fc is provided which comprises the sequences:

| | |
|---|---|
| QGDSLRYYYAT; | (SEQ ID NO.:143) |
| GQNNRPS; | (SEQ ID NO.:144) |
| and | |
| GTWDSSVSASWV. | (SEQ ID NO.:145) |

In certain embodiments, a single chain variable fragment fused to an Fc is provided which comprises the sequences:

| | |
|---|---|
| GYYMH; | (SEQ ID NO.:140) |
| WINPNSGGTNYAQKFQGR; | (SEQ ID NO.:141) |
| GGHMTTVTRDAFDI; | (SEQ ID NO.:142) |
| QGDSLRYYYAT; | (SEQ ID NO.:143) |
| GQNNRPS; | (SEQ ID NO.:144) |
| and | |
| GTWDSSVSASWV. | (SEQ ID NO.:145) |

In certain embodiments, a single chain variable fragment fused to an Fc is provided which comprises the sequences:

| | |
|---|---|
| GYYMH; | (SEQ ID NO.:146) |
| WINPNSGSTNYAQKFLG; | (SEQ ID NO.:147) |
| and | |
| GHSGDYFDY. | (SEQ ID NO.:148) |

In certain embodiments, a single chain variable fragment fused to an Fc is provided which comprises the sequences:

| | |
|---|---|
| RASQSVSSWLA; | (SEQ ID NO.:149) |
| AARLRG; | (SEQ ID NO.:150) |
| and | |
| QQSYSTPIS. | (SEQ ID NO.:151) |

In certain embodiments, a single chain variable fragment fused to an Fc is provided which comprises the sequences:

| | |
|---|---|
| GYYMH; | (SEQ ID NO.:146) |
| WINPNSGSTNYAQKFLG; | (SEQ ID NO.:147) |
| GHSGDYFDY; | (SEQ ID NO.:148) |
| RASQSVSSWLA; | (SEQ ID NO.:149) |
| AARLRG; | (SEQ ID NO.:150) |
| and | |
| QQSYSTPIS. | (SEQ ID NO.:151) |

In certain embodiments, a single chain variable fragment fused to an Fc is provided which comprises the sequences:

| | |
|---|---|
| SSAFSWN; | (SEQ ID NO.:152) |
| YIYHTGITDYNPSLKS; | (SEQ ID NO.:153) |
| and | |
| GHGSDPAWFDP. | (SEQ ID NO.:154) |

In certain embodiments, a single chain variable fragment fused to an Fc is provided which comprises the sequences:

| | |
|---|---|
| SGDKLGDKYAS; | (SEQ ID NO.:155) |
| RDTKRPS; | (SEQ ID NO.:156) |
| and | |
| QAWDSTTSLV. | (SEQ ID NO.:157) |

In certain embodiments, a single chain variable fragment fused to an Fc is provided which comprises the sequences:

| | |
|---|---|
| SSAFSWN; | (SEQ ID NO.:152) |
| YIYHTGITDYNPSLKS; | (SEQ ID NO.:153) |
| GHGSDPAWFDP; | (SEQ ID NO.:154) |
| SGDKLGDKYAS; | (SEQ ID NO.:155) |
| RDTKRPS; | (SEQ ID NO.:156) |
| and | |
| QAWDSTTSLV. | (SEQ ID NO.:157) |

In certain embodiments, a single chain variable fragment fused to an Fc is provided which comprises the sequences:

| | |
|---|---|
| SYWMS; | (SEQ ID NO.:158) |
| NIKPDGSEKYYVDSVKG; | (SEQ ID NO.:159) |
| and | |
| VSRGGSYSD. | (SEQ ID NO.:160) |

In certain embodiments, a single chain variable fragment fused to an Fc is provided which comprises the sequences:

| | |
|---|---|
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

In certain embodiments, a single chain variable fragment fused to an Fc is provided which comprises the sequences:

| | |
|---|---|
| SYWMS; | (SEQ ID NO.:158) |
| NIKPDGSEKYYVDSVKG; | (SEQ ID NO.:159) |
| VSRGGSYSD; | (SEQ ID NO.:160) |
| TGTSSDVGGFNYVS; | (SEQ ID NO.:161) |
| EVSKRPS; | (SEQ ID NO.:162) |
| and | |
| SSWAPGKNL. | (SEQ ID NO.:163) |

In certain embodiments, a single chain variable fragment fused to an Fc is provided which comprises the sequences:

| | |
|---|---|
| SYAMS; | (SEQ ID NO.:164) |
| GISGSGSSEGGTYYADSVKG; | (SEQ ID NO.:165) |
| and | |
| DRPSRYSFGYYFDY. | (SEQ ID NO.:166) |

In certain embodiments, a single chain variable fragment fused to an Fc is provided which comprises the sequences:

| | |
|---|---|
| SGNKLGDKYVS; | (SEQ ID NO.:167) |
| QDTKRPS; | (SEQ ID NO.:168) |
| and | |
| QAWDSSTDVV. | (SEQ ID NO.:169) |

In certain embodiments, a single chain variable fragment fused to an Fc is provided which comprises the sequences:

| | |
|---|---|
| SYAMS; | (SEQ ID NO.:164) |
| GISGSGSSEGGTYYADSVKG; | (SEQ ID NO.:165) |
| DRPSRYSFGYYFDY; | (SEQ ID NO.:166) |
| SGNKLGDKYVS; | (SEQ ID NO.:167) |
| QDTKRPS; | (SEQ ID NO.:168) |
| and | |
| QAWDSSTDVV. | (SEQ ID NO.:169) |

In certain embodiments, a single chain variable fragment fused to an Fc is provided which comprises the sequences:

| | |
|---|---|
| KYWMT; | (SEQ ID NO.:170) |
| NIKPDGSEKYYVESVKG; | (SEQ ID NO.:171) |
| and | |
| VSRGGSFSD. | (SEQ ID NO.:172) |

In certain embodiments, a single chain variable fragment fused to an Fc is provided which comprises the sequences:

| | |
|---|---|
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

In certain embodiments, a single chain variable fragment fused to an Fc is provided which comprises the sequences:

| | |
|---|---|
| KYWMT; | (SEQ ID NO.:170) |
| NIKPDGSEKYYVESVKG; | (SEQ ID NO.:171) |
| VSRGGSFSD; | (SEQ ID NO.:172) |
| TGTSSDVGGYNYVS; | (SEQ ID NO.:173) |
| DVNKRPS; | (SEQ ID NO.:174) |
| and | |
| NSYAGSNNWV. | (SEQ ID NO.:175) |

In certain embodiments, a single chain variable fragment fused to an Fc is provided which comprises the sequences:

| | |
|---|---|
| KYWMT; | (SEQ ID NO.:176) |
| NIKPDGSEKYYVESVKG; | (SEQ ID NO.:177) |
| and | |
| VSRGGSFSD. | (SEQ ID NO.:178) |

In certain embodiments, a single chain variable fragment fused to an Fc is provided which comprises the sequences:

| | |
|---|---|
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

In certain embodiments, a single chain variable fragment fused to an Fc is provided which comprises the sequences:

| | |
|---|---|
| KYWMT; | (SEQ ID NO.:176) |
| NIKPDGSEKYYVESVKG; | (SEQ ID NO.:177) |
| VSRGGSFSD; | (SEQ ID NO.:178) |
| TGTSSDVGGYNYVS; | (SEQ ID NO.:179) |
| EVSKRPS; | (SEQ ID NO.:180) |
| and | |
| NSYAGSIYV. | (SEQ ID NO.:181) |

In certain embodiments, a single chain variable fragment fused to an Fc is provided which comprises the sequences:

| | |
|---|---|
| TNDIH; | (SEQ ID NO.:182) |
| IIDTSGAMTRYAQKFQG; | (SEQ ID NO.:183) |
| and | |
| EGCTNGVCYDNGFDI. | (SEQ ID NO.:184) |

In certain embodiments, a single chain variable fragment fused to an Fc is provided which comprises the sequences:

| | |
|---|---|
| RASEGIYHWLA; | (SEQ ID NO.:185) |
| KASSLAS; | (SEQ ID NO.:186) |
| and | |
| QQYSNYPLT. | (SEQ ID NO.:187) |

In certain embodiments, a single chain variable fragment fused to an Fc is provided which comprises the sequences:

| | |
|---|---|
| TNDIH; | (SEQ ID NO.:182) |
| IIDTSGAMTRYAQKFQG; | (SEQ ID NO.:183) |
| EGCTNGVCYDNGFDI; | (SEQ ID NO.:184) |
| RASEGIYHWLA; | (SEQ ID NO.:185) |
| KASSLAS; | (SEQ ID NO.:186) |
| and | |
| QQYSNYPLT. | (SEQ ID NO.:187) |

In certain embodiments, a single chain variable fragment fused to an Fc is provided which comprises the sequences:

| | |
|---|---|
| KYWMT; | (SEQ ID NO.:188) |
| NIKPDGSEKYYVESVKG; | (SEQ ID NO.:189) |
| and | |
| VSRGGSFSD. | (SEQ ID NO.:190) |

In certain embodiments, a single chain variable fragment fused to an Fc is provided which comprises the sequences:

| | |
|---|---|
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

In certain embodiments, a single chain variable fragment fused to an Fc is provided which comprises the sequences:

| | |
|---|---|
| KYWMT; | (SEQ ID NO.:188) |
| NIKPDGSEKYYVESVKG; | (SEQ ID NO.:189) |
| VSRGGSFSD; | (SEQ ID NO.:190) |
| TGTSSDVGSYNLVS; | (SEQ ID NO.:191) |
| EVSNRPS; | (SEQ ID NO.:192) |
| and | |
| SSLTSSGTWV. | (SEQ ID NO.:193) |

In certain embodiments, a single chain variable fragment fused to an Fc is provided which comprises the sequences:

| | |
|---|---|
| KYWMT; | (SEQ ID NO.:194) |
| NIKPDGSEKYYVESVKG; | (SEQ ID NO.:195) |
| and | |
| VSRGGSFSD. | (SEQ ID NO.:196) |

In certain embodiments, a single chain variable fragment fused to an Fc is provided which comprises the sequences:

| | |
|---|---|
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

In certain embodiments, a single chain variable fragment fused to an Fc is provided which comprises the sequences:

| | |
|---|---|
| KYWMT; | (SEQ ID NO.:194) |
| NIKPDGSEKYYVESVKG; | (SEQ ID NO.:195) |
| VSRGGSFSD; | (SEQ ID NO.:196) |
| TGTSSDVGAYNYVS; | (SEQ ID NO.:197) |
| EVARRPS; | (SEQ ID NO.:198) |
| and | |
| SSYAGSNNFAV. | (SEQ ID NO.:199) |

In certain embodiments, a single chain variable fragment fused to an Fc is provided which comprises the sequences:

| | |
|---|---|
| SYWMT; | (SEQ ID NO.:200) |
| NIKPDGSEKYYVDSVKG; | (SEQ ID NO.:201) |
| and | |
| VSRGGSFSD. | (SEQ ID NO.:202) |

In certain embodiments, a single chain variable fragment fused to an Fc is provided which comprises the sequences:

| | |
|---|---|
| TGTSSDIGTYDYVS; | (SEQ ID NO.:203) |
| EVTNRPS; | (SEQ ID NO.:204) |
| and | |
| NSFTKNNTWV. | (SEQ ID NO.:205) |

In certain embodiments, a single chain variable fragment fused to an Fc is provided which comprises the sequences:

| | |
|---|---|
| SYWMT; | (SEQ ID NO.: 200) |
| NIKPDGSEKYYVDSVKG; | (SEQ ID NO.: 201) |
| VSRGGSFSD; | (SEQ ID NO.: 202) |
| TGTSSDIGTYDYVS; | (SEQ ID NO.: 203) |
| EVTNRPS; | (SEQ ID NO.: 204) |
| and | |
| NSFTKNNTWV. | (SEQ ID NO.: 205) |

In certain embodiments, a single chain variable fragment fused to an Fc is provided which comprises the sequences:

| | |
|---|---|
| KYWMT; | (SEQ ID NO.: 206) |
| NIKPDGSEKYYVESVKG; | (SEQ ID NO.: 207) |
| and | |
| VSRGGSFSD. | (SEQ ID NO.: 208) |

In certain embodiments, a single chain variable fragment fused to an Fc is provided which comprises the sequences:

| | |
|---|---|
| TGTSGDVGAYNYVS; | (SEQ ID NO.: 209) |
| EVSKRPS; | (SEQ ID NO.: 210) |
| and | |
| NSYRGSNGPWV. | (SEQ ID NO.: 211). |

In certain embodiments, a single chain variable fragment fused to an Fc is provided which comprises the sequences:

| | |
|---|---|
| KYWMT; | (SEQ ID NO.: 206) |
| NIKPDGSEKYYVESVKG; | (SEQ ID NO.: 207) |
| VSRGGSFSD; | (SEQ ID NO.: 208) |
| TGTSGDVGAYNYVS; | (SEQ ID NO.: 209) |
| EVSKRPS; | (SEQ ID NO.: 210) |
| and | |
| NSYRGSNGPWV. | (SEQ ID NO.: 211) |

In certain embodiments, an antibody is provided which comprises the sequence:

(SEQ ID NO.: 45)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSVLTQPPSASGSPGQ
SVTISCTGTSSDVGGYNYVSWYQQHPGKAPKLMIYEVSKRPSGVPDRFSG
SKSGNTASLTVSGLQPEDEADYYCSSYAGRNWVFGGGTQLTVLGAAAEPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK.

In certain embodiments, an antibody is provided which comprises the sequence:

(SEQ ID NO.: 46)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSALTQPASVSGSPGQ
SITISCTGTSSDVGGYIYVSWYQQHPGKAPKLMIYDVSRRPSGISDRFSG
SKSGNTASLTISGLQAEDEADYYCNSYTTLSTWLFGGGTKVTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In certain embodiments, an antibody is provided which comprises the sequence:

(SEQ ID NO.: 47)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSSGGGGSGGGGSGGGGSAQSALTQPASVSGSPGQ
SIIISCTGTRSDIGGYNYVSWYQHHPGRAPKLIIFDVNNRPSGVSHRFSG
SKSGNTASLTISGLQAEDEADYYCNSFTDSRTWLFGGGTKLTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In certain embodiments, an antibody is provided which comprises the sequence:

(SEQ ID NO.: 48)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSSGGGGSGGGGSGGGGSAQSVLTQPPSV
SEAPGQRVTIACSGSSSNIGNNAVSWYQQLPGKAPTLLIYYDNLLPSGVS
DRFSGSKSGTSASLAISGLQSEDEADYYCAAWDDSLNDWVFGGGTKVTVL
GAAAEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLT
VDKSRWQQGNVFSCSVMHEALHNHYTQKSLSLSPGK.

In certain embodiments, an antibody is provided which comprises the sequence:

(SEQ ID NO.: 49)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSAGGGGSGGGGSGGGGSGAPQAVLTQPSSVSGA
PGQRVTISCTGSSSNLGTGYDVHWYQQLPGTAPKLLIYGNSNRPSGVPDR
FSGSKSDTSGLLAITGLQAEDEATYYCQSYDFSLSAMVFGGGTKVTVLAA
AEPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDK
SRWQQGNVFSCSVMHEALHNHYTQKSLSLSPGK.

In certain embodiments, a single chain variable fragment fused to an Fc is provided which specifically binds to amino acids F93 and H114 of the extracellular domain of the human Epo Receptor.

In certain embodiments, a single chain variable fragment fused to an Fc is provided which specifically binds to amino acids S91, F93, and H114 of the extracellular domain of the human Epo Receptor.

In certain embodiments, a single chain variable fragment fused to an Fc is provided which specifically binds to amino acid F93 of the extracellular domain of the human Epo Receptor.

In certain embodiments, a single chain variable fragment fused to an Fc is provided which specifically binds to amino acids E62, F93, and M150 of the extracellular domain of the human Epo Receptor.

In certain embodiments, a single chain variable fragment fused to an Fc is provided which specifically binds to amino acids V48, E62, L66, R68, and H70 of the extracellular domain of the human Epo Receptor.

In certain embodiments, a single chain variable fragment fused to an Fc is provided which specifically binds to amino acids V48, W64, L66, R68, and H70 of the extracellular domain of the human Epo Receptor.

In certain embodiments, a single chain variable fragment fused to an Fc is provided which specifically binds to amino acids A44, V48, P63, L66, R68, and H70 of the extracellular domain of the human Epo Receptor.

In certain embodiments, a single chain variable fragment fused to an Fc is provided which specifically binds to amino acids L66 and R99 of the extracellular domain of the human Epo Receptor.

In certain embodiments, an antibody is provided which specifically binds to amino acids F93 and H114 of the extracellular domain of the human Epo Receptor.

In certain embodiments, an antibody is provided which specifically binds to amino acids S91, F93, and H114 of the extracellular domain of the human Epo Receptor.

In certain embodiments, an antibody is provided which specifically binds to amino acid F93 of the extracellular domain of the human Epo Receptor.

In certain embodiments, an antibody is provided which specifically binds to amino acids E62, F93, and M150 of the extracellular domain of the human Epo Receptor.

In certain embodiments, an antibody is provided which specifically binds to amino acids V48, E62, L66, R68, and H70 of the extracellular domain of the human Epo Receptor.

In certain embodiments, an antibody is provided which specifically binds to amino acids V48, W64, L66, R68, and H70 of the extracellular domain of the human Epo Receptor.

In certain embodiments, an antibody is provided which specifically binds to amino acids A44, V48, P63, L66, R68, and H70 of the extracellular domain of the human Epo Receptor.

In certain embodiments, an antibody is provided which specifically binds to amino acids L66 and R99 of the extracellular domain of the human Epo Receptor.

In certain embodiments, a single chain variable fragment fused to an Fc is provided which specifically binds to amino acids F93, E60, and H114 of the extracellular domain of the human Epo Receptor.

In certain embodiments, a single chain variable fragment fused to an Fc is provided which specifically binds to amino acid V48 of the extracellular domain of the human Epo Receptor.

In certain embodiments, a single chain variable fragment fused to an Fc is provided which specifically binds to amino acid L66 of the extracellular domain of the human Epo Receptor.

In certain embodiments, a single chain variable fragment fused to an Fc is provided which specifically binds to amino acid W64 of the extracellular domain of the human Epo Receptor.

In certain embodiments, a single chain variable fragment fused to an Fc is provided which specifically binds to amino acid H70 of the extracellular domain of the human Epo Receptor.

In certain embodiments, a single chain variable fragment fused to an Fc is provided which specifically binds to amino acids V48 and W64 of the extracellular domain of the human Epo Receptor.

In certain embodiments, a single chain variable fragment fused to an Fc is provided which specifically binds to amino acids V48 and L66 of the extracellular domain of the human Epo Receptor.

In certain embodiments, a single chain variable fragment fused to an Fc is provided which specifically binds to amino acids V48 and R68 of the extracellular domain of the human Epo Receptor.

In certain embodiments, a single chain variable fragment fused to an Fc is provided which specifically binds to amino acids V48 and H70 of the extracellular domain of the human Epo Receptor.

In certain embodiments, a single chain variable fragment fused to an Fc is provided which specifically binds to amino acids W64 and R68 of the extracellular domain of the human Epo Receptor.

In certain embodiments, a single chain variable fragment fused to an Fc is provided which specifically binds to amino acids W64 and H70 of the extracellular domain of the human Epo Receptor.

In certain embodiments, a single chain variable fragment fused to an Fc is provided which specifically binds to amino acids L66 and R68 of the extracellular domain of the human Epo Receptor.

In certain embodiments, a single chain variable fragment fused to an Fc is provided which specifically binds to amino acids L66 and H70 of the extracellular domain of the human Epo Receptor.

In certain embodiments, a single chain variable fragment fused to an Fc is provided which specifically binds to amino acids R68 and H70 of the extracellular domain of the human Epo Receptor.

In certain embodiments, a single chain variable fragment fused to an Fc is provided which specifically binds to one or more of amino acids A44, V48, E62, P63, W64, L66, R68, H70, S91, F93, R99, H114, and M150 of the extracellular domain of the human Epo Receptor.

In certain embodiments, an antibody is provided which specifically binds to amino acids F93, E60, and H114 of the extracellular domain of the human Epo Receptor.

In certain embodiments, an antibody is provided which specifically binds to amino acid V48 of the extracellular domain of the human Epo Receptor.

In certain embodiments, an antibody is provided which specifically binds to amino acid L66 of the extracellular domain of the human Epo Receptor.

In certain embodiments, an antibody is provided which specifically binds to amino acid W64 of the extracellular domain of the human Epo Receptor.

In certain embodiments, an antibody is provided which specifically binds to amino acid H70 of the extracellular domain of the human Epo Receptor.

In certain embodiments, an antibody is provided which specifically binds to amino acids V48 and W64 of the extracellular domain of the human Epo Receptor.

In certain embodiments, an antibody is provided which specifically binds to amino acids V48 and L66 of the extracellular domain of the human Epo Receptor.

In certain embodiments, an antibody is provided which specifically binds to amino acids V48 and R68 of the extracellular domain of the human Epo Receptor.

In certain embodiments, an antibody is provided which specifically binds to amino acids V48 and H70 of the extracellular domain of the human Epo Receptor.

In certain embodiments, an antibody is provided which specifically binds to amino acids W64 and R68 of the extracellular domain of the human Epo Receptor.

In certain embodiments, an antibody is provided which specifically binds to amino acids W64 and H70 of the extracellular domain of the human Epo Receptor.

In certain embodiments, an antibody is provided which specifically binds to amino acids L66 and R68 of the extracellular domain of the human Epo Receptor.

In certain embodiments, an antibody is provided which specifically binds to amino acids L66 and H70 of the extracellular domain of the human Epo Receptor.

In certain embodiments, an antibody is provided which specifically binds to amino acids R68 and H70 of the extracellular domain of the human Epo Receptor.

In certain embodiments, an antibody is provided which specifically binds to one or more of amino acids A44, V48, E62, P63, W64, L66, R68, H70, S91, F93, R99, H114, and M150 of the extracellular domain of the human Epo Receptor.

In certain embodiments, the effects of an antibody may be evaluated by measuring a reduction in the amount of symptoms of a disease of interest. In certain embodiments, the disease of interest may be caused by a pathogen. In certain embodiments, a disease may be established in an animal host by other methods including introduction of a substance (such as a carcinogen) and genetic manipulation. In certain embodiments, effects may be evaluated by detecting one or more adverse events in the animal host. The term “adverse event” includes, but is not limited to, an adverse reaction in an animal host that receives an antibody that is not present in an animal host that does not receive the antibody. In certain embodiments, adverse events include, but are not limited to, a fever, an immune response to an antibody, inflammation, and/or death of the animal host.

In certain embodiments, the composition further comprises an EREDLA and at least one sugar. As used herein, the term “sugar” refers to monosaccharides such as glucose and mannose, or polysaccharides including disaccharides such as sucrose and lactose, as well as sugar derivatives including sugar alcohols and sugar acids. Sugar alcohols include, but are not limited to, mannitol, xylitol, erythritol, threitol, sorbitol and glycerol. A non-limiting example of a sugar acid is L-gluconate. Certain exemplary sugars include, but are not limited to, trehalose, fucose, and glycine.

In certain embodiments, the composition further comprises at least one bulking/osmolarity regulating agent. Such agents may be either crystalline (for example, glycine, mannitol) or amorphous (for example, L-histidine, sucrose, polymers such as dextran, polyvinylpyrolidone, carboxymethylcellulose, and lactose). In certain embodiments, a bulking/osmolarity regulating agent is provided at a concentration between 2% and 5%. In certain embodiments, a bulking/osmolarity regulating agent is provided at a concentration between 2.5% and 4.5%.

In certain embodiments, EREDLAs which bind to a particular protein and block interaction with other binding compounds may have therapeutic use. In this application, when discussing the use of EREDLAs to treat diseases or conditions, such use may include use of compositions comprising antibodies; and/or combination therapies comprising antibodies and one or more additional active ingredients. When EREDLAs are used to “treat” a disease or condition, such treatment may or may not include prevention of the disease or condition.

In certain embodiments, an EREDLA is administered alone. In certain embodiments, an EREDLA is administered prior to the administration of at least one other therapeutic agent. In certain embodiments, an EREDLA is administered concurrent with the administration of at least one other therapeutic agent. In certain embodiments, an EREDLA is administered subsequent to the administration of at least one other therapeutic agent.

In certain embodiments, EREDLAs may be used to treat non-human animals, such as pets (dogs, cats, birds, primates, etc.), and domestic farm animals (horses cattle, sheep, pigs, birds, etc.). In certain such instances, an appropriate dose may be determined according to the animal's body weight. For example, in certain embodiments, a dose of 0.2-1 mg/kg may be used. In certain embodiments, the dose may be determined according to the animal's surface area, an exemplary dose ranging from 0.1 to 20 mg/in², or from 5 to 12 mg/m². For small animals, such as dogs or cats, in certain embodiments, a suitable dose is 0.4 mg/kg. In certain embodiments, EREDLAs are administered by injection or other suitable route one or more times per week until the animal's condition is improved, or it may be administered indefinitely.

It is understood that the response by individual patients to the aforementioned medications or combination therapies may vary, and an appropriate efficacious combination of drugs for each patient may be determined by his or her physician.

In certain embodiments, an EREDLA may be part of a conjugate molecule comprising all or part of the EREDLA and a prodrug. In certain embodiments, the term “prodrug” refers to a precursor or derivative form of a pharmaceutically active substance. In certain embodiments, a prodrug is less cytotoxic to cells compared to the parent drug and is capable of being enzymatically activated or converted into the more active cytotoxic parent form. Exemplary prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prod rugs, optionally substituted phenoxyacetamide-containing prodrugs and optionally substituted phenylacetamide-containing prodrugs, 5-fluorocytosine and other 5-fluorouridine prodrugs which can be converted into a more active cytotoxic free drug. Examples of cytotoxic drugs that can be derivatized into a prodrug form include, but are not limited to, those cytotoxic agents described above. See, e.g., U.S. Pat. No. 6,702,705.

In certain embodiments, EREDLA conjugates function by having the antibody portion of the molecule target the cytotoxic portion or prodrug portion of the molecule to a specific population of cells in the patient.

In certain embodiments, methods of treating a patient comprising administering a therapeutically effective amount of an EREDLA are provided. In certain embodiments, methods of treating a patient comprising administering a therapeutically effective amount of an EREDLA conjugate are provided. In certain embodiments, an EREDLA is used in conjunction with a therapeutically effective amount of at least one additional therapeutic agent, as discussed above.

As discussed above, in certain embodiments, EREDLAs may be administered concurrently with one or more other drugs that are administered to the same patient, each drug being administered according to a regimen suitable for that medicament. Such treatment encompasses pre-treatment, simultaneous treatment, sequential treatment, and alternating regimens. Additional examples of such drugs include, but are not limited to, antivirals, antibiotics, analgesics, corticosteroids, antagonists of inflammatory cytokines, DMARDs, nonsteroidal anti-inflammatories, chemotherapeutics, inhibitors of angiogenesis, and stimulators of angiogenesis.

In certain embodiments, a composition comprises a therapeutically effective amount of an EREDLA and a pharmaceutically acceptable diluent, carrier, solubilizer, emulsifier, preservative and/or adjuvant.

In certain embodiments, pharmaceutical compositions are provided comprising a therapeutically effective amount of an EREDLA and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1990).

In certain embodiments, an EREDLA and/or an additional therapeutic molecule is linked to a half-life extending vehicle known in the art. Such vehicles include, but are not limited to, the Fc domain, polyethylene glycol, and dextran. Such vehicles are described, e.g., in U.S. Pat. No. 6,660,843 and published PCT Application No. WO 99/25044.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies.

In certain embodiments, the primary vehicle or carrier in a pharmaceutical composition may be either aqueous or non-aqueous in nature. For example, in certain embodiments, a suitable vehicle or carrier may be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which may further include sorbitol or a suitable substitute therefor. In certain embodiments, a pharmaceutical composition is an aqueous or liquid formulation comprising an acetate buffer of about pH 4.0-5.5, a polyol (polyalcohol), and optionally, a surfactant, wherein the composition does not comprise a salt, e.g., sodium chloride, and wherein the composition is isotonic for the patient. Exemplary polyols include, but are not limited to, sucrose, glucose, sorbitol, and mannitol. An exemplary surfactant includes, but is not limited to, polysorbate. In certain embodiments, a pharmaceutical composition is an aqueous or liquid formulation comprising an acetate buffer of about pH 5.0, sorbitol, and a polysorbate, wherein the composition does not comprise a salt, e.g., sodium chloride, and wherein the composition is isotonic for the patient. Certain exemplary compositions are found, for example, in U.S. Pat. No. 6,171,586. Additional pharmaceutical carriers include, but are not limited to, oils, including petroleum oil, animal oil, vegetable oil, peanut oil, soybean oil, mineral oil, sesame oil, and the like. In certain embodiments, aqueous dextrose and glycerol solutions can also be employed as liquid carriers, particularly for injectable solutions. In certain embodiments, a composition comprising an antibody, with or without at least one additional therapeutic agent, may be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an antibody, with or without at least one additional therapeutic agent, may be formulated as a lyophilizate using appropriate excipient solutions (e.g., sucrose) as diluents.

In certain embodiments, EREDLAs are administered in the form of a physiologically acceptable composition comprising purified recombinant protein in conjunction with physiologically acceptable carriers, excipients or diluents. In certain embodiments, such carriers are nontoxic to recipients at the dosages and concentrations employed. In certain embodiments, preparing such compositions may involve combining the antibodies with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and/or other stabilizers, and excipients. In certain embodiments, appropriate dosages are determined in standard dosing trials, and may vary according to the chosen route of administration. In certain embodiments, in accordance with appropriate industry standards, preservatives may also be added, which include, but are not limited to, benzyl alcohol. In certain embodiments, the amount and frequency of administration may be determined based on such factors as the nature and severity of the disease being treated, the desired response, the age and condition of the patient, and so forth.

In certain embodiments, pharmaceutical compositions can be selected for parenteral delivery. The preparation of certain such pharmaceutically acceptable compositions is within the skill of the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition may be in the form of a pyrogen-free, parenterally acceptable aqueous solution comprising the desired antibody, with or without additional therapeutic agents, in a pharmaceutically acceptable vehicle. In certain embodiments, a vehicle for parenteral injection is sterile distilled water in which the antibody, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads, or liposomes, that may provide for the controlled or sustained release of the product which may then be delivered via a depot injection. In certain embodiments, hyaluronic acid may also be used, and may have the effect of promoting sustained duration in the circulation. In certain embodiments, implantable drug delivery devices may be used to introduce the desired molecule.

In certain embodiments, a pharmaceutical composition may be formulated for inhalation. In certain embodiments, administration by inhalation is beneficial when treating diseases associated with pulmonary disorders. In certain embodiments, an antibody, with or without at least one additional therapeutic agent, may be formulated as a dry powder for inhalation. In certain embodiments, an inhalation solution comprising an antibody, with or without at least one additional therapeutic agent, may be formulated with a propellant for aerosol delivery. In certain embodiments, solutions may be nebulized. Pulmonary administration is further described in PCT publication no. WO94/20069, which describes pulmonary delivery of chemically modified proteins.

In certain embodiments, it is contemplated that formulations may be administered orally. In certain embodiments, an EREDLA, with or without at least one additional therapeutic agent, that is administered in this fashion may be formulated with or without those carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. In certain embodiments, at least one additional agent can be included to facilitate absorption of the antibody and/or any additional therapeutic agents. In certain embodiments, diluents, flavorings, low melting point waxes, vegetable oils, lubricants, suspending agents, tablet disintegrating agents, and/or binders may also be employed.

In certain embodiments, a pharmaceutical composition may involve an effective quantity of an EREDLA, with or without at least one additional therapeutic agent, in a mixture with non-toxic excipients which are suitable for the manufacture of tablets. In certain embodiments, by dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; and binding agents, such as starch, gelatin, and acacia; and lubricating agents such as magnesium stearate, stearic acid, and talc.

Additional pharmaceutical compositions will be evident to those skilled in the art, including formulations involving antibodies, with or without at least one additional therapeutic agent, in sustained- or controlled-delivery formulations. In certain exemplary sustained- or controlled-delivery formulations include, but are not limited to, liposome carriers, bio-erodible microparticles, porous beads, and depot injections. Certain exemplary techniques for preparing certain formulations are known to those skilled in the art. See for example, PCT publication no. WO93/15722, which describes the controlled release of porous polymeric microparticles for the delivery of pharmaceutical compositions. In certain embodiments, sustained-release preparations may include semipermeable polymer matrices in the form of shaped articles, e.g. films, or microcapsules. Sustained release matrices include, but are not limited to, polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra), and poly-D(−)-3-hydroxybutyric acid (EP 133,988). In certain embodiments, sustained release compositions may also include liposomes, which can be prepared, in certain embodiments, by any of several methods known in the art. See e.g., Eppstein et al., Proc. Natl. Acad. Sci. USA, 82:3688-3692 (1985); EP 036,676; EP 088,046 and EP 143,949.

In certain embodiments, the pharmaceutical composition to be used for in vivo administration is sterile. In certain embodiments, the pharmaceutical composition to be used for in vivo administration is made sterile by filtration through sterile filtration membranes. In certain embodiments, where the composition is lyophilized, sterilization using sterile filtration membranes may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, after the pharmaceutical composition has been formulated, it may be stored in sterile vials as a solution, suspension, gel, emulsion, solid, or as a dehydrated or lyophilized powder. In certain embodiments, such formulations may be stored either in a ready-to-use form or in a form (e.g., a lyophilized form) that is reconstituted prior to administration.

In certain embodiments, kits for producing a single-dose administration unit are provided. In certain embodiments, the kits may each contain both a first container having a dried protein and a second container having an aqueous formulation. In certain embodiments, kits containing single and/or multi-chambered pre-filled syringes (e.g., liquid syringes and lyosyringes) are included.

In certain embodiments, the effective amount of a pharmaceutical composition comprising an EREDLA, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the antibody, with or without at least one additional therapeutic agent, is being used, the route of administration, and the size (body weight, body surface or organ size) and/or condition (the age and general health) of the patient. In certain embodiments, the clinician may titer the dosage and modify the route of administration to obtain the optimal therapeutic effect. In certain embodiments, a typical dosage may range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg; or 0.1 mg/kg up to about 100 mg/kg.

In certain embodiments, the frequency of dosing will take into account the pharmacokinetic parameters of the antibody and/or any additional therapeutic agents in the formulation used. In certain embodiments, a clinician will administer the composition until a dosage is reached that achieves the desired effect. In certain embodiments, the composition may therefore be administered as a single dose, or as two or more doses (which may or may not contain the same amount of the desired molecule) over time, or as a continuous infusion via an implantation device or catheter. Certain methods of further refining the appropriate dosage are within the skill in the art. In certain embodiments, appropriate dosages may be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

As discussed above, in various embodiments, any efficacious route of administration may be used to administer antibodies. If injected, in certain embodiments, antibodies may be administered, for example, via intra-articular, intravenous, intramuscular, intralesional, intraperitoneal, intracranial, intranasal, inhalation or subcutaneous routes by bolus injection or by continuous infusion. Exemplary methods of administration include, but are not limited to, sustained release from implants, aerosol inhalation, eyedrops, oral preparations, and topical preparations such as lotions, gels, sprays, ointments, and other suitable techniques.

When EREDLAs are administered in combination with one or more other biologically active compounds, in certain embodiments, these may be administered by the same or by different routes, and may be administered together, separately, or sequentially.

In certain embodiments, the composition may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

In certain embodiments, it may be desirable to use a pharmaceutical composition comprising an EREDLA, with or without at least one additional therapeutic agent, in an ex vivo manner. In such embodiments, cells, tissues and/or organs that have been removed from the patient are exposed to a pharmaceutical composition comprising an antibody, with or without at least one additional therapeutic agent, after which the cells, tissues and/or organs are subsequently implanted back into the patient.

In certain embodiments, a first EREDLA binds to a first epitope on the Epo receptor and a second EREDLA binds to a second epitope on the same molecule. In certain such embodiments, the first epitope overlaps with the second epitope such that binding of either the first EREDLA or second EREDLA to the molecule inhibits binding of the other antibody to the Epo receptor. In certain embodiments, the first epitope does not overlap with the second epitope such that binding of the first EREDLA or the second EREDLA to the Epo receptor does not inhibit binding of the other EREDLA.

In certain embodiments, an epitope on the Epo receptor overlaps with a ligand binding site on the Epo receptor. In certain such embodiments, binding of an EREDLA to the Epo receptor inhibits binding of the ligand (e.g., Epo) to the Epo receptor. In certain embodiments, binding of an EREDLA to the Epo receptor blocks binding of the ligand to the Epo receptor. In certain embodiments, binding of an EREDLA partially inhibits binding of the ligand to the Epo receptor.

In certain embodiments, an epitope on an Epo receptor molecule does not overlap with a ligand binding site on the receptor. In certain such embodiments, binding of an EREDLA to the epitope at least partially activates the Epo receptor. In certain other embodiments, binding of an EREDLA to the epitope does not activate the Epo receptor.

In certain embodiments, an epitope on an Epo receptor molecule overlaps with a ligand binding site on the receptor. In certain such embodiments, binding of an EREDLA to the epitope at least partially activates the Epo receptor. In certain other embodiments, binding of an EREDLA to the epitope does not activate the Epo receptor. In certain embodiments, binding of an EREDLA to the epitope on the receptor inhibits activation of the receptor by the receptor ligand. In certain embodiments, binding of an EREDLA to the epitope on the Epo receptor blocks activation of the Epo receptor by the receptor ligand.

In certain embodiments, dimerization of the Epo receptor increases its activation. In certain embodiments, a bivalent EREDLA facilitates Epo receptor dimerization. In certain embodiments, a monovalent EREDLA is crosslinked with another monovalent antibody to create a bivalent molecule.

In certain embodiments, an EpoR agonist is an antibody which activates huEpoR. In certain embodiments, an antibody that activates huEpoR (a huEpoR antibody) is an EREDLA. In certain embodiments, an EREDLA is administered less frequently than an erythropoiesis stimulating protein (ESP). Examples of ESPs include epoietin alfa, epoietin beta and darbepoietin alfa. In certain embodiments, an EREDLA is administered about once per month, or about once every two months, or about once every three months, or about once every four months, or about once every five months, or about once every six months.

In certain embodiments, an EREDLA is administered at low frequency compared to traditional erythropoietic agents that share sequence homology with the native erythropoietin molecule. In certain embodiments, antibodies against an EREDLA are unable to cross-react with native erythropoietin (Epo) and thus are unable to induce Pure Red Cell Aplasia (PRCA). As a consequence, administration of an EREDLA carries a reduced risk of inducing PRCA when compared with administration of other erythropoiesis stimulating proteins. In certain embodiments, an EREDLA with a reduced risk of inducing PRCA is used to treat a disease or condition using a method of administration to allow for controlled release over an extended period of time. For example, and not limitation, an EREDLA could be administered orally or with non-invasive delivery devices without increasing the risk of PRCA.

In certain embodiments, at least one EREDLA is used to treat a disease or condition in a mammal, which includes humans. In certain embodiments, an EREDLA comprising an amino acid sequence comprising SEQ ID NO.: 1 and SEQ ID NO.: 2 is used to treat a disease or condition. In certain embodiments, an EREDLA comprising an amino acid sequence comprising SEQ ID NO.: 3 and SEQ ID NO.: 4 is used to treat a disease or condition. In certain embodiments, an EREDLA comprising an amino acid sequence comprising SEQ ID NO.: 5 and SEQ ID NO.: 6 is used to treat a disease or condition. In certain embodiments, an EREDLA comprising an amino acid sequence comprising SEQ ID NO.: 7 and SEQ ID NO.: 8 is used to treat a disease or condition. In certain embodiments, an EREDLA comprising an amino acid sequence comprising SEQ ID NO.: 9 and SEQ ID NO.: 10 is used to treat a disease or condition. In certain embodiments, an EREDLA comprising an amino acid sequence comprising SEQ ID NO. 56 and SEQ ID NO. 58 is used to treat a disease or condition. In certain embodiments, an EREDLA comprising an amino acid sequence comprising SEQ ID NO. 60 and SEQ ID NO. 62 is used to treat a disease or condition. In certain embodiments, an EREDLA comprising an amino acid sequence comprising SEQ ID NO. 64 and SEQ ID NO. 66 is used to treat a disease or condition. In certain embodiments, an EREDLA comprising an amino acid sequence comprising SEQ ID NO. 68 and SEQ ID NO. 70 is used to treat a disease or condition. In certain embodiments, an EREDLA comprising an amino acid sequence comprising SEQ ID NO. 72 and SEQ ID NO. 74 is used to treat a disease or condition. In certain embodiments, an EREDLA comprising an amino acid sequence comprising SEQ ID NO. 76 and SEQ ID NO. 78 is used to treat a disease or condition. In certain embodiments, an EREDLA comprising an amino acid sequence comprising SEQ ID NO. 80 and SEQ ID NO. 82 is used to treat a disease or condition. In certain embodiments, an EREDLA comprising an amino acid sequence comprising SEQ ID NO. 84 and SEQ ID NO. 86 is used to treat a disease or condition. In certain embodiments, an EREDLA comprising an amino acid sequence comprising SEQ ID NO. 88 and SEQ ID NO: 90 is used to treat a disease or condition. In certain embodiments, an EREDLA comprising an amino acid sequence comprising SEQ ID NO. 92 and SEQ ID NO. 94 is used to treat a disease or condition. In certain embodiments, an EREDLA comprising an amino acid sequence comprising SEQ ID NO. 96 and SEQ ID NO. 98 is used to treat a disease or condition. In certain embodiments, an EREDLA comprising an amino acid sequence comprising SEQ ID NO: 100 and SEQ ID NO. 102 is used to treat a disease or condition. In certain embodiments, an EREDLA comprising an amino acid sequence comprising SEQ ID NO. 104 and SEQ ID NO. 106 is used to treat a disease or condition. In certain embodiments, an EREDLA comprising an amino acid sequence comprising SEQ ID NO. 108 and SEQ ID NO. 110 is used to treat a disease or condition. In certain embodiments, an EREDLA comprising an amino acid sequence comprising SEQ ID NO: 112 and SEQ ID NO: 114 is used to treat a disease or condition.

In certain embodiments, an EREDLA that specifically binds to amino acids F93 and H114 of the extracellular domain of the human Epo Receptor is used to treat a disease or condition. In certain embodiments, an EREDLA that specifically binds to amino acids S91, F93, and H114 of the extracellular domain of the human Epo Receptor is used to treat a disease or condition. In certain embodiments, an EREDLA that specifically binds to amino acid F93 of the extracellular domain of the human Epo Receptor is used to treat a disease or condition. In certain embodiments, an EREDLA that specifically binds to amino acids E62, F93, and M150 of the extracellular domain of the human Epo Receptor is used to treat a disease or condition. In certain embodiments, an EREDLA that specifically binds to amino acids V48, E62, L66, R68, and H70 of the extracellular domain of the human Epo Receptor is used to treat a disease or condition. In certain embodiments, an EREDLA that specifically binds to amino acids V48, W64, L66, R68, and H70 of the extracellular domain of the human Epo Receptor is used to treat a disease or condition. In certain embodiments, an EREDLA that specifically binds to amino acids A44, V48, P63, L66, R68, and H70 of the extracellular domain of the human Epo Receptor is used to treat a disease or condition. In certain embodiments, an EREDLA that specifically binds to amino acids L66 and R99 of the extracellular domain of the human Epo Receptor is used to treat a disease or condition.

In certain embodiments, the disease or condition treated is associated with decreased red blood cell and/or hemoglobin levels. In certain embodiments, the disease or condition treated is anemia. In certain embodiments, treatment of anemia with an EREDLA is characterized by a longer-duration erythropoietic response than is observed with other ESPs.

In certain embodiments, an EREDLA is used to treat anemia of chronic diseases or conditions. Chronic means persistent or lasting. In certain embodiments, a chronic disease or condition may worsen over time. In certain embodiments, a chronic disease or condition may not worsen over time. Exemplary chronic diseases include, but are not limited to, chronic kidney disease, congestive heart failure, and myelodysplastic syndromes.

In certain embodiments, an EREDLA possesses a pharmacokinetic profile appropriate for treating a chronic disease or condition. In certain such embodiments, an EREDLA possesses a phramacokinetic profile that comprises an erythropoietic response extending over a longer duration than the erythropoietic response that is observed with other ESPs.

In certain embodiments, an EREDLA is used to treat anemia of cancer, chemotherapy-induced anemia, anemia of the elderly, or other anemias, such as but not limited to, anemia due to infection, inflammation, iron deficiency, blood loss, hemolysis, secondary hyperparathyroidism, inadequate dialysis, protein energy malnutrition, vitamin deficiencies, or metal toxicity (e.g., aluminum). In certain embodiments, an EREDLA is used to treat PRCA in patients that develop this condition as a result of disease or in response to the administration of erythropoietic drugs.

In certain embodiments, an EREDLA is used to promote tissue protection in erythropoietin-responsive cells, tissues, and organs. For example, and without limitation, in certain embodiments, an EREDLA is used to promote tissue protection during or after a myocardial infarction or a stroke. In certain embodiments, an EREDLA is used to promote tissue protection in tissues that can be protected by administration of erythropoietin. Certain examples of cells, tissues, and organs that can be protected by administration of erythropoietin are described in PCT Publications WO 02/053580 and WO 00/61164.

In certain embodiments, an EREDLA is used to increase hematocrit in a patient in need thereof. In certain embodiments, an EREDLA is administered once to increase hematocrit for a period of about 30 days, or about 60 days, or about 90 days, or about 120 days, or about 150 days, or about 180 days.

### EXAMPLES

### Example 1

### Identification of Anti-huEpoR Antibodies from Naïve Human scFv Phage-Display Libraries

Selection Strategy 1

In a first round of selection, approximately 10¹² human scFv phage from naïve phage libraries were incubated with 200 nM biotinylated huEpoR in 1 ml 2% non fat dry milk in PBS/0.1% tween 20 (PBS/T) for 1 hour at room temperature followed by 5 washes using PBS/T. The scFv phage that bound to huEpoR were captured using streptavidin coated magnetic beads. Bound phage were released from magnetic beads by incubation with 1 ml trypsinization solution (50 μg/ml porcine trypsin in 50 mM Tris HCl/1 mM CaCl2 at pH 8.0) at 37° C. for 10 minutes.

To re-introduce the released phage to E. coli cells, 10 ml of log phase TG1 cells were used for incubation with the entire population of phage released from the magnetic beads at 37° C. for 30 minutes without shaking and another 30 minutes with slow shaking. Gently pelleted TG1 cells were re-suspended into approximately 1.5 ml of 2×YT media, spread on 2 Nunc plates (25 cm×25 cm) with 2×YT media supplemented with 100 μg/ml carbenicillin and 4% glucose and amplified overnight at 30° C. Amplified cells were then scraped from the plates and pooled. Approximately 10-100 μl of the pooled cells, covering greater than 10 fold of the released phage particles, were used to inoculate 25 ml of 2×YT media/100 μg/ml carbenicillin and 2% glucose and grown at 37° C. with shaking to an OD₆₀₀ of 0.5. This log phase culture was then super-infected with approximately 10¹¹ M13KO7 helper phage at 37° C. for 30 minutes and another 30 minutes with gentle shaking. Cells were pelleted and resuspended into 25 ml of 2×YT media supplemented with 100 μg/ml carbenicillin and 25 μg/ml of kanamycin. Cells were shaken at 250 rpm at 25° C. overnight. The supernatant of the culture was harvested by centrifugation at 10,000 rpm for 10 minutes. The phage in the supernatant were precipitated by adding ⅕ volume of 20% PEG8000/2.5 M NaCl incubated on ice for greater than 30 minutes. The phage were then pelleted by centrifugation at 10,000 rpm for 10 minutes and resuspended into TE buffer (10 mM Tris and 1 mM EDTA, pH7.5).

In a second round of selection, the resuspended scFv phage were incubated with 50 nM biotinylated huEpoR for 1 hour at room temperature followed by 10 washes using PBS/0.1% tween 20. huEpoR binding scFv phage were captured using streptavidin coated magnetic beads. Bound phage were released from magnetic beads by incubation with 1 ml trypsinization solution at 37° C. for 10 minutes. Half of the released phage were used in the Selection Strategy 2 described below.

A small fraction of the released phage from the second round of selection were reintroduced into TG1 by incubating properly diluted phage with mid log phase E coli cells. The TG1 cells were then plated on 2×YT 100 μg/ml carbenicillin petridish plates to generate single colonies. 384 randomly selected single colonies were individually picked off the petridish plates and placed into separate wells of 96-well plates containing 100 μl of 2×YT media supplemented with 100 μg/ml carbenicillin and 2% glucose to create 96-well experimental plates. The 96-well experimental plates were incubated at 37° C. with shaking until TG1 cells reached an OD₆₀₀ of approximately 0.5 (mid log phase).

As a separate step, a new set of 96-well culture plates containing the same culture media described above were inoculated with a small fraction of the growing cultures in the 96-well experimental plates to create duplicate plates. These duplicate plates were grown at 37° C. overnight. 20 μl of a 50% glycerol solution was then added to each well of the plates and the plates were frozen on dry ice and stored at −70° C. as master plates.

The mid log phase cultures in the 96-well experimental plates were then super-infected with approximately 10⁹ M13KO7 helper phage at 37° C. for 30 minutes and another 30 minutes with gentle shaking. The 96-well plates were then centrifuged at 3000 rpm for 5 minutes and the supernatants in the wells were removed by flipping the plates. 200 μl of 2×YT media supplemented with 100 μg/ml carbenicillin and 25 μg/ml of Kanamycin were then added to each well and the plates were incubated with shaking at 250 rpm at 30° C. overnight. The overnight phage culture was centrifuged at 3,000 rpm for 5 minutes and the resultant supernatant samples were used for ELISA experiments.

A new set of Nunc-Immuno Polysorp 96-well ELISA plates (Nalge Nunc International) were prepared by adding huEpoR at 1 μg/ml to the wells of the plates and incubating the plates overnight at 4° C. A 1/20 dilution of culture supernatant containing one of the 384 different monoclonal phage in 2% non-fat dry milk solution in PBS/T was added to each separate well of the 96-well plates containing the huEpoR coated on the surface. The plates were incubated for 1 hour followed by 3 washes in PBS/T. Detection of the bound phage was performed using anti-M13 mAb/HRP conjugate (Amersham Biosciences) followed by 3 washes in PBS/T. ABTS was used as the substrate and absorption at 405 nm detected. A total of 96 phage that bind to huEpoR were identified from the ELISA screening of the 384 randomly picked phage clones.

Selection Strategy 2

Half of the eluted phage from the round 2 selection in Selection Strategy 1 described above in paragraph 527 were reintroduced to TG1 cells and a phage preparation was made using the same procedure as described above in paragraph 526 of Selection Strategy 1. Approximately 10¹² amplified scFv phage were used for cell panning by incubating the scFv phage with huEpoR expressing UT-7 cells (2×10⁶ cells in 1 ml PBS/2% BSA) at 4° C. for 2 hours followed by 10 washes with PBS/T.

UT-7 binding phage were eluted from the cell surface by incubation with 1 ml glycine/HCl buffer (100 mM glycine/HCl at pH2.5) for 10 minutes followed by centrifugation at 3,000 rpm for 5 minutes. The acidic supernatant containing the eluted phage was neutralized with 50 μl of 1 M Tris base solution.

A small aliquot of the eluted phage from the UT-7 cell panning was introduced into TG1 cells through phage infection. The phage infected TG1 cells were then plated on 2×YT 100 μg/ml carbenicillin petridish plates to generate single colonies. 192 randomly selected single colonies were picked off the petridish plates and individually placed into separate wells of two 96-deep well plates containing 1 ml of 2×YT media supplemented with 100 μg/ml carbenicillin and 2% glucose. The two 96-deep well plates were incubated at 37° C. with shaking until the culture reached an OD₆₀₀ of approximately 0.5

As a separate step, a new set of 96-well culture plates containing the same culture media described above were inoculated with a small fraction of the growing cultures in the 96-deep well plates to create duplicate plates. These duplicate plates were grown at 37° C. overnight. 20 μl of a 50% glycerol solution was then added to each well of the plates and the plates were frozen on dry ice and stored at −70° C. as master plates.

After inoculating the master plates, the two 96-deep well plates with cultures at an OD₆₀₀ of approximately 0.5 were used in a FACS experiment as described below.

Screening of UT-7 Cell Binding Phage by FACS

1 ml of 2×YT/2×YT media supplemented with 100 μg/ml carbenicillin and 2% glucose was placed in each well of a 96-deep well plate. New phage samples of the 96 positive clones identified by ELISA in Selection Strategy 1 were prepared by inoculating the media in each well of the 96-deep well plate with cells from the corresponding wells on the master plates. The 96-deep well plate was incubated at 37° C. with shaking until the culture reached an OD₆₀₀ of approximately 0.5.

As discussed in Selection Strategy 2, cultures containing 192 different phage from Selection Strategy 2 were incubated in two 96-deep well plates at 37° C. with shaking until the cultures reached an OD₆₀₀ of approximately 0.5.

The three 96-deep well plates containing log phase cultures (described in the two preceeding paragraphs) were then super-infected with approximately 10⁹ M13KO7 helper phage at 37° C. for 30 minutes and another 30 minutes with gentle shaking. The plates were then centrifuged at 3000 rpm for 5 minutes and the supernatants were removed by flipping the plates. 1 ml of 2×YT media supplemented with 100 μg/ml carbenicillin and 25 μg/ml of kanamycin were then added to each well and the plates were incubated by shaking at 250 rpm at 30° C. overnight. The supernatants containing phage were prepared by centrifugation of the overnight culture at 3000 rpm for 5 minutes. The phage were purified from the supernatant by adding ⅕ vol of 20% PEG8000/2.5 M NaCl solution. The precipitated phage were pelleted by centrifugation and the resultant phage pellets in each well of the 96-deep well plates were resuspended into 100 μl of TE buffer (10 mM tris HCl, 1 mM EDTA, pH7.5) for use in FACS experiments.

In each well of a new set of three 96-well plates, UT-7 cells were incubated with a 10 μl aliquot of a single phage and 90 μl of 2% BSA PBS/T for 1 hour at 4° C. After 2 quick washes using cold PBS, cells were then incubated with 100 μl of 1 μg/ml anti-M13 mouse monoclonal antibody (Amersham Biosciences) in 2% BSA PBS/T at 4° C. for 1 hour Following 2 quick washes with cold PBS, 100 μl of 1 μg/ml phycoerythrin-conjugated goat F(ab′)2 anti-mouse IgG Fc (Jackson Immuno Research Laboratories) was added to each well on the plates. The plates were then incubated for 1 hour at 4° C. The cells were washed twice again using cold PBS and were resuspended in 1 ml of fixation buffer (2% paraformaldehyde PBS pH 7.4). FACS was done using a Multiwell Caliber flow cytometer.

14 phage clones from Selection Strategy 1 and 38 from Selection Strategy 2 were identified as binders of UT-7 cells expressing EpoR. DNA sequencing analysis of those scFv phage samples resulted in a total of 29 unique scFv sequences.

### Example 2

### Conversion of Phage scFv to scFv-Fc, IgG₂, and IgG₁ Protein Expression and Purification

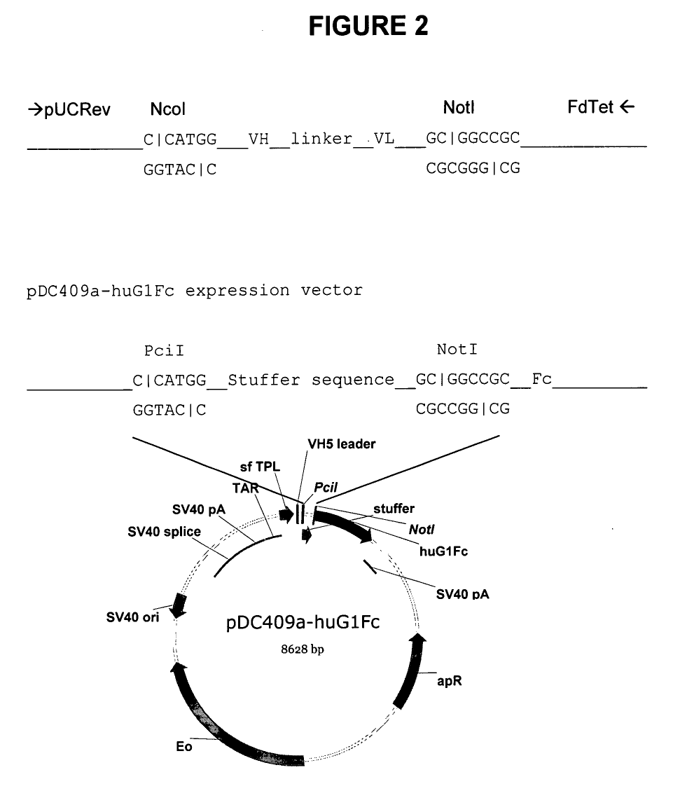

All 29 phage scFv clones were converted to scFv-Fc fusion proteins using a streamlined subcloning procedure (FIG. 2). DNA encoding the scFv was amplified the phagemid encoding the clones by PCR using a pair of vector-specific primers (pUCRev/FdTet). Ligation of the NcoI and NotI restriction fragments of scFv into a PciI (creates a cohesive end with NcoI) and NotI digested mammalian expression vector, pDC409a-G1 Fc, resulted in fusion of the scFv to the human IgG₁ Fc. pDC409a-huG1 Fc contains a human IgG₁ Fc after the NotI site. NcoI and PciI restriction fragments have the same cohesive end. The secretion of scFv-Fc protein is mediated by a VH5α signal sequence. Maxibodies derived from individual phage clones are referred to by the designation “Mxb x” where x represents the clone number.

For converting scFv clones to IgG₂ expression constructs, DNA fragments encoding a VH or VL region were PCR amplified from phagemids encoding the clones using primers specific for each variable domain. Ligation of the VH (Nhe/Ascl) fragment to a similarly restriction digested IgG2 heavy chain expression vector, pVE414NhulgG2 resulted in an antibody heavy chain expression construct. Ligation of the Vλ NheI/NarI fragment to a similarly restriction digested light chain expression vector pVE414NhuλLC resulted in an antibody lambda light chain expression construct. Ligation of the Vκ NheI/Bsi WI fragment to a similarly restriction digested light chain expression vector pVE414NhuκLC resulted in antibody kappa light chain expression constructs. The choice of light chain constant type matches the variable light chain isotypes.

For generation of the IgG₁ expression constructs, the same VH Nhe/Ascl fragment used for the IgG₂ expression construct was ligated into a similarly restriction digested pVE414NhulgGl vector. The light chain expression constructs described in the preceeding paragraph were used to express the IgG₁ light chains as well as the IgG₂ light chains.

scFv-Fc proteins were expressed transiently in mammalian COS-1 PKB E5 cells by cotransfection of antibody heavy and light chain expression constructs. IgG₁ proteins were also expressed transiently in mammalian COS-1 PKB E5 cells by cotransfection of antibody heavy and light chain expression constructs. IgG₂ proteins were also expressed transiently in mammalian COS-1 PKB E5 cells by cotransfection of antibody heavy and light chain expression constructs. The expressed antibodies were purified to greater than 95% purity from the conditioned media using protein A affinity chromatography. Protein identities were verified by N-terminal amino acid sequencing and concentrations were determined by absorption at 280 nm.

### Example 3

### Antibody Binding to Cell Surface huEpoR Analysis by FACS

The binding of the scFv-Fc protein to a cell surface expressed huEpoR was analyzed using FACS. UT-7 cells were incubated with either 5 nM scFv-Fc protein alone or with 5 nM scFv-Fc protein plus 0.5 μg/ml of rHuEpo for 1 hour at 4° C. After 2 quick washes using cold PBS, UT-7 cells were then incubated with 1 μg/ml phycoerythrin-conjugated goat F(ab′)2 anti-human IgG Fc (Jackson Immuno Research Laboratories) for 1 hour at 4° C. The cells were washed twice using cold PBS and resuspended into 1 ml of fixation buffer (2% paraformaldehyde PBS pH 7.4). FACS was done using a FACSCaliber flow cytometer (Becton-Dickinson)

The FACS traces of the proteins expressed from the scFv-Fc expression vectors are shown in FIG. 3. Clone 2, clone 5, clone 7, clone 10, and clone 30 all bind to huEpoR expressing UT-7 cells (FIG. 3A) but not to the negative control cells (FIG. 3B). UT-7 cell surface binding of clone 2, clone 5, clone 7, and clone 10 was blocked by an excess amount of rHuEpo (FIG. 3A). rHuEpo did not block the binding of clone 30 (FIG. 3A).

### Example 4

### Sequences of Clones 2, 5, 7, 10, and 30

Clone 2, clone 5, clone 7, clone 10, and clone 30 were sequenced using standard techniques. Nucleic acid and amino acid sequences for the variable heavy chains and variable light chains of clone 2, clone 5, clone 7, clone 10 and clone 30 appear below. Heavy chain and light chain CDR1, CDR2, and CDR3 are underlined in order within each amino acid sequence.

>Clone #2VH nucleic acid sequence

(SEQ ID NO.: 35)

GAGGTCCAGCTGGTGCAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTACTCGGACTGGGGCCAAGGCACCCTGGTCACCGTCTC
GAGT
>Clone #2VH amino acid sequence

(SEQ ID NO.: 1)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS
>Clone #2VL nucleic acid sequence

(SEQ ID NO.: 36)

CAGTCTGTGCTGACTCAGCCACCCTCCGCGTCCGGGTCTCCTGGACAGTC
AGTCACCATCTCCTGCACTGGAACCAGCAGTGACGTTGGTGGTTATAACT
ATGTCTCCTGGTACCAACAGCACCCAGGCAAAGCCCCCAAACTCATGATT
TATGAGGTCAGTAAGCGGCCCTCAGGGGTCCCTGATCGCTTCTCTGGCTC
CAAGTCTGGCAACACGGCCTCCCTGACCGTCTCTGGGCTCCAGCCTGAGG
ATGAGGCTGATTATTACTGCAGCTCATATGCAGGCAGGAACTGGGTGTTC
GGCGGAGGGACCCAGCTCACCGTTTTA
>Clone #2VL amino acid sequence

(SEQ ID NO.: 2)

QSVLTQPPSASGSPGQSVTISCTGTSSDVGGYNYVSWYQQHPGKAPKLMI
YEVSKRPSGVPDRFSGSKSGNTASLTVSGLQPEDEADYYCSSYAGRNWVF
GGGTQLTVL
>Clone #5VH nucleic acid sequence

(SEQ ID NO.: 37)

GAGGTGCAGCTGGTGGAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCAAGAGTTTCG
AGGGGTGGGAGCTACTCGGACTGGGGCCAGGGAACCCTGGTCACCGTCTC
GAGT
>Clone #5VH amino acid sequence

(SEQ ID NO.: 3)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSS
>Clone #5VL nucleic acid sequence

(SEQ ID NO.: 38)

CAGTCTGCCCTGACTCAGCCTGCCTCCGTGTCTGGGTCTCCTGGACAGTC
GATCACCATCTCCTGCACTGGAACCAGCAGTGACGTTGGTGGCTATATTT
ATGTCTCCTGGTACCAACAACACCCAGGCAAAGCCCCCAAACTCATGATT
TATGATGTCAGTCGTCGGCCCTCAGGGATTTCTGATCGCTTCTCTGGCTC
CAAGTCTGGCAACACGGCCTCCCTGACCATCTCTGGGCTCCAGGCTGAGG
ACGAGGCTGATTATTACTGCAACTCATATACAACCCTCAGCACCTGGCTC
TTCGGCGGAGGGACCAAGGTCACCGTCCTA
>Clone #5VL amino acid sequence

(SEQ ID NO.: 4)

QSALTQPASVSGSPGQSITISCTGTSSDVGGYIYVSWYQQHPGKAPKLMI
YDVSRRPSGISDRFSGSKSGNTASLTISGLQAEDEADYYCNSYTTLSTWL
FGGGTKVTVL
>Clone #7VH nucleic acid sequence

(SEQ ID NO.: 39)

GAGGTGCAGCTGGTGCAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTACTCGGACTGGGGCAAAGGAACCCTGGTCACCGTCTC
GAGT
>Clone #7VH amino acid sequence

(SEQ ID NO.: 5)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSS
>Clone #7VL nucleic acid sequence

(SEQ ID NO.: 40)

CAGTCTGCCCTGACTCAGCCTGCCTCCGTGTCTGGGTCTCCTGGACAGTC
GATCATCATCTCCTGCACTGGAACCCGCAGTGACATTGGTGGTTACAACT
ATGTCTCCTGGTACCAACACCACCCAGGCAGAGCCCCCAAACTCATCATT
TTTGATGTCAATAATCGGCCCTCAGGAGTCTCTCACCGCTTCTCTGGCTC
CAAGTCTGGCAACACGGCCTCCCTGACCATCTCTGGGCTCCAGGCTGAGG
ACGAGGCTGATTATTACTGCAATTCATTTACAGACAGCCGGACTTGGCTG
TTCGGCGGAGGGACCAAGCTGACCGTCCTA
>Clone #7VL amino acid sequence

(SEQ ID NO.: 6)

QSALTQPASVSGSPGQSIIISCTGTRSDIGGYNYVSWYQHHPGRAPKLII
FDVNNRPSGVSHRFSGSKSGNTASLTISGLQAEDEADYYCNSFTDSRTWL
FGGGTKLTVL
>Clone #10VH nucleic acid sequence

(SEQ ID NO.: 41)

GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTGTATTACTGTGTAAAAGATAGG
GTTGCTGTAGCTGGTAAGGGTTCGTATTACTTTGACTCTTGGGGGAGGGG
GACCACGGTCACCGTCTCGAGT
>Clone #10VH amino acid sequence

(SEQ ID NO.: 7)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSS
>Clone #10VL nucleic acid sequence

(SEQ ID NO.: 42)

CAGTCTGTGCTGACGCAGCCGCCCTCGGTGTCTGAAGCCCCCGGGCAGAG
GGTCACCATCGCCTGTTCTGGAAGCAGCTCCAACATCGGAAATAATGCTG
TAAGTTGGTACCAGCAACTCCCAGGAAAGGCTCCCACACTCCTCATCTAT
TATGATAATCTGCTGCCCTCAGGGGTCTCTGACCGATTCTCTGGCTCCAA
GTCTGGCACCTCAGCCTCCCTGGCCATCAGTGGGCTCCAGTCTGAGGATG
AGGCTGATTATTACTGTGCTGCATGGGATGACAGCCTGAATGATTGGGTG
TTCGGCGGTGGGACCAAGGTCACCGTCCTA
>Clone #10VL amino acid sequence

(SEQ ID NO.: 8)

QSVLTQPPSVSEAPGQRVTIACSGSSSNIGNNAVSWYQQLPGKAPTLLIY
YDNLLPSGVSDRFSGSKSGTSASLAISGLQSEDEADYYCAAWDDSLNDWV
FGGGTKVTVL
>Clone #30VH nucleic acid sequence

(SEQ ID NO.: 43)

CAGGTGCAGCTGCAGGAGTCGGGTCCAGGACTGGTGAAGCCCTCGCAGAC
CCTCTCACTCACCTGTGCCATCTCCGGGGACAGTGTCTCTAGCAACAGTG
CTGCTTGGAACTGGATCAGGCAGTCCCCATCGAGAGGCCTTGAGTGGCTG
GGAAGGACATACTACAGGTCCAAGTGGTATAATGATTATGCAGTATCTGT
GAAAAGTCGAATGACCATAAAAGCAGACACATCCAAGAACCAGTTCTCCC
TGCAACTGAACTCTGTGACTCCCGAAGACACGGCTGTGTATTACTGTGCA
AGAGATGAGGGACCGCTTGACTACTGGGGCCAGGGAACCCTGGTCACCGT
CTCGGCC
>Clone #30VH amino acid sequence

(SEQ ID NO.: 9)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSA
>Clone #30VL nucleic acid sequence

(SEQ ID NO.: 44)

CAGGCTGTGCTCACTCAGCCGTCCTCAGTGTCTGGGGCCCCAGGGCAGAG
GGTCACCATCTCCTGCACTGGGAGCAGCTCCAACCTCGGGACAGGTTATG
ATGTACACTGGTACCAGCAGCTTCCAGGAACAGCCCCCAAACTCCTCATC
TATGGTAACAGCAATCGGCCCTCAGGGGTCCCTGACCGATTCTCGGGCTC
CAAGTCTGACACCTCAGGTTTGCTGGCCATCACTGGGCTCCAGGCTGAGG
ATGAGGCTACTTATTACTGCCAGTCCTATGACTTCAGCCTGAGTGCTATG
GTATTCGGCGGAGGGACCAAGGTCACCGTCCTA
>Clone #30VL amino acid sequence

(SEQ ID NO.: 10)

QAVLTQPSSVSGAPGQRVTISCTGSSSNLGTGYDVHWYQQLPGTAPKLLI
YGNSNRPSGVPDRFSGSKSDTSGLLAITGLQAEDEATYYCQSYDFSLSAM
VFGGGTKVTVL

Clones 2, 5, 7, 10, and 30 were used to make scFv-Fc proteins. The nucleic acid sequences and the amino acid sequences of the scFv-Fc proteins that they encode are shown below:

>Mxb #2 scFv-Fc nucleic acid sequence:

(SEQ ID NO.: 50)

GAGGTCCAGCTGGTGCAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTACTCGGACTGGGGCCAAGGCACCCTGGTCACCGTCTC
GAGTGGAGGCGGCGGTTCAGGCGGAGGTGGCTCTGGCGGTGGCGGAAGTG
CACAGTCTGTGCTGACTCAGCCACCCTCCGCGTCCGGGTCTCCTGGACAG
TCAGTCACCATCTCCTGCACTGGAACCAGCAGTGACGTTGGTGGTTATAA
CTATGTCTCCTGGTACCAACAGCACCCAGGCAAAGCCCCCAAACTCATGA
ATTTATGAGGTCAGTAAGCGGCCCTCAGGGGTCCCTGATCGCTTCTCTGG
CTCCAAGTCTGGCAACACGGCCTCCCTGACCGTCTCTGGGCTCCAGCCTG
AGGATGAGGCTGATTATTACTGCAGCTCATATGCAGGCAGGAACTGGGTG
TTCGGCGGAGGGACCCAGCTCACCGTTTTAGGTGCGGCCGCAGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCTCCGGGTAAA

>Mxb #2 scFv-Fc amino acid sequence:

(SEQ ID NO.: 45)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSVLTQPPSASGSPGQ
SVTISCTGTSSDVGGYNYVSWYQQHPGKAPKLMIYEVSKRPSGVPDRFSG
SKSGNTASLTVSGLQPEDEADYYCSSYAGRNWVFGGGTQLTVLGAAAEPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

>Mxb #5 scFv-Vc nucleic acid sequence:

(SEQ ID NO.: 51)

GAGGTGCAGCTGGTGGAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCAAGAGTTTCG
AGGGGTGGGAGCTACTCGGACTGGGGCCAGGGAACCCTGGTCACCGTCTC
GAGTGGAGGCGGCGGTTCAGGCGGAGGTGGCTCTGGCGGTGGCGGAAGTG
CACAGTCTGCCCTGACTCAGCCTGCCTCCGTGTCTGGGTCTCCTGGACAG
TCGATCACCATCTCCTGCACTGGAACCAGCAGTGACGTTGGTGGCTATAT
TTATGTCTCCTGGTACCAACAACACCCAGGCAAAGCCCCCAAACTCATGA
TTTATGATGTCAGTCGTCGGCCCTCAGGGATTTCTGATCGCTTCTCTGGC
TCCAAGTCTGGCAACACGGCCTCCCTGACCATCTCTGGGCTCCAGGCTGA
GGACGAGGCTGATTATTACTGCAACTCATATACAACCCTCAGCACCTGGC
TCTTCGGCGGAGGGACCAAGGTCACCGTCCTAGGTGCGGCCGCAGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGAGGAGATGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGG
CAGGAGGGGAACGTCTTCTCATGCTCCGTGATGCATGAGGCTCTGCACAA
CCACTACACGCAGAAGAGCCTCTCCCTGTCTCCGGGTAAA

>Mxb #5 scFv-Fc amino acid sequence:

(SEQ ID NO.: 46)

EVQLVESGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGQGTLVTVSSGGGGSGGGGSGGGGSAQSALTQPASVSGSPGQ
SITISCTGTSSDVGGYIYVSWYQQHPGKAPKLMIYDVSRRPSGISDRFSG
SKSGNTASLTISGLQAEDEADYYCNSYTTLSTWLFGGGTKVTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

>Mxb #7 scFv-Fc nucleic acid sequence:

(SEQ ID NO.: 52)

GAGGTGCAGCTGGTGCAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTACTCGGACTGGGGCAAAGGAACCCTGGTCACCGTCTC
GAGTGGAGGCGGCGGTTCAGGCGGAGGTGGCTCTGGCGGTGGCGGAAGTG
CACAGTCTGCCCTGACTCAGCCTGCCTCCGTGTCTGGGTCTCCTGGACAG
TCGATCATCATCTCCTGCACTGGAACCCGCAGTGACATTGGTGGTTACAA
CTATGTCTCCTGGTACCAACACCACCCAGGCAGAGCCCCCAAACTCATCA
TTTTTGATGTCAATAATCGGCCCTCAGGAGTCTCTCACCGCTTCTCTGGC
TCCAAGTCTGGCAACACGGCCTCCCTGACCATCTCTGGGCTCCAGGCTGA
GGACGAGGCTGATTATTACTGCAATTCAUTACAGACAGCCGGACTTGGCT
GTTCGGCGGAGGGACCAAGCTGACCGTCCTAGGTGCGGCCGCAGAGCCCA
AATCTTGTGACAAAACTCACACATGCCCACCGTGCCCAGCACCTGAACTC
CTGGGGGGACCGTCAGTCTTCCTCTTCCCCCCAAAACCCAAGGACACCCT
CATGATCTCCCGGACCCCTGAGGTCACATGCGTGGTGGTGGACGTGAGCC
ACGAAGACCCTGAGGTCAAGTTCAACTGGTACGTGGACGGCGTGGAGGTG
CATAATGCCAAGACAAAGCCGCGGGAGGAGCAGTACAACAGCACGTACCG
TGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCT
GCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGC
AGCAGGGGAACGTCTTCTCATGCTCCGTGATGCATGAGGCTCTGCACAAC
CACTACACGCAGAAGAGCCTCTCCCTGTCTCCGGGTAAA

>Mxb #7 scFv-Vc amino acid sequence:

(SEQ ID NO.: 47)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGKGTLVTVSSGGGGSGGGGSGGGGSAQSALTQPASVSGSPGQ
SIIISCTGTRSDIGGYNYVSWYQHHPGRAPKLIIFDVNNRPSGVSHRFSG
SKSGNTASLTISGLQAEDEADYYCNSFTDSRTWLFGGGTKLTVLGAAAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

>Mxb #10 scFv-Fc nucleic acid sequence:

(SEQ ID NO.: 53)

GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTGTATTACTGTGTAAAAGATAGG
GTTGCTGTAGCTGGTAAGGGTTCGTATTACTTTGACTCTTGGGGGAGGGG
GACCACGGTCACCGTCTCGAGTGGAGGCGGCGGTTCAGGCGGAGGTGGCT
CTGGCGGTGGCGGAAGTGCACAGTCTGTGCTGACGCAGCCGCCCTCGGTG
TCTGAAGCCCCCGGGCAGAGGGTCACCATCGCCTGTTCTGGAAGCAGCTC
CAACATCGGAAATAATGCTGTAAGTTGGTACCAGCAACTCCCAGGAAAGG
CTCCCACACTCCTCATCTATTATGATAATCTGCTGCCCTCAGGGGTCTCT
GACCGATTCTCTGGCTCCAAGTCTGGCACCTCAGCCTCCCTGGCCATCAG
TGGGCTCCAGTCTGAGGATGAGGCTGATTATTACTGTGCTGCATGGGATG
ACAGCCTGAATGATTGGGTGTTCGGCGGTGGGACCAAGGTCACCGTCCTA
GGTGCGGCCGCAGAGCCCAAATCTTGTGACAAAACTCACACATGCCCACC
GTGCCCAGCACCTGAACTCCTGGGGGGACCGTCAGTCTTCCTCTTCCCCC
CAAAACCCAAGGACACCCTCATGATCTCCCGGACCCCTGAGGTCACATGC
GTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGC
AGTACAACAGCACGTACCGTGTGGTCAGCGTCCTCACCGTCCTGCACCAG
GACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAG
AACCACAGGTGTACACCCTGCCCCCATCCCGGGAGGAGATGACCAAGAAC
CAGGTCAGCCTGACCTGCCTGGTCAAAGGCTTCTATCCCAGCGACATCGC
CGTGGAGTGGGAGAGCAATGGGCAGCCGGAGAACAACTACAAGACCACGC
CTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTC
CGGGTAAA

>Mxb #10 scFv-Fc amino acid sequence:

(SEQ ID NO.: 48)

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCVKDR
VAVAGKGSYYFDSWGRGTTVTVSSGGGGSGGGGSGGGGSAQSVLTQPPSV
SEAPGQRVTIACSGSSSNIGNNAVSWYQQLPGKAPTLLIYYDNLLPSGVS
DRFSGSKSGTSASLAISGLQSEDEADYYCAAWDDSLNDWVFGGGTKVTVL
GAAAEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLT
VDKSRWQQGNVFSCSVMHEALHNHYTQKSLSLSPGK

(SEQ ID NO.: 54)

CAGGTGCAGCTGCAGGAGTCGGGTCCAGGACTGGTGAAGCCCTCGCAGAC
CCTCTCACTCACCTGTGCCATCTCCGGGGACAGTGTCTCTAGCAACAGTG
CTGCTTGGAACTGGATCAGGCAGTCCCCATCGAGAGGCCTTGAGTGGCTG
GGAAGGACATACTACAGGTCCAAGTGGTATAATGATTATGCAGTATCTGT
GAAAAGTCGAATGACCATAAAAGCAGACACATCCAAGAACCAGTTCTCCC
TGCAACTGAACTCTGTGACTCCCGAAGACACGGCTGTGTATTACTGTGCA
AGAGATGAGGGACCGCTTGACTACTGGGGCCAGGGAACCCTGGTCACCGT
CTCGGCCGGTGGCGGTGGCAGCGGCGGTGGTGGGTCCGGTGGCGGCGGAT
CTGGCGCGCCACAGGCTGTGCTCACTCAGCCGTCCTCAGTGTCTGGGGCC
CCAGGGCAGAGGGTCACCATCTCCTGCACTGGGAGCAGCTCCAACCTCGG
GACAGGTTATGATGTACACTGGTACCAGCAGCTTCCAGGAACAGCCCCCA
AACTCCTCATCTATGGTAACAGCAATCGGCCCTCAGGGGTCCCTGACCGA
TTCTCGGGCTCCAAGTCTGACACCTCAGGTTTGCTGGCCATCACTGGGCT
CCAGGCTGAGGATGAGGCTACTTATTACTGCCAGTCCTATGACTTCAGCC
TGAGTGCTATGGTATTCGGCGGAGGGACCAAGGTCACCGTCCTAGCGGCC
GCAGAGCCCAAATCTTGTGACAAAACTCACACATGCCCACCGTGCCCAGC
ACCTGAACTCCTGGGGGGACCGTCAGTCTTCCTCTTCCCCCCAAAACCCA
AGGACACCCTCATGATCTCCCGGACCCCTGAGGTCACATGCGTGGTGGTG
GACGTGAGCCACGAAGACCCTGAGGTCAAGTTCAACTGGTACGTGGACGG
CGTGGAGGTGCATAATGCCAAGACAAAGCCGCGGGAGGAGCAGTACAACA
GCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGG
TGTACACCCTGCCCCCATCCCGGGAGGAGATGACCAAGAACCAGGTCAGC
CTGACCTGCCTGGTCAAAGGCTTCTATCCCAGCGACATCGCCGTGGAGTG
GGAGAGCAATGGGCAGCCGGAGAACAACTACAAGACCACGCCTCCCGTGC
TGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGC
TCTGCACAACCACTACACGCAGAAGAGCCTCTCCCTGTCTCCGGGTAAA

(SEQ ID NO.: 49)

QVQLQESGPGLVKPSQTLSLTCAISGDSVSSNSAAWNWIRQSPSRGLEWL
GRTYYRSKWYNDYAVSVKSRMTIKADTSKNQFSLQLNSVTPEDTAVYYCA
RDEGPLDYWGQGTLVTVSAGGGGSGGGGSGGGGSGAPQAVLTQPSSVSGA
PGQRVTISCTGSSSNLGTGYDVHWYQQLPGTAPKLLIYGNSNRPSGVPDR
FSGSKSDTSGLLAITGLQAEDEATYYCQSYDFSLSAMVFGGGTKVTVLAA
AEPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDK
SRWQQGNVFSCSVMHEALHNHYTQKSLSLSPGK

### Example 5

### Competitive Binding of Clones 2, 5, 7, 10 and 30 to huEpoR

Clone 2, clone 5, clone 7, clone 10, and clone 30 scFv-Fc proteins were tested for their ability to compete with clone 5 and clone 30 scFv phage for binding to huEpoR using a plate-based ELISA. Biotinylated huEpoR was immobilized on a streptavidin plate. A scFv-Fc protein and a scFv phage were added to the plate. Binding of the scFv phage was then detected using an anti-M13 mouse monoclonal antibody followed by a phycoerythrin-conjugated goat F(ab′)2 anti-mouse IgG Fc (Jackson Immuno Research Laboratories). The inhibition of phage binding by clone 2, clone 5, clone 7, clone 10 and clone 30 scFv-Fc protein was tested by using a series of 8 concentrations for each scFv-Fc protein (0, 0.032, 0.16, 0.8, 4, 20, 100, and 500 nM). Clone 2, clone 5, clone 7, and clone 10 scFv-Fc proteins demonstrated a dose dependent inhibition of binding of clone 5 scFv phage to huEpoR (FIG. 4A). However, clone 30 scFv-Fc protein did not inhibit binding of clone 5 scFv phage to huEpoR at concentrations up to 500 nM (FIG. 4A). Binding of clone 30 scFv phage to huEpoR was inhibited by clone 30 scFv-Fc protein in a dose dependent fashion, but not by clone 2, clone 5, clone 7, or clone 10 scFv-Fc proteins at concentrations up to 500 nM (FIG. 4B). Those results suggest that the epitopes for clone 2, clone, 7, and clone 10 scFv-Fc proteins overlap with the epitope of clone 5 scFv-Fc protein, but that clone 30 scFv-Fc protein binds to an epitope that does not overlap with the epitopes of clone 2, clone 5, clone 7, and clone 10 scFv-Fc proteins.

### Example 6

### Antibody Binding to Mouse EpoR-Fc protein (muEpoR-Fc)

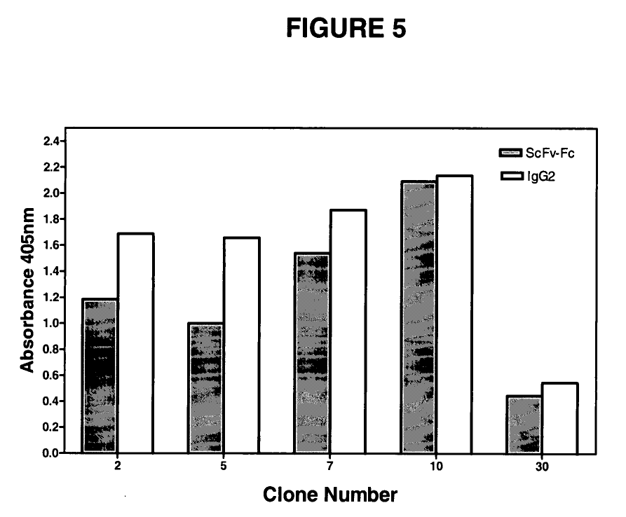

The cross reactivity of clone 2, clone 5, clone 7, clone 10, and clone 30 scFv-Fc proteins and clone 2, clone 5, clone 7, clone 10, and clone 30 IgG₂ proteins with mouse EpoR (muEpoR) was determined using an ELISA assay. Individual scFv-Fc proteins or IgG₂ proteins (100 μl of a 1 μg/ml antibody stock in 50 mM NaHCO₃, pH8.5) were added to each well on a Nunc-Immuno Polysorp ELISA plate (Nalge Nunc International) such that each well comprised only a single clone. The plate was incubated at 4° C. overnight. After blocking the wells with 4% milk/PBS/0.1% tween 20 for 1 hour at room temperature, plates were washed three times with PBS/0.1% tween 20. 100 μl of 5 μg/ml biotinylated muEpoR-Fc protein was added to each well and incubated for 1 hour at 25° C. The bound muEpoR-Fc was detected using streptavidin-HRP conjugate (1:1000 dilution in 4% milk PBS/0.1% tween 20). 2,2′-azino-bis(3-ethylbenzthiazoline-6-sulphonic acid) (ABTS) was used as a substrate and the absorption was measured at 405 nm on a plate reader. All of the antibodies (clone 2, clone 5, clone 7, clone 10, and clone 30 scFv-Fc proteins and clone 2, clone 5, clone 7, clone 10, and clone 30 IgG₂ proteins) showed significant levels of cross reactivity to muEpoR-Fc (FIG. 5).

### Example 7

### Measurement of Binding Kinetics to huEpoR Using BIAcore

The affinities for clone 2, clone 5, clone 7, clone 10, and clone 30 scFv-Fc proteins were determined on a BIAcore 3000 instrument (BIAcore International AB). Goat anti-human Fc antibody (Jackson Immuno Research Laboratories) was immobilized on a CM4 chip (BIAcore International AB) activated through N-hydroxyl succinamide chemistry. An scFv-Fc protein solution was flowed over the chip and the scFv-Fc protein in the solution was captured on the chip through Fc binding to the immobilized goat anti human Fc antibody. Each kinetics run used a 50 μl/min flow rate at 25° C. Each run used huEpoR protein at concentrations up to 1000 nM as analyte. An association phase of 1 minute and dissociation phase of 5 minutes were used for data analysis by 1:1 Langmuir with mass transfer+local Rmax fit using BIAevaluation software version 3 provided by BIAcore. Flowing low pH glycine buffer (50 mM glycine HCl, pH 1.5) over the chip to remove the captured scFv-Fc protein regenerated the goat anti-human Fc antibody CM4 chip surface. This same chip surface was used for separately capturing each of the five scFv-Fc proteins.

BIAcore kinetic binding sensograms are shown in FIG. 6 and the binding parameters are summarized in Table 2 below. The affinities for the five different scFv-Fc proteins varied from 1.1 nM to 14,900 nM. The association and dissociation rate (kₒₙ and k_{off}, respectively) for all five scFv-Fc proteins were within typical ranges for antibodies. The highest affinity scFv-Fc protein, the clone 10 scFv-Fc protein, had the slowest k_{off} (2.2×10⁻⁴ s⁻¹). The lowest affinity scFv-Fc protein, the clone 30 scFv-Fc protein, had the slowest kₒₙ (1.8×10⁴ M⁻¹s⁻¹) and fastest k_{off} (2,740×10⁻⁴ s⁻¹).

TABLE 2
Summary of scFv-Fc BIAcore binding kinetics to huEpoR

| ScFv-Fc clone | kₒₙ (10⁵, 1/Ms) | k_{off} (10⁻⁴, 1/s) | K_{D} (10⁻⁹, M) |
|---|---|---|---|
| #2 | 4.1 | 1,360 | 334 |
| #5 | 2.8 | 612 | 217 |
| #7 | 2.0 | 541 | 271 |
| #10 | 2.0 | 2.2 | 1.1 |
| #30 | .18 | 2,740 | 14,900 |

### Example 8

### Screening of scFv-Fc Proteins in vitro for the Activation of the Human Erythropoietin Receptor

The twenty-nine scFv sequences identified in Example 1 were screened as either scFv-Fc proteins or as IgG proteins for the activation of the huEpoR. The in vitro screening of the scFv-Fc proteins and IgG proteins was done by a luciferase-based reporter assay (luciferase assay) in UT-7 cells (human megakaryoblasts) transfected with a construct containing nine STAT5 binding sites in front of a luciferase reporter (UT-7-LUC cells). All cells were maintained and all cellular assays were conducted at 37° C. in a humidified incubator at 5% CO₂/95% atmospheric air, unless otherwise noted. All fetal bovine serum (FBS) was heat inactivated at 55° C. for 45 minutes prior to usage. All Dulbecco's Phosphate-Buffered Saline (PBS) used for cell manipulation was without calcium chloride and magnesium chloride. UT-7-LUC cells (Amgen, Inc.; Thousand Oaks, Calif.) were maintained in growth media comprising IMDM (Invitrogen; Carlsbad, Calif.) containing 10% FBS (HyClone; Logan, Utah), 500 μg/mL hygromycin (Roche; Penzberg, Germany), 100 U/mL penicillin, 100 μg/mL streptomycin, 292 μg/mL L-glutamine (1×PSG; Invitrogen) and 1 U/mL recombinant human erythropoietin (Epoetin Alpha, rHuEpo; Amgen, Inc.). The cells were washed two times in PBS (Invitrogen) and resuspended at 400,000 cells per mL in assay media (RPMI Medium 1640 with 1% FBS, 1×PSG, and 12.5 mM HEPES (Invitrogen)). Following an overnight incubation, cell number and viability were determined, and the cells were resuspended at 200,000 cells per mL in assay media.

Each scFv-Fc protein was serially diluted in a 96-well opaque plate (Corning; Corning, N.Y.). Each dilution was run in triplicate and the following concentrations of scFv-Fc protein were used: Mxb 5, Mxb 10, and Mxb 30: 1000, 333, 111, 37.04, 12.35, 4.115, 1.372, 0.457, 0.152, 0.051, 0.017, and 0.006 nM. For Mxb 2 and Mxb 7: 2500, 1250, 625, 312.5, 156.25, 78.125, 39.0625, 19.53125, 9.765625, 4.882813, 2.441-406, 1.220703, 0.610352, 0.3051758, 0.1525879, 0.76294, 0.038147, 0.019073, 0.009537, 0.004768, 0.002384, 0.001192, 0.000596, 0.000298 nM. To serve as a control standard, rHuEpo was serially diluted in the same plate used to test each scFv-Fc protein. Each Epo dilution was run in triplicate and the following concentrations of Epo were used: for the plates with Mxb 2, Mxb 5, Mxb 10, and Mxb 30: 100, 10, 1, 0.1, 0.01, and 0.001 nM. For the plate testing Mxb 7: 1488, 744, 372, 186, 93, 46.5, 23.2, 11.6, 5.8, 2.9, 1.5, 0.71, 0.36, 0.18, 0.09, 0.045, 0.023, 0.011, 0.006, 0.003, 0.0015, 0.0007, 0.0004, 0.0002 nM. Approximately 10,000 cells were added to each well. The cells were then cultured for six hours. The plates were removed from the incubator and allowed to equilibrate to room temperature for 30 minutes. 100 μl of the Steady-Glo Luciferase Assay reagent. (Promega Corporation) were added to each well, and the plates were wrapped in aluminum foil and placed on a plate shaker for 2 minutes. The plates were then held at room temperature for 10 minutes prior to reading the luciferase activity on a 96-well plate luminometer (Victor², PerkinElmer; Boston, Mass.). Raw data was processed by subtracting the background luminescence (values from wells containing media only) and presented as the average of three values ± the standard deviation.

Twenty-two of the twenty-nine maxibodies identified in Example 1 were shown to bind the huEpoR and induce a response in the UT-7-Luc cells of varying degrees. The results for Mxb 2, Mxb 5, Mxb 7, Mxb 10, Mxb 30 are represented graphically in FIG. 7.

### Example 9

### Screening of Antibodies in vitro for the Activation of the huEpoR

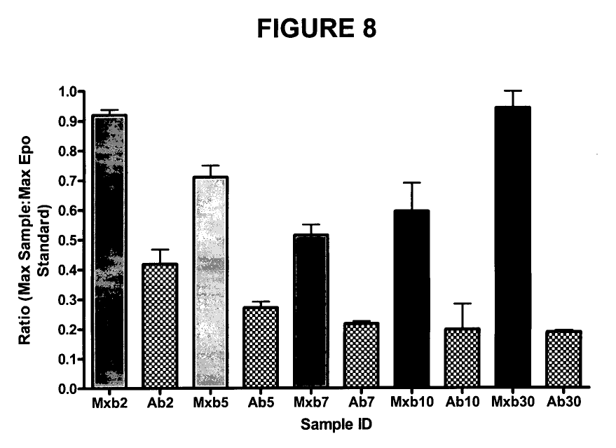

The twenty-nine scFv-Fc proteins described in Example 2 and the twenty-nine IgG₂ proteins also described in Example 2 were individually used to activate the huEpoR using a luciferase-based reporter assay as reported above for the scFv-Fc proteins in Example 8. The resulting dose-titrations were converted to ratios of the maximal luciferase signal of the antibody (scFv-Fc protein or IgG₂ protein) to the maximal luciferase signal of the recombinant human erythropoietin (rHuEpo) standard. The results for clone 2, clone 5, clone 7, clone 10, and clone 30 scFv-Fc proteins and clone 2, clone 5, clone 7, clone 10, and clone 30 IgG₂ proteins are represented graphically in FIG. 8. The clone 2, clone 5, clone 7, clone 10, and clone 30 scFv-Fc proteins were more potent agonists of the huEpoR than the corresponding clone 2, clone 5, clone 7, clone 10, and clone 30 IgG₂ proteins.

### Example 10

### In vitro Signaling Experiments

UT-7 cells were maintained in growth media consisting of IMDM (Invitrogen) containing 10% FBS (HyClone), 100 U/mL penicillin, 100 μg/mL streptomycin, 292 μg/mL L-glutamine (1×PSG; Invitrogen) and 1 U/mL rHuEpo (Epoetin Alpha, rHuEpo; Amgen Inc.). The cells were washed two times in PBS (Invitrogen) and resuspended in starvation media consisting of IMDM and 0.5% FBS. Following an overnight incubation, cell number and viability were determined, and the cells were resuspended at 3,000,000 cells per mL in IMDM containing either 50 ng/mL rHuEpo, 1 μg/mL Mxb2, 1 μg/mL Mxb5, 1.54 μg/mL clone 2 IgG₂ protein (IgG₂2), 1.54 μg/mL clone 5 IgG₂ protein (IgG₂5), or PBS. Cells were stimulated for 0, 2, 15, or 60 minutes in a 37° C. heat block. Activation of these cells by rHuEpo engages the huEpoR and induces phosphorylation of the signaling molecules Stat5 and Akt. The cell suspensions were then centrifuged for 1 minute, 7000 rpm, at 4° C. and the supernatant was removed. The cell pellet was washed with ice-cold PBS and centrifuged for 1 minute, 7000 rpm, at 4° C. The supernatant was removed and cell lysates were generated using M-PER mammalian protein extraction reagent (Pierce Biotechnology, Inc.; Rockford, Ill.) supplemented with Complete (EDTA-free) protease inhibitor cocktail tablets (Roche Diagnostics). All of the samples were then vortexed for 10 seconds, and the lysates were incubated at room temperature for 5 minutes with occasional vortexing. The lysates were then centrifuged at 2000 rpm for 5 minutes, and the supernatants were transferred into aliquots and snap frozen in a dry ice/ethanol bath and stored at −80° C. until used.

Western Blotting: All protein samples were combined with 1× NuPAGE Sample Reducing Agent (Invitrogen) and 1× NuPAGE LDS sample buffer (Invitrogen), incubated at 100° C. for 5 minutes, and run on pre-cast 4-20% Tris-Glycine gels (Invitrogen). All gels were loaded with the SeeBlue Plus2 protein ladder (Invitrogen). Proteins were then transferred to a nitrocellulose membrane filter paper sandwich with 0.45 μm pore size (Invitrogen). Following the protein transfer, the membranes were blocked in 5% blotting grade blocker non-fat dry milk (milk; Bio-Rad Laboratories; Hercules, Calif.) in tris-buffered saline with tween 20, pH 8.0 (TBS-T; SIGMA) for at least one hour at room temperature. The membranes were first blotted with an anti-phosphorylated Stat5 A/B antibody (Upstate; Charlottesville, Va.) at 1 μg/mL in 2.5% bovine serum albumin (BSA; SIGMA) in TBS-T. Incubations with the anti-phosphorylated Stat5 A/B antibody were conducted for one hour at room temperature on a shaking platform, followed by three rinses and three washes for 15 minutes in TBS-T. The membranes were then blotted with a goat anti-mouse—horseradish peroxidase (HRP) conjugated antibody (Pierce Biotechnology, Inc.) diluted to 1:2000 in 1.25% BSA in TBS-T. All of the incubations with the goat anti-mouse—HRP conjugated antibody were performed for one hour at room temperature on a shaking platform, followed by three rinses and three washes for 15 minutes in TBS-T. Enhanced chemiluminescence (ECL) western blotting detection system (Amersham Bioscience) was used to detect the proteins on the nitrocellulose membranes. The membranes were then exposed to Kodak BIOMAX Light Film for chemiluminescence (Kodak; Rochester, N.Y.). Following detection, the membranes were stripped in Restore Western Blot Stripping Buffer (PIERCE) for 20 minutes.

Blotting was repeated using the same process described above for the following antibodies: Total Stat5: primary antibody—anti-Stat5 (Cell Signaling Technology; Danvers, Mass.) at 1:1000, secondary antibody—goat anti-rabbit-HRP (Pierce Biotechnology, Inc.) at 1:2000 dilution. Phosphorylated Akt: primary antibody—anti-phosphorylated Akt (Thr308) (Cell Signaling Technology) 1:1000 dilution, secondary antibody—goat anti-rabbit-HRP 1:2000 dilution. Total Akt: primary antibody—anti-Akt (Cell Signaling Technology) at 1:1000 dilution, secondary antibody—goat anti-rabbit HRP 1:2000.

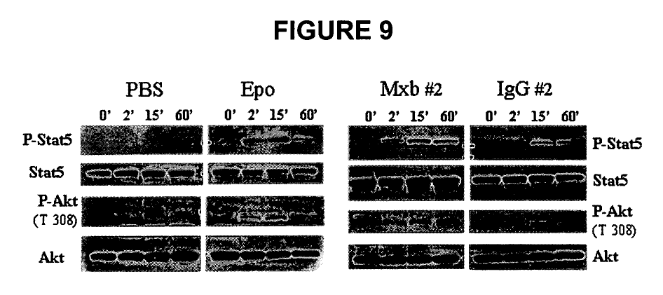

The results of this experiment demonstrated that Mxb 2, Mxb 5, IgG₂ 2, and IgG₂ 5 activated the huEpoR and induced phosphorylation of both Stat5 and Akt. The kinetics of phosphorylation by Mxb 2, Mxb 5, IgG₂2, and IgG₂5 were slightly delayed in relation to rHuEpo. The results for Mxb 2 and IgG₂2 are shown in FIG. 9. FIG. 9 shows that after rHuEpo stimulation of UT-7 cells, strong phosphorylation of Stat5 was detected within 2 minutes and reached a maximum at 15 minutes, whereas, in the case of Mxb 2 and IgG₂2, the level of Stat5 phosphorylation was low at 2 minutes after stimulation. The same was true for Akt phosphorylation. The level of Stat5 and Akt phosphorylation was lower in cells stimulated by IgG₂ 2 compared to cells stimulated by Mxb 2. This signaling experiment indicated that Mxb 2 and IgG₂ 2 were weaker agonists of the huEpoR than rHuEpo.

### Example 11

### BFU-E Assays

The activity of a subset of Mxbs including Mxb 2, Mxb 5, Mxb 7, and Mxb 30 was evaluated on CD34+ human peripheral blood progenitor cells (CD34+PBPC) using a Burst Forming Unit-Erythroid (BFU-E) assay. The BFU-E assay is generally described in Elliott et al., Activation of the Erythropoietin(EPO) receptor by bivalent anti-EPO receptor antibodies, J. Biol. Chem. 271(40), 24691-24697. In this case, the BFU-E assay tested the ability of scFv-Fc proteins to stimulate the production of erythroid colonies from human primary cells isolated from the blood of healthy volunteers. Certain agents that promote erythroid colony formation also promote proliferation of erythroid progenitor cells, prevent apoptosis, and induce cellular differentiation.

For this assay, CD34+PBPC were purified from apheresis products obtained from rhG-CSF mobilized hematologically normal donors. One thousand CD34+PBPC per mL were cultured in 35 mm petri dishes in a methylcellulose-based medium (METHOCULT™ H4230, StemCell Technologies, Vancouver, BC, Canada) containing 100 ng/mL each of rhSCF, rhIL-3, and rhIL-6 with log escalating doses from 0.1 to 1,000 ng/mL of rHuEpo or 1 to 10,000 ng/mL of either Mxb 2, Mxb 5, Mxb 7, or Mxb 30, all in triplicate. Cultures were incubated at 37° C. in 5% CO₂/95% atmospheric air in a humidified chamber, and 14 days later, the number of BFU-E derived colonies was counted. Each culture was observed and enumerated with a dissecting microscope at 20×. BFU-E derived colonies were defined as uni- or multi-focal hemoglobinized cellular clusters containing greater than 50 cells.

Mxb 2, Mxb 5, Mxb 7, and Mxb30 induced the formation of hemoglobin-containing erythroid colonies, but all maxibodies were significantly less potent than rHuEpo in inducing BFU-E-derived colonies. The maximal number of colonies induced by any of the maxibodies was significantly lower than the number induced by rHuEpo, and this maximal number was induced at significantly higher concentrations than in the case of rHuEpo as seen in FIG. 10. These data suggest that the scFv-Fc proteins are low potency agonists of the huEpoR compared to rHuEpo.

### Example 12

### In vivo Experiments

The effect of a single injection of Mxb 2, Mxb 5, Mxb 7, or Mxb 10 was tested in several experiments in mice.

### Example 12A

### Mxb 5 Dose Titration Experiment in Mice

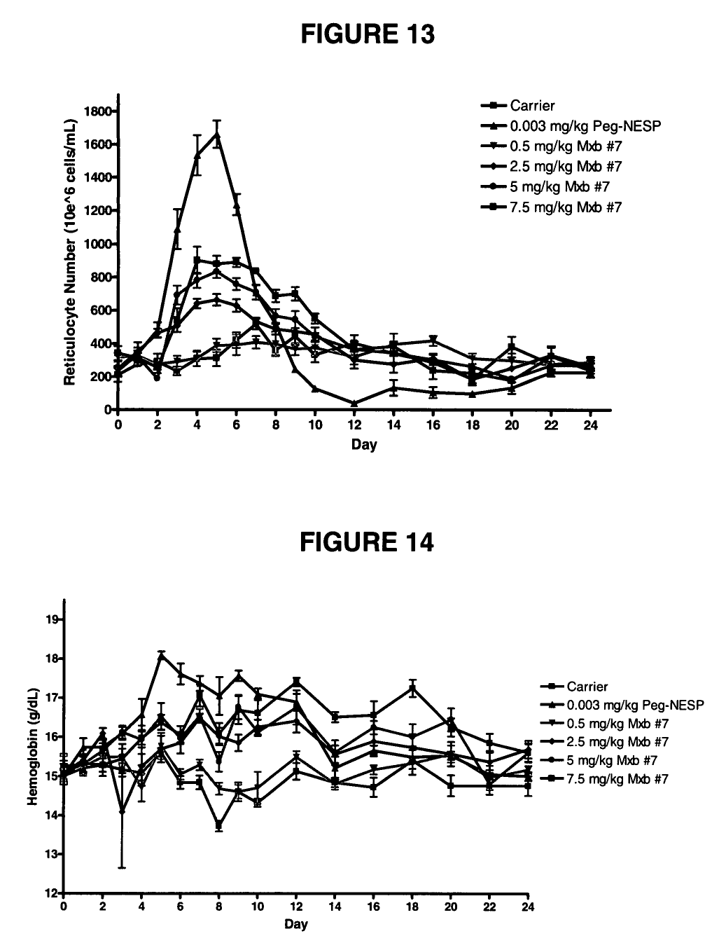

2-month-old female BDF-1 mice were injected subcutaneously with carrier (PBS with 0.1% BSA), 3 μg/kg PEG-NESP (PEG-NESP and methods of preparing PEG-NESP are generally described in PCT publication no. WO01/76640), or 0.5, 2.5, 5, or 7.5 mg/kg Mxb 5 in a final volume of 200 μl. Blood was collected from the retro-orbital sinus at numerous time-points for up to 60 days and evaluated for CBC parameters using an ADVIA blood analyzer. Data are presented in FIGS. 12 and 13 with n=5 at each time point.

There was a clear dose effect of Mxb 5 with very limited activity at 0.5 mg/kg, but significant erythropoietic activity was observed in mice injected with doses of Mxb 5 between 2.5 and 7.5 mg/kg. The activity profile of Mxb 5 was different from that of PEG-NESP; the peak reticulocyte number was achieved on day 4 after an injection of either PEG-NESP or Mxb 5, but the duration of the reticulocyte response was significantly increased in the mice that received doses of Mxb 5 between 2.5 and 7.5 mg/kg. The reticulocyte numbers returned to baseline on day 8 in the PEG-NESP-treated mice, but it took 14 to 18 days for the reticulocytes to return to baseline in the Mxb 5-treated mice. In mice injected with Mxb 5 at doses between 5 and 7.5 mg/kg, the hemoglobin levels stayed above baseline for 46 to 52 days. In contrast, the hemoglobin level in the PEG-NESP-treated mice returned to baseline at day 16, thus showing a very significant difference in the duration and magnitude of the hemoglobin response in the mice treated with Mxb 5 or PEG-NESP. This experiment demonstrates that a single injection of Mxb 5 increases hemoglobin levels above baseline for a period of time that is longer than the total life span of the red blood cells in mice (40 days). Since the rate of hemoglobin decline after the administration of an erythropoietic agent is related to the life span of erythrocytes (120 days in humans), a single administration of Mxb 5 in humans could potentially be enough to correct anemia over a period of 2-4 months.

### Example 12B

### Mxb 7 Dose Titration Experiment in Mice

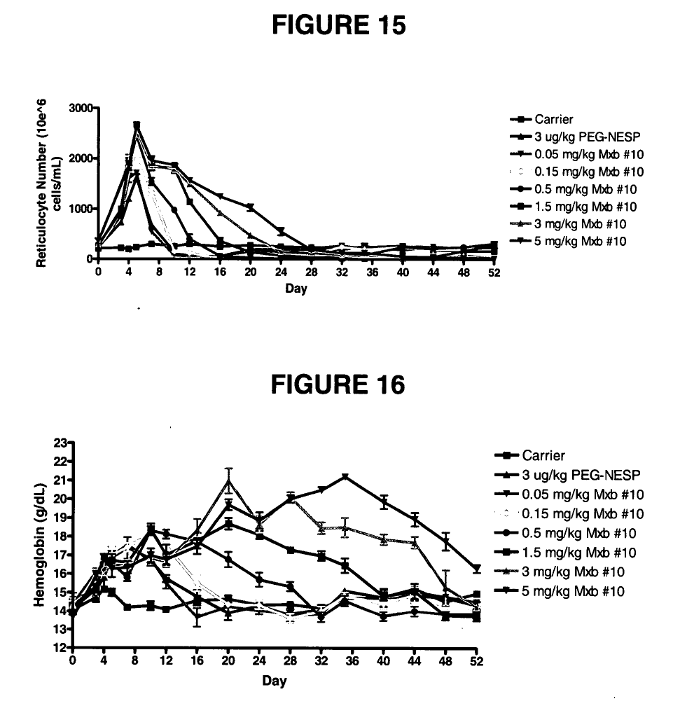

2-month-old female BDF-1 mice were injected subcutaneously with carrier (PBS with 0.1% BSA), 3 μg/kg PEG-NESP (Amgen, Inc.), or 0.5, 2.5, 5, or 7.5 mg/kg Mxb 7 (Amgen, Inc.) in a final volume of 200 μl. Blood was collected from the retro-orbital sinus at numerous time-points for up to 24 days and evaluated for CBC parameters using an ADVIA blood analyzer. Data are presented in FIGS. 14 and 15 with n=5 at each time point.

A single injection of Mxb 7 produced an increase in reticulocyte numbers and hemoglobin levels that were dose-dependent and sustained over a long period of time. After a single subcutaneous (SC) injection of Mxb 7 at 7.5 mg/kg, the reticulocyte numbers stayed above baseline for 12 days while in the mice injected with PEG-NESP, the reticulocyte numbers stayed above baseline for 8 days. In this experiment, hemoglobin levels were measured for 24 days, and during this time, the increase in hemoglobin was sustained at higher levels and for a longer period of time in the mice that received Mxb 7 at 7.5 mg/kg compared to the PEG-NESP-treated mice. After a single PEG-NESP injection, the hemoglobin peak was reached on day 5, and hemoglobin was back to baseline on day 14. In contrast, after a single injection of Mxb 7 (7.5 mg/kg), the hemoglobin peak was reached on day 12, and hemoglobin returned to baseline on day 24. This experiment indicates that Mxb 7 had very different properties from PEG-NESP. After a single administration, the mice treated with Mxb 7 had a longer-duration erythropoietic response than PEG-NESP-treated mice as demonstrated by the increase in reticulocyte numbers and hemoglobin levels.

### Example 12C

### Mxb 10 Dose Titration Experiment in Mice

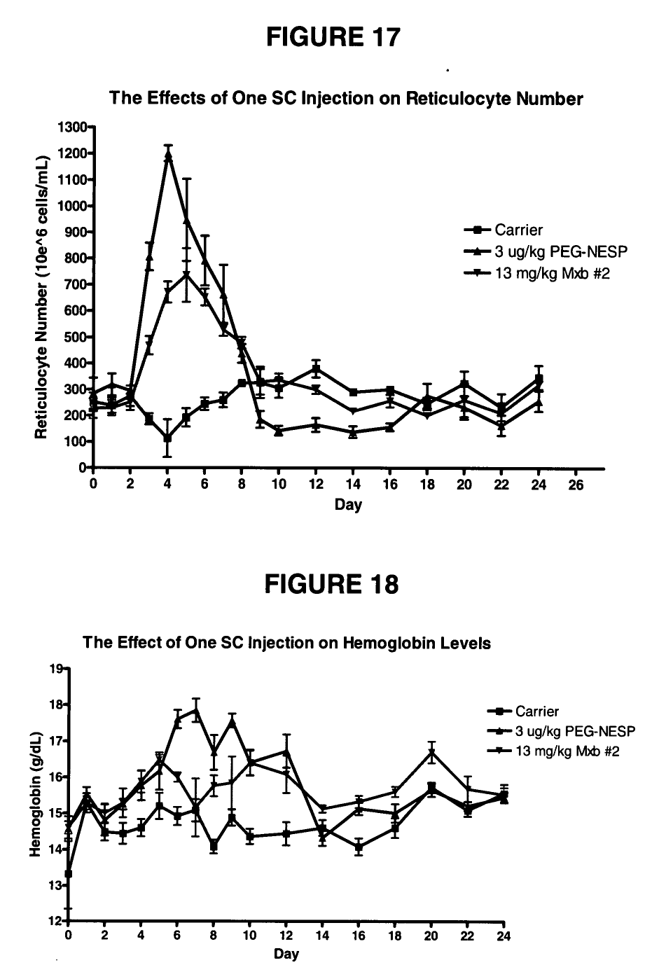

2-month-old female BDF-1 mice were injected subcutaneously with carrier (PBS with 0.1% BSA), 3 μg/kg PEG-NESP (Amgen, Inc.), or 0.05, 0.15, 0.5, 1.5, 3, or 5 mg/kg Mxb 10 (Amgen, Inc.) in a final volume of 200 μl. Blood was collected from the retro-orbital sinus at numerous time-points for up to 52 days and evaluated for CBC parameters using an ADVIA blood analyzer. Data are presented in FIGS. 16 and 17 with n=5 at each time point.

There was a very clear dose-dependent effect of Mxb 10. Changes in reticulocyte numbers and hemoglobin levels were evident even at the lowest dose (0.05 mg/kg) of Mxb 10, which had an activity very similar to 3 μg/kg of PEG-NESP. Mxb 10 was a more potent agent than Mxb 2, Mxb 7, and Mxb 5. In the mice that were treated with 0.15 mg/kg of Mxb 2, the reticulocyte numbers stayed above baseline for 10 days and hemoglobin levels were above baseline for 19 days. At the dose of 0.5 mg/kg of Mxb 10, the reticulocyte numbers stayed above baseline for 13 days and hemoglobin levels were above baseline for 31 days. At the dose of 1.5 mg/kg of Mxb 10, the reticulocyte numbers stayed above baseline for 18 days and hemoglobin levels were above baseline for 40 days. At the dose of 3 mg/kg of Mxb 10, the reticulocyte numbers stayed above baseline for 23 days and hemoglobin levels were above baseline for 50 days. Finally, at the dose of 5 mg/kg of Mxb 10, the reticulocyte numbers stayed above baseline for 28 days and hemoglobin levels were still above baseline at day 52 when the experiment was terminated. In another experiment with mice dosed at 5 mg/kg of Mxb 10, the hemoglobin level returned to baseline at day 56 after a single subcutanious injection of Mxb 10.

### Example 12D

### Mxb 2 Single Dose Experiment in Mice

3-month-old female BDF-1 mice were injected subcutaneously with carrier (PBS with 0.1% BSA), 3 μg/kg PEG-NESP (Amgen, Inc.), or 13 mg/kg Mxb 2 (Amgen, Inc.) in a final volume of 200 μl. Blood was collected from the retro-orbital sinus at numerous time-points for up to 24 days and evaluated for CBC parameters using an ADVIA blood analyzer (Bayer; Germany). Data are presented in FIGS. 18 and 19 with n=5 at each time point.

In this experiment, the erythropoietic effects of a single dose of Mxb 2 were compared to those induced by the control agent PEG-NESP. Reticulocyte numbers stayed above baseline for an additional day in the animals that received Mxb 2 (8 days in the PEG-NESP-treated animals versus 9 days in the Mxb 2-treated mice), but the magnitude of the differences in the erythropoietic responses were significantly accentuated when considering the hemoglobin response. Hemoglobin levels returned to baseline 14 days after PEG-NESP treatment, whereas it took 24 days for the hemoglobin to drop to baseline in the mice treated with Mxb 2. These data further demonstrated that the erythropoietic response induced by Mxb 2 was significantly longer than that induced by PEG-NESP.

### Example 13

### Phamacokinetics Study of Mxb 5 and IgG₁5

The pharmacokinetic (PK) profiles of Mxb 5 and IgG₁5 were characterized in female BDF-1 mice. The animals were injected intravenously with either 3.75 mg/kg Mxb 5 or 5.7 mg/kg IgG₁5 (equimolar amounts). Blood was collected from either the retro-orbital sinus or by cardiac puncture at numerous time points for 100 days with n=4 at each time point. The blood samples were transferred to Costar microcentrifuge tubes and allowed to clot. The samples were then centrifuged at 11,500 rpm for 10 minutes at 4° C. The resulting serum samples were then transferred into individual tubes and stored at −70° C. prior to analysis. Mxb 5 and IgG₁5 concentrations in the samples were measured by ELISA using immobilized huEpoR protein and an anti-human Fc/HRP conjugate. Pharmacokinetic analysis was carried out using serum concentration values over time.

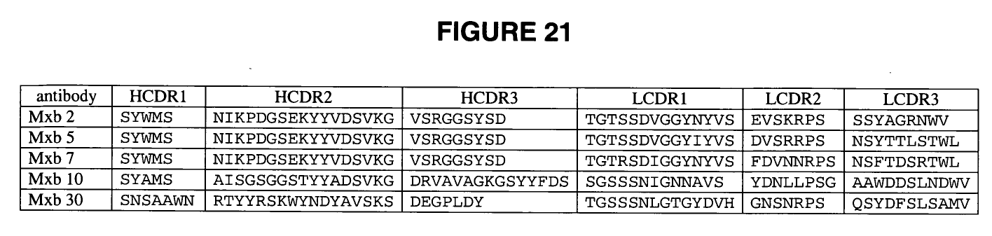

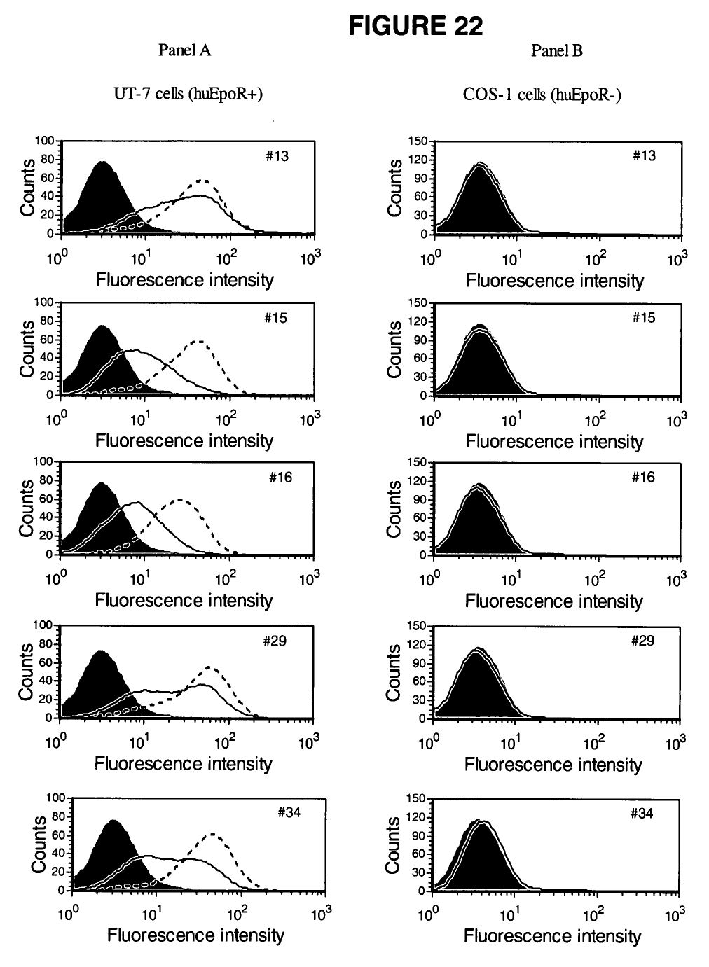

The average and standard deviation of the serum concentration for each protein at each time-point (mean composite) used for this analysis is depicted in FIG. 19. Some pharmacokinetic parameters of IgG₁5 and Mxb 5 are shown in FIGS. 21A, 21B, and 22. IgG₁5 showed a longer half-life than Mxb 5 (320.1 vs. 158.3 hours, respectively). Consistently, the clearance is slower for IgG₁5 than for the Mxb 5 (0.0071 vs. 0.012 mL/hour, respectively) and the Mean Residence Time is greater for IgG₁5 than the Mxb 5 (482.27 vs. 217.51 hours, respectively) This analysis suggests significant differences in the pharmacokinetic profile of these two proteins, with a longer residence time for IgG₁5 versus the Mxb 5 due to its slower elimination.

### Example 14

### Screening and Identification of Additional Clones

scFv phage from naïve phage libraries were put through two rounds of selection on soluble huEpoR using the selection strategies described in Example 1. 2,000 scFv phage were randomly picked from the phage pool after the two rounds of selection. The 2,000 phage were used in an ELISA screen, which identified 960 scFv phage that appeared to specifically bind to huEpoR.

Plasmid DNA minipreps from the 960 scFv phage clones were made and pooled. The DNA pool from the 960 scFv phage clones was digested with NcoI and NotI. The resulting 0.75 kb fragments were ligated to a PciI and NotI digested mammalian expression vector, pDC409a-G1Fc. pDC409a-G1Fc is described in Example 2. Ligation products were transformed into TG1 cells. After ligation, 1,920 single colonies were picked and plasmid DNA minipreps from each of the 1,920 colonies were made in 96-well plates using a Qiagen BioRobot 3000. These 96-well plates served as stock plates. The DNA concentration of each well in the stock plates was between 50 and 200 ng/ul.

Aliquots of DNA from the stock plates were combined with Lipofectamine 2000 (Invitrogen) in a new set of 96-well plates (first set of test plates). Lipid/DNA complexes were formed by incubation at room temperature for 30 minutes in the wells of the first set of test plates. Lipid/DNA complexes were then added to a second set of 96-well plates (second set of test plates) containing Cos PKB cells. Lipid DNA complexes were transfected into the Cos PKB cells.

5 days after transfection, cultured supernatant containing expressed protein was collected from the second set of test plates. The cultured supernatants were tested for the ability to bind EpoR using an in vitro EpoR activation assay. Two in vitro EpoR activation assays were performed for each protein being tested. The first assay used culture supernatant at a final dilution of 1:2. The second assay used a culture supernatant at a final dilution of 1:20.

The supernatants from the second set of test plates were also tested for protein titer by Fc ELISA. The concentration ranges from the Fc ELISA were between 5-20 μg/ml.

These screens identified a second set of clones: clone 201, clone 276, clone 295, clone 307, clone 318, clone 319, clone 323, clone 330, clone 352, and clone 378.

Clone 13, clone 15, clone 16, clone 29, and clone 34 were isolated as generally described in Example 1.

IgG2 and Fab expression constructs containing the second set of clones were constructed using the cloning strategy described in Example 2.

Protein identities were verified by N-terminal amino acid sequencing and concentrations determined on a Spectrophotometer by absorption at 280 nm.

The second set of clones were sequenced. DNA and amino acid sequences for the variable heavy chains and variable light chains for clone 13, clone 15, clone 16, clone 29, clone 34, clone 201, clone 276, clone 295, clone 307, clone 318, clone 319, clone 323, clone 330, clone 352, and clone 378 are shown below. Heavy chain and light chain CDR1, CDR2 and CDR3 are underlined in order within each sequence.

>#13VH nucleic acid sequence

(SEQ ID NO.: 55)

CAGGTACAGCTGCAGCAGTCAGGGGGAGGCGTGGTCCAGCCTGGGAGGTC
CCTGAGACTCTCCTGTGCAGCCTCTGGATTCACCTTCAGTGACTATGCTA
TGCACTGGGTCCGCCAGGCTCCAGGCAAGGGGCTAGAGTGGGTGGCAGTT
ATATCAAATCATGGAAAGAGCACATACTACGCAGACTCCGTGAAGGGCCG
ATTCACCATCTCCAGAGACAATTCCAAGCACATGCTGTATCTGCAAATGA
ACAGCCTGAGAGCTGACGACACGGCTCTATATTACTGTGCGAGAGATATA
GCATTGGCTGGGGACTACTGGGGCCAGGGCACCCTGGTCACCGTCTCT
GCC
>#13VH amino acid sequence

(SEQ ID NO.: 56)

QVQLQQSGGGVVQPGRSLRLSCAASGFTFSDYAMHWVRQAPGKGLEWVAV
ISNHGKSTYYADSVKGRFTISRDNSKHMLYLQMNSLRADDTALYYCARDI
ALAGDYWGQGTLVTVSA
>#13VL nucleic acid sequence

(SEQ ID NO.: 57)

GACATCCAGATGACCCAGTCTCCATCCTCCCTGTCTGCATCTGTAGGAGA
CAGAGTCACCATCACTTGCCGGGCAAGTCAGAGCATTAGCAGCTATCTTA
ATTGGTATCAGCAACTACCAGGGAAAGTCCCTAAACTCCTGATCTATGGT
GCATCGAAGTTGCAAAGTGGGGTCCCCTCCAGGTTCAGTGGCAGTGGATC
TGGGACAGATTTCACTCTCACCATCAGCAGCCTGCAGCCTGAAGATTTTG
CAACTTATTACTGTCTCCAAGATTACAATTATCCTCTCACTTTCGGCCCT
GGGACACGACTGGAGATCAAA
>#13VL amino acid sequence

(SEQ ID NO.: 58)

DIQMTQSPSSLSASVGDRVTITCRASQSISSYLNWYQQLPGKVPKLLIYG
ASKLQSGVPSRFSGSGSGTDFTLTISSLQPEDFATYYCLQDYNYPLTFGP
GTRLEIK
>#15VH nucleic acid sequence

(SEQ ID NO.: 59)

CAGGTGCAGCTGCAGGAGTCGGGCCCAGGACTGGTGAGGCCTTCGGGGAC
CCTGTCCCTCACCTGCGCTGTCTCTGGTGGCTCCATCGGCAGTAGTAACT
GGTGGAGTTGGGTCCGCCAGGCCCCAGGGAAGGGGCTGGAGTGGATTGGG
GAAATCTCTCAGAGTGGGAGCACCAACTACAACCCGTCCCTCAAGGGTCG
AGTCACCATATCACTAGACAGGTCCAGGAACCAGTTGTCCCTGAAGTTGA
GTTCTGTGACCGCCGCGGACACGGCCGTGTATTACTGTGCGAGACAGCTG
CGGTCGATTGATGCTTTTGATATCTGGGGCCCAGGGACCACGGTCACCGT
CTCGGCC
>#15VH amino acid sequence

(SEQ ID NO.: 60)

QVQLQESGPGLVRPSGTLSLTCAVSGGSIGSSNWWSWVRQAPGKGLEWIG
EISQSGSTNYNPSLKGRVTISLDRSRNQLSLKLSSVTAADTAVYYCARQL
RSIDAFDIWGPGTTVTVSA
>#15VL nucleic acid sequence

(SEQ ID NO.: 61)

TCCTATGTGCTGACTCAGCCACCCTCAGTGTCCGTGTCCCCAGGACTGAC
AGCCACCATCACCTGCTCTGGAGATAAATTGGGGGACAAATATGCTTCCT
GGTATCAGCAGAAGCCAGGCCAGTCCCCTGTGTTGGTCATCTATCAAGAT
AGGAAGCGACCCTCAGGGATCCCTGAGCGATTCTCTGGGTCCAATTCTGG
GAACACAGCCACTCTGACCATCAGCGGGACCCAGGCTGTGGATGAGGCTG
ACTATTACTGTCAGGCGTGGGACAGCGACACTTCTTATGTCTTCGGAACT
GGGACCCAGCTCACCGTTTTA
>#15VL amino acid sequence

(SEQ ID NO.: 62)

SYVLTQPPSVSVSPGLTATITCSGDKLGDKYASWYQQKPGQSPVLVIYQD
RKRPSGIPERFSGSNSGNTATLTISGTQAVDEADYYCQAWDSDTSYVFGT
GTQLTVL
>#16VH nucleic acid sequence

(SEQ ID NO.: 63)

CAGGTGCAGCTGCAGGAGTCGGGCCCAGGACTGGTGAAGCCTTCGGAGAC
CCTGTCCCTCACCTGCACTGTCTCTGGTGGCTACATCAATAATTACTACT
GGAGCTGGATCCGGCAGCCCCCAGGGAAGGGCCTGGAGTGGATTGGGTAC
ATCCATTACAGTGGGAGCACCTACTACAACCCGTCCCTCAAGAGTCGAGT
CACCATATCAGAAGACACGTCCAAGAACCAGTTCTCCCTGAAGCTGAGCT
CTGCGACCGCTGCGGACACGGCCGTGTATTACTGTGCGAGAGTTGGGTAT
TACTATGATAGTAGTGGTTATAATCTTGCCTGGTACTTCGATCTCTGGGG
CCGTGGAACCCTGGTCACCGTCTCGGCC
>#16VH amino acid sequence

(SEQ ID NO.: 64)

QVQLQESGPGLVKPSETLSLTCTVSGGYINNYYWSWIRQPPGKGLEWIGY
IHYSGSTYYNPSLKSRVTISEDTSKNQFSLKLSSATAADTAVYYCARVGY
YYDSSGYNLAWYFDLWGRGTLVTVSA
>#16VL nucleic acid sequence

(SEQ ID NO.: 65)

TCTTCTGAGCTGACTCAGGACCCTGCTGTGTCTGTGGCCTTGGGACAGAC
GGTCAGGATCACATGCCAGGGAGACAACCTCAGAAGTTATTCTGCAACTT
GGTACCAACAGAAGCCAGGACAGGCCCCTGTCCTTGTCCTCTTTGGTGAA
AACAACCGGCCCTCAGGGATCCCAGACCGATTCTCTGGCTCCAAGTCAGG
GGACACAGCTGTCTTGACCATCACTGGGACTCAGACCCAAGATGAGGCTG
ACTATTATTGCACTTCCAGGGTCAATAGCGGGAACCATCTGGGGGTGTTC
GGCCCAGGGACCCAGCTCACCGTTTTA
>#16VL amino acid sequence

(SEQ ID NO.: 66)

SSELTQDPAVSVALGQTVRITCQGDNLRSYSATWYQQKPGQAPVLVLFGE
NNRPSGIPDRFSGSKSGDTAVLTITGTQTQDEADYYCTSRVNSGNHLGVF
GPGTQLTVL
>#29VH nucleic acid sequence

(SEQ ID NO.: 67)

GAGGTGCAGCTGGTGGAGTCTGGGGCTGAGGTGAAGAAGCCTGGGGCCTC
AGTGAAGGTCTCCTGCAAGGCTTCTGGATACACCTTCACCGGCTACTATA
TGCACTGGGTGCGACAGGCCCCTGGACAAGGGCTTGAGTGGATGGGATGG
ATCAACCCTAACAGTGGTGGCACAAACTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGGGGGG
CACATGACTACGGTGACCCGTGATGCTTTTGATATCTGGGGCCAAGGGAC
AATGGTCACCGTCTCTGCC
>#29VH amino acid sequence

(SEQ ID NO.: 68)

| | |
|---|---|
| EVQLVESGAEVKKPGASVKVSCKASGYTFTGYYMHWVRQAPGQGLEWMGW | / |

INPNSGGTNYAQKFQGRVTMTRDTSISTAYMELSRLRSDDTAVYYCARGG
HMTTVTRDAFDIWGQGTMVTVSA
>#29VL nucleic acid sequence

(SEQ ID NO.: 69)

TCTTCTGAGCTGACTCAGGACCCTGCTGTGTCTGTGGCCTTGGGACAGAC
AATCAGGATCACATGCCAAGGAGACAGCCTCAGATACTATTATGCAACCT
GGTATCAGCAGAAGCCAGGACAGGCCCCTATACTTGTCATCTATGGTCAG
AATAATCGGCCCTCAGGGGTCCCAGACCGATTCTCTGGCTCCAGCTCAGG
AAACACAGCTTCCTTGACCATCACTGGGGCTCAGGCGGAAGATGAGGCTG
ACTATTACTGCGGAACATGGGATAGCAGTGTGAGTGCCTCTTGGGTGTTC
GGCGGAGGGACCAAGGTCACCGTCCTA
>#29VL amino acid sequence

(SEQ ID NO.: 70)

SSELTQDPAVSVALGQTIRITCQGDSLRYYYATWYQQKPGQAPILVIYGQ
NNRPSGVPDRFSGSSSGNTASLTITGAQAEDEADYYCGTWDSSVSASWVF
GGGTKVTVL
>#34VH nucleic acid sequence

(SEQ ID NO.: 71)

CAGGTACAGCTGCAGCAGTCAGGGGCTGAGGTGAAGAAGCCTGGGGCCTC
AGTGAAGGTCTCCTGCAAGGCTTCTGGATACACCTTCAGCGGCTATTATA
TGCACTGGGTGCGACAGGCCCCTGGACAAGGGCTTGAGTGGATGGGATGG
ATCAACCCTAACAGTGGCAGCACAAATTATGCACAGAAGTTTCTGGGCAG
GGTCACCATGACCAGGGACACGTCCATCAGCACAGCCTACATGGAACTGA
GCAGCCTGAGATCTGACGACACGGCCGTGTATTACTGTGCGAGGGGACAC
TCCGGTGACTATTTTGACTACTGGGGCCAGGGAACCCTGGTCACCGTCTC
GGCC
>#34VH amino acid sequence

(SEQ ID NO.: 72)

QVQLQQSGAEVKKPGASVKVSCKASGYTFSGYYMHWVRQAPGQGLEWMGW
INPNSGSTNYAQKFLGRVTMTRDTSISTAYMELSSLRSDDTAVYYCARGH
SGDYFDYWGQGTLVTVSA
>#34VL nucleic acid sequence

(SEQ ID NO.: 73)

GAAATTGTGTTGACGCAGTCTCCATCCTCCCTGTCTGCATCTGTTGGAGA
CAGAGTCACCATCACTTGCCGGGCCAGTCAGAGTGTTAGCAGCTGGTTGG
CCTGGTATCAACAGAGACCAGGGCAAGCCCCTAAACTGCTGATCTATGCT
GCACGTTTGCGAGGTGGAGGCCCTTCAAGGTTCAGTGGCAGCGGCTCTGG
GACAGAATTCACTCTCACCATCAGCAGTCTGCAACCTGAAGACTTTGCGA
CTTACTTCTGTCAACAGAGTTACAGTACCCCGATCAGTTTCGGCGGAGGG
ACCAAGCTGGAGATCAAA
>#34VL amino acid sequence

(SEQ ID NO.: 74)

EIVLTQSPSSLSASVGDRVTITCRASQSVSSWLAWYQQRPGQAPKLLIYA
ARLRGGGPSRFSGSGSGTEFTLTISSLQPEDFATYFCQQSYSTPISFGGG
TKLEIK
>#201VH nucleic acid sequence

(SEQ ID NO.: 75)

CAGGTGCAGCTGCAGGAGTCGGGCTCAGGACTGGCGAGGCCTTCACAGAC
CCTGTCCCTCACCTGCGCTGTCTCTGGTGGCTCCATCAGCAGTAGTGCTT
TCTCCTGGAATTGGATCCGGCAGCCACCAGGGAAGGGCCTGGAGTGGATT
GGATACATCTATCATACTGGGATCACCGATTATAACCCGTCCCTCAAGAG
TCGAGTCACCATATCAGTGGACAGGTCCAAGAACCAGTTCTCCCTGAACG
TGAACTCTGTGACCGCCGCGGACACGGCCGTGTATTATTGTGCCAGAGGA
CACGGTTCGGACCCCGCCTGGTTCGACCCCTGGGGCAAGGGCACCCTGGT
CACCGTCTCGAGT
>#201VH amino acid sequence

(SEQ ID NO.: 76)

QVQLQESGSGLARPSQTLSLTCAVSGGSISSSAFSWNWIRQPPGKGLEWI
GYIYHTGITDYNPSLKSRVTISVDRSKNQFSLNVNSVTAADTAVYYCARG
HGSDPAWFDPWGKGTLVTVSS
>#201VL nucleic acid sequence

(SEQ ID NO.: 77)

CAATCTGTGCTGACTCAGCCACCCTCAGTGTCCGTGTCCCCAGGACAGAC
AGCCAGCATCACCTGCTCTGGAGATAAATTGGGGGATAAATATGCTTCCT
GGTATCAGCAGAGGCCAGGCCAGTCCCCTGTTCTGGTCATCTATCGAGAC
ACCAAGCGGCCCTCAGGGATCCCTGAGCGATTCTCTGGCTCCAACTCTGG
GAACACAGCCACTCTGACCATCAGCGGGACCCAGGCTGTGGATGAGGCTG
ACTATTACTGTCAGGCGTGGGACAGCACCACCTCCCTGGTTTTCGGCGGA
GGGACCAAGCTGACCGTCCTA
>#201VL amino acid sequence

(SEQ ID NO.: 78)

QSVLTQPPSVSVSPGQTASITCSGDKLGDKYASWYQQRPGQSPVLVIYRD
TKRPSGIPERFSGSNSGNTATLTISGTQAVDEADYYCQAWDSTTSLVFGG
GTKLTVL
>#276VH nucleic acid sequence

(SEQ ID NO.: 79)

GAGGTCCAGCTGGTACAGTCTGGGGGAGGCTTGGTCCAGCCTGGGGGGTC
CCTGAGACTCTCCTGTGCAGCCTCTGGATTCACCTTTAGTAGCTATTGGA
TGAGCTGGGTCCGCCAGGCTCCTGGGAAGGGGCTGGAGTGGGTGGCCAAC
ATAAAGCCAGATGGAAGTGAGAAATACTATGTGGACTCTGTGAAGGGCCG
ATTCACCATCTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTACTCGGACTGGGGCCGAGGGACAATGGTCACCGTCTC
GAGT
>#276VH amino acid sequence

(SEQ ID NO.: 80)

EVQLVQSGGGLVQPGGSLRLSCAASGFTFSSYWMSWVRQAPGKGLEWVAN
IKPDGSEKYYVDSVKGRFTISRDNAKNSVYLQMNSLRAEDTAVYYCARVS
RGGSYSDWGRGTMVTVSS
>#276VL nucleic acid sequence

(SEQ ID NO.: 81)

CAGTCTGTGCTGACTCAGCCACCCTCCGCGTCCGGGTCTCCTGGACAGTC
AGTCACCATCTCCTGCACTGGAACCAGCAGTGACGTTGGCGGTTTTAACT
ATGTCTCCTGGTACCAAAAGTACCCAGGCAAAGCCCCCAAACTCGTCATT
TATGAGGTCAGTAAGCGGCCCTCAGGGGTCCCTGATCGCTTCTCTGGCTC
CAAGTCCGGCAACACGGCCTCCCTGACCGTCTCTGGGCTCCAGGCTGAGG
ATGAGGCTGATTATTACTGCAGCTCATGGGCACCTGGTAAAAACTTATTC
GGCGGAGGGACCAAGCTGACCGTCCTA
>#276VL amino acid sequence

(SEQ ID NO.: 82)

QSVLTQPPSASGSPGQSVTISCTGTSSDVGGFNYVSWYQKYPGKAPKLVI
YEVSKRPSGVPDRFSGSKSGNTASLTVSGLQAEDEADYYCSSWAPGKNLF
GGGTKLTVL
>#295VH nucleic acid sequence

(SEQ ID NO.: 83)

GAGGTGCAGCTGGTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAGGT
ATTAGTGGTAGTGGTAGTAGTGAAGGTGGCACATACTACGCAGACTCCGT
GAAGGGCCGGTTCACCCTCTCCAGAGACAATTCCAAGAATACCCTGTATC
TGCAAATGAACAGCCTGAGAGCCGAGGACACGGCCTTATATTACTGTGTG
AAAGATCGCCCTAGTCGATACAGCTTTGGTTATTACTTTGACTACTGGGG
CCGGGGAACCCTGGTCACCGTCTCGAGT
>#295VH amino acid sequence

(SEQ ID NO.: 84)

EVQLVESGGGLVQPGGSLRLSCAASGFTFSSYAMSWVRQAPGKGLEWVSG
ISGSGSSEGGTYYADSVKGRFTLSRDNSKNTLYLQMNSLRAEDTALYYCV
KDRPSRYSFGYYFDYWGRGTLVTVSS
>#295VL nucleic acid sequence

(SEQ ID NO.: 85)

CTGCCTGTGCTGACTCAGCCACCCTCAGTGTCCGTGTCCCCAGGACAGAC
AGCCAGCATCGCCTGCTCTGGAAATAAATTGGGGGATAAATATGTTTCCT
GGTATCAGCAGAAGCCAGGCCAGTCCCCTCTGCTGGTCATCTATCAAGAT
ACCAAGCGGCCCTCAGGGATCCCTGAGCGATTCTCTGGCTCCAACTCAGG
GAACACAGCCACTCTGACCATCAGCGGGACCCAGGCTATGGATGAGGCTG
ACTATTACTGTCAGGCGTGGGACAGCAGCACTGATGTGGTATTCGGCGGA
GGGACCAAGCTGACCGTCCTA
>#295VL amino acid sequence

(SEQ ID NO.: 86)

LPVLTQPPSVSVSPGQTASIACSGNKLGDKYVSWYQQKPGQSPLLVIYQD
TKRPSGIPERFSGSNSGNTATLTISGTQAMDEADYYCQAWDSSTDVVFGG
GTKLTVL
>#307VH nucleic acid sequence

(SEQ ID NO.: 87)

GAGGTGCAGCTGGTGGAGTCTGGGGGAGGCTTGGTCCAGCCTGGGGGGTC
CCTGAGACTCTCCTGTGCGGTCTCTGGGTTCACCTTTAGTAAGTATTGGA
TGACCTGGGTCCGCCAGGCTCCAGGGAAGGGACTGGAGTGGGTGGCCAAC
ATAAAGCCAGATGGAAGTGAGAAATACTATGTGGAGTCTGTGAAGGGCCG
ATTCACCATCTCCAGAGACAACGCCAAGAATTCAGTGTATCTGCAAATGA
ACAGTGTGAGAGCCGAAGACACGGCCGTGTATTACTGTGCGAGAGTTTCG
AGGGGTGGGAGCTTCTCGGACTGGGGCCAGGGGACAATGGTCACCGTCTC
GAGT
>#307VH amino acid sequence

(SEQ ID NO.: 88)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTMVTVSS
>#307VL nucleic acid sequence

(SEQ ID NO.: 89)

CAGTCTGTGCTGACTCAGCCACCCTCCGCGTCCGGGTCTCCTGGACAGTC
AGTCACCATCTCCTGCACTGGAACCAGCAGCGACGTTGGTGGTTATAACT
ATGTCTCCTGGTACCAACAACACCCAGACAAAGCCCCCAGACTCATGATT
TATGACGTCAATAAGCGGCCCTCAGGGGTCCCTGATCGCTTCTCTGGCTC
CAAGTCTGGCAACACGGCCTCCCTGACCGTCTCTGGGCTCCAGGCTGAGG
ATGAGGCTCATTATTACTGCAACTCATATGCAGGCAGCAACAATTGGGTG
TTCGGCGGAGGGACCCAGCTCACCGTTTTA
>#307VL amino acid sequence

(SEQ ID NO.: 90)

QSVLTQPPSASGSPGQSVTISCTGTSSDVGGYNYVSWYQQHPDKAPRLMI
YDVNKRPSGVPDRFSGSKSGNTASLTVSGLQAEDEAHYYCNSYAGSNNWV
FGGGTQLTVL
>#318VH nucleic acid sequence

(SEQ ID NO.: 91)

CAGGTGCAGCTGGTGGAGTCTGGGGGAGGCTTGGTCCAGCCTGGGGGGTC
CCTGAGACTCTCCTGTGCGGTCTCTGGGTTCACCTTTAGTAAGTATTGGA
TGACCTGGGTCCGCCAGGCTCCAGGGAAGGGACTGGAGTGGGTGGCCAAC
ATAAAGCCAGATGGAAGTGAGAAATACTATGTGGAGTCTGTGAAGGGCCG
ATTCACCATCTCCAGAGACAACGCCAAGAATTCAGTGTATCTGCAAATGA
ACAGTGTGAGAGCCGAAGACACGGCCGTGTATTACTGTGCGAGAGTTTCG
AGGGGTGGGAGCTTCTCGGACTGGGGCCAAGGAACCCTGGTCACCGTCTC
GAGT
>#318VH amino acid sequence

(SEQ ID NO.: 92)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS
>#318VL nucleic acid sequence

(SEQ ID NO.: 93)

CAGTCTGTGCTGACTCAGCCACCCTCCGCGTCCGGGTCTCCTGGACAGTC
AGTCACCATCTCCTGCACTGGAACCAGCAGTGACGTTGGTGGTTATAATT
ATGTCTCCTGGTACCAACAACACCCAGGCAGAGCCCCCAAACTCATCATT
TATGAGGTCAGTAAGCGGCCCTCAGGGGTCCCTGATCGCTTCTCTGGCTC
CAAGTCTGGCAACACGGCCTCCCTGACCGTCTCTGGGCTCCAGGCTGACG
ATGAGGCTGATTATTACTGCAACTCATATGCAGGCAGCATTTATGTCTTC
GGGAGTGGGACCAAGGTCACCGTCCTA
>#318VL amino acid sequence

(SEQ ID NO.: 94)

QSVLTQPPSASGSPGQSVTISCTGTSSDVGGYNYVSWYQQHPGRAPKLII
YEVSKRPSGVPDRFSGSKSGNTASLTVSGLQADDEADYYCNSYAGSIYVF
GSGTKVTVL
>#319VH nucleic acid sequence

(SEQ ID NO.: 95)

CAGGTGCAGCTGGTGCAATCTGGGGCTGAAATTAAGAAGCCTGGGGCCTC
AGTGAAGGTTTCCTGCAAGACATTTGGATCCCCCTTCAGCACGAATGACA
TACACTGGGTGCGACAGGCCCCTGGACAAGGGCTTGAGTGGATGGGAATA
ATCGACACTAGTGGCGCCATGACAAGGTACGCACAGAAGTTCCAGGGCAG
AGTCACCGTGACCAGGGAAACGTCCACGAGCACAGTCTACATGGAGCTGA
GCAGCCTGAAATCTGAAGACACGGCTGTGTACTACTGTGCGAGAGAGGGT
TGTACTAATGGTGTATGCTATGATAATGGTTTTGATATCTGGGGCCAAGG
CACCCTGGTCACCGTCTCGAGT
>#319VH amino acid sequence

(SEQ ID NO.: 96)

QVQLVQSGAEIKKPGASVKVSCKTFGSPFSTNDIHWVRQAPGQGLEWMGI
IDTSGAMTRYAQKFQGRVTVTRETSTSTVYMELSSLKSEDTAVYYCAREG
CTNGVCYDNGFDIWGQGTLVTVSS
>#319VL nucleic acid sequence

(SEQ ID NO.: 97)

GATATCCAGATGACCCAGTCTCCTTCCACCCTGTCTGCATCTATTGGAGA
CAGAGTCACCATCACCTGCCGGGCCAGTGAGGGTATTTATCATTGGTTGG
CCTGGTATCAGCAGAAGCCAGGGAAAGCCCCTAAACTCCTGATCTATAAG
GCCTCTAGTTTAGCCAGTGGGGCCCCATCAAGGTTCAGCGGCAGTGGATC
TGGGACAGATTTCACTCTCACCATCAGCAGCCTGCAGCCTGATGATTTTG
CAACTTATTACTGCCAACAATATAGTAATTATCCGCTCACTTTCGGCGGA
GGGACCAAGCTGGAGATCAAA
>#319VL amino acid sequence

(SEQ ID NO.: 98)

DIQMTQSPSTLSASIGDRVTITCRASEGIYHWLAWYQQKPGKAPKLLIYK
ASSLASGAPSRFSGSGSGTDFTLTISSLQPDDFATYYCQQYSNYPLTFGG
GTKLEIK
>#323VH nucleic acid sequence

(SEQ ID NO.: 99)

CAGGTGCAGCTGGTGGAGTCTGGGGGAGGCTTGGTCCAGCCTGGGGGGTC
CCTGAGACTCTCCTGTGCGGTCTCTGGGTTCACCTTTAGTAAGTATTGGA
TGACCTGGGTCCGCCAGGCTCCAGGGAAGGGACTGGAGTGGGTGGCCAAC
ATAAAGCCAGATGGAAGTGAGAAATACTATGTGGAGTCTGTGAAGGGCCG
ATTCACCATCTCCAGAGACAACGCCAAGAATTCAGTGTATCTGCAAATGA
ACAGTGTGAGAGCCGAAGACACGGCCGTGTATTACTGTGCGAGAGTTTCG
AGGGGTGGGAGCTTCTCGGACTGGGGCCGGGGGACAATGGTCACCGTCTC
GAGT
>#323VH amino acid sequence

(SEQ ID NO.: 100)

QVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGRGTMVTVSS
>#323VL nucleic acid sequence

(SEQ ID NO.: 101)

CAATCTGCCCTGACTCAGCCTGCCTCCGTGTCTGGGTCTCCTGGACAGTC
GATCACCATCTCCTGCACTGGAACCAGCAGTGATGTTGGGAGTTATAACC
TTGTCTCCTGGTACCAACAACACCCAGGCAAAGTCCCCAAACTCATCATT
TATGAGGTCAGTAATCGGCCCTCAGGGGTTTCTCATCGCTTCTCTGGCTC
CAAGTCTGGCAACACGGCCTCCCTGACCATCTCTGGACTCCAGGCTGAGG
ACGAGGCTGATTATTACTGCAGCTCATTGACAAGCAGCGGCACTTGGGTG
TTCGGCGGAGGGACCAAGGTCACCGTCCTA
>#323VL amino acid sequence

(SEQ ID NO.: 102)

QSALTQPASVSGSPGQSITISCTGTSSDVGSYNLVSWYQQHPGKVPKLII
YEVSNRPSGVSHRFSGSKSGNTASLTISGLQAEDEADYYCSSLTSSGTWV
FGGGTKVTVL
>#330VH nucleic acid sequence

(SEQ ID NO.: 103)

GAGGTGCAGCTGGTGGAGTCCGGGGGAGGCTTGGTCCAGCCCGGGGGGTC
CCTGAGACTCTCCTGTGCGGTCTCTGGGTTCACCTTTAGTAAGTATTGGA
TGACCTGGGTCCGCCAGGCTCCAGGGAAGGGACTGGAGTGGGTGGCCAAC
ATAAAGCCAGATGGAAGTGAGAAATACTATGTGGAGTCTGTGAAGGGCCG
ATTCACCATCTCCAGAGACAACGCCAAGAATTCAGTGTATCTGCAAATGA
ACAGTGTGAGAGCCGAAGACACGGCCGTGTATTACTGTGCGAGAGTTTCG
AGGGGTGGGAGCTTCTCGGACTGGGGCCAGGGCACCCTGGTCACCGTCTC
GAGT
>#330VH amino acid sequence

(SEQ ID NO.: 104)

EVQLVESGGGLVQPGGSLRLSCAVSGFTFSKYWMTWVRQAPGKGLEWVAN
IKPDGSEKYYVESVKGRFTISRDNAKNSVYLQMNSVRAEDTAVYYCARVS
RGGSFSDWGQGTLVTVSS
>#330VL nucleic acid sequence

(SEQ ID NO.: 105)

CAGTCTGCCCTGACTCAGCCTCCCTCCGCGTCCGGGTCTCCTGGGCAGTC
AGTCACCATCTCCTGCACTGGAACCAGCAGTGACGTTGGTGCTTATAACT
ATGTCTCCTGGTACCAACAGCACCCAGGCAAAGCCCCCAAACTCATGATT
TATGAGGTCGCTAGGCGGCCCTCAGGGGTCCCTGATCGCTTCTCTGGCTC
TAAGTCTGGCAACACGGCCTCCCTGACCGTCTCTGGGCTCCAGGCTGAGG
ATGAGGCTGATTATTATTGCAGCTCATATGCAGGCAGCAACAATTTCGCG
GTCTTCGGCAGAGGGACCAAGCTGACCGTCCTA
>#330VL amino acid sequence

(SEQ ID NO.: 106)

QSALTQPPSASGSPGQSVTISCTGTSSDVGAYNYVSWYQQHPGKAPKLMI
YEVARRPSGVPDRFSGSKSGNTASLTVSGLQAEDEADYYCSSYAGSNNFA
VFGRGTKLTVL
>#352VH nucleic acid sequence

(SEQ ID NO.: 107)

GAGGTGCAGCTGGTGCAGTCTGGGGGAGGCTTGGTCCAGCCGGGGGGGTC
CCTGAGACTCTCCTGTGCAGCCTCTGGATTCAGGTTTAGTAGCTATTGGA
TGACCTGGGTCCGCCAGGCTCCAGGGAAGGGGCTGGAGTGGGTGGCCAAC
ATAAAGCCAGATGGAAGTGAGAAATACTATGTGGACTCTGTGAAGGGCCG
ATTCACCATGTCCAGAGACAACGCCAAGAATTCAGTGTATCTGCAAATGA
ACAGCCTGAGAGCCGAGGACACGGCCGTGTATTACTGTGCGAGAGTTTCG
AGGGGTGGGAGCTTCTCGGACTGGGGCCAAGGAACCCTGGTCACCGTCTC
GAGT
>#352VH amino acid sequence

(SEQ ID NO.: 108)

EVQLVQSGGGLVQPGGSLRLSCAASGFRFSSYWMTWVRQAPGKGLEWVAN
IKPDGSEKYYVDSVKGRFTMSRDNAKNSVYLQMNSLRAEDTAVYYCARVS
RGGSFSDWGQGTLVTVSS
>#352VL nucleic acid sequence

(SEQ ID NO.: 109)

CAGTCTGCCCTGACTCAGCCTGCCTCCGTGTCTGGGTCTCCTGGACAGTC
GATCACCATCCCCTGCACTGGAACCAGCAGTGACATTGGTACTTATGACT
ATGTCTCCTGGTACCAACAACACCCAGGCAAAGTCCCCAAAGTCATTATT
TATGAGGTCACCAATCGGCCCTCAGGGGTTTCTAATCGCTTCTCTGGCTC
CAAGTCTGGCAACACGGCCTCCCTGACCATCTCTGGGCTCCAGGCTGACG
ACGAGGCTGATTATTACTGCAACTCATTTACAAAGAACAACACTTGGGTG
TTCGGCGGAGGGACCAAGCTGACCGTCCTA
>#352VL amino acid sequence

(SEQ ID NO.: 110)

QSALTQPASVSGSPGQSITIPCTGTSSDIGTYDYVSWYQQHPGKVPKVII
YEVTNRPSGVSNRFSGSKSGNTASLTISGLQADDEADYYCNSFTKNNTWV
FGGGTKLTVL
>#378VH nucleic acid sequence

(SEQ ID NO.: 111)

CAGGTGCAGCTGGTGGAGTCTGGGGGAGGCTTGGTCCAGCCTGGGAGGTC
CCTGATACTCTCCTGTGCGGTCTCTGGGTTCACCTTTAGTAAGTATTGGA
TGACCTGGGTCCGCCAGGCTCCAGGGAAGGGACTGGAGTGGGTGGCCAAC
ATAAAGCCAGATGGAAGTGAGAAATACTATGTGGAGTCTGTGAAGGGCCG
ATTCACCATCTCCAGAGACAACGCCAAGAATTCAGTGTATCTGCAAATGA
ACAGTGTGAGAGCCGAAGACACGGCCGTGTATTACTGTGCGAGAGTTTCG
AGGGGTGGGAGCTTCTCGGACTGGAGCCAAGGAACCTTGGTCACCGTCTC
GAGT
>#378VH amino acid sequence

(SEQ ID NO.: 112)

QVQLVESGGGLVQPGRSLILSCAVSGFTFSKYWMTWVRQAPGKGLEWVAN
IKPDGSEKYYVESVKGRFTISRDNAKNSVYLQMNSVRAEDTAVYYCARVS
RGGSFSDWSQGTLVTVSS
>#378VL nucleic acid sequence

(SEQ ID NO.: 113)

CAGTCTGCCCTGACTCAGCCTCCCTCCGCGTCCGGGTCTCCTGGGCAGTC
AGTCACCATCTCCTGCACTGGAACCAGCGGTGACGTTGGTGCTTATAACT
ATGTCTCCTGGTACCAACAGTACCCAGGCAAAGCCCCCAAACTCATGATT
TATGAGGTCAGTAAGAGGCCCTCCGGGGTCCCTGATCGCTTCTCTGGCTC
CAAGTCTGGCAACACGGCCTCCCTGACCGTCTCTGGGCTCCAGGCTGAGG
ATGAGGCTGATTATTACTGCAACTCATATAGGGGCAGCAACGGTCCTTGG
GTGTTCGGCGGAGGGACCAAGGTCACCGTCCTA
>#378VL amino acid sequence

(SEQ ID NO.: 114)

QSALTQPPSASGSPGQSVTISCTGTSGDVGAYNYVSWYQQYPGKAPKLMI
YEVSKRPSGVPDRFSGSKSGNTASLTVSGLQAEDEADYYCNSYRGSNGPW
VFGGGTKVTVL

### Example 15

### Antibody Binding to Cell Surface huEpoR Analysis by FACS

The binding of scFv-Fc protein to a cell surface expressed huEpoR was analyzed using FACS. All scFv-Fc proteins used had an Fc derived from IgG1. UT-7 cells were incubated with either 5 nM scFv-Fc protein alone or with 5 nM scFv-Fc protein plus 0.5 μg/ml of rHuEpo for 1 hour at 4° C. After 2 quick washes using cold PBS, UT-7 cells were then incubated with 1 μg/ml phycoerythrin-conjugated goat F(ab′)2 anti-human IgG Fc (Jackson Immuno Research Laboratories) for 1 hour at 4° C. The cells were washed twice using cold PBS and resuspended into 1 ml of fixation buffer (2% paraformaldehyde PBS pH 7.4). FACS was done using a FACSCaliber flow cytometer (Becton-Dickinson)

The FACS traces of the proteins expressed from the scFv-Fc expression vectors are shown in FIG. 22. Clone 13, clone 15, clone 16, clone 29, and clone 34 all bound to huEpoR expressing UT-7 cells (FIG. 22A) but not to the negative control cells (FIG. 22B). UT-7 cell surface binding of clone 15, clone 16, and clone 34, was blocked by an excess amount of rHuEpo (FIG. 22A). rHuEpo did not block the binding of clone 13 or clone 29 (FIG. 22A).

### Example 16

### Competitive Binding of Clone 201, Clone 276, Clone 295, Clone 307, Clone 318, Clone 319, Clone 323, Clone 330, Clone 352, and Clone 378 to huEpoR

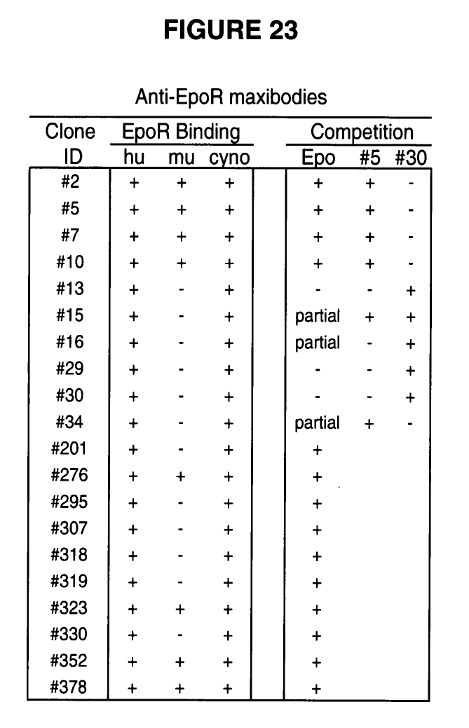

Clone 201, clone 276, clone 295, clone 307, clone 318, clone 319, clone 323, clone 330, clone 352, and clone 378 were tested for their ability to compete with Epo for binding to huEpoR.Fc using a plate-based ELISA. All scFv-Fc proteins used had an Fc derived from IgG1. Biotinylated Epo, which binds to huEpoR.Fc, was used as the competitor. huEpoR.Fc was immobilized on the polysorp ELISA plate. Inhibition of Epo binding by clone 201, clone 276, clone 295, clone 307, clone 318, clone 319, clone 323, clone 330, clone 352 and clone 378 in scFv-Fc was tested by concentration titration with each protein at 0 to 50 μg/ml, using streptavidin-HRP conjugate. All of the clones except clone 13, clone 15, clone 16, clone 29, clone 30, and clone 34 substantially blocked the Epo binding at high concentrations (FIG. 23). Clone 2, clone 5, clone 7, clone 10, clone 13, clone 15, clone 16, clone 29, clone 30 and clone 34 in phage format were tested for their ability to compete with clone 5 and clone 30 in maxibody format for binding to EpoR as generally described in Example 5.

### Example 17

### Antibody Binding to Mouse EpoR (muEpoR) and Cynomolgus Monkey EpoR (cynoEpoR)

The cross reactivity of certain clones in scFv-Fc format was tested using an ELISA Assay. All scFv-Fc proteins used had an Fc derived from IgG1. The clones tested were: clone 13, clone 15, clone 16, clone 29, clone 34, clone 201, clone 276, clone 295, clone 307, clone 318, clone 319, clone 323, clone 330, clone 352 and clone 378. 100 μl of 1 μg/ml (in 50 mM NaHCO3, pH8.5) cynoEpoR or muEpoR was added to each well on a polysorp ELISA plate and incubated at 4° C. overnight. After blocking the wells with 4% milk/PBS/0.1% Tween20 for 1 hour at room temperature, plates were washed three times with PBS/0.1% Tween20. 100 μl of 5 μg/ml scFv-Fc was added to each well and incubated for 1 hour at 25° C. The bound cynoEpoR or muEpoR was detected using anti-human IgG Fc-HRP conjugate (1:1000 dilution in 4% milk PBS/0.1% Tween20). ABTS (2,2′-azino-bis(3-ethylbenzthiazoline-6-sulfonic acid)) was used as a substrate and the absorption was measured at 405 nm on a plate reader. All clones showed a significant level of cross reactivity to cynoEpoR (FIG. 23). Clone 276, clone 323, clone 352, and clone 378 showed a substantial level of cross reactivity to muEpoR (FIG. 23).

### Example 18

### Measurement of Rate and Affinity Constants for Human and Cyno EpoR Using Biacore

Surface plasmon resonance experiments were conducted at 25° C. using a Biacore T100 instrument (Biacore AB, Uppsala, Sweden) equipped with a CM5 sensor chip. Each flow cell on the CM5 chips was activated with a 1:1 (v/v) mixture of 0.1 M N-hydroxysuccinimide (NHS) and 0.4 M1-ethyl-3-(3-dimethylaminopropyl) carbodiimide hydrochloride (EDC). Fcγ Fragment Specific AffiniPure Goat Anti-Human IgG antibody at 30 μg/ml in 10 mM sodium acetate, pH 5.0 was immobilized to two flow cells on the CM5 chips using standard amine coupling chemistry with a target level of 10,000 Resonance Units (RU). Residual reactive surfaces were deactivated with an injection of 1 M ethanolamine. The running buffer was then switched to HBS-EP+0.1 mg/ml BSA for all remaining steps.

For each scFv-Fc protein to be tested, the scFv-Fc protein was diluted in running buffer to 200 ng/ml and injected over the test flow cell at 10 μl/min for 2 minutes to capture the maxibody. All scFv-Fc proteins used had an Fc derived from IgG1. No scFv-Fc protein was captured on the control flow cell surface. Either human or cyno EpoR was then flown over the two flow cells at concentrations ranging from 24.7-6000 nM along with buffer blanks. A flow rate of 50 μl/min was used and a 1 minute association phase followed by a 5 minute (for cyno EpoR) or 10 minute (for hu EpoR) dissociation phase. After each cycle the surfaces were regenerated with a 30 second injection of 10 mM glycine pH 1.5. Fresh scFv-Fc protein was then captured on the test flow cell to prepare for the next cycle.

Data was double referenced by subtracting the control surface responses to remove bulk refractive index changes, and then the averaged buffer blank response was subtracted to remove systematic artifacts from the experimental flow cells. The EpoR data were processed and globally fit to a 1:1 interaction model with mass transfer and a local Rmax in Biacore T100 Evaluation Software v 1.1. (Biacore AB, Uppsala, Sweden). The measured interactions between clone 30 and human EpoR; clone 34 and cyno EpoR; and clone 318 and cyno EpoR had off-rates that were too rapid to measure accurately so the data was instead fit to a steady state model. The steady state model results in only an affinity determination and not kinetic values.

The rate and affinity constants are summarized in Table 3. The calculated affinities for hu EpoR to the scFv-Fc proteins varied from 1.1 nM for clone #10 (previous data shown in Table 2) to 4030 nM for clone # 201. For the Cyno EpoR the range was from 6.83 nM for clone #10 to 18,600 for clone #201. Clone #10 had the slowest k_{off}, while clone #201 had the slowest kₒₙ. In general, the calculated affinities were quite similar for the human and cynomolgus monkey EpoR with only three scFv-Fc proteins (clones #34, #307, and #330) showing greater than a 10× variation between the species.

TABLE 3
Summary of Human and Cyno EpoR Binding
Kinetics to scFv-Fc Proteins

| scFv-Fc | | | | |
|---|---|---|---|---|
| protein clone | EpoR Used | kₒₙ (10⁵, 1/Ms) | k_{off} (10⁻⁴, 1/s) | K_{D} (nM) |
| #5 | Human | Not repeated, see previous data | | |
| | Cynomolgus | 4.37 | 611 | 140 |
| #10 | Human | Not repeated, see previous data | | |
| | Cynomolgus | 1.56 | 10.7 | 6.83 |
| #13 | Human | 0.55 | 568 | 1,040 |
| | Cynomolgus | 0.65 | 597 | 920 |
| #15 | Human | 0.61 | 1,190 | 1,950 |
| | Cynomolgus | 0.37 | 1,150 | 3,130 |
| #16 | Human | 0.65 | 1,420 | 2,190 |
| | Cynomolgus | 0.65 | 2,830 | 4,360 |
| #29 | Human | 1.29 | 629 | 487 |
| | Cynomolgus | 1.90 | 504 | 265 |
| #30 | Human | Fit to steady-state model | | 3,690 |
| | Cynomolgus | 2.11 | 4,850 | 2,310 |
| #34 | Human | 5.36 | 2,030 | 378 |
| | Cynomolgus | Fit to steady-state model | | 5,810 |
| #201 | Human | 0.046 | 187 | 4,030 |
| | Cynomolgus | 0.027 | 508 | 18,600 |
| #295 | Human | 0.18 | 29.6 | 163 |
| | Cynomolgus | 0.41 | 221 | 539 |
| #307 | Human | 22.8 | 2,460 | 108 |
| | Cynomolgus | 2.99 | 3,610 | 1,210 |
| #318 | Human | 6.59 | 5,580 | 847 |
| | Cynomolgus | Fit to steady-state model | | 4890 |
| #319 | Human | 1.58 | 335 | 212 |
| | Cynomolgus | 2.13 | 258 | 121 |
| #330 | Human | 8.22 | 373 | 45.4 |
| | Cynomolgus | 1.08 | 965 | 890 |

### Example 19

### Screening of scFv-Fc Proteins in vitro for the Activation of the Human Erythropoietin Receptor

scFv-Fc proteins were screened for the activation of the huEpoR. The in vitro screening of the scFv-Fc proteins was done by a luciferase-based reporter assay (luciferase assay) in UT-7 cells (human megakaryoblasts) transfected with a construct containing 9 STAT5 binding sites in front of a luciferase reporter gene (UT-7-LUC cells). All scFv-Fc proteins used had an Fc derived from IgG1. All cells were maintained and all cellular assays were conducted at 37° C. in a humidified incubator at 5% CO₂/95% atmospheric air, unless otherwise noted. All fetal bovine serum (FBS) was heat inactivated at 55° C. for 45 minutes prior to usage. All Dulbecco's Phosphate-Buffered Saline (PBS) used for cell manipulation was without calcium chloride and magnesium chloride. UT-7-LUC cells (Amgen, Inc.; Thousand Oaks, Calif.) were maintained in growth media comprising IMDM (Invitrogen; Carlsbad, Calif.) containing 10% FBS (HyClone; Logan, Utah), 500 μg/mL hygromycin (Roche; Penzberg, Germany), 100 U/mL penicillin, 100 μg/mL streptomycin, 292 μg/mL L-glutamine (1×PSG; Invitrogen) and 0.5 U/mL recombinant human erythropoietin (Epoetin Alpha, rHuEpo; Amgen, Inc.). The cells were washed two times in assay media (RPMI Medium 1640 with 1% FBS, 1×PSG, and 12.5 mM HEPES (Invitrogen)) and resuspended at 400,000 cells per mL in assay media. Following an overnight incubation, cell number and viability were determined, and the cells were resuspended at 200,000 cells per mL in assay media.

Each scFv-Fc protein was serially diluted in a 96-well opaque plate (Corning; Corning, N.Y.). The concentration range, fold dilution, number of dilutions and number of replicates varied with each experiment and are indicated in Table 4. To serve as a control standard, recombinant human EPO was serially diluted in 7 wells of every 96-well plate, in duplicate, for a final concentration of 0.82 nM to 5.25E-05 nM. Approximately 10,000 cells were added to each well. The cells were then cultured for 18 to 24 hours (note that Example 7 used a 6-hour incubation period), and the assay was performed according to the manufacturer's protocol for the Steady-Glo Luciferase Assay. (Promega Corporation). Luciferase activity was read on a 96-well plate luminometer. The data were plotted to generate binding curves and EC₅₀ values using GraphPad Prism® software. The data is presented in Table 5 as average EC₅₀±the standard deviation.

TABLE 4
Summary of Mxb concentrations used in UT-7-luciferase assays.

| | Concentration range | | | | |
|---|---|---|---|---|---|
| | highest conc | lowest conc | fold | | # of |
| maxibody | (nM) | (nM) | dilution | # replicates | assays |
| Mxb#2 | 2,500 | 0.032 | 5 | 1 | 1 |
| Mxb#5 | 5,000 | 6.86 | 3 | 1 | 1 |
| ″ | 5,000 | 0.028 | 3 | 3 | 1 |
| ″ | 2,500 | 0.16 | 5 | 1 | 1 |
| ″ | 2,500 | 0.16 | 5 | 3 | 1 |
| ″ | 2,500 | 0.16 | 5 | 2 | 1 |
| ″ | 2,500 | 0.032 | 5 | 1 | 1 |
| ″ | 2,500 | 1.143 | 3 | 1 | 1 |
| ″ | 1,000 | 0.457 | 3 | 2 | 1 |
| Mxb#7 | 2,500 | 0.032 | 5 | 1 | 1 |
| Mxb#10 | 5,000 | 6.859 | 3 | 1 | 1 |
| ″ | 5,000 | 0.0282 | 3 | 3 | 1 |
| ″ | 2,500 | 0.032 | 5 | 1 | 1 |
| Mxb#13 | 5,000 | 6.859 | 3 | 1 | 1 |
| Mxb#15 | 5,000 | 6.859 | 3 | 1 | 1 |
| Mxb#29 | 5,000 | 6.859 | 3 | 1 | 1 |
| Mxb#30 | 2,500 | 1.143 | 3 | 1 | 1 |
| Mxb#34 | 5,000 | 6.859 | 3 | 1 | 1 |
| ″ | 25 | 0.034 | 3 | 3 | 1 |
| Mxb#201 | 5,000 | 6.859 | 3 | 1 | 1 |
| Mxb#276 | 5,000 | 0.028 | 3 | 3 | 1 |
| ″ | 5,000 | 6.859 | 3 | 2 | 1 |
| ″ | 2,500 | 0.032 | 5 | 1 | 1 |
| ″ | 2,500 | 1.143 | 3 | 1 | 1 |
| Mxb#295 | 5,000 | 6.859 | 3 | 1 | 1 |
| Mxb#307 | 5,000 | 6.859 | 3 | 1 | 1 |
| Mxb#318 | 25 | 0.034 | 3 | 3 | 1 |
| Mxb#319 | 5,000 | 6.859 | 3 | 1 | 1 |
| Mxb#323 | 5,000 | 6.859 | 3 | 2 | 1 |
| ″ | 2,500 | 0.032 | 5 | 1 | 1 |
| ″ | 2,500 | 1.143 | 3 | 1 | 1 |
| Mxb#330 | 25 | 0.034 | 3 | 3 | 1 |
| Mxb#352 | 5,000 | 0.028 | 3 | 3 | 1 |
| ″ | 5,000 | 6.859 | 3 | 2 | 1 |
| ″ | 2,500 | 0.032 | 5 | 1 | 1 |
| ″ | 2,500 | 1.143 | 3 | 1 | 1 |
| Mxb#378 | 2,500 | 0.032 | 5 | 1 | 1 |
| ″ | 2,500 | 1.143 | 3 | 1 | 1 |

TABLE 5
in Vitro activity (UT-7-luciferase assay)

| | Average EC50 | | |
|---|---|---|---|
| clone | (nM) | Std Dev | Ratio |
| #2 | 0.6035 | N/A | 0.016 |
| #5 | 0.7911 | 0.4156 | 0.012 |
| #7 | 0.4683 | N/A | 0.02 |
| #10 | 0.2955 | 0.2416 | 0.033 |
| #13 | 4.0250 | N/A | 0.002 |
| #15 | 2.8025 | N/A | 0.003 |
| #16 | N/A | N/A | N/A |
| #29 | 1.5215 | N/A | 0.006 |
| #30 | 0.6705 | N/A | 0.014 |
| #34 | 0.1095 | 0.0916 | 0.088 |
| #201 | 8.2755 | N/A | 0.001 |
| #276 | 0.3215 | 0.4016 | 0.03 |
| #295 | 0.6065 | N/A | 0.016 |
| #307 | 0.3810 | N/A | 0.025 |
| #318 | 0.0154 | N/A | 0.623 |
| #319 | 5.8655 | N/A | 0.002 |
| #323 | 0.6133 | 0.5003 | 0.016 |
| #330 | 0.0075 | N/A | 1.28 |
| #352 | 2.1560 | 1.2868 | 0.004 |
| #378 | 0.0550 | 0.0210 | 0.175 |

Table 5 shows EC₅₀ values of huEpoR activation levels for Mxb 2, Mxb 5, Mxb 7, Mxb 10, Mxb 13, Mxb 15, Mxb 16, Mxb 29, Mxb 30, Mxb 34, Mxb 201, Mxb 276, Mxb 295, Mxb 307, Mxb 318, Mxb 319, Mxb 323, Mxb 330, Mxb 352, and Mxb 378. The results are presented as average EC₅₀ values calculated using GraphPad Prism software (without any background subtraction) ± the standard deviation. When only one experiment was done, standard deviation is presented as N/A. Table 5 also shows the ratio of the EC₅₀ values of huEpoR activation by Epo divided by the EC₅₀ values of huEpoR activation by the various EREDLAs. All members of the EREDLA genus have a ratio of less than 1. All species listed in Table 5 are considered an EREDLA based on the EC₅₀ ratio criteria except for #330. The data in Table 5 was generated using the assay described immediately above, whereas the assay used to generate the titration curves shown in FIG. 7 (from which EC₅₀ values may be derived) had a slightly different protocol that used a 6-hour incubation period (see Example 8).

### Example 20

### In vivo Experiments with Mxb 276, Mxb 323, Mxb 352, and Mxb 378

The effect of a single injection of scFv-Fc proteins Mxb 276, Mxb 323, Mxb 352, or Mxb 378 was tested in mice. The scFv-Fc proteins were tested with either a IgG1fc or a IgG2fc. scFv-Fc proteins with an IgG1fc were abbreviated Mxb X_G1MB or X_G1MB, where “X” is the clone number. scFv-Fc proteins with an IgG2fc were abbreviated Mxb X_G2 MB or X_G2 MB, where “X” is the clone number. PEG-NESP was used as a positive control in this experiment. Carrier (10 mM Potassium Phosphate, 161 mM L-Arginine, pH 7.5) was used as a negative control.

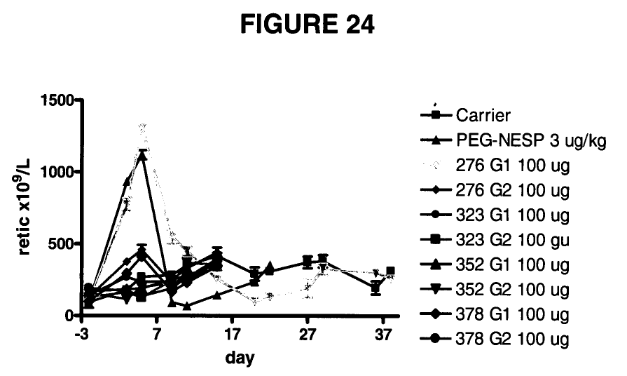

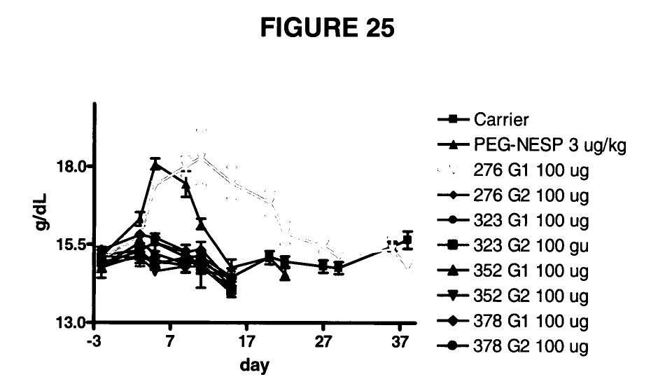

2-month-old female BDF-1 mice were injected subcutaneously with carrier (PBS with 0.1% BSA), 3 μg/kg PEG-NESP (Amgen, Inc.), or 100 μg of a scFv-Fc protein in a final volume of 200 μl. The following scFv-Fc proteins were tested at a single bolus dose of 100 μg/mouse: Mxb 276_G1 MB, Mxb 323_G1 MB, Mxb 352_G1 MB, Mxb 378_G1 MB, Mxb 276_G2MB, Mxb 323_G2MB, Mxb 352_G2MB, and Mxb 378_G2MB. Blood was collected from the retro-orbital sinus at numerous time-points and evaluated for CBC (Compete Blood Count) parameters using an ADVIA blood analyzer. For the first experiment, blood was collected on days −2, 3, 5, 9, 11, 15, 20, 22, 27, 29, 36, and 38 for the carrier and 276_Mxb groups. For the group of mice treated with PEG-NESP, blood was collected on days −2, 3, 5, 9, 11, 15, 20 and 22. For all other groups, blood was collected on days −2, 3, 5, 9, 11 and 16. In the second experiment, blood was collected on days −2, 3, 5, 9, 11 and 16 for all groups. As seen in FIGS. 24 and 25, not all mice were monitored for the full 38 days. Collections were stopped when the CBC parameter returned to a baseline level. Collections were made from five mice at each time point. Data are presented in FIGS. 24 and 25.

Mxb 276_G1MB had an erythropoietic stimulatory effect as observed by the increase in hemoglobin and reticulocyte numbers at 100 μg/mouse dose. There was no significant effect observed at this dose for any of the other Mxbs tested in this experiment. PEG-NESP acted as a positive control and performed as predicted. The activity profile of Mxb 276_G1MB was different from that of PEG-NESP; the peak reticulocyte number was achieved on day 5 after an injection of either PEG-NESP or Mxb 276_G1MB, but the duration of the reticulocyte response was significantly increased in the mice that received a dose of Mxb 276_G1MB. The reticulocyte numbers returned to baseline on day 9 in the PEG-NESP-treated mice, but it took 19 to 20 days for the reticulocytes to return to baseline in the Mxb 276_G1MB-treated mice. In mice injected with Mxb 276_G1MB at this dose, the hemoglobin levels stayed above baseline for 22 to 29 days. In contrast, the hemoglobin level in the PEG-NESP-treated mice returned to baseline at day 15, thus showing a very significant difference in the duration and magnitude of the hemoglobin response in the mice treated with Mxb 276_G1MB versus mice treated with PEG-NESP. This experiment demonstrates that a single injection of Mxb 276_G1MB increases hemoglobin levels above baseline for a significant period of time that is close to the total life span of the red blood cells in mice (approximately 40 days). Since the rate of hemoglobin decline after the administration of an erythropoietic agent is related to the life span of erythrocytes (approximately 120 days in humans), it is possible that a single administration of Mxb 276_G1MB in humans could potentially be enough to correct anemia over a period of 2-3 months.

### Example 21

### Generation of Mxb Human Point Mutant Fc and Mxb Cynomolgus Point Mutant Fc

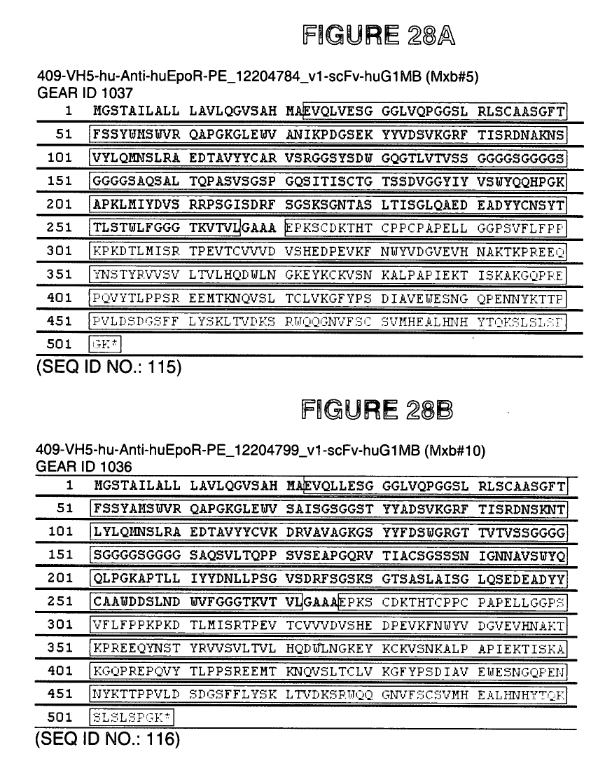

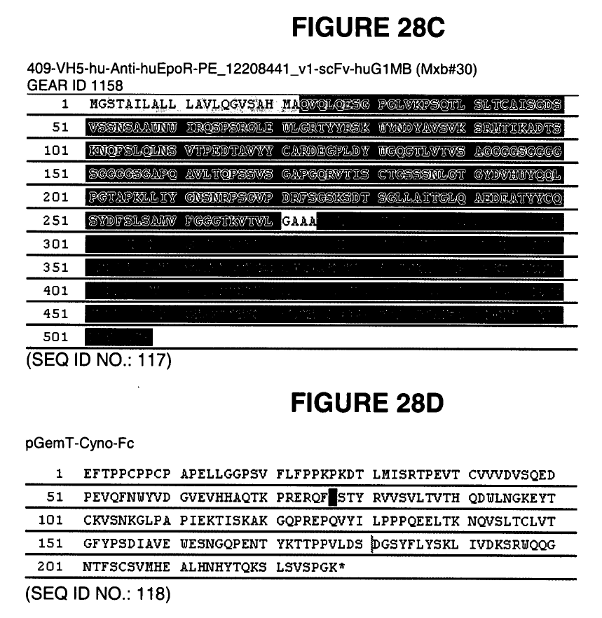

Mxb 5, Mxb 10, and Mxb 30 (with human Fc) and Mxb 5 (with cynomolgus Fc) were mutated at asparagine 297 of the Fc portion of the proteins. The mutated asparagine is in the position equivalent to asparagine 297 of the CH2 domain of human IgG. The asparagine at position 297 was replaced by a serine residue in all of the mutants (N297S) using Stratagene's QuikChange II Site-Directed Mutagenesis Kit. For the human Fc mutagenesis, primers 4606-78 (CGG GAG GAG CAG TAC AGC AGC ACG TAC CGT GTG) and 4606-79 (CAC ACG GTA CGT GCT GCT GTA CTG CTC CTC CCG) were used in the reaction. For the cynomolgus Fc mutagenesis, primers 4606-76 (GGG AGA GGC AGT TCA GCA GCA CGT ACC GCG) and 4606-77 (CGC GGT ACG TGC TGC TGA ACT GCC TCT CCC) were used. Mutagenesis was carried out according to the manufacturer's instructions. The template DNAs are shown in FIG. 28.

The mutation to asparagine 297 was made to inhibit binding of the Mxb to the Fc Gamma Receptor III (“FcgRIII”) on effector cells present in vivo. The goal was to minimize any killing of the hematopoietic progenitor cells in the bone marrow by immune effector cells expressing FcgRIII. Engagement of this receptor in effector cells triggers ADCC (antibody dependent cell-mediated cytotoxicity). See, e.g., Radaev et al., J Biol. Chem. 2001 May 11; 276(19):16478-83 and Radaev et al., J Biol. Chem. 2001 May 11; 276(19):16469-77.

After the mutagenesis, colonies were picked and the correct DNA sequence was confirmed via sequence analysis.

DNA maxipreps of clones Mxb#5-huFc-N297S (21457), Mxb#10-huFc-N297S (21480), Mxb#30-huFc-N297S (21481) and cyno-Fc N297S (21456) were prepared using the Qiagen Compact Prep Kit according to the manufacturers instructions. A 5′ Hind III site and 3′ Bam HI site were added to each of the clones via polymerase chain reaction (PCR). The maxipreps mentioned above were used as the template DNA for the PCR reactions.

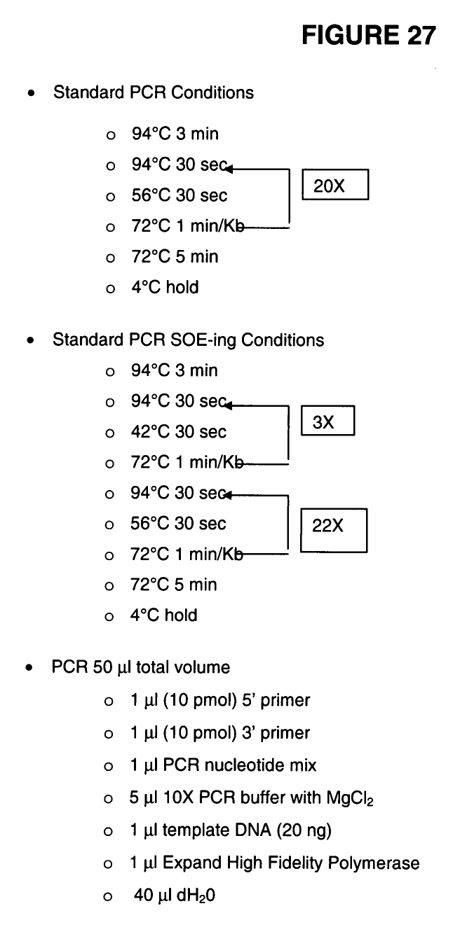

Primers 4611-63 (GAC TGC AAG CTT GAC ACC ATG GGG TCA ACC GCC) and 4611-64 (GCA TAC GGA TCC TCA TTT ACC CGG AGA CAG) were used in the PCR's for Mxb#5-huFc-N297S, Mxb#10-huFc-N297S, and Mxb#30-huFc-N297S (FIG. 27).

For the Mxb 5 (with cynomolgus Fc), primers 4611-63 and 4606-84 (CAT GGG GGT GTG AAC TCT GCG GCC GCT AGG ACG G) were used to amplify clone 5 scFv and add the 5′ Hind III site in a PCR reaction. Primers 4606-83 (CCG TCC TAG CGG CCG CAG AGT TCA CAC CCC CAT G) and 4611-65 (GCA TCA GGA TCC TCA TTT ACC CGG AGA CAC) were used to amplify the cyno-Fc N297S and add a 3′ Bam HI site in a PCR reaction. The clone 5 scFv amplified product and cyno-Fc N297S amplified product were then used as templates in a Gene Splicing by Overlap Extension “SOE-ing” PCR reaction (FIG. 27). Primers 4611-63 and 4611-65 were used in that reaction.

All PCR reactions were run in a MJ Research Peltier Thermal Cycler (PTC, Waltham, Mass.) using an Expand High Fidelity PCR System (Roche, Indianapolis, Ind., cat. no. 11732650001). The reaction and conditions for the PCR are shown in FIG. 27.

After PCR amplification, all of the amplification products were column purified using a Qiagen's Qiaquik Gel Extraction Kit following the manufacturer's instructions. The amplification products were then cut with Hind III for 90 minutes. The amplification products were column purified using a Qiagen Qiaquik Gel Extraction Kit according to the manufacturer's instructions. The amplification products were then cut with Bam HI for 90 minutes. The cut products were gel purified using a Qiagen Qiaquik Gel Extraction Kit according to the manufacturer's instructions and then ligated into pTT5 BamHI/HindIII using New England Biolab's T4 ligase overnight.

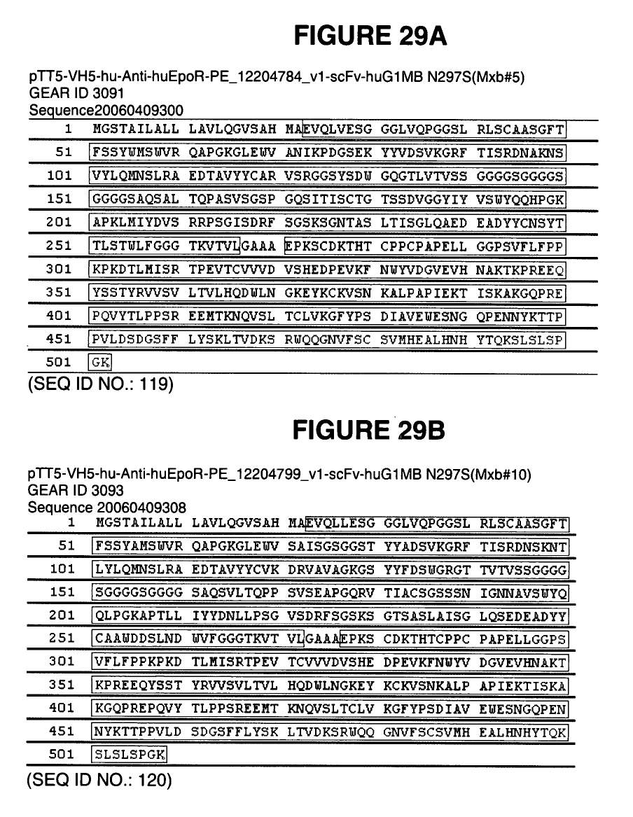

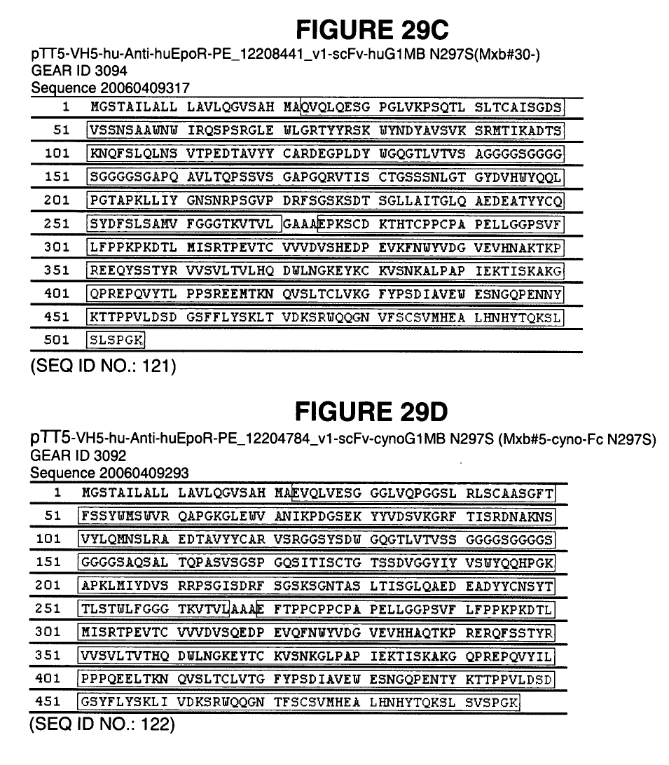

The ligation products were column purified the next day and transformed via electroporation into DH10B cells. Colonies were then picked for sequencing and were sequenced. The four scFv-Fc protein sequences are presented in FIG. 29.

### Example 22

### Dose Escalation Study of Mxb 5, Mxb 10, and Mxb 30 in Cynomolgus Monkeys

Each of the four scFv-Fc proteins described in Example 21 was intravenously administered to cynomolgus monkeys, and the pharmacodynamics (hematological effects) and pharmacokinetics (PK) effects after intravenous administration were measured. As noted in Example 21, the Fc regions of the scFv-Fc proteins tested lacked the ability to bind to FcgRIII. The human point mutant Fc used in the scFv-Fc proteins was a human IgG1 point mutant Fc that lacks a glycosylation site required for FcgRIII binding. The cynomolgus point mutant Fc used in the scFv-Fc proteins was a cyno IgG1 Fc that also lacks a glycosylation site required for FcgRIII binding. The scFv-Fc proteins tested were a Mxb 5 human point mutant Fc (un-glycosylated Fc), a Mxb 5 cynomolgus point mutant Fc (un-glycosylated Fc), a Mxb 10 human point mutant Fc (un-glycosylated Fc), and a Mxb 30 human point mutant Fc (un-glycosylated Fc).

A total of 18 female cynomolgus monkeys weighing between 2 and 4 kg were used in the study. The monkeys were divided into the following 6 experimental groups:

1. Vehicle control (10 mM potassium phosphate, 161 mM L-Arginine, pH 7.5)

2. Positive control group (Peg-NESP)

3. Mxb#5 human point mutant Fc

4. Mxb#10 human point mutant Fc

5. Mxb#30 human point mutant Fc

6. Mxb#5 cynomolgus point mutant Fc

The study had a duration of 31 days and scFv-Fc proteins or control samples were administered to each animal twice by IV injection. The administration of the scFv-Fc proteins, vehicle control, and positive control (Peg-NESP) occurred on day 1 and day 15 of the study. Each scFv-Fc protein injection was dosed at 0.5 mg/kg in 10 mM potassium phosphate, 161 mM L-Arginine, pH 7.5 for the first administration on day 1 and at 5 mg/kg in 10 mM potassium phosphate, 161 mM L-Arginine, pH 7.5 for the second administration on day 15. Peg-Nesp was dosed at 0.03 mg/kg for both injections. The vehicle control (10 mM potassium phosphate, 161 mM L-Arginine, pH 7.5) was dosed at 1 ml/kg for both injections.

Following intravenous administration, blood (approximately 1 mL) was collected from each animal for PK and hematological analysis at predose (Day-2), predose (Day 1) and 120, 192, 288, 360, 456, 528, 624, and 696 hours after the first dose was administered.

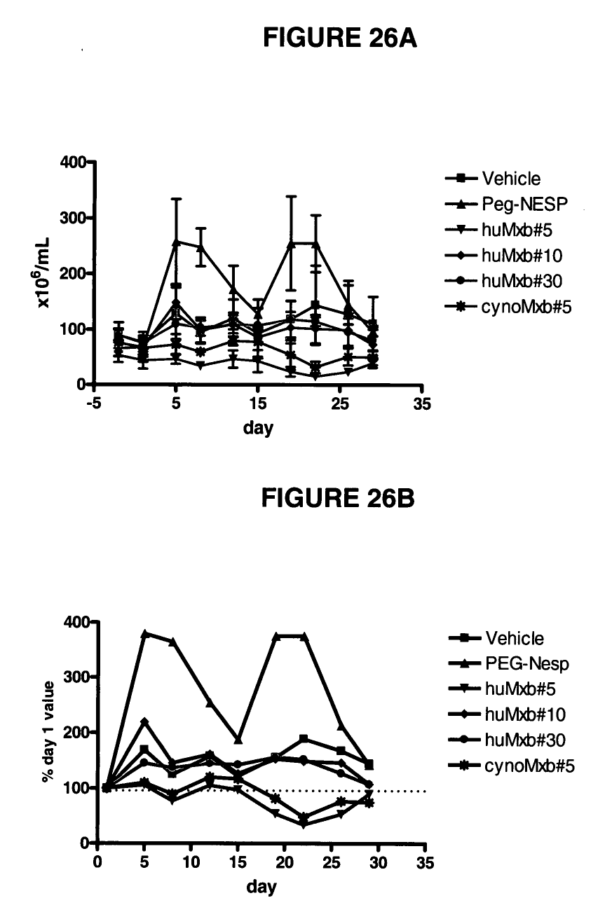

Preliminary analysis of the data showed differences among Mxb 5, Mxb 10, and Mxb 30. See FIGS. 26A and 26B. The 2 variants of Mxb 5 induced a drop in reticulocyte and hemoglobin levels when dosed at 5 mg/kg, but Mxb 30 and Mxb 10 did not induce any drop in reticulocytes or hemoglobin. In addition, at day 5 after administration of the first dose, the increase in reticulocyte levels in monkeys administered Mxb 10 was statistically significant when compared to the pre-dose baseline reticulocyte level (p=0.029, F-test).

### Example 23

### Epitope Mapping of Anti-EpoR scFv-Fc Proteins Alanine Scanning of EpoR

A crystal structure of the extracellular ligand-binding domain of EpoR complexed to the ligand has been determined (Syed et al., Nature 395, 511-6 (1998)). This information was used to create a panel of mutants which could be used to map individual surface residues involved in antibody binding. An alanine-scanning strategy was pursued for EpoR. The method used to choose residues to mutate involved both computational mechanisms and interactive structure analysis. All residues were colored red. Next, the solvent exposures of all residues in the dimer were calculated. Residues with ≧60 Å² surface area or with solvent exposure ratios ≧50% were colored green. Next, glycines with positive φ angles were colored magenta, as were Asp8 and Pro9 since they cap the N-terminal helix. Residues (colored blue) were then chosen to fill in the surface gaps. Further residues were then chosen by viewing the structure for residues that point toward the surface but were excluded in the solvent exposure calculations. These were colored cyan. To bring the number of mutations down to 95, prolines in turns, specifically residues 23, 50 and 203, were colored magenta. The cyan residues were then sorted by solvent exposure and solvent exposure ratio. The top six of each measure were kept while the rest were colored magenta. Non-alanine residues were mutated to alanine, and alanine mutated to serine.

The binding of an antibody to an antigen covers the antigen surface area in the region of antibody binding. This covered patch of antigen residues includes both residues that are directly involved in antibody binding and those that are in the region of antibody binding but may not directly contribute to binding. The covered patch of antigen residues-defines a structural epitope on the antigen. Residues within this covered patch that are not seen as directly involved in binding the antibody by alanine scanning may-be contributing to overall antibody binding through other interactions.

Alanine scanning is a method that tests whether the mutated residue is part of a functional epitope. The functional epitope describes those residues in the antigen which are directly involved in antibody binding. Single site alanine mutants were used to determine those residues in the antigen with side chains that are directly involved in antibody binding; alanine has a smaller side chain than all other residues except glycine and would therefore cause the loss of a side chain binding site and affect antibody binding.

A different type of epitope map is the structural epitope, or those residues in the antigen which are contacting or buried by the antibody. Introducing arginine mutants into the antigen is a method that tests whether a residue is part of the structural epitope. The arginine sidechain is large and bulky, effectively blocking antibody binding regardless of whether the wild type residue is directly involved in antibody binding. Accordingly, single site arginine mutants were used to determine those residues in the antigen that are in the covered patch. If an antigen residue mutated to arginine modulates the binding of the antibody, it suggest that the residue is part of the structural epitope. If the antigen wild type residue is arginine, it is mutated to glutamate.

Construction, Expression and Characterization of Alanine Mutants

95 individual alanine or serine mutants were produced according to standard techniques. Sense and anti-sense oligonucleotides containing the mutated residues were synthesized in a 96 well format. Mutagenesis of the wild-type (WT) huEpoR was performed using a Quickchange II kit (Stratagene) following the manufacturer's instructions. All mutants were constructed in a pTT5 vector, and were tagged with 6×His-Avitag (Avidity, LLC, Denver, Colo.) on the C-terminus. Mutagenesis reactions and transformations were performed in a 96 well format. 2936-E suspension cells (NRCC) were transiently transfected. The expression levels and integrity of the recombinant proteins in conditioned media were checked by Western analysis. The average expression level was estimated to be ˜5 μg/mL; 6 mutants did not express, while another 8 mutants expressed poorly.

All amino acid residues were identified by their position in the extracellular domain of the human Epo Receptor. The following mutants were not able to be epitope mapped due to non-expression or poor expression: R32A, S54A, K65A, Q71A, W82A, R108A, W209A and W212A. Finally, mutated residues F208A and P86A affected binding of all of the scFv-Fc proteins, and are likely to be incorrectly folded. Thus even though they diminish antibody binding, they were not considered to be part of the epitope. Where possible, mutants were checked for the ability to bind to Epo in order to confirm that they were correctly folded.

Assay Methodology

1. ELISA binding assay.

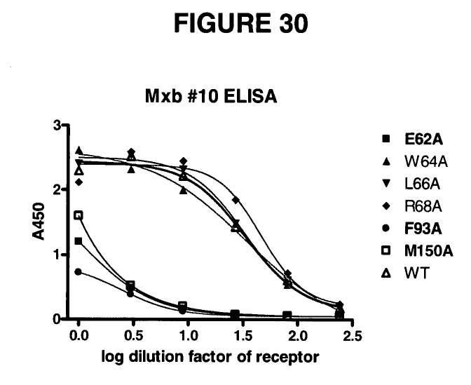

An ELISA binding assay was used to measure binding of the anti-EpoR antibodies to conditioned supernatants containing the mutant protein of interest. 100 μl of purified scFv-Fc protein at 1 μg/mL in 1×PBS was coated upon a Nunc Maxisorp plate, and incubated at 4 degrees overnight. All scFv-Fc proteins used had an Fc derived from IgG1. After blocking the wells with 2% BSA/PBS/0.1% Tween20 for 1 hour at room temperature, plates were washed three times with PBS/0.1% Tween20. EpoR mutant protein concentrations were normalized based on gel densitometry relative to the WT protein. The EpoR mutant proteins were serially diluted 3-fold in 0.1% BSA/PBS/0.1% Tween20, which also contained a constant 1:5000 dilution of anti-6×His mAb-HRP (R&DSystems). The EpoR mutant/anti-6×His mAb-HRP mixture was captured for 2 hours at room temperature. TMB (3,3′,5,5′-Tetramethylbenzidine) was used as a substrate and the absorption was measured at 450 nm on a plate reader. Binding data were analyzed by non-linear regression analysis (sigmoidal dose-response, variable slope) to generate EC₅₀ values using GraphPad Prism® software. It was suggested that mutations which abolished binding, or decreased binding by 50% relative to wild type were part of the epitope. Representative data is shown in FIG. 30.

2. EpoR LANCE Binding Assay

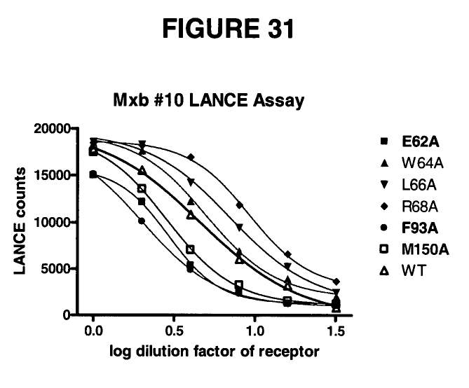

A homogeneous LANCE FRET (Fluorescence Resonance Energy Transfer) assay for EpoR-Ab binding was also used, using an Eu-chelate-conjugated anti-IgG mAb and an APC-conjugated anti-pHis mAb. EpoR mutant concentrations were normalized based on gel densitometry relative to the wild type protein. Mutant EpoR proteins were serially diluted 2-fold in a mixture of purified anti-EpoR scFv-Fc protein (1.5 nM), 0.75 nM Eu chelate labeled-anti-IgG mAb (Perkin Elmer) and 35 nM APC-anti-His mAb Ab (Perkin Elmer). The samples were incubated for 2 hours at room temperature before excitation at 535 nm and detection at 655 nm in a fluorescent plate reader. EpoR mutants which were suggested to be part of the epitope diminish or abolish the FRET signal. The binding data were plotted to generate binding curves and EC₅₀ values using GraphPad Prism® software. It was suggested that mutations which abolished binding, or decreased binding by 50% relative to wild type were part of the epitope. Representative data is shown in FIG. 31.

Arginine Scanning

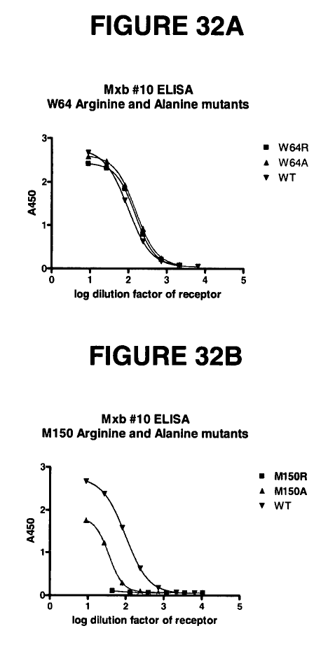

As noted above, all amino acid residues were identified by their position in the extracellular domain of the human Epo Receptor. The following mutants: E34R, E60R, P63R, W64R, T87R, A88R, R99E, A103R, V112R, M150R, H153R and A166R were also made by the same method as the alanine mutants. The arginine mutants were expected to introduce a greater structural perturbation than the alanine mutants, thus confirming our assignments for these residues (FIG. 32).

Eight candidate agonistic scFv-Fc proteins, Mxb #2, #5, #7, #10, #13, #15, #29 and #30, were mapped. A summary of alanine mutations which diminish binding by >50% relative to WT or abolish binding by both the LANCE and ELISA assays is shown in Table 6 Also shown in Table 6 is a summary of arginine mutations which diminish binding by >50% relative to WT or abolish binding by the ELISA assay. That table does not exclude other residues not listed in the table from being part of the epitope; those residues may not have been mutated, or the assays may not have been sensitive enough to identify them as being part of the epitope.

TABLE 6
Summary of residues that are affected part of the human
EpoR epitope of 8 anti-EpoR agonistic scFv-Fc proteins.

| | Residues in the Extracellular | Residues in the Extracellular |
|---|---|---|
| scFv-Fc | Domain of EpoR Changed to | Domain of EpoR Changed to |
| protein | Alanine | Arginine |
| Mxb #2 | F93, H114 | E34, E60 |
| Mxb #5 | S91, F93, H114 | E60 |
| Mxb #7 | F93 | E60 |
| Mxb #10 | E62, F93, M150 | A88, M150 |
| Mxb #13 | V48, E62, L66, R68, H70 | |
| Mxb #15 | V48, W64, L66, R68, H70 | T87 |
| Mxb #29 | A44, V48, P63, L66, R68, | P63, W64, R99 |
| | H70 | |
| Mxb #30 | L66, R99 | R99 |

The epitopes for these antibodies fall into two distinct classes. The first class is the Epo competitive scFv-Fc proteins (Mxb 2, Mxb 5, Mxb 7 and Mxb 10). The second class are those scFv-Fc proteins that do not compete with Epo (Mxb 30, Mxb 13, Mxb 15, and Mxb 29). Those data are consistent with the hypothesis that the non-Epo competitive scFv-Fc proteins agonise the EpoR receptor by binding to regions which are distal to the ligand-binding pocket of the dimer.

### Example 24

### Sequence Alignments and Phylogenetic Analysis of scFv-Fc Proteins Variable Heavy Chain and Variable Light Chain CDR Regions

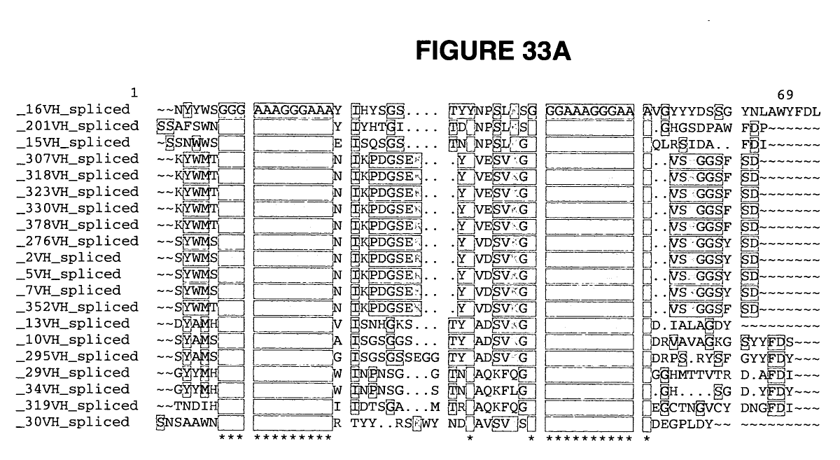

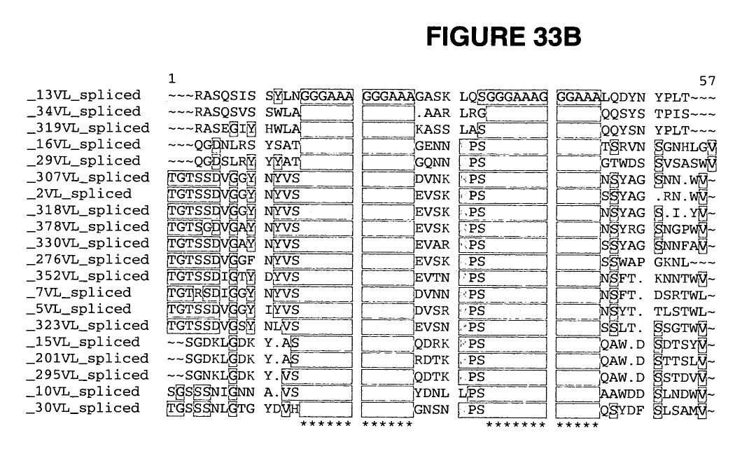

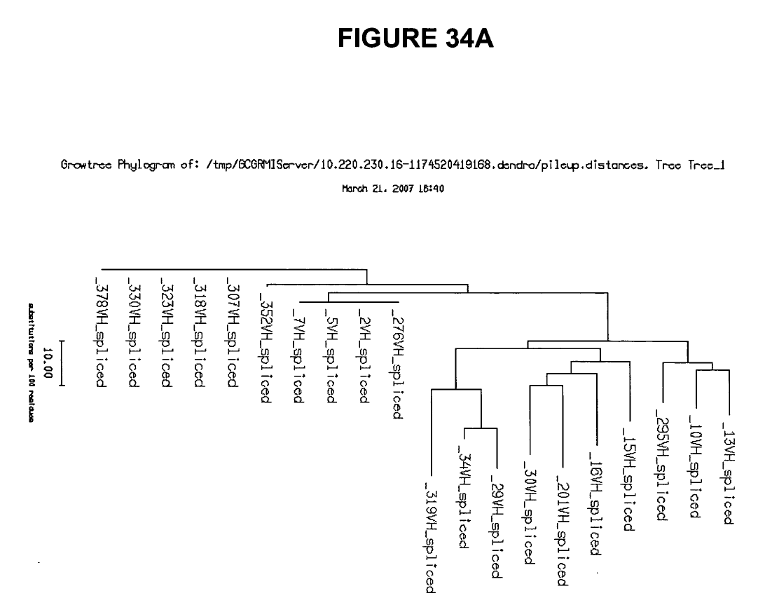

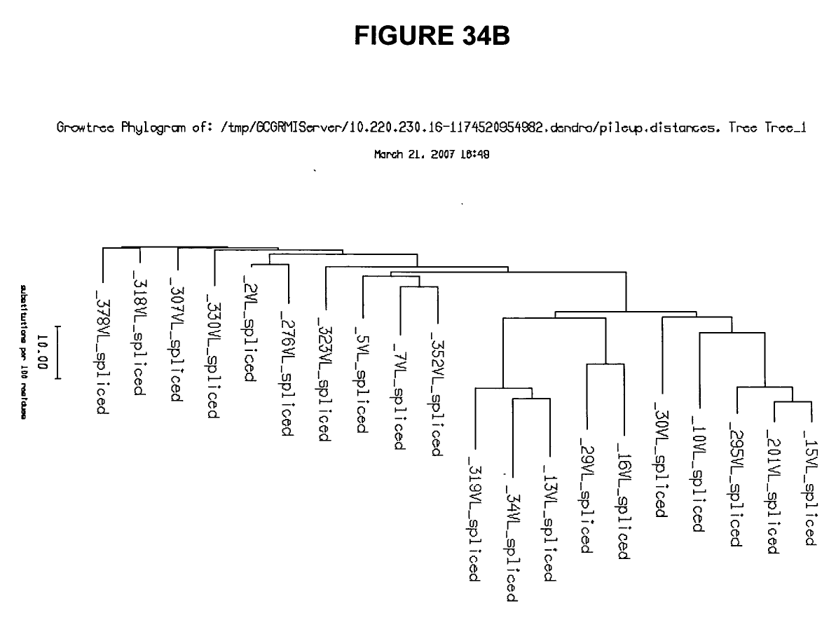

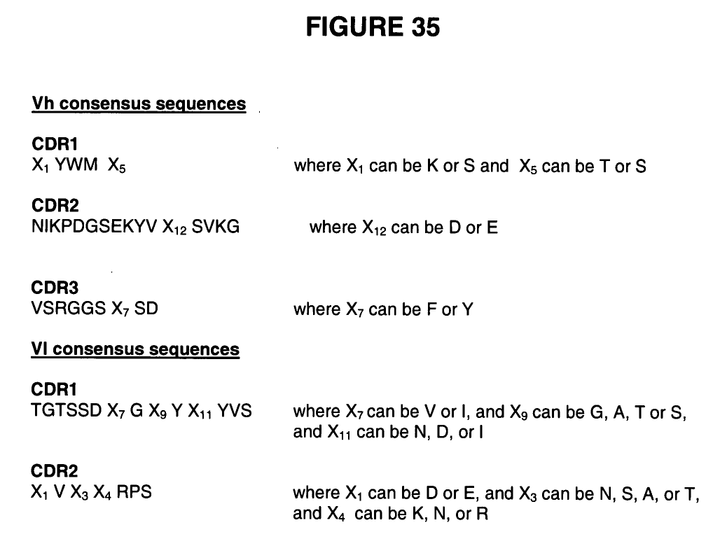

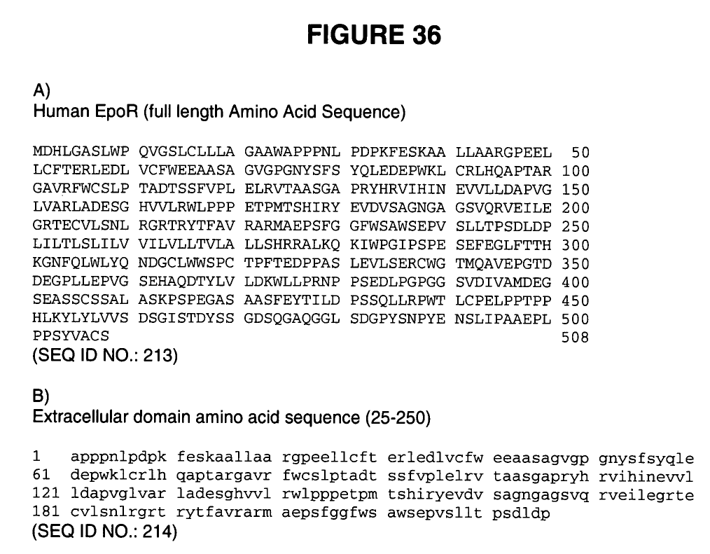

To determine the diversity among the scFv-Fc proteins' CDRs, electronic splicing of the CDRs was used. First the CDR regions were identified. Then the framework regions were removed from the sequences and small peptide sequences were used as linkers between the CDRs. A multiple alignment of the electronically spliced sequences was used to create phylogenetic trees. The process was used for both the variable heavy and variable light chain sequences. The MiniPileup program (CGC software) was used to produce the multiple alignments and phylogenetic trees (FIGS. 33 and 34). The results are summarized in the phylogenetic neighbor joining analysis (FIG. 34). Clone 307, clone 2, clone 318, clone 378, clone 330, clone 276, clone 352, clone 7, clone 5, and clone 323 share a relatively high level of identity in the variable heavy CDR regions. Among these clones, the diversity in amino acid sequence of the variable light chain is seen mainly in the CDR3 region. Clone 16, clone 201, clone 15, clone 13, clone 10, clone 295, clone 29, clone 34, clone 319 and clone 30 show higher level of sequence variation in both the variable heavy and variable light CDRs.

We claim:

1. An Erythropoietin Receptor Extended Duration Limited Agonist, comprising an antibody that:
(a) binds the erythropoietin receptor in a population of cells expressing the erythropoietin receptor and activates the erythropoietin receptor to a lesser degree than erythropoietin, or recombinant equivalents or analogs of erythropoietin, when used at the same or higher concentrations than erythropoietin, or recombinant equivalents or analogs of erythropoietin;
(b) binds to the human erythropoietin receptor with a lower affinity than erythropoietin;
(c) raises the hemoglobin concentration in a treated mammal and induces an initial peak concentration of erythropoietin that is comparable to the peak hemoglobin attainable with erythropoietin, or recombinant equivalents or analogs of erythropoietin, but maintains the hemoglobin concentration in said mammal over a period of time that is longer than that attainable with erythropoietin, or recombinant equivalents or analogs of erythropoietin; and
(d) possesses an extended half-life in vivo beyond that of erythropoietin, or recombinant equivalents or analogs of erythropoietin.

2. The Erythropoietin Receptor Extended Duration Limited Agonist of claim 1, wherein (a) is the EC₅₀ ratio of: the EC₅₀ values derived from an in vitro assay measuring the relative readout of Epo, or recombinant equivalents or analogs of Epo, activating the erythropoietin receptor/the EC₅₀ values derived from said assay measuring the relative readout of an Erythropoietin Receptor Extended Duration Limited Agonist activating the erythropoietin receptor, wherein the ratio is less than 1.

3. The Erythropoietin Receptor Extended Duration Limited Agonist of claim 2, wherein the EC₅₀ ratio is in the range of about 0.001 to about 0.623.

4. The Erythropoietin Receptor Extended Duration Limited Agonist of claim 1, wherein in (a) about 200 to 2,000 fold more of the Erythropoietin Receptor Extended Duration Limited Agonist is required to achieve maximum colony number in a Burst Forming Unit-Erythroid assay in relation to the amount of erythropoietin, or recombinant equivalents or analogs of erythropoietin, required to achieve maximum colony number in said assay.

5. The Erythropoietin Receptor Extended Duration Limited Agonist of claim 1, wherein in (a) the Erythropoietin Receptor Extended Duration Limited Agonist elicits about 15 to 50% of the maximum colony number in a Burst Forming Unit-Erythroid assay in relation to the maximum colony number achieved by erythropoietin, or recombinant equivalents or analogs of erythropoietin, in said assay.

6. The Erythropoietin Receptor Extended Duration Limited Agonist of claim 1, wherein the colonies elicited in a Burst Forming Unit-Erythroid assay by the Erythropoietin Receptor Extended Duration Limited Agonist are at least 25% smaller in diameter than the colonies achieved by erythropoietin, or recombinant equivalents or analogs of erythropoietin, in said assay.

7. The Erythropoietin Receptor Extended Duration Limited Agonist of claim 1, wherein in (b) the Kd is greater than 0.25 nM.

8. The Erythropoietin Receptor Extended Duration Limited Agonist of claim 1, wherein in (b) the Kd is from about 1.1 nM to 14,900 nM.

9. The Erythropoietin Receptor Extended Duration Limited Agonist of claim 1, wherein in (c) the Erythropoietin Receptor Extended Duration Limited Agonist maintains in vivo hemoglobin concentrations above baseline at least about 200 to 300% longer than erythropoietin, or recombinant equivalents or analogs of erythropoietin.

10. The Erythropoietin Receptor Extended Duration Limited Agonist of claim 1, wherein in (c) the Erythropoietin Receptor Extended Duration Limited Agonist maintains in vivo hemoglobin concentrations above baseline about 120 days +/−20 days.

11. The Erythropoietin Receptor Extended Duration Limited Agonist of claim 1, wherein in (c) the Erythropoietin Receptor Extended Duration Limited Agonist maintains in vivo hemoglobin concentrations above baseline for about two to four months.

12. The Erythropoietin Receptor Extended Duration Limited Agonist of claim 1, wherein in (d) the Erythropoietin Receptor Extended Duration Limited Agonist has an in vivo half-life that is about 13 to 80 times longer than erythropoietin, or recombinant equivalents or analogs of erythropoietin.

13. A composition, comprising the Erythropoietin Receptor Extended Duration Limited Agonist of claim 1 and at least one pharmaceutically acceptable vehicle, buffer, excipient, or carrier.

14. A method of activating endogenous activity of an erythropoietin receptor in a patient in need thereof, comprising administering an effective amount of the Erythropoietin Receptor Extended Duration Limited Agonist of claim 1.

15. A method of treating anemia in a patient in need thereof, comprising administering the Erythropoietin Receptor Extended Duration Limited Agonist of claim 1.

16. The method of claim 15, wherein the anemia is associated with a chronic disease or condition.

17. The method of claim 16, wherein the chronic disease or condition is chronic kidney disease, congestive heart failure, or myelodysplastic syndrome.

18. The method of claim 16, wherein the anemia is associated with cancer.

19. The method of claim 18, wherein the anemia associated with cancer is chemotherapy-induced anemia or cancer-induced anemia.

20. The method of claim 15, wherein the anemia is anemia of the elderly, anemia due to infection, anemia associated with inflammation, anemia associated with iron deficiency, anemia associated with blood loss, anemia associated with hemolysis, anemia associated with secondary hyperparathyroidism, anemia associated with inadequate dialysis, anemia associated with protein energy malnutrition, anemia associated with vitamin deficiencies, or anemia associated with metal toxicity.

21. A method of treating pure red blood cell aplasia in a patient in need thereof, comprising administering an effective amount of the Erythropoietin Receptor Extended Duration Limited Agonist of claim 1.

22. A method of promoting tissue protection in erythropoietin-responsive cells, tissues, and organs in a patient in need thereof, comprising administering the Erythropoietin Receptor Extended Duration Limited Agonist of claim 1.

23. The method of claim 14, wherein the Erythropoietin Receptor Extended Duration Limited Agonist of claim 1 is administered to said patient less frequently than epoietin alfa, epoietin beta, darbepoietin alfa, or derivatives thereof.

24. The method of claim 23, wherein said Erythropoietin Receptor Extended Duration Limited Agonist of claim 1 is administered to said patient as needed according to the schedule of: once per month, once every two months, once every three months, or once every four months, once every five months, or once every six months.

25. The method of claim 15, wherein the Erythropoietin Receptor Extended Duration Limited Agonist of claim 1 is administered to said patient less frequently than epoietin alfa, epoietin beta, darbepoietin alfa, or derivatives thereof.

26. The method of claim 25, wherein said Erythropoietin Receptor Extended Duration Limited Agonist of claim 1 is administered to said patient as needed according to the schedule of: once per month, once every two months, once every three months, or once every four months, once every five months, or once every six months.

27. The method of claim 16, wherein the Erythropoietin Receptor Extended Duration Limited Agonist of claim 1 is administered to said patient less frequently than epoietin alfa, epoietin beta, darbepoietin alfa, or derivatives thereof.

28. The method of claim 27, wherein said Erythropoietin Receptor Extended Duration Limited Agonist of claim 1 is administered to said patient as needed according to the schedule of: once per month, once every two months, once every three months, or once every four months, once every five months, or once every six months.

29. The method of claim 17, wherein the Erythropoietin Receptor Extended Duration Limited Agonist of claim 1 is administered to said patient less frequently than epoietin alfa, epoietin beta, darbepoietin alfa, or derivatives thereof.

30. The method of claim 29, wherein said Erythropoietin Receptor Extended Duration Limited Agonist of claim 1 is administered to said patient as needed according to the schedule of: once per month, once every two months, once every three months, or once every four months, once every five months, or once every six months.

31. The method of claim 18, wherein the Erythropoietin Receptor Extended Duration Limited Agonist of claim 1 is administered to said patient less frequently than epoietin alfa, epoietin beta, darbepoietin alfa, or derivatives thereof.

32. The method of claim 31, wherein said Erythropoietin Receptor Extended Duration Limited Agonist of claim 1 is administered to said patient as needed according to the schedule of: once per month, once every two months, once every three months, or once every four months, once every five months, or once every six months.

33. The method of claim 19, wherein the Erythropoietin Receptor Extended Duration Limited Agonist of claim 1 is administered to said patient less frequently than epoietin alfa, epoietin beta, darbepoietin alfa, or derivatives thereof.

34. The method of claim 33, wherein said Erythropoietin Receptor Extended Duration Limited Agonist of claim 1 is administered to said patient as needed according to the schedule of: once per month, once every two months, once every three months, or once every four months, once every five months, or once every six months.

35. The method of claim 20, wherein the Erythropoietin Receptor Extended Duration Limited Agonist of claim 1 is administered to said patient less frequently than epoietin alfa, epoietin beta, darbepoietin alfa, or derivatives thereof.

36. The method of claim 35, wherein said Erythropoietin Receptor Extended Duration Limited Agonist of claim 1 is administered to said patient as needed according to the schedule of: once per month, once every two months, once every three months, or once every four months, once every five months, or once every six months.

37. The method of claim 21, wherein the Erythropoietin Receptor Extended Duration Limited Agonist of claim 1 is administered to said patient less frequently than epoietin alfa, epoietin beta, darbepoietin alfa, or derivatives thereof.

38. The method of claim 37, wherein said Erythropoietin Receptor Extended Duration Limited Agonist of claim 1 is administered to said patient as needed according to the schedule of: once per month, once every two months, once every three months, or once every four months, once every five months, or once every six months.

39. The method of claim 22, wherein the Erythropoietin Receptor Extended Duration Limited Agonist of claim 1 is administered to said patient less frequently than epoietin alfa, epoietin beta, darbepoietin alfa, or derivatives thereof.

40. The method of claim 39, wherein said Erythropoietin Receptor Extended Duration Limited Agonist of claim 1 is administered to said patient as needed according to the schedule of: once per month, once every two months, once every three months, or once every four months, once every five months, or once every six months.


SEQUENCE LISTING (267 sequences)

SEQ ID NO: 1 - protein, 118 aa, Artificial Sequence
  Description of Artificial Sequence Synthetic protein sequence
EVQLVQSGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSS

SEQ ID NO: 2 - protein, 109 aa, Artificial Sequence
  Description of Artificial Sequence Synthetic protein sequence
QSVLTQPPSASGSPGQSVTISCTGTSSDVGGYNYVSWYQQHPGKAPKLMIYEVSKRPSGVPDRFSGSKSGNTASLTVSGLQPEDEADYYCSSYAGRNWVFGGGTQLTVL

SEQ ID NO: 3 - protein, 118 aa, Artificial Sequence
  Description of Artificial Sequence Synthetic protein sequence
EVQLVESGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSS

SEQ ID NO: 4 - protein, 110 aa, Artificial Sequence
  Description of Artificial Sequence Synthetic protein sequence
QSALTQPASVSGSPGQSITISCTGTSSDVGGYIYVSWYQQHPGKAPKLMIYDVSRRPSGISDRFSGSKSGNTASLTISGLQAEDEADYYCNSYTTLSTWLFGGGTKVTVL

SEQ ID NO: 5 - protein, 118 aa, Artificial Sequence
  Description of Artificial Sequence Synthetic protein sequence
EVQLVQSGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGKGTLVTVSS

SEQ ID NO: 6 - protein, 110 aa, Artificial Sequence
  Description of Artificial Sequence Synthetic protein sequence
QSALTQPASVSGSPGQSIIISCTGTRSDIGGYNYVSWYQHHPGRAPKLIIFDVNNRPSGVSHRFSGSKSGNTASLTISGLQAEDEADYYCNSFTDSRTWLFGGGTKLTVL

SEQ ID NO: 7 - protein, 124 aa, Artificial Sequence
  Description of Artificial Sequence Synthetic protein sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSS

SEQ ID NO: 8 - protein, 110 aa, Artificial Sequence
  Description of Artificial Sequence Synthetic protein sequence
QSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 9 - protein, 119 aa, Artificial Sequence
  Description of Artificial Sequence Synthetic protein sequence
QVQLQESGPGLVKPSQTLSLTCAISGDSVSSNSAAWNWIRQSPSRGLEWLGRTYYRSKWYNDYAVSVKSRMTIKADTSKNQFSLQLNSVTPEDTAVYYCARDEGPLDYWGQGTLVTVSA

SEQ ID NO: 10 - protein, 111 aa, Artificial Sequence
  Description of Artificial Sequence Synthetic protein sequence
QAVLTQPSSVSGAPGQRVTISCTGSSSNLGTGYDVHWYQQLPGTAPKLLIYGNSNRPSGVPDRFSGSKSDTSGLLAITGLQAEDEATYYCQSYDFSLSAMVFGGGTKVTVL

SEQ ID NO: 11 - protein, 5 aa, Artificial Sequence
  Description of Artificial Sequence Synthetic peptide
SYWMS

SEQ ID NO: 12 - protein, 17 aa, Artificial Sequence
  Description of Artificial Sequence Synthetic peptide
NIKPDGSEKYYVDSVKG

SEQ ID NO: 13 - protein, 9 aa, Artificial Sequence
  Description of Artificial Sequence Synthetic peptide
VSRGGSYSD

SEQ ID NO: 14 - protein, 14 aa, Artificial Sequence
  Description of Artificial Sequence Synthetic peptide
TGTSSDVGGYNYVS

SEQ ID NO: 15 - protein, 7 aa, Artificial Sequence
  Description of Artificial Sequence Synthetic peptide
EVSKRPS

SEQ ID NO: 16 - protein, 9 aa, Artificial Sequence
  Description of Artificial Sequence Synthetic peptide
SSYAGRNWV

SEQ ID NO: 17 - protein, 14 aa, Artificial Sequence
  Description of Artificial Sequence Synthetic peptide
TGTSSDVGGYIYVS

SEQ ID NO: 18 - protein, 7 aa, Artificial Sequence
  Description of Artificial Sequence Synthetic peptide
DVSRRPS

SEQ ID NO: 19 - protein, 10 aa, Artificial Sequence
  Description of Artificial Sequence Synthetic peptide
NSYTTLSTWL

SEQ ID NO: 20 - protein, 14 aa, Artificial Sequence
  Description of Artificial Sequence Synthetic peptide
TGTRSDIGGYNYVS

SEQ ID NO: 21 - protein, 8 aa, Artificial Sequence
  Description of Artificial Sequence Synthetic peptide
FDVNNRPS

SEQ ID NO: 22 - protein, 10 aa, Artificial Sequence
  Description of Artificial Sequence Synthetic peptide
NSFTDSRTWL

SEQ ID NO: 23 - protein, 5 aa, Artificial Sequence
  Description of Artificial Sequence Synthetic peptide
SYAMS

SEQ ID NO: 24 - protein, 17 aa, Artificial Sequence
  Description of Artificial Sequence Synthetic peptide
AISGSGGSTYYADSVKG

SEQ ID NO: 25 - protein, 15 aa, Artificial Sequence
  Description of Artificial Sequence Synthetic peptide
DRVAVAGKGSYYFDS

SEQ ID NO: 26 - protein, 13 aa, Artificial Sequence
  Description of Artificial Sequence Synthetic peptide
SGSSSNIGNNAVS

SEQ ID NO: 27 - protein, 8 aa, Artificial Sequence
  Description of Artificial Sequence Synthetic peptide
YDNLLPSG

SEQ ID NO: 28 - protein, 11 aa, Artificial Sequence
  Description of Artificial Sequence Synthetic peptide
AAWDDSLNDWV

SEQ ID NO: 29 - protein, 7 aa, Artificial Sequence
  Description of Artificial Sequence Synthetic peptide
SNSAAWN

SEQ ID NO: 30 - protein, 17 aa, Artificial Sequence
  Description of Artificial Sequence Synthetic peptide
RTYYRSKWYNDYAVSKS

SEQ ID NO: 31 - protein, 7 aa, Artificial Sequence
  Description of Artificial Sequence Synthetic peptide
DEGPLDY

SEQ ID NO: 32 - protein, 14 aa, Artificial Sequence
  Description of Artificial Sequence Synthetic peptide
TGSSSNLGTGYDVH

SEQ ID NO: 33 - protein, 7 aa, Artificial Sequence
  Description of Artificial Sequence Synthetic peptide
GNSNRPS

SEQ ID NO: 34 - protein, 11 aa, Artificial Sequence
  Description of Artificial Sequence Synthetic peptide
QSYDFSLSAMV

SEQ ID NO: 35 - DNA, 354 bp, Artificial Sequence
  Description of Artificial Sequence Synthetic polynucleotide sequence
gaggtccagctggtgcagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcgagagtttcgaggggtgggagctactcggactggggccaaggcaccctggtcaccgtctcgagt

SEQ ID NO: 36 - DNA, 327 bp, Artificial Sequence
  Description of Artificial Sequence Synthetic polynucleotide sequence
cagtctgtgctgactcagccaccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccagcctgaggatgaggctgattattactgcagctcatatgcaggcaggaactgggtgttcggcggagggacccagctcaccgtttta

SEQ ID NO: 37 - DNA, 354 bp, Artificial Sequence
  Description of Artificial Sequence Synthetic polynucleotide sequence
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcaagagtttcgaggggtgggagctactcggactggggccagggaaccctggtcaccgtctcgagt

SEQ ID NO: 38 - DNA, 330 bp, Artificial Sequence
  Description of Artificial Sequence Synthetic polynucleotide sequence
cagtctgccctgactcagcctgcctccgtgtctgggtctcctggacagtcgatcaccatctcctgcactggaaccagcagtgacgttggtggctatatttatgtctcctggtaccaacaacacccaggcaaagcccccaaactcatgatttatgatgtcagtcgtcggccctcagggatttctgatcgcttctctggctccaagtctggcaacacggcctccctgaccatctctgggctccaggctgaggacgaggctgattattactgcaactcatatacaaccctcagcacctggctcttcggcggagggaccaaggtcaccgtccta

SEQ ID NO: 39 - DNA, 354 bp, Artificial Sequence
  Description of Artificial Sequence Synthetic polynucleotide sequence
gaggtgcagctggtgcagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcgagagtttcgaggggtgggagctactcggactggggcaaaggaaccctggtcaccgtctcgagt

SEQ ID NO: 40 - DNA, 330 bp, Artificial Sequence
  Description of Artificial Sequence Synthetic polynucleotide sequence
cagtctgccctgactcagcctgcctccgtgtctgggtctcctggacagtcgatcatcatctcctgcactggaacccgcagtgacattggtggttacaactatgtctcctggtaccaacaccacccaggcagagcccccaaactcatcatttttgatgtcaataatcggccctcaggagtctctcaccgcttctctggctccaagtctggcaacacggcctccctgaccatctctgggctccaggctgaggacgaggctgattattactgcaattcatttacagacagccggacttggctgttcggcggagggaccaagctgaccgtccta

SEQ ID NO: 41 - DNA, 372 bp, Artificial Sequence
  Description of Artificial Sequence Synthetic polynucleotide sequence
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgtaaaagatagggttgctgtagctggtaagggttcgtattactttgactcttgggggagggggaccacggtcaccgtctcgagt

SEQ ID NO: 42 - DNA, 330 bp, Artificial Sequence
  Description of Artificial Sequence Synthetic polynucleotide sequence
cagtctgtgctgacgcagccgccctcggtgtctgaagcccccgggcagagggtcaccatcgcctgttctggaagcagctccaacatcggaaataatgctgtaagttggtaccagcaactcccaggaaaggctcccacactcctcatctattatgataatctgctgccctcaggggtctctgaccgattctctggctccaagtctggcacctcagcctccctggccatcagtgggctccagtctgaggatgaggctgattattactgtgctgcatgggatgacagcctgaatgattgggtgttcggcggtgggaccaaggtcaccgtccta

SEQ ID NO: 43 - DNA, 357 bp, Artificial Sequence
  Description of Artificial Sequence Synthetic polynucleotide sequence
caggtgcagctgcaggagtcgggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaatgaccataaaagcagacacatccaagaaccagttctccctgcaactgaactctgtgactcccgaagacacggctgtgtattactgtgcaagagatgagggaccgcttgactactggggccagggaaccctggtcaccgtctcggcc

SEQ ID NO: 44 - DNA, 333 bp, Artificial Sequence
  Description of Artificial Sequence Synthetic polynucleotide sequence
caggctgtgctcactcagccgtcctcagtgtctggggccccagggcagagggtcaccatctcctgcactgggagcagctccaacctcgggacaggttatgatgtacactggtaccagcagcttccaggaacagcccccaaactcctcatctatggtaacagcaatcggccctcaggggtccctgaccgattctcgggctccaagtctgacacctcaggtttgctggccatcactgggctccaggctgaggatgaggctacttattactgccagtcctatgacttcagcctgagtgctatggtattcggcggagggaccaaggtcaccgtccta

SEQ ID NO: 45 - protein, 479 aa, Artificial Sequence
  Description of Artificial Sequence Synthetic protein sequence
EVQLVQSGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSSGGGGSGGGGSGGGGSAQSVLTQPPSASGSPGQSVTISCTGTSSDVGGYNYVSWYQQHPGKAPKLMIYEVSKRPSGVPDRFSGSKSGNTASLTVSGLQPEDEADYYCSSYAGRNWVFGGGTQLTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 480 aa, Artificial Sequence
  Description of Artificial Sequence Synthetic protein sequence
EVQLVESGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSSGGGGSGGGGSGGGGSAQSALTQPASVSGSPGQSITISCTGTSSDVGGYIYVSWYQQHPGKAPKLMIYDVSRRPSGISDRFSGSKSGNTASLTISGLQAEDEADYYCNSYTTLSTWLFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 480 aa, Artificial Sequence
  Description of Artificial Sequence Synthetic protein sequence
EVQLVQSGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGKGTLVTVSSGGGGSGGGGSGGGGSAQSALTQPASVSGSPGQSIIISCTGTRSDIGGYNYVSWYQHHPGRAPKLIIFDVNNRPSGVSHRFSGSKSGNTASLTISGLQAEDEADYYCNSFTDSRTWLFGGGTKLTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 486 aa, Artificial Sequence
  Description of Artificial Sequence Synthetic protein sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSAQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 483 aa, Artificial Sequence
  Description of Artificial Sequence Synthetic protein sequence
QVQLQESGPGLVKPSQTLSLTCAISGDSVSSNSAAWNWIRQSPSRGLEWLGRTYYRSKWYNDYAVSVKSRMTIKADTSKNQFSLQLNSVTPEDTAVYYCARDEGPLDYWGQGTLVTVSAGGGGSGGGGSGGGGSGAPQAVLTQPSSVSGAPGQRVTISCTGSSSNLGTGYDVHWYQQLPGTAPKLLIYGNSNRPSGVPDRFSGSKSDTSGLLAITGLQAEDEATYYCQSYDFSLSAMVFGGGTKVTVLAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1437 bp, Artificial Sequence
  Description of Artificial Sequence Synthetic polynucleotide sequence
gaggtccagctggtgcagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcgagagtttcgaggggtgggagctactcggactggggccaaggcaccctggtcaccgtctcgagtggaggcggcggttcaggcggaggtggctctggcggtggcggaagtgcacagtctgtgctgactcagccaccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccagcctgaggatgaggctgattattactgcagctcatatgcaggcaggaactgggtgttcggcggagggacccagctcaccgttttaggtgcggccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 51 - DNA, 1440 bp, Artificial Sequence
  Description of Artificial Sequence Synthetic polynucleotide sequence
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcaagagtttcgaggggtgggagctactcggactggggccagggaaccctggtcaccgtctcgagtggaggcggcggttcaggcggaggtggctctggcggtggcggaagtgcacagtctgccctgactcagcctgcctccgtgtctgggtctcctggacagtcgatcaccatctcctgcactggaaccagcagtgacgttggtggctatatttatgtctcctggtaccaacaacacccaggcaaagcccccaaactcatgatttatgatgtcagtcgtcggccctcagggatttctgatcgcttctctggctccaagtctggcaacacggcctccctgaccatctctgggctccaggctgaggacgaggctgattattactgcaactcatatacaaccctcagcacctggctcttcggcggagggaccaaggtcaccgtcctaggtgcggccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 52 - DNA, 1440 bp, Artificial Sequence
  Description of Artificial Sequence Synthetic polynucleotide sequence
gaggtgcagctggtgcagtctgggggaggcttggtccagcctggggggtccctgagactctcctgtgcagcctctggattcacctttagtagctattggatgagctgggtccgccaggctccagggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcgagagtttcgaggggtgggagctactcggactggggcaaaggaaccctggtcaccgtctcgagtggaggcggcggttcaggcggaggtggctctggcggtggcggaagtgcacagtctgccctgactcagcctgcctccgtgtctgggtctcctggacagtcgatcatcatctcctgcactggaacccgcagtgacattggtggttacaactatgtctcctggtaccaacaccacccaggcagagcccccaaactcatcatttttgatgtcaataatcggccctcaggagtctctcaccgcttctctggctccaagtctggcaacacggcctccctgaccatctctgggctccaggctgaggacgaggctgattattactgcaattcatttacagacagccggacttggctgttcggcggagggaccaagctgaccgtcctaggtgcggccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 53 - DNA, 1458 bp, Artificial Sequence
  Description of Artificial Sequence Synthetic polynucleotide sequence
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgtaaaagatagggttgctgtagctggtaagggttcgtattactttgactcttgggggagggggaccacggtcaccgtctcgagtggaggcggcggttcaggcggaggtggctctggcggtggcggaagtgcacagtctgtgctgacgcagccgccctcggtgtctgaagcccccgggcagagggtcaccatcgcctgttctggaagcagctccaacatcggaaataatgctgtaagttggtaccagcaactcccaggaaaggctcccacactcctcatctattatgataatctgctgccctcaggggtctctgaccgattctctggctccaagtctggcacctcagcctccctggccatcagtgggctccagtctgaggatgaggctgattattactgtgctgcatgggatgacagcctgaatgattgggtgttcggcggtgggaccaaggtcaccgtcctaggtgcggccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 54 - DNA, 1449 bp, Artificial Sequence
  Description of Artificial Sequence Synthetic polynucleotide sequence
caggtgcagctgcaggagtcgggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaatgaccataaaagcagacacatccaagaaccagttctccctgcaactgaactctgtgactcccgaagacacggctgtgtattactgtgcaagagatgagggaccgcttgactactggggccagggaaccctggtcaccgtctcggccggtggcggtggcagcggcggtggtgggtccggtggcggcggatctggcgcgccacaggctgtgctcactcagccgtcctcagtgtctggggccccagggcagagggtcaccatctcctgcactgggagcagctccaacctcgggacaggttatgatgtacactggtaccagcagcttccaggaacagcccccaaactcctcatctatggtaacagcaatcggccctcaggggtccctgaccgattctcgggctccaagtctgacacctcaggtttgctggccatcactgggctccaggctgaggatgaggctacttattactgccagtcctatgacttcagcctgagtgctatggtattcggcggagggaccaaggtcaccgtcctagcggccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 55 - DNA, 351 bp, Artificial Sequence
  Description of Artificial Sequence Synthetic polynucleotide sequence
caggtacagctgcagcagtcagggggaggcgtggtccagcctgggaggtccctgagactctcctgtgcagcctctggattcaccttcagtgactatgctatgcactgggtccgccaggctccaggcaaggggctagagtgggtggcagttatatcaaatcatggaaagagcacatactacgcagactccgtgaagggccgattcaccatctccagagacaattccaagcacatgctgtatctgcaaatgaacagcctgagagctgacgacacggctctatattactgtgcgagagatatagcattggctggggactactggggccagggcaccctggtcaccgtctctgcc

SEQ ID NO: 56 - protein, 117 aa, Artificial Sequence
  Description of Artificial Sequence Synthetic protein sequence
QVQLQQSGGGVVQPGRSLRLSCAASGFTFSDYAMHWVRQAPGKGLEWVAVISNHGKSTYYADSVKGRFTISRDNSKHMLYLQMNSLRADDTALYYCARDIALAGDYWGQGTLVTVSA

SEQ ID NO: 57 - DNA, 321 bp, Artificial Sequence
  Description of Artificial Sequence Synthetic polynucleotide sequence
gacatccagatgacccagtctccatcctccctgtctgcatctgtaggagacagagtcaccatcacttgccgggcaagtcagagcattagcagctatcttaattggtatcagcaactaccagggaaagtccctaaactcctgatctatggtgcatcgaagttgcaaagtggggtcccctccaggttcagtggcagtggatctgggacagatttcactctcaccatcagcagcctgcagcctgaagattttgcaacttattactgtctccaagattacaattatcctctcactttcggccctgggacacgactggagatcaaa

SEQ ID NO: 58 - protein, 107 aa, Artificial Sequence
  Description of Artificial Sequence Synthetic protein sequence
DIQMTQSPSSLSASVGDRVTITCRASQSISSYLNWYQQLPGKVPKLLIYGASKLQSGVPSRFSGSGSGTDFTLTISSLQPEDFATYYCLQDYNYPLTFGPGTRLEIK

SEQ ID NO: 59 - DNA, 357 bp, Artificial Sequence
  Description of Artificial Sequence Synthetic polynucleotide sequence
caggtgcagctgcaggagtcgggcccaggactggtgaggccttcggggaccctgtccctcacctgcgctgtctctggtggctccatcggcagtagtaactggtggagttgggtccgccaggccccagggaaggggctggagtggattggggaaatctctcagagtgggagcaccaactacaacccgtccctcaagggtcgagtcaccatatcactagacaggtccaggaaccagttgtccctgaagttgagttctgtgaccgccgcggacacggccgtgtattactgtgcgagacagctgcggtcgattgatgcttttgatatctggggcccagggaccacggtcaccgtctcggcc

SEQ ID NO: 60 - protein, 119 aa, Artificial Sequence
  Description of Artificial Sequence Synthetic protein sequence
QVQLQESGPGLVRPSGTLSLTCAVSGGSIGSSNWWSWVRQAPGKGLEWIGEISQSGSTNYNPSLKGRVTISLDRSRNQLSLKLSSVTAADTAVYYCARQLRSIDAFDIWGPGTTVTVSA

SEQ ID NO: 61 - DNA, 321 bp, Artificial Sequence
  Description of Artificial Sequence Synthetic polynucleotide sequence
tcctatgtgctgactcagccaccctcagtgtccgtgtccccaggactgacagccaccatcacctgctctggagataaattgggggacaaatatgcttcctggtatcagcagaagccaggccagtcccctgtgttggtcatctatcaagataggaagcgaccctcagggatccctgagcgattctctgggtccaattctgggaacacagccactctgaccatcagcgggacccaggctgtggatgaggctgactattactgtcaggcgtgggacagcgacacttcttatgtcttcggaactgggacccagctcaccgtttta

SEQ ID NO: 62 - protein, 107 aa, Artificial Sequence
  Description of Artificial Sequence Synthetic protein sequence
SYVLTQPPSVSVSPGLTATITCSGDKLGDKYASWYQQKPGQSPVLVIYQDRKRPSGIPERFSGSNSGNTATLTISGTQAVDEADYYCQAWDSDTSYVFGTGTQLTVL

SEQ ID NO: 63 - DNA, 378 bp, Artificial Sequence
  Description of Artificial Sequence Synthetic polynucleotide sequence
caggtgcagctgcaggagtcgggcccaggactggtgaagccttcggagaccctgtccctcacctgcactgtctctggtggctacatcaataattactactggagctggatccggcagcccccagggaagggcctggagtggattgggtacatccattacagtgggagcacctactacaacccgtccctcaagagtcgagtcaccatatcagaagacacgtccaagaaccagttctccctgaagctgagctctgcgaccgctgcggacacggccgtgtattactgtgcgagagttgggtattactatgatagtagtggttataatcttgcctggtacttcgatctctggggccgtggaaccctggtcaccgtctcggcc

SEQ ID NO: 64 - protein, 126 aa, Artificial Sequence
  Description of Artificial Sequence Synthetic protein sequence
QVQLQESGPGLVKPSETLSLTCTVSGGYINNYYWSWIRQPPGKGLEWIGYIHYSGSTYYNPSLKSRVTISEDTSKNQFSLKLSSATAADTAVYYCARVGYYYDSSGYNLAWYFDLWGRGTLVTVSA

SEQ ID NO: 65 - DNA, 327 bp, Artificial Sequence
  Description of Artificial Sequence Synthetic polynucleotide sequence
tcttctgagctgactcaggaccctgctgtgtctgtggccttgggacagacggtcaggatcacatgccagggagacaacctcagaagttattctgcaacttggtaccaacagaagccaggacaggcccctgtccttgtcctctttggtgaaaacaaccggccctcagggatcccagaccgattctctggctccaagtcaggggacacagctgtcttgaccatcactgggactcagacccaagatgaggctgactattattgcacttccagggtcaatagcgggaaccatctgggggtgttcggcccagggacccagctcaccgtttta

SEQ ID NO: 66 - protein, 109 aa, Artificial Sequence
  Description of Artificial Sequence Synthetic protein sequence
SSELTQDPAVSVALGQTVRITCQGDNLRSYSATWYQQKPGQAPVLVLFGENNRPSGIPDRFSGSKSGDTAVLTITGTQTQDEADYYCTSRVNSGNHLGVFGPGTQLTVL

SEQ ID NO: 67 - DNA, 369 bp, Artificial Sequence
  Description of Artificial Sequence Synthetic polynucleotide sequence
gaggtgcagctggtggagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggggggcacatgactacggtgacccgtgatgcttttgatatctggggccaagggacaatggtcaccgtctctgcc

SEQ ID NO: 68 - protein, 123 aa, Artificial Sequence
  Description of Artificial Sequence Synthetic protein sequence
EVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGGHMTTVTRDAFDIWGQGTMVTVSA

SEQ ID NO: 69 - DNA, 327 bp, Artificial Sequence
  Description of Artificial Sequence Synthetic polynucleotide sequence
tcttctgagctgactcaggaccctgctgtgtctgtggccttgggacagacaatcaggatcacatgccaaggagacagcctcagatactattatgcaacctggtatcagcagaagccaggacaggcccctatacttgtcatctatggtcagaataatcggccctcaggggtcccagaccgattctctggctccagctcaggaaacacagcttccttgaccatcactggggctcaggcggaagatgaggctgactattactgcggaacatgggatagcagtgtgagtgcctcttgggtgttcggcggagggaccaaggtcaccgtccta

SEQ ID NO: 70 - protein, 109 aa, Artificial Sequence
  Description of Artificial Sequence Synthetic protein sequence
SSELTQDPAVSVALGQTIRITCQGDSLRYYYATWYQQKPGQAPILVIYGQNNRPSGVPDRFSGSSSGNTASLTITGAQAEDEADYYCGTWDSSVSASWVFGGGTKVTVL

SEQ ID NO: 71 - DNA, 354 bp, Artificial Sequence
  Description of Artificial Sequence Synthetic polynucleotide sequence
caggtacagctgcagcagtcaggggctgaggtgaagaagcctggggcctcagtgaaggtctcctgcaaggcttctggatacaccttcagcggctattatatgcactgggtgcgacaggcccctggacaagggcttgagtggatgggatggatcaaccctaacagtggcagcacaaattatgcacagaagtttctgggcagggtcaccatgaccagggacacgtccatcagcacagcctacatggaactgagcagcctgagatctgacgacacggccgtgtattactgtgcgaggggacactccggtgactattttgactactggggccagggaaccctggtcaccgtctcggcc

SEQ ID NO: 72 - protein, 118 aa, Artificial Sequence
  Description of Artificial Sequence Synthetic protein sequence
QVQLQQSGAEVKKPGASVKVSCKASGYTFSGYYMHWVRQAPGQGLEWMGWINPNSGSTNYAQKFLGRVTMTRDTSISTAYMELSSLRSDDTAVYYCARGHSGDYFDYWGQGTLVTVSA

SEQ ID NO: 73 - DNA, 318 bp, Artificial Sequence
  Description of Artificial Sequence Synthetic polynucleotide sequence
gaaattgtgttgacgcagtctccatcctccctgtctgcatctgttggagacagagtcaccatcacttgccgggccagtcagagtgttagcagctggttggcctggtatcaacagagaccagggcaagcccctaaactgctgatctatgctgcacgtttgcgaggtggaggcccttcaaggttcagtggcagcggctctgggacagaattcactctcaccatcagcagtctgcaacctgaagactttgcgacttacttctgtcaacagagttacagtaccccgatcagtttcggcggagggaccaagctggagatcaaa

SEQ ID NO: 74 - protein, 106 aa, Artificial Sequence
  Description of Artificial Sequence Synthetic protein sequence
EIVLTQSPSSLSASVGDRVTITCRASQSVSSWLAWYQQRPGQAPKLLIYAARLRGGGPSRFSGSGSGTEFTLTISSLQPEDFATYFCQQSYSTPISFGGGTKLEIK

SEQ ID NO: 75 - DNA, 363 bp, Artificial Sequence
  Description of Artificial Sequence Synthetic polynucleotide sequence
caggtgcagctgcaggagtcgggctcaggactggcgaggccttcacagaccctgtccctcacctgcgctgtctctggtggctccatcagcagtagtgctttctcctggaattggatccggcagccaccagggaagggcctggagtggattggatacatctatcatactgggatcaccgattataacccgtccctcaagagtcgagtcaccatatcagtggacaggtccaagaaccagttctccctgaacgtgaactctgtgaccgccgcggacacggccgtgtattattgtgccagaggacacggttcggaccccgcctggttcgacccctggggcaagggcaccctggtcaccgtctcgagt

SEQ ID NO: 76 - protein, 121 aa, Artificial Sequence
  Description of Artificial Sequence Synthetic protein sequence
QVQLQESGSGLARPSQTLSLTCAVSGGSISSSAFSWNWIRQPPGKGLEWIGYIYHTGITDYNPSLKSRVTISVDRSKNQFSLNVNSVTAADTAVYYCARGHGSDPAWFDPWGKGTLVTVSS

SEQ ID NO: 77 - DNA, 321 bp, Artificial Sequence
  Description of Artificial Sequence Synthetic polynucleotide sequence
caatctgtgctgactcagccaccctcagtgtccgtgtccccaggacagacagccagcatcacctgctctggagataaattgggggataaatatgcttcctggtatcagcagaggccaggccagtcccctgttctggtcatctatcgagacaccaagcggccctcagggatccctgagcgattctctggctccaactctgggaacacagccactctgaccatcagcgggacccaggctgtggatgaggctgactattactgtcaggcgtgggacagcaccacctccctggttttcggcggagggaccaagctgaccgtccta

SEQ ID NO: 78 - protein, 107 aa, Artificial Sequence
  Description of Artificial Sequence Synthetic protein sequence
QSVLTQPPSVSVSPGQTASITCSGDKLGDKYASWYQQRPGQSPVLVIYRDTKRPSGIPERFSGSNSGNTATLTISGTQAVDEADYYCQAWDSTTSLVFGGGTKLTVL

SEQ ID NO: 79 - DNA, 354 bp, Artificial Sequence
  Description of Artificial Sequence Synthetic polynucleotide sequence
gaggtccagctggtacagtctgggggaggcttggtccagcctggggggtccctgagactctcctgtgcagcctctggattcacctttagtagctattggatgagctgggtccgccaggctcctgggaaggggctggagtgggtggccaacataaagccagatggaagtgagaaatactatgtggactctgtgaagggccgattcaccatctccagagacaacgccaagaattcagtgtatctgcaaatgaacagcctgagagccgaggacacggccgtgtattactgtgcgagagtttcgaggggtgggagctactcggactggggccgagggacaatggtcaccgtctcgagt

SEQ ID NO: 80 - protein, 118 aa, Artificial Sequence
  Description of Artificial Sequence Synthetic protein sequence
EVQLVQSGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGRGTMVTVSS

SEQ ID NO: 81 - DNA, 327 bp, Artificial Sequence
  Description of Artificial Sequence Synthetic polynucleotide sequence
cagtctgtgctgactcagccaccctccgcgtccgggtctcctggacagtcagtcaccatctcctgcactggaaccagcagtgacgttggcggttttaactatgtctcctggtaccaaaagtacccaggcaaagcccccaaactcgtcatttatgaggtcagtaagcggccctcaggggtccctgatcgcttctctggctccaagtccggcaacacggcctccctgaccgtctctgggctccaggctgaggatgaggctgattattactgcagctcatgggcacctggtaaaaacttattcggcggagggaccaagctgaccgtccta

SEQ ID NO: 82 - protein, 109 aa, Artificial Sequence
  Description of Artificial Sequence Synthetic protein sequence
QSVLTQPPSASGSPGQSVTISCTGTSSDVGGFNYVSWYQKYPGKAPKLVIYEVSKRPSGVPDRFSGSKSGNTASLTVSGLQAEDEADYYCSSWAPGKNLFGGGTKLTVL

SEQ ID NO: 83 - DNA, 378 bp, Artificial Sequence
  Description of Artificial Sequence Synthetic polynucleotide sequence
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaggtattagtggtagtggtagtagtgaaggtggcacatactacgcagactccgtgaagggccggttcaccctctccagagacaattccaagaataccctgtatctgcaaatgaacagcctgagagccgaggacacggccttatattactgtgtgaaagatcgccctagtcgatacagctttggttattactttgactactggggccggggaaccctggtcaccgtctcgagt

SEQ ID NO: 84 - protein, 126 aa, Artificial Sequence
  Description of Artificial Sequence Synthetic protein sequence
EVQLVESGGGLVQPGGSLRLSCAASGFTFSSYAMSWVRQAPGKGLEWVSGISGSGSSEGGTYYADSVKGRFTLSRDNSKNTLYLQMNSLRAEDTALYYCVKDRPSRYSFGYYFDYWGRGTLVTVSS

SEQ ID NO: 85 - DNA, 321 bp, Artificial Sequence
  Description of Artificial Sequence Synthetic polynucleotide sequence
ctgcctgtgctgactcagccaccctcagtgtccgtgtccccaggacagacagccagcatcgcctgctctggaaataaattgggggataaatatgtttcctggtatcagcagaagccaggccagtcccctctgctggtcatctatcaagataccaagcggccctcagggatccctgagcgattctctggctccaactcagggaacacagccactctgaccatcagcgggacccaggctatggatgaggctgactattactgtcaggcgtgggacagcagcactgatgtggtattcggcggagggaccaagctgaccgtccta

SEQ ID NO: 86 - protein, 107 aa, Artificial Sequence
  Description of Artificial Sequence Synthetic protein sequence
LPVLTQPPSVSVSPGQTASIACSGNKLGDKYVSWYQQKPGQSPLLVIYQDTKRPSGIPERFSGSNSGNTATLTISGTQAMDEADYYCQAWDSSTDVVFGGGTKLTVL

SEQ ID NO: 87 - DNA, 354 bp, Artificial Sequence
  Description of Artificial Sequence Synthetic polynucleotide sequence
gaggtgcagctggtggagtctgggggaggcttggtccagcctggggggtccctgagactctcctgtgcggtctctgggttcacctttagtaagtattggatgacctgggtccgccaggctccagggaagggactggagtgggtggccaacataaagccagatggaagtgagaaatactatgtggagtctgtgaagggccgattcaccatctccagagacaacgccaagaattcagtgtatctgcaaatgaacagtgtgagagccgaagacacggccgtgtattactgtgcgagagtttcgaggggtgggagcttctcggactggggccaggggacaatggtcaccgtctcgagt

SEQ ID NO: 88 - protein, 118 aa, Artificial Sequence
  Description of Artificial Sequence Synthetic protein sequence
EVQLVESGGGLVQPGGSLRLSCAVSGFTFSKYWMTWVRQAPGKGLEWVANIKPDGSEKYYVESVKGRFTISRDNAKNSVYLQMNSVRAEDTAVYYCARVSRGGSFSDWGQGTMVTVSS

SEQ ID NO: 89 - DNA, 330 bp, Artificial Sequence
  Description of Artificial Sequence Synthetic polynucleotide sequence
cagtctgtgctgactcagccaccctccgcgtccgggtctcctggacagtcagtcaccatctcctgcactggaaccagcagcgacgttggtggttataactatgtctcctggtaccaacaacacccagacaaagcccccagactcatgatttatgacgtcaataagcggccctcaggggtccctgatcgcttctctggctccaagtctggcaacacggcctccctgaccgtctctgggctccaggctgaggatgaggctcattattactgcaactcatatgcaggcagcaacaattgggtgttcggcggagggacccagctcaccgtttta

SEQ ID NO: 90 - protein, 110 aa, Artificial Sequence
  Description of Artificial Sequence Synthetic protein sequence
QSVLTQPPSASGSPGQSVTISCTGTSSDVGGYNYVSWYQQHPDKAPRLMIYDVNKRPSGVPDRFSGSKSGNTASLTVSGLQAEDEAHYYCNSYAGSNNWVFGGGTQLTVL

SEQ ID NO: 91 - DNA, 354 bp, Artificial Sequence
  Description of Artificial Sequence Synthetic polynucleotide sequence
caggtgcagctggtggagtctgggggaggcttggtccagcctggggggtccctgagactctcctgtgcggtctctgggttcacctttagtaagtattggatgacctgggtccgccaggctccagggaagggactggagtgggtggccaacataaagccagatggaagtgagaaatactatgtggagtctgtgaagggccgattcaccatctccagagacaacgccaagaattcagtgtatctgcaaatgaacagtgtgagagccgaagacacggccgtgtattactgtgcgagagtttcgaggggtgggagcttctcggactggggccaaggaaccctggtcaccgtctcgagt

SEQ ID NO: 92 - protein, 118 aa, Artificial Sequence
  Description of Artificial Sequence Synthetic protein sequence
QVQLVESGGGLVQPGGSLRLSCAVSGFTFSKYWMTWVRQAPGKGLEWVANIKPDGSEKYYVESVKGRFTISRDNAKNSVYLQMNSVRAEDTAVYYCARVSRGGSFSDWGQGTLVTVSS

SEQ ID NO: 93 - DNA, 327 bp, Artificial Sequence
  Description of Artificial Sequence Synthetic polynucleotide sequence
cagtctgtgctgactcagccaccctccgcgtccgggtctcctggacagtcagtcaccatctcctgcactggaaccagcagtgacgttggtggttataattatgtctcctggtaccaacaacacccaggcagagcccccaaactcatcatttatgaggtcagtaagcggccctcaggggtccctgatcgcttctctggctccaagtctggcaacacggcctccctgaccgtctctgggctccaggctgacgatgaggctgattattactgcaactcatatgcaggcagcatttatgtcttcgggagtgggaccaaggtcaccgtccta

SEQ ID NO: 94 - protein, 109 aa, Artificial Sequence
  Description of Artificial Sequence Synthetic protein sequence
QSVLTQPPSASGSPGQSVTISCTGTSSDVGGYNYVSWYQQHPGRAPKLIIYEVSKRPSGVPDRFSGSKSGNTASLTVSGLQADDEADYYCNSYAGSIYVFGSGTKVTVL

SEQ ID NO: 95 - DNA, 372 bp, Artificial Sequence
  Description of Artificial Sequence Synthetic polynucleotide sequence
caggtgcagctggtgcaatctggggctgaaattaagaagcctggggcctcagtgaaggtttcctgcaagacatttggatcccccttcagcacgaatgacatacactgggtgcgacaggcccctggacaagggcttgagtggatgggaataatcgacactagtggcgccatgacaaggtacgcacagaagttccagggcagagtcaccgtgaccagggaaacgtccacgagcacagtctacatggagctgagcagcctgaaatctgaagacacggctgtgtactactgtgcgagagagggttgtactaatggtgtatgctatgataatggttttgatatctggggccaaggcaccctggtcaccgtctcgagt

SEQ ID NO: 96 - protein, 124 aa, Artificial Sequence
  Description of Artificial Sequence Synthetic protein sequence
QVQLVQSGAEIKKPGASVKVSCKTFGSPFSTNDIHWVRQAPGQGLEWMGIIDTSGAMTRYAQKFQGRVTVTRETSTSTVYMELSSLKSEDTAVYYCAREGCTNGVCYDNGFDIWGQGTLVTVSS

SEQ ID NO: 97 - DNA, 321 bp, Artificial Sequence
  Description of Artificial Sequence Synthetic polynucleotide sequence
gatatccagatgacccagtctccttccaccctgtctgcatctattggagacagagtcaccatcacctgccgggccagtgagggtatttatcattggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 98 - protein, 107 aa, Artificial Sequence
  Description of Artificial Sequence Synthetic protein sequence
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 99 - DNA, 354 bp, Artificial Sequence
  Description of Artificial Sequence Synthetic polynucleotide sequence
caggtgcagctggtggagtctgggggaggcttggtccagcctggggggtccctgagactctcctgtgcggtctctgggttcacctttagtaagtattggatgacctgggtccgccaggctccagggaagggactggagtgggtggccaacataaagccagatggaagtgagaaatactatgtggagtctgtgaagggccgattcaccatctccagagacaacgccaagaattcagtgtatctgcaaatgaacagtgtgagagccgaagacacggccgtgtattactgtgcgagagtttcgaggggtgggagcttctcggactggggccgggggacaatggtcaccgtctcgagt

SEQ ID NO: 100 - protein, 118 aa, Artificial Sequence
  Description of Artificial Sequence Synthetic protein sequence
QVQLVESGGGLVQPGGSLRLSCAVSGFTFSKYWMTWVRQAPGKGLEWVANIKPDGSEKYYVESVKGRFTISRDNAKNSVYLQMNSVRAEDTAVYYCARVSRGGSFSDWGRGTMVTVSS

SEQ ID NO: 101 - DNA, 330 bp, Artificial Sequence
  Description of Artificial Sequence Synthetic polynucleotide sequence
caatctgccctgactcagcctgcctccgtgtctgggtctcctggacagtcgatcaccatctcctgcactggaaccagcagtgatgttgggagttataaccttgtctcctggtaccaacaacacccaggcaaagtccccaaactcatcatttatgaggtcagtaatcggccctcaggggtttctcatcgcttctctggctccaagtctggcaacacggcctccctgaccatctctggactccaggctgaggacgaggctgattattactgcagctcattgacaagcagcggcacttgggtgttcggcggagggaccaaggtcaccgtccta

SEQ ID NO: 102 - protein, 110 aa, Artificial Sequence
  Description of Artificial Sequence Synthetic protein sequence
QSALTQPASVSGSPGQSITISCTGTSSDVGSYNLVSWYQQHPGKVPKLIIYEVSNRPSGVSHRFSGSKSGNTASLTISGLQAEDEADYYCSSLTSSGTWVFGGGTKVTVL

SEQ ID NO: 103 - DNA, 354 bp, Artificial Sequence
  Description of Artificial Sequence Synthetic polynucleotide sequence
gaggtgcagctggtggagtccgggggaggcttggtccagcccggggggtccctgagactctcctgtgcggtctctgggttcacctttagtaagtattggatgacctgggtccgccaggctccagggaagggactggagtgggtggccaacataaagccagatggaagtgagaaatactatgtggagtctgtgaagggccgattcaccatctccagagacaacgccaagaattcagtgtatctgcaaatgaacagtgtgagagccgaagacacggccgtgtattactgtgcgagagtttcgaggggtgggagcttctcggactggggccagggcaccctggtcaccgtctcgagt

SEQ ID NO: 104 - protein, 118 aa, Artificial Sequence
  Description of Artificial Sequence Synthetic protein sequence
EVQLVESGGGLVQPGGSLRLSCAVSGFTFSKYWMTWVRQAPGKGLEWVANIKPDGSEKYYVESVKGRFTISRDNAKNSVYLQMNSVRAEDTAVYYCARVSRGGSFSDWGQGTLVTVSS

SEQ ID NO: 105 - DNA, 333 bp, Artificial Sequence
  Description of Artificial Sequence Synthetic polynucleotide sequence
cagtctgccctgactcagcctccctccgcgtccgggtctcctgggcagtcagtcaccatctcctgcactggaaccagcagtgacgttggtgcttataactatgtctcctggtaccaacagcacccaggcaaagcccccaaactcatgatttatgaggtcgctaggcggccctcaggggtccctgatcgcttctctggctctaagtctggcaacacggcctccctgaccgtctctgggctccaggctgaggatgaggctgattattattgcagctcatatgcaggcagcaacaatttcgcggtcttcggcagagggaccaagctgaccgtccta

SEQ ID NO: 106 - protein, 111 aa, Artificial Sequence
  Description of Artificial Sequence Synthetic protein sequence
QSALTQPPSASGSPGQSVTISCTGTSSDVGAYNYVSWYQQHPGKAPKLMIYEVARRPSGVPDRFSGSKSGNTASLTVSGLQAEDEADYYCSSYAGSNNFAVFGRGTKLTVL

SEQ ID NO: 107 - DNA, 354 bp, Artificial Sequence
  Description of Artificial Sequence Synthetic polynucleotide sequence
gaggtgcagctggtgcagtctgggggaggcttggtccagccgggggggtccctgagactctcctgtgcagcctctggattcaggtttagtagctattggatgacctgggtccgccaggctccagggaaggggctggagtgggtggccaacataaagccagatggaagtgagaaatactatgtggactctgtgaagggccgattcaccatgtccagagacaacgccaagaattcagtgtatctgcaaatgaacagcctgagagccgaggacacggccgtgtattactgtgcgagagtttcgaggggtgggagcttctcggactggggccaaggaaccctggtcaccgtctcgagt

SEQ ID NO: 108 - protein, 118 aa, Artificial Sequence
  Description of Artificial Sequence Synthetic protein sequence
EVQLVQSGGGLVQPGGSLRLSCAASGFRFSSYWMTWVRQAPGKGLEWVANIKPDGSEKYYVDSVKGRFTMSRDNAKNSVYLQMNSLRAEDTAVYYCARVSRGGSFSDWGQGTLVTVSS

SEQ ID NO: 109 - DNA, 330 bp, Artificial Sequence
  Description of Artificial Sequence Synthetic polynucleotide sequence
cagtctgccctgactcagcctgcctccgtgtctgggtctcctggacagtcgatcaccatcccctgcactggaaccagcagtgacattggtacttatgactatgtctcctggtaccaacaacacccaggcaaagtccccaaagtcattatttatgaggtcaccaatcggccctcaggggtttctaatcgcttctctggctccaagtctggcaacacggcctccctgaccatctctgggctccaggctgacgacgaggctgattattactgcaactcatttacaaagaacaacacttgggtgttcggcggagggaccaagctgaccgtccta

SEQ ID NO: 110 - protein, 110 aa, Artificial Sequence
  Description of Artificial Sequence Synthetic protein sequence
QSALTQPASVSGSPGQSITIPCTGTSSDIGTYDYVSWYQQHPGKVPKVIIYEVTNRPSGVSNRFSGSKSGNTASLTISGLQADDEADYYCNSFTKNNTWVFGGGTKLTVL

SEQ ID NO: 111 - DNA, 354 bp, Artificial Sequence
  Description of Artificial Sequence Synthetic polynucleotide sequence
caggtgcagctggtggagtctgggggaggcttggtccagcctgggaggtccctgatactctcctgtgcggtctctgggttcacctttagtaagtattggatgacctgggtccgccaggctccagggaagggactggagtgggtggccaacataaagccagatggaagtgagaaatactatgtggagtctgtgaagggccgattcaccatctccagagacaacgccaagaattcagtgtatctgcaaatgaacagtgtgagagccgaagacacggccgtgtattactgtgcgagagtttcgaggggtgggagcttctcggactggagccaaggaaccttggtcaccgtctcgagt

SEQ ID NO: 112 - protein, 118 aa, Artificial Sequence
  Description of Artificial Sequence Synthetic protein sequence
QVQLVESGGGLVQPGRSLILSCAVSGFTFSKYWMTWVRQAPGKGLEWVANIKPDGSEKYYVESVKGRFTISRDNAKNSVYLQMNSVRAEDTAVYYCARVSRGGSFSDWSQGTLVTVSS

SEQ ID NO: 113 - DNA, 333 bp, Artificial Sequence
  Description of Artificial Sequence Synthetic polynucleotide sequence
cagtctgccctgactcagcctccctccgcgtccgggtctcctgggcagtcagtcaccatctcctgcactggaaccagcggtgacgttggtgcttataactatgtctcctggtaccaacagtacccaggcaaagcccccaaactcatgatttatgaggtcagtaagaggccctccggggtccctgatcgcttctctggctccaagtctggcaacacggcctccctgaccgtctctgggctccaggctgaggatgaggctgattattactgcaactcatataggggcagcaacggtccttgggtgttcggcggagggaccaaggtcaccgtccta

SEQ ID NO: 114 - protein, 111 aa, Artificial Sequence
  Description of Artificial Sequence Synthetic protein sequence
QSALTQPPSASGSPGQSVTISCTGTSGDVGAYNYVSWYQQYPGKAPKLMIYEVSKRPSGVPDRFSGSKSGNTASLTVSGLQAEDEADYYCNSYRGSNGPWVFGGGTKVTVL

SEQ ID NO: 115 - protein, 502 aa, Artificial Sequence [sequence omitted]

SEQ ID NO: 116 - protein, 508 aa, Artificial Sequence
  Description of Artificial Sequence Synthetic protein sequence
MGSTAILALLLAVLQGVSAHMAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSAQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 506 aa, Artificial Sequence
  Description of Artificial Sequence Synthetic protein sequence
MGSTAILALLLAVLQGVSAHMAQVQLQESGPGLVKPSQTLSLTCAISGDSVSSNSAAWNWIRQSPSRGLEWLGRTYYRSKWYNDYAVSVKSRMTIKADTSKNQFSLQLNSVTPEDTAVYYCARDEGPLDYWGQGTLVTVSAGGGGSGGGGSGGGGSGAPQAVLTQPSSVSGAPGQRVTISCTGSSSNLGTGYDVHWYQQLPGTAPKLLIYGNSNRPSGVPDRFSGSKSDTSGLLAITGLQAEDEATYYCQSYDFSLSAMVFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 227 aa, Artificial Sequence
  Description of Artificial Sequence Synthetic protein sequence
EFTPPCPPCPAPELLGGPSVFLFPPKPKDTLMISRTPEVTCVVVDVSQEDPEVQFNWYVDGVEVHHAQTKPRERQFSSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYFLYSKLIVDKSRWQQGNTFSCSVMHEALHNHYTQKSLSVSPGK

SEQ ID NO: 119 - protein, 502 aa, Artificial Sequence
  Description of Artificial Sequence Synthetic protein sequence
MGSTAILALLLAVLQGVSAHMAEVQLVESGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSSGGGGSGGGGSGGGGSAQSALTQPASVSGSPGQSITISCTGTSSDVGGYIYVSWYQQHPGKAPKLMIYDVSRRPSGISDRFSGSKSGNTASLTISGLQAEDEADYYCNSYTTLSTWLFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 508 aa, Artificial Sequence
  Description of Artificial Sequence Synthetic protein sequence
MGSTAILALLLAVLQGVSAHMAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSAQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGAAAEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 506 aa, Artificial Sequence [sequence omitted]

SEQ ID NO: 122 - protein, 496 aa, Artificial Sequence
  Description of Artificial Sequence Synthetic protein sequence
MGSTAILALLLAVLQGVSAHMAEVQLVESGGGLVQPGGSLRLSCAASGFTFSSYWMSWVRQAPGKGLEWVANIKPDGSEKYYVDSVKGRFTISRDNAKNSVYLQMNSLRAEDTAVYYCARVSRGGSYSDWGQGTLVTVSSGGGGSGGGGSGGGGSAQSALTQPASVSGSPGQSITISCTGTSSDVGGYIYVSWYQQHPGKAPKLMIYDVSRRPSGISDRFSGSKSGNTASLTISGLQAEDEADYYCNSYTTLSTWLFGGGTKVTVLAAAEFTPPCPPCPAPELLGGPSVFLFPPKPKDTLMISRTPEVTCVVVDVSQEDPEVQFNWYVDGVEVHHAQTKPRERQFSSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYFLYSKLIVDKSRWQQGNTFSCSVMHEALHNHYTQKSLSVSPGK

SEQ ID NO: 123 - protein, 5 aa, Artificial Sequence
  Description of Artificial Sequence Synthetic peptide
DYAMH

SEQ ID NO: 124 - protein, 17 aa, Artificial Sequence
  Description of Artificial Sequence Synthetic peptide
VISNHGKSTYYADSVKG

SEQ ID NO: 125 - protein, 8 aa, Artificial Sequence
  Description of Artificial Sequence Synthetic peptide
DIALAGDY

SEQ ID NO: 126 - protein, 11 aa, Artificial Sequence
  Description of Artificial Sequence Synthetic peptide
RASQSISSYLN

SEQ ID NO: 127 - protein, 7 aa, Artificial Sequence
  Description of Artificial Sequence Synthetic peptide
GASKLQS

SEQ ID NO: 128 - protein, 9 aa, Artificial Sequence
  Description of Artificial Sequence Synthetic peptide
LQDYNYPLT

SEQ ID NO: 129 - protein, 6 aa, Artificial Sequence
  Description of Artificial Sequence Synthetic peptide
SSNWWS

SEQ ID NO: 130 - protein, 16 aa, Artificial Sequence
  Description of Artificial Sequence Synthetic peptide
EISQSGSTNYNPSLKG

SEQ ID NO: 131 - protein, 10 aa, Artificial Sequence
  Description of Artificial Sequence Synthetic peptide
QLRSIDAFDI

SEQ ID NO: 132 - protein, 5 aa, Artificial Sequence
  Description of Artificial Sequence Synthetic peptide
DKYAS

SEQ ID NO: 133 - protein, 10 aa, Artificial Sequence
  Description of Artificial Sequence Synthetic peptide
YQDRKRPSGI

SEQ ID NO: 134 - protein, 8 aa, Artificial Sequence
  Description of Artificial Sequence Synthetic peptide
WDSDTSYV

SEQ ID NO: 135 - protein, 5 aa, Artificial Sequence
  Description of Artificial Sequence Synthetic peptide
NYYWS

SEQ ID NO: 136 - protein, 17 aa, Artificial Sequence
  Description of Artificial Sequence Synthetic peptide
YIHYSGSTYYNPSLKSR

SEQ ID NO: 137 - protein, 11 aa, Artificial Sequence
  Description of Artificial Sequence Synthetic peptide
QGDNLRSYSAT

SEQ ID NO: 138 - protein, 7 aa, Artificial Sequence
  Description of Artificial Sequence Synthetic peptide
GENNRPS

SEQ ID NO: 139 - protein, 12 aa, Artificial Sequence
  Description of Artificial Sequence Synthetic peptide
TSRVNSGNHLGV

SEQ ID NO: 140 - protein, 5 aa, Artificial Sequence
  Description of Artificial Sequence Synthetic peptide
GYYMH

SEQ ID NO: 141 - protein, 18 aa, Artificial Sequence
  Description of Artificial Sequence Synthetic peptide
WINPNSGGTNYAQKFQGR

SEQ ID NO: 142 - protein, 14 aa, Artificial Sequence
  Description of Artificial Sequence Synthetic peptide
GGHMTTVTRDAFDI

SEQ ID NO: 143 - protein, 11 aa, Artificial Sequence
  Description of Artificial Sequence Synthetic peptide
QGDSLRYYYAT

SEQ ID NO: 144 - protein, 7 aa, Artificial Sequence
  Description of Artificial Sequence Synthetic peptide
GQNNRPS

SEQ ID NO: 145 - protein, 12 aa, Artificial Sequence
  Description of Artificial Sequence Synthetic peptide
GTWDSSVSASWV

SEQ ID NO: 146 - protein, 5 aa, Artificial Sequence
  Description of Artificial Sequence Synthetic peptide
GYYMH

SEQ ID NO: 147 - protein, 17 aa, Artificial Sequence
  Description of Artificial Sequence Synthetic peptide
WINPNSGSTNYAQKFLG

SEQ ID NO: 148 - protein, 9 aa, Artificial Sequence
  Description of Artificial Sequence Synthetic peptide
GHSGDYFDY

SEQ ID NO: 149 - protein, 11 aa, Artificial Sequence
  Description of Artificial Sequence Synthetic peptide
RASQSVSSWLA

SEQ ID NO: 150 - protein, 6 aa, Artificial Sequence
  Description of Artificial Sequence Synthetic peptide
AARLRG

SEQ ID NO: 151 - protein, 9 aa, Artificial Sequence
  Description of Artificial Sequence Synthetic peptide
QQSYSTPIS

SEQ ID NO: 152 - protein, 7 aa, Artificial Sequence
  Description of Artificial Sequence Synthetic peptide
SSAFSWN

SEQ ID NO: 153 - protein, 16 aa, Artificial Sequence
  Description of Artificial Sequence Synthetic peptide
YIYHTGITDYNPSLKS

SEQ ID NO: 154 - protein, 11 aa, Artificial Sequence
  Description of Artificial Sequence Synthetic peptide
GHGSDPAWFDP

SEQ ID NO: 155 - protein, 11 aa, Artificial Sequence
  Description of Artificial Sequence Synthetic peptide
SGDKLGDKYAS

SEQ ID NO: 156 - protein, 7 aa, Artificial Sequence
  Description of Artificial Sequence Synthetic peptide
RDTKRPS

SEQ ID NO: 157 - protein, 10 aa, Artificial Sequence
  Description of Artificial Sequence Synthetic peptide
QAWDSTTSLV

SEQ ID NO: 158 - protein, 5 aa, Artificial Sequence
  Description of Artificial Sequence Synthetic peptide
SYWMS

SEQ ID NO: 159 - protein, 17 aa, Artificial Sequence
  Description of Artificial Sequence Synthetic peptide
NIKPDGSEKYYVDSVKG

SEQ ID NO: 160 - protein, 9 aa, Artificial Sequence
  Description of Artificial Sequence Synthetic peptide
VSRGGSYSD

SEQ ID NO: 161 - protein, 14 aa, Artificial Sequence
  Description of Artificial Sequence Synthetic peptide
TGTSSDVGGFNYVS

SEQ ID NO: 162 - protein, 7 aa, Artificial Sequence
  Description of Artificial Sequence Synthetic peptide
EVSKRPS

SEQ ID NO: 163 - protein, 9 aa, Artificial Sequence
  Description of Artificial Sequence Synthetic peptide
SSWAPGKNL

SEQ ID NO: 164 - protein, 5 aa, Artificial Sequence
  Description of Artificial Sequence Synthetic peptide
SYAMS

SEQ ID NO: 165 - protein, 20 aa, Artificial Sequence
  Description of Artificial Sequence Synthetic peptide
GISGSGSSEGGTYYADSVKG

SEQ ID NO: 166 - protein, 14 aa, Artificial Sequence
  Description of Artificial Sequence Synthetic peptide
DRPSRYSFGYYFDY

SEQ ID NO: 167 - protein, 11 aa, Artificial Sequence
  Description of Artificial Sequence Synthetic peptide
SGNKLGDKYVS

SEQ ID NO: 168 - protein, 7 aa, Artificial Sequence
  Description of Artificial Sequence Synthetic peptide
QDTKRPS

SEQ ID NO: 169 - protein, 10 aa, Artificial Sequence
  Description of Artificial Sequence Synthetic peptide
QAWDSSTDVV

SEQ ID NO: 170 - protein, 5 aa, Artificial Sequence
  Description of Artificial Sequence Synthetic peptide
KYWMT

SEQ ID NO: 171 - protein, 17 aa, Artificial Sequence
  Description of Artificial Sequence Synthetic peptide
NIKPDGSEKYYVESVKG

SEQ ID NO: 172 - protein, 9 aa, Artificial Sequence
  Description of Artificial Sequence Synthetic peptide
VSRGGSFSD

SEQ ID NO: 173 - protein, 14 aa, Artificial Sequence
  Description of Artificial Sequence Synthetic peptide
TGTSSDVGGYNYVS

SEQ ID NO: 174 - protein, 7 aa, Artificial Sequence
  Description of Artificial Sequence Synthetic peptide
DVNKRPS

SEQ ID NO: 175 - protein, 10 aa, Artificial Sequence
  Description of Artificial Sequence Synthetic peptide
NSYAGSNNWV

SEQ ID NO: 176 - protein, 5 aa, Artificial Sequence
  Description of Artificial Sequence Synthetic peptide
KYWMT

SEQ ID NO: 177 - protein, 17 aa, Artificial Sequence
  Description of Artificial Sequence Synthetic peptide
NIKPDGSEKYYVESVKG

SEQ ID NO: 178 - protein, 9 aa, Artificial Sequence
  Description of Artificial Sequence Synthetic peptide
VSRGGSFSD

SEQ ID NO: 179 - protein, 14 aa, Artificial Sequence
  Description of Artificial Sequence Synthetic peptide
TGTSSDVGGYNYVS

SEQ ID NO: 180 - protein, 7 aa, Artificial Sequence
  Description of Artificial Sequence Synthetic peptide
EVSKRPS

SEQ ID NO: 181 - protein, 9 aa, Artificial Sequence
  Description of Artificial Sequence Synthetic peptide
NSYAGSIYV

SEQ ID NO: 182 - protein, 5 aa, Artificial Sequence
  Description of Artificial Sequence Synthetic peptide
TNDIH

SEQ ID NO: 183 - protein, 17 aa, Artificial Sequence
  Description of Artificial Sequence Synthetic peptide
IIDTSGAMTRYAQKFQG

SEQ ID NO: 184 - protein, 15 aa, Artificial Sequence
  Description of Artificial Sequence Synthetic peptide
EGCTNGVCYDNGFDI

SEQ ID NO: 185 - protein, 11 aa, Artificial Sequence
  Description of Artificial Sequence Synthetic peptide
RASEGIYHWLA

SEQ ID NO: 186 - protein, 7 aa, Artificial Sequence
  Description of Artificial Sequence Synthetic peptide
KASSLAS

SEQ ID NO: 187 - protein, 9 aa, Artificial Sequence
  Description of Artificial Sequence Synthetic peptide
QQYSNYPLT

SEQ ID NO: 188 - protein, 5 aa, Artificial Sequence
  Description of Artificial Sequence Synthetic peptide
KYWMT

SEQ ID NO: 189 - protein, 17 aa, Artificial Sequence
  Description of Artificial Sequence Synthetic peptide
NIKPDGSEKYYVESVKG

SEQ ID NO: 190 - protein, 9 aa, Artificial Sequence
  Description of Artificial Sequence Synthetic peptide
VSRGGSFSD

SEQ ID NO: 191 - protein, 14 aa, Artificial Sequence
  Description of Artificial Sequence Synthetic peptide
TGTSSDVGSYNLVS

SEQ ID NO: 192 - protein, 7 aa, Artificial Sequence
  Description of Artificial Sequence Synthetic peptide
EVSNRPS

SEQ ID NO: 193 - protein, 10 aa, Artificial Sequence
  Description of Artificial Sequence Synthetic peptide
SSLTSSGTWV

SEQ ID NO: 194 - protein, 5 aa, Artificial Sequence
  Description of Artificial Sequence Synthetic peptide
KYWMT

SEQ ID NO: 195 - protein, 17 aa, Artificial Sequence
  Description of Artificial Sequence Synthetic peptide
NIKPDGSEKYYVESVKG

SEQ ID NO: 196 - protein, 9 aa, Artificial Sequence
  Description of Artificial Sequence Synthetic peptide
VSRGGSFSD

SEQ ID NO: 197 - protein, 14 aa, Artificial Sequence
  Description of Artificial Sequence Synthetic peptide
TGTSSDVGAYNYVS

SEQ ID NO: 198 - protein, 7 aa, Artificial Sequence
  Description of Artificial Sequence Synthetic peptide
EVARRPS

SEQ ID NO: 199 - protein, 11 aa, Artificial Sequence
  Description of Artificial Sequence Synthetic peptide
SSYAGSNNFAV

SEQ ID NO: 200 - protein, 5 aa, Artificial Sequence
  Description of Artificial Sequence Synthetic peptide
SYWMT

SEQ ID NO: 201 - protein, 17 aa, Artificial Sequence
  Description of Artificial Sequence Synthetic peptide
NIKPDGSEKYYVDSVKG

SEQ ID NO: 202 - protein, 9 aa, Artificial Sequence
  Description of Artificial Sequence Synthetic peptide
VSRGGSFSD

SEQ ID NO: 203 - protein, 14 aa, Artificial Sequence
  Description of Artificial Sequence Synthetic peptide
TGTSSDIGTYDYVS

SEQ ID NO: 204 - protein, 7 aa, Artificial Sequence
  Description of Artificial Sequence Synthetic peptide
EVTNRPS

SEQ ID NO: 205 - protein, 10 aa, Artificial Sequence
  Description of Artificial Sequence Synthetic peptide
NSFTKNNTWV

SEQ ID NO: 206 - protein, 5 aa, Artificial Sequence
  Description of Artificial Sequence Synthetic peptide
KYWMT

SEQ ID NO: 207 - protein, 17 aa, Artificial Sequence
  Description of Artificial Sequence Synthetic peptide
NIKPDGSEKYYVESVKG

SEQ ID NO: 208 - protein, 9 aa, Artificial Sequence
  Description of Artificial Sequence Synthetic peptide
VSRGGSFSD

SEQ ID NO: 209 - protein, 14 aa, Artificial Sequence
  Description of Artificial Sequence Synthetic peptide
TGTSGDVGAYNYVS

SEQ ID NO: 210 - protein, 7 aa, Artificial Sequence
  Description of Artificial Sequence Synthetic peptide
EVSKRPS

SEQ ID NO: 211 - protein, 11 aa, Artificial Sequence
  Description of Artificial Sequence Synthetic peptide
NSYRGSNGPWV

SEQ ID NO: 212 - protein, 18 aa, Artificial Sequence
  Description of Artificial Sequence Synthetic peptide
VGYYYDSSGYNLAWYFDL

SEQ ID NO: 213 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 214 - protein, 226 aa, Homo sapiens
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 215 - DNA, 33 bp, Artificial Sequence
  Description of Artificial Sequence Synthetic primer
cgggaggagcagtacagcagcacgtaccgtgtg

SEQ ID NO: 216 - DNA, 33 bp, Artificial Sequence
  Description of Artificial Sequence Synthetic primer
cacacggtacgtgctgctgtactgctcctcccg

SEQ ID NO: 217 - DNA, 30 bp, Artificial Sequence
  Description of Artificial Sequence Synthetic primer
gggagaggcagttcagcagcacgtaccgcg

SEQ ID NO: 218 - DNA, 30 bp, Artificial Sequence
  Description of Artificial Sequence Synthetic primer
cgcggtacgtgctgctgaactgcctctccc

SEQ ID NO: 219 - DNA, 33 bp, Artificial Sequence
  Description of Artificial Sequence Synthetic primer
gactgcaagcttgacaccatggggtcaaccgcc

SEQ ID NO: 220 - DNA, 30 bp, Artificial Sequence
  Description of Artificial Sequence Synthetic primer
gcatacggatcctcatttacccggagacag

SEQ ID NO: 221 - DNA, 34 bp, Artificial Sequence
  Description of Artificial Sequence Synthetic primer
catgggggtgtgaactctgcggccgctaggacgg

SEQ ID NO: 222 - DNA, 34 bp, Artificial Sequence
  Description of Artificial Sequence Synthetic primer
ccgtcctagcggccgcagagttcacacccccatg

SEQ ID NO: 223 - DNA, 30 bp, Artificial Sequence
  Description of Artificial Sequence Synthetic primer
gcatcaggatcctcatttacccggagacac

SEQ ID NO: 224 - protein, 16 aa, Artificial Sequence
  Description of Artificial Sequence Synthetic peptide
  MOD_RES (12)..(12): Asp or Glu
NIKPDGSEKYVXSVKG

SEQ ID NO: 225 - protein, 9 aa, Artificial Sequence
  Description of Artificial Sequence Synthetic peptide
  MOD_RES (7)..(7): Phe or Tyr
VSRGGSXSD

SEQ ID NO: 226 - protein, 14 aa, Artificial Sequence
  Description of Artificial Sequence Synthetic peptide
  MOD_RES (7)..(7): Val or Ile
  MOD_RES (9)..(9): Gly, Ala, Thr or Ser
  MOD_RES (11)..(11): Asn, Asp or Ile
TGTSSDXGXYXYVS

SEQ ID NO: 227 - protein, 7 aa, Artificial Sequence
  Description of Artificial Sequence Synthetic peptide
  MOD_RES (1)..(1): Asp or Glu
  MOD_RES (3)..(3): Asn, Ser, Ala or Thr
  MOD_RES (4)..(4): Lys, Asn or Arg
XVXXRPS

SEQ ID NO: 228 - protein, 63 aa, Artificial Sequence
  Description of Artificial Sequence Synthetic polypeptide
NYYWSGGGAAAGGGAAAYIHYSGSTYYNPSLKSGGGAAAGGGAAAVGYYYDSSGYNLAWYFDL

SEQ ID NO: 229 - protein, 57 aa, Artificial Sequence
  Description of Artificial Sequence Synthetic polypeptide
SSAFSWNGGGAAAGGGAAAYIYHTGITDNPSLKSGGGAAAGGGAAAGHGSDPAWFDP

SEQ ID NO: 230 - protein, 55 aa, Artificial Sequence
  Description of Artificial Sequence Synthetic polypeptide
SSNWWSGGGAAAGGGAAAEISQSGSTNNPSLKGGGGAAAGGGAAAQLRSIDAFDI

SEQ ID NO: 231 - protein, 54 aa, Artificial Sequence
  Description of Artificial Sequence Synthetic polypeptide
KYWMTGGGAAAGGGAAANIKPDGSEKYVESVKGGGGAAAGGGAAAVSRGGSFSD

SEQ ID NO: 232 - protein, 54 aa, Artificial Sequence; identical to SEQ ID NO: 231
  Description of Artificial Sequence Synthetic polypeptide

SEQ ID NO: 233 - protein, 54 aa, Artificial Sequence; identical to SEQ ID NO: 231
  Description of Artificial Sequence Synthetic polypeptide

SEQ ID NO: 234 - protein, 54 aa, Artificial Sequence; identical to SEQ ID NO: 231
  Description of Artificial Sequence Synthetic polypeptide

SEQ ID NO: 235 - protein, 54 aa, Artificial Sequence; identical to SEQ ID NO: 231
  Description of Artificial Sequence Synthetic polypeptide

SEQ ID NO: 236 - protein, 54 aa, Artificial Sequence
  Description of Artificial Sequence Synthetic polypeptide
SYWMSGGGAAAGGGAAANIKPDGSEKYVDSVKGGGGAAAGGGAAAVSRGGSYSD

SEQ ID NO: 237 - protein, 54 aa, Artificial Sequence; identical to SEQ ID NO: 236
  Description of Artificial Sequence Synthetic polypeptide

SEQ ID NO: 238 - protein, 54 aa, Artificial Sequence; identical to SEQ ID NO: 236
  Description of Artificial Sequence Synthetic polypeptide

SEQ ID NO: 239 - protein, 54 aa, Artificial Sequence; identical to SEQ ID NO: 236
  Description of Artificial Sequence Synthetic polypeptide

SEQ ID NO: 240 - protein, 54 aa, Artificial Sequence
  Description of Artificial Sequence Synthetic polypeptide
SYWMTGGGAAAGGGAAANIKPDGSEKYVDSVKGGGGAAAGGGAAAVSRGGSFSD

SEQ ID NO: 241 - protein, 53 aa, Artificial Sequence
  Description of Artificial Sequence Synthetic polypeptide
DYAMHGGGAAAGGGAAAVISNHGKSTYADSVKGGGGAAAGGGAAADIALAGDY

SEQ ID NO: 242 - protein, 60 aa, Artificial Sequence
  Description of Artificial Sequence Synthetic polypeptide
SYAMSGGGAAAGGGAAAAISGSGGSTYADSVKGGGGAAAGGGAAADRVAVAGKGSYYFDS

SEQ ID NO: 243 - protein, 62 aa, Artificial Sequence
  Description of Artificial Sequence Synthetic polypeptide
SYAMSGGGAAAGGGAAAGISGSGSSEGGTYADSVKGGGGAAAGGGAAADRPSRYSFGYYFDY

SEQ ID NO: 244 - protein, 59 aa, Artificial Sequence
  Description of Artificial Sequence Synthetic polypeptide
GYYMHGGGAAAGGGAAAWINPNSGGTNAQKFQGGGGAAAGGGAAAGGHMTTVTRDAFDI

SEQ ID NO: 245 - protein, 54 aa, Artificial Sequence
  Description of Artificial Sequence Synthetic polypeptide
GYYMHGGGAAAGGGAAAWINPNSGSTNAQKFLGGGGAAAGGGAAAGHSGDYFDY

SEQ ID NO: 246 - protein, 60 aa, Artificial Sequence
  Description of Artificial Sequence Synthetic polypeptide
TNDIHGGGAAAGGGAAAIIDTSGAMTRAQKFQGGGGAAAGGGAAAEGCTNGVCYDNGFDI

SEQ ID NO: 247 - protein, 55 aa, Artificial Sequence
  Description of Artificial Sequence Synthetic polypeptide
SNSAAWNGGGAAAGGGAAARTYYRSKWYNDAVSVKSGGGAAAGGGAAADEGPLDY

SEQ ID NO: 248 - protein, 51 aa, Artificial Sequence
  Description of Artificial Sequence Synthetic polypeptide
RASQSISSYLNGGGAAAGGGAAAGASKLQSGGGAAAGGGAAALQDYNYPLT

SEQ ID NO: 249 - protein, 50 aa, Artificial Sequence
  Description of Artificial Sequence Synthetic polypeptide
RASQSVSSWLAGGGAAAGGGAAAAARLRGGGGAAAGGGAAAQQSYSTPIS

SEQ ID NO: 250 - protein, 51 aa, Artificial Sequence
  Description of Artificial Sequence Synthetic polypeptide
RASEGIYHWLAGGGAAAGGGAAAKASSLASGGGAAAGGGAAAQQYSNYPLT

SEQ ID NO: 251 - protein, 54 aa, Artificial Sequence
  Description of Artificial Sequence Synthetic polypeptide
QGDNLRSYSATGGGAAAGGGAAAGENNRPSGGGAAAGGGAAATSRVNSGNHLGV

SEQ ID NO: 252 - protein, 54 aa, Artificial Sequence
  Description of Artificial Sequence Synthetic polypeptide
QGDSLRYYYATGGGAAAGGGAAAGQNNRPSGGGAAAGGGAAAGTWDSSVSASWV

SEQ ID NO: 253 - protein, 55 aa, Artificial Sequence
  Description of Artificial Sequence Synthetic polypeptide
TGTSSDVGGYNYVSGGGAAAGGGAAADVNKRPSGGGAAAGGGAAANSYAGSNNWV

SEQ ID NO: 254 - protein, 54 aa, Artificial Sequence
  Description of Artificial Sequence Synthetic polypeptide
TGTSSDVGGYNYVSGGGAAAGGGAAAEVSKRPSGGGAAAGGGAAASSYAGRNWV

SEQ ID NO: 255 - protein, 54 aa, Artificial Sequence
  Description of Artificial Sequence Synthetic polypeptide
TGTSSDVGGYNYVSGGGAAAGGGAAAEVSKRPSGGGAAAGGGAAANSYAGSIYV

SEQ ID NO: 256 - protein, 56 aa, Artificial Sequence
  Description of Artificial Sequence Synthetic polypeptide
TGTSGDVGAYNYVSGGGAAAGGGAAAEVSKRPSGGGAAAGGGAAANSYRGSNGPWV

SEQ ID NO: 257 - protein, 56 aa, Artificial Sequence
  Description of Artificial Sequence Synthetic polypeptide
TGTSSDVGAYNYVSGGGAAAGGGAAAEVARRPSGGGAAAGGGAAASSYAGSNNFAV

SEQ ID NO: 258 - protein, 54 aa, Artificial Sequence
  Description of Artificial Sequence Synthetic polypeptide
TGTSSDVGGFNYVSGGGAAAGGGAAAEVSKRPSGGGAAAGGGAAASSWAPGKNL

SEQ ID NO: 259 - protein, 55 aa, Artificial Sequence
  Description of Artificial Sequence Synthetic polypeptide
TGTSSDIGTYDYVSGGGAAAGGGAAAEVTNRPSGGGAAAGGGAAANSFTKNNTWV

SEQ ID NO: 260 - protein, 55 aa, Artificial Sequence
  Description of Artificial Sequence Synthetic polypeptide
TGTRSDIGGYNYVSGGGAAAGGGAAADVNNRPSGGGAAAGGGAAANSFTDSRTWL

SEQ ID NO: 261 - protein, 55 aa, Artificial Sequence
  Description of Artificial Sequence Synthetic polypeptide
TGTSSDVGGYIYVSGGGAAAGGGAAADVSRRPSGGGAAAGGGAAANSYTTLSTWL

SEQ ID NO: 262 - protein, 55 aa, Artificial Sequence
  Description of Artificial Sequence Synthetic polypeptide
TGTSSDVGSYNLVSGGGAAAGGGAAAEVSNRPSGGGAAAGGGAAASSLTSSGTWV

SEQ ID NO: 263 - protein, 52 aa, Artificial Sequence
  Description of Artificial Sequence Synthetic polypeptide
SGDKLGDKYASGGGAAAGGGAAAQDRKRPSGGGAAAGGGAAAQAWDSDTSYV

SEQ ID NO: 264 - protein, 52 aa, Artificial Sequence
  Description of Artificial Sequence Synthetic polypeptide
SGDKLGDKYASGGGAAAGGGAAARDTKRPSGGGAAAGGGAAAQAWDSTTSLV

SEQ ID NO: 265 - protein, 52 aa, Artificial Sequence
  Description of Artificial Sequence Synthetic polypeptide
SGNKLGDKYVSGGGAAAGGGAAAQDTKRPSGGGAAAGGGAAAQAWDSSTDVV

SEQ ID NO: 266 - protein, 55 aa, Artificial Sequence
  Description of Artificial Sequence Synthetic polypeptide
SGSSSNIGNNAVSGGGAAAGGGAAAYDNLLPSGGGAAAGGGAAAAAWDDSLNDWV

SEQ ID NO: 267 - protein, 56 aa, Artificial Sequence
  Description of Artificial Sequence Synthetic polypeptide
TGSSSNLGTGYDVHGGGAAAGGGAAAGNSNRPSGGGAAAGGGAAAQSYDFSLSAMV